SEQUENCE LISTING (38 sequences)

SEQ ID NO: 1 - DNA, 267966 bp, Homo sapiens
gcaaacaaacgacgtccgtgattggctccgttcgggcttcggctcccagccgaagcgggcgagcgtggggctcggccggcgattcccagacgcctgttacgcgggcggcggggcgctgggcggtgtaaggctgggtgggggaggaaggaggtggaggacgagtaggaggggggaggaggagtggggaagtgcaaggcggctgcgcagacagcgctcctcacacagagcagcycctgacccgggcgaatgcgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagtgagtattggttttattttacaccccttttccacctcgcccttaaaataagaaacctcgaaataccaggatctgaaattattagacctcctgataaacaggcaatggaaaagggcaaaacctatcttcagtcaaggatgaagcagctccctttttactcctcccataccccctcccccggtctcattaaccttgaattgagataatatgatatttatttggatgattcgatttcaaactgctttcttgtacttttcttgctgttggtagtcagtttcatcaagtgctcgagtgtttccacgttaaagtagtgatgacagtgtttacttgcccatcctttagaataaagagaaggacaagatttgattactaaaccattatgcacatttgcagaatttttggaatttggaatgagagaatattttaggtctcaggggttatcatagtttgatatacttaatagagacgttaaatgtcccaccctcccaacatttttttcttgtttgtatgactacacataatatttgttcttaatctgtggatgaacttcctcagtaaccattcacatttggaggctgccattgatttctctctttagaagggactagtgggagccaataaatacctagcagcagtaacatctctgtatgcatttgtgttcaattggataagtggtttgatgtgttatctcttaaccttcctgttttgacattaccaacctgccaccgtataatttatgtttaatcaaaagttaacttggaagtattaataggtccaagttttgattgagatttgttttgtcatagtgccaaatactaagttaaatgttgtcactggctcattacatttaagttcaattttggagtgctactgcattcattacctgttaaatgttctggcgggttccttaaaaaggacgatttaaaacactctagcgttagtttccatcatatttagataactgtggatttagttagaagaatgtggatttagtcctttccattttggcttttgcagatggctatggtcctagaaataatagggatatttgggctgtgttcagggttgtggatagactactcttgtcctgggtttgtgctgaaaagtatgctcttgaagtgtttaatatatactaagtatctaatgttccagtgtatctctatttatggagaagttcatgtggtatttaatgatagcttgtttaaacatacttgcagaatttaacaaaaatatctatattgtttccttattgtgggaaagaggggcatcatgagggcaaagacaaattaatttgttgcctagaagaactgagagaactaaagaactgagattttaagaacccacacacacgcaaaacatgatgttatgtgaaattgattatattcttaggcaacataaaatgatcacttttagttgaactcaagatttcaattcatgttggctttgggaactagcctgaaacagggtagtctaggtaatagctgaatgggagataatttttgaaagttaattttttttttttcttgtggcttgcaagtagagaatgcccgggtaggtaaagaggaaagctgattatgcatctttcagggaagggtcgtgctttctaagcacttcatggagtgatttgaaaaatattttaaaattccggggtaattttcagctatgatgatacaaatcttataatagctacttaagagcctcttgtggtactggagtgtttgtatgttttacatcttggtaatatagtttgaaaatcagtcagtctatttcagctaattgatttattagctttatttcagagatctgtaatgctagattataatgctacattttgggacttttcattttcttttcaggattttctttgaaaatgactcttgaattcttggtaatacagatactaactaaggatggagtaactcttgtaaaatatatagatgaaaatgtttaattccgattaccatataggtcaatttagaattttaaaagttggagtttagtgttaattatgtgtttttgacagctttgactttaaaagcatttaaaagaaaggggagtctttaagcatttgttaggatgctagcattctaaggaacccctgttgaaaacagacataagaaattgtagtccttgctctcaaaagcttatcctttagttgtagagggaggctatggggaaaaaaaaattaaaacaatagcgtgatgactgcaagattgtaaaatacaaattgaatcatttgttcagagtggcaaagagtaaaggggtgagactgatggttgggagaggaaagacttcctgcggacaaggtgtattaggactgatgttaaaggaggtggtaacttgaactggtagaaaagcatgaaaggtctaggtggaaggagtgtaccaaggaacaatgatggtgagttttgcatgagagacaataatcaagttgacaaatctgaattaaaggggcctaaataagaacataagaacagatggaatttgagaaataagagaaagttagctgatggagggcagaggggttctgtacaagtttgattgagattcagaagagatctggatttattgttggttgctgaataagacgtaagatgatttttgcagttgaattagtgaattatagtagagagatgctagagcctttttagggagctagtagggtatgttgtgaatcatgagggaataaagtaaaatgtgaaagtggggtgaaaggaacaggtttgagagacagtgtgaatgaagaactggcagaacttcatactggactgaagtggaatatttgtgaaggagtcaagagttaaaaataaattctgatgttactggcctggaagacttgactgctaagttatattgttaattgtaatgggaaaattgagaaagtgctgaggtgagagggtaggtgagttcactttcttaaaatttaatatacaataagttcccttgaaattcagacagcagttagttactggcaatgatatttgacttcctagtctattaattgcacagtttctaatttttctacacctaaagatgttggatttatattcgggagcagtaaactaagggcttttatttaaatgcttctcatcaaagtcctaattttttttttttttatatacagcttcaaaagcctcacacttgaaacctggttataatagtttcatgatggcttatttcagatcaatgaaaatacttttgggactaaactgtgactgtaacattttagatatgtagccattaatctttgatatatgcttaaaacttgttttttataaataatgattattactattattttaaatgcaaaatttatctaagaataaagtattgagagccttcactttgtgaagactccagaagaaattaaaagttgattttataacaagtattttttgccctttttgttgagtttaggcattaatcccaaatttaaatcagtatatttgaaatactactttttacttgctatagtgaaaccaatgtttcatttttgttcacttccaagtttacatgtaattaaataaatgtttataaagtttttctttagaatcacatgatattttactccatagggaaagtaagggactgtttgtggcacttttggttaaaatattagtaatccttttggtatatacaattggagaaaatttagctagttgtttaggtttaatacttaaatataagatttagaaatatgaaaataagtctgtatcacttaagttgtttcctcatataaaacttttttagatcaaaatttctattagcaaagagagaataatagtagctgcatattattctgcagtattacgtcgtatggtgattaaagccaaagagtaggtagcaacttagagtttccgtattcccctgggaagtttaatctctttatgacatctaaaaattaggttattagagtttagtccatttcttgataatcctgtgttaatgtggaaaagagttcatgtgtgagactgaaaaccagaactttattttcagtcatttcccaacccgacttccccagagttgttaaaacgtttaagatatttaagaattgcatgctttcttgttgcatgcacctgtagaatgctaattgtgaagtgacagtaagtcacaggcaaaaggaaaaacttcaagggaacccagtgattcacaaatcaccttatggctgattgggaactgcatttagaaaatgaggtgacatcttctcatttcactggcaaagttcagctgaaaatcagtacttgcacgctttactgtgtagcccttatattagaattgagttgcaggtgtgagataatattttggagaaacagcaaggctgggaaagtcagagattttattccagcagcagagtctaaagagtgggagagctctatttgtgtgtgcactttatgcatagagaaggctgtgaaacatgcatatgttgaccctattgtcacagttccatacttttaaataagacccatgaagttgagacagggttggagatgacagacaggatgtgagattgtggtgcttcttgtgatgctccaccctataatggagaatggaggggcagaacgcttgtgtgggtgagacaagagtgtgtgttggtactaacaggaccaaccgggttccccggatattgttcttctaagaacataatcaccataagtaaattaatttgttggaatgtttatcattttctagctgattttttgtgtgtgtgtgtggggtggtggtggtggaaggggagttttccccgatttcaagaataaaagcattttcattgttcagaagaagagcattttcattaaagcggaggttgcactcttggcattttatcttgagttatacaaatcatccttggttatacaaatcaaattgcttttgaaatttgttgagtgtaatgtataaatgtatatttatataaaaacctatgtgaatagaacagcaaggaaaaactgagtagagttaaagaagtttgttttccctgagattttctgcctttaaatttatataaaaaaggaatattccattttacgttattgagttttctatttttttaaatgagctaatactcagtacataatggacatttttagtaaattattccaatatacttgtgtctgtaatgcatttgatgtggtataagatttagaaacacaatttagttcggctggcacggtggctcacgcctgtaatcccagcactttgggaggccgaggcgggtggatcacgaggtcaggagatcaagaccatcctggccaacgtggggaaaccccgtctctactaaaaatacaaaaattagcgggggcgtggtggtgtgcgtctgtagtcccagctactcaggaggctgaggcaggagaatcgtttgaacccatgaggcggagattgcagtgagctgagatcgtgccactgcactccagcctgggcgacagagagagactctgtctcaaaaaaaaaacaaaacaaaagacaatttagtcgatttgggttcttagtttataatcccccttaccatttttgcttatattttgggaagagaggtcaacttccaaacaatgtgcaggttccaattttataagtgattaaatcttatattttttgtattgaatagttttcttagcatgtttggtttgatcacttgaagtaatgatgtatttgtcttgaaactagttttgtcaaaccaactttttatttccagagtaccatggttaacaacagcacaaagccaagtccttaacgtgttcccttaataacatggtctaaggttaatttcttgattcttatttcaaatcaattcagtaataaaaagtacactttttttttttttgagatggagtctcgcactgtttcctgggctggagtgccatggcgtgatctgggcttactgcaacctctgccacccaggttcaagcgattttcctgcctcagcctcccaagtagttgggattacaggtgcctgccaccacacctggctaattttttgtatttttagtagagacggagtttcactgtgttggccaggctggtctcaaaatcctgaccttgtgatcttcccacctcggcctcccaaagtgctgggattacaggcgtgaaccaccacatgtggccgaactaaattgtgtttctaatttagttttcctgaagaataacagctatgcatcaatgtggaattgtgttatttggaatctcaccttattggaattggctggtaattgacagtaaaccagaagtgctcctgagcagctaatatctgcagacaaatggagtgtaaactttgctcataggttagttttcaggcttttattaacggttccttgttattatttaacatatagttgtaattcattgagttgtctgggtttttttgaaatcacagatcaggggaatggataggtactgattaagcattatgctactgaaaagaatggatgattaggggaaacaagcatattgggattacttagtgtatgtagggtatgtaggttcatatagttccagggtaaatcattctgataattgttgaaaatgttcacccagagaaatttttttttttttttggagacagggtctcgctgtgttgcctatgcctagcctggagtgcagtggcacagtcttggctcactgcagcctctgcctcctgggctcgggtgatcttttaacctcaacctctggagtaactgggactataggcatgcgccaacatgcctggctagtttttgtagtttttatagagagggggtttcgccatcttgcccaggctggtcttgaactcctggaatcaagcaatcctgcctcagcctcccacagtgctgggattacagtcatgagccactgcacccatctgagaatattaattctcttttaaagaggcaggaggcagaaaaatctcttgtggaaacctgggcttctgcataaactacataaaatatctgtaatttgttgatcactgaagtgatgaagagaactgggtcacctctgtctgattcaccatcttctagttggatttcagcagtatggataaaggtactaaagtctttagtgtgctagcccagggtattgtttccaactctttcttccattgtccatctacatttattaactcagcagcagagttttatctgatttaagatttgttgtcttactatttctgtttttttttttttcctccttagtatatatggaattgctgcttggattttggctggcttgtaccttttgtttgtggactgcttagaaggcactgcatggagtcatttaagctttcttatgaattatgaatagctatgtagagaggaacttttctagatacatttatggcctatgagaattgaaaagctgggtatcttaattgttaggctgaagaaaaagggtgatagtggcaaagtagtttttcttttggaggtctgagaaattcttaggggcttaattggctaatataactagaaaggttgggtgatttaggtttttttccaaggaaaggtatcttatttaaatgcaacactgtctgcttcctgttattttataaaattagttaatacttttttttttttctcaagacggaatgtcactgtgtcgcccaggctggggtgcagtggcacaatcttggctcactgcaaccgccacctcgaggcctggttcaaggaattcccctgcctcagcctcctgagtagctgggattacaggcatatgccaccattccccgctaatttttttgtatttgtagtagagatggggtttcaccatgttggccagactagtcctgaactcctgacctcaggcaatctgctggcctcagcctcgcaaagtacagcaaaatcagttaatatttttaattgattaacactttcttcatgacctgtgttgaagttattgagttgagtcaattgaatttctaatgctagattttgagtatgtggtgtgagcacctgaagagttggaggtactggaattatgtctgacatcagactgatagtatttgcagggacatttgccctagtggaattcagggttttcacattctatacattacttaaattatgaaggaaagggggaagctttgagttggtagacttttaataataagatataattacatgtatatagaagagcaacgtactgtatatcatattctcataatatatggtaataacggcagtaattattgagtgcggtgtgccagataattgcatgttttatttacctattttgctttaaattttaaaaaatgtaatttactcacatggttcaaaattttaaagatattcagtgaagtcttctctcctgctctgcaacaaccagttctctttcctggagaccactggcattacaatttcttatgtgtcctttcataggtattctgaatctttacggattaatgatttctatagtagacttgcattttgtgtatacatacataaattaacaatagctttagcagaagtgttagaattaaaacaaatttaaatcagtaactctagaaaattgctattttttaacctattaaattagtaaatacttatttctctaaatataagttagttggagaataagtgcaaagatgacagagctcctctcgtggcaattagaggtaaaatgttaatagtcttaaccaatgaacaattaatagcacatttaaaatagatgtttatttcataataaaaagacaggataattttttaactttctatttttctaacatcttagatgagaaaacctgttctcttgagagatcagaggcgcagagtgcgtcagtgtcaatgaagcctgtgccttttacttctttaagagcgtacggtttcattctgctcctaaatcaagcaccccttagttgttgaataaaaattacttttataaaggaatatatgatttaaagaaatcaaatagtgcagaagggcttatgatggaaagtaatcttctgtcctccagtcctacttcttagaagcaacattggctatgacagctgtttcctctagtagtttttgtcatatttttaattatatgctttttatctatttcttaaaattaatttttttgtcttattgtggtagacatcatttattgtcatgcccatctcccttttgctttcatactgatataattatgccactatttaaaaactattatttatttaaattattattcaaattattctcagtatgtagacatggaacagtattctatgtagaaacaagtagtatacagtgatcacatttccttatgagaatagctttattgagatataattcacataccgtaacattcattttttcattactcaggtgttcatagacttgtgtaaccatcccaaccatctaattttagagcacttttatcatttcagagataaagcttatacccattagcagccattccctatgttccccttccccacagctctaggcaaccactaatttactctctgtctttatagatttgcctgttctggacatttcatataaatggatttatgtaatacatggccatttatgactgttttctttctcccatgttatagcatgtattagtacttcattcctttttattgctgaataacatttcattgtatggaaatcctgcgatttgtttattagttgatggacatttgggttgtttctgctttttgactagtatgaatatgctgctgtgaacatttgtgtgcaagtttttgtgtggacatatgttttcagatcttttggggtatatacctaagtagtggaatggctgggtgatatggtaactctctgtttaaagtgttaaggagctattaagctgtttcaaagtgacagtcacattttttcctttgcttttaatctttttagactttattgttgccattttctttctttttttttaaatactgtttgtaataattataatctcaattcttccccccaaatgtaccaatttatcagacattctctggatttctacctgcctcctgcccctttcttttcctggagtgtcctttttcctggagccctcgtccaactgctgggatctgatctagttgttctccaggccccgttgtacagctgtcatcctctgacttctttcgctgctcttcagggttggctctccagtttcctggatcccatatctctaaataacatttcattgtatggaaatcccatgatttgtttgttagttgaatagttcttatctgataactgtttgataaatatacctggaaaagttgttcattgtttttttccatatttgttccaaaagggcagttaagaagctatatactttttgggcagataccctcacccttgcagtatctagatactgagaaatagatgttggaaggttactttgtatcattttattaagtgattaccatgcaaacataaggcaagtacaaaaatctgaaaaatattaatattaagtaattgtttagactagagctattcagaagaaatattatataagccaaaaaggtgaattatatatgtaattttaattttctatgaaccactttgaaaaatagacacaagtgaaattaattctagtaatatattttatttaacctagtgtatctaaactaataatatatcaagatgtaatcaatataaaaattattgagatattttacattctttttttgggtagtaaatctttataacctagtgagtgttttacacttaaagaacatactaatttgggggccgggcgcggtggctcatgcctgtaatcccagcacattgggagaccgaggcgggtggatcacgaggtcaggagttcaagaccaacctggccaagatggtgaaaccccgtctctactaaaaatacgaaaattagctgggcgtggtggcagacgcctataatcccagctacttgggaggctgaggcagagaattgcttgaacctgggaggcagaggttgcagtgagccgagattgtgccactgcactccagcctgggtgacacagcaagactctttctcaaaaaaaaaaaaaaaaaaaaaaaaaaaaagaacatactaatttgggctagccacatttcaagggttcattagctacatgttgctagtggttattgtattggacagtgcaggcttagaattttgtccaggaattatattatttttgttatttttaagtaatagaaaaggtgtgtgtggtttaaaaacacttcattgatttatgcctttgtttttaatcatgttttcagcttattttgagatttaaaagaggttgaaaattgttacgtttttcagaagggggcttgctgtagggtgggtagtgattctttccacccttattttagtaaaactgttaaaataggtatttaaatttttgctgttaagataattaggtgttgtccttaccatgattgccttggcctccgtggaattttttcgatgcaaatattctttgtcatatattatttccccttgctgtttcaaagaccttagctggctttcagtctcaaatgccacctctttagtcattcatgcttatctcaaggcatgaaattcactttcatataactccttcagcactctctgtgacccttctaatgttcttccttgtattcttgttaatgtgtcttgtttctccgtaggagatcataagttccaacatgacataaaaatattttatgcatctttgtattttctgcagtgtggagcacagtcctttttgtattgggtcacttagtacatattctagttgaaggcagtttgtcatgtgatatggtgtctagtggtatgatagtttacaggtcaccaagctattgctgatgtttctaggaacatttaagttttgatgtaagaaattaagttaaatttgtgttttgtttgtttgtttttttgagatggagtctcgctctgttgccaggctggagtgcagtggcgcgatcttggctcactgcaacctccacctcctgggttcaagcgattctcctgcctcagcctccccagtagctgggactacaggcatgcaccaccacgcccagctagtttttgtatttttagtagagacagggtttcaccatgttgaccaggatggtctctatctcctgatcttgtgatccgcccgcctcccaaagtgttgggattacaggtgtgagccaccgtgcccgaccctgttttttttttttttcttaaatgtgaaggaatgatagtagggaaaggaaagagacatagagtatacagacacttctgttgcactgtgtgcttagcactgagtggctctgaaatctttggatggatgactgaaagaaaggacttgggtgctaggttgcactggttggtaagcacagtacctttgaaacattgagcatatgacgctactacggtatccccttacattattttatttggagtgggtaaggactgaagagagatttataacagtgaaacctgacagttcaatttctttcctttcaccaagtagcttactctgcaaaagaataaagtaaacagacagtatttaaatatgagaagtcttttcatcatggatgtacttaggtatgtaattgtattgtgggggagttgtgaaactgtaattttttttctttttttttttgagactgagtcttgctgtattgcccaggatggagtgcagtggcgtgatttggctcactgcaatctctgcctcccaggttcaagcaattctcatgcttcagcctcccgagtagctgggattataggtatccaccaccacgcctggctaatttttgtatttttagtagaagcagggtttcataatgctggccaggctggtctcaaactcctgtcctcaaatgatctggatgctttggcctcccaaggtgctgggattacaggcatgagccactgcacctggccgaaactaatttttacaagacctctttttgggtctttaagatgatgtttgtttgtttgatatgttagtatgtcagttctagagctttcccagagagggtgagaaatattaataaacaatagggtttctattgctaaataatacaacttcttttttttttaattagaggaaagagtgtcgtcgtcttctccttcttccttctaccttcttccttcttccttcgtcttccttcttccctccccctcctcccaccttttttggagacagaatcttgctctgtttatcagattgtagtacagtggtgtgatcttggctcactgcagcctcaacctcctgggctcaagcaatcctcccacctcagcatccacattagttgggtccacaggagtgcgctaccatgcctggctcatttttgtattttttgtagagacgagattttgctatattacccaggctggtctcgaattcctgagctcagtccagccaccttggtctctcaaagtgctgggattgcaggtgtgtgccacctcacctggccataatacaaattcttaaatagtggtcgaatatgactttctgtatttggttttgaaaaataaattgctttccacatatcagcatggtatttccttctgtgattaaatgtgtgcattgtgagatgtttggctggtatagggtacaattcttagacatggaagaaatttactttctctgtaagtagcaaatacttgttatgcccttcttgaaccaagaggatttactaaagaacaaggtataattctatttaaattcagttgaacatcaggaataacttttactgagagactctgaggcctttaaaaaatatgtttatataggccgggtgcggtggctcactcctgtaatcccagtactttggaaggttgagatgggtggatcacttgaggtcaggagttggagaccagcctgagcagcatggtgaaacctcatatctactaaaaatacaaaaattagccgggtacggtggcaggtgcctgtaattctagctactagggaggctaaggcaggagaatcgtttggacctgggaagtggaggttgcaatgagccgatactgcactactgcgctccagcctgggcaagagagcaagactgtctcaaaaagaaaaaaatgtttaatatgtatctacttgtggcagtagtctacttattaaaaaaaaaaatgtgtgtgtgtgtgtgtgtgtgtgtgtgtgtgtgtgtgtgtgtgtaatttcctgtcaaggttgtagaaaattaaaggagagttgtgctttttcaaaggatagaggagacaggccaggtgcagtggctcatgtctgtaatccctcccagagggagactgaggaggggcggatcgctgaggtcaggagttcgagaccaccctggccaacatggtgaaaccctgtctctaccaaaaatacaaaaattagctgggcatggtggtgcatgcttgtaatcccagctacacgggaggctgaggcaggagaatcgtttgaacctggcaaagcagaggttgcagtgagctgataccgtgccactgtactccagcctgggcgagacagcgagactgtctcaaaaagaaaaaaaagtgtgtgtgtgtgtgtgtgtgtgtgtgtgtgtaatttcctgtcaaattgtagaaaattaaagaagagctgtgctttttgaaaggatagaggagacaggccaggtgcggtggctcacgtctgtaatccctcccagagggaggctgaggctggcagatccctgaggtcaggagtttgagaccaccctggctaacatggagaaaccctgtctctactaaaaatacaaaaattagctgggcatggtggtgcacgcttgtaatcccagctacacgggaggctgaggcagaagaatcgcttgaaccccagaggcagaggttgtagtgagccgagatcgtgccacttgctctccagcctgggaaacagcaagactccatctcagaaaaaaaagaaaaaaaaaaaaaaagaggagaagttctagtatgatatttctgatatttctgctctacattggagaaggagaacttagttagaaacaaagggtgtgtttgtcgtatttgtcatgacttccaaaatgggagcaggtgggccaattcctgggtcgttagaaaagaagacccagagacaggagattgtcttccggcactgtgtgggctatggtatcaatataagttgtatagcttgttggctttcagtatttatttaggagtaggaaattgttctaaaatgcatttcacattatatctaattaaaaacacatctttttttgtgttaatttcttacatatagctgagaaagcctagactttctaaagtctaggaaagaggtgagaaatgctgctgtggaccataggagttggggtgggggaagaagctaattagaaaaaagtttgtggtcatgatagattttgagtttctctggatgctgttaatgatggccggaattttagaattcagattcagctactctttgaatgtttattgtgtgccaagcactctcattccatttaactttcataatgctctgtgacaatatttgccccatttaatgagtgaagaatctgaggttaaaaaaaatatatttttttaattgatttattccggggccttacagttagtagggaataggttccagatgttgatcccaggccctccagacttgaagtaaaggaagaaaattttttgagtacttaccatgtcattgtacaatagtaagcaattaatttaaaataaatgttactatgtatatttaagatatacaacatgatgttaatatgatacatattataataaaatggctactgtagtaaaagaaattaacatctccatctcacatagttacccaccccctctccctcgcccctaccaagagcaactgtaatctactcaactcttaacaaaagcctagatacaatacactattattaactatagtcttcatgttgtacattagatcttttgacatgtttatcccaggtatttgctacttcctatactttgacctacatctccccatttcctcccctgctcaacctctgctaaccagtttactctctatctctgtatatttgacctttttttgaaaaaagatttcacatataattgagatcatgatatatatttttttctgtgtatagtttatttcagttaatgtccttcaggtccatccatatgtgtgtgtacacacacacatatatatatacacacactgaagtttaaccattcgtccattgatggacacctagggttgtttccatatgttggctgttgtgaataatgctgtagtgaacgtgggaatgcagatatctttacgaagtgatgatttcataatattttgtatatacccaaagaataattactgggtcatatgatagttctatttttagtttctttagaaacctccatactgtttccagagtggctgcactaatctgcattctcaccaacagtgtacagaggttctcttttctctacacctctaccaacacttaatctcttattttttatttttatttttttgataatagatatcctaacaggggtgaggtggtatctcatcatggttttgatttccatttctctgatgataagtgatgttgagcaccttttcatatacctgttggccgttttttaggtcacctttagggaaatgtctattcacctcctttgcccattttttttttttttttttttttttttgagacggagtttcgctctgtcgcccaggctggagtgcagtggcgcgatctcgactcactgcaagctccgcctcccgggttcacgccattctcctgcctcagcctcccgtgtagctgggactacaggcgtgcgccaccatgcccggctaatttttgtatttttagtagagacggggtttcaccgtgttagccaggatggtctcgatctcctgacctcgtgatccgcccgtctcggcctcccaaagtgctgggattacaggcgtgagccaccgcgcccagccgcccatttttaaatagagttatatgttttcttgctgttgagttgtatgatgtttttataaatcttgaatattaaccccttatcagatatatggtttgccaaaatgttttcccagtctgtaggttgtctttttattttgttgtttcctttgctgtgcagaagctttttagtttgatgtagtctcatctatttttacatttgtttcctgtgttttggtgtcatggaaaaagtcattgccaagaccaatgtcaaggagatttctccctatgttttcttctaggaattttgtggcttcaagtcttatgtttaggtttttatccattttgagttggtttttgtgtctggtgtaagatgaggattcagtttcattattttgcatgtggaaatccatttttcccaacaccatttattgaagaattgtgttgtcttggtgcctttgttgaaacatagttgacggtctatgtttggatttgttgctgggctctctattctgttgcactggtctgtgtgtctgtttttatggcagtactaaactattttgattatagttgctttgtaatttaattttaactcaggaagcgtgaagcccccaacttttttttttcctcagaattgccttggctgttcagagtctggtggttccatgtgaattttaggattgctttttctactcttgtgaagaatgccattgggattttgataggaattgcgttgaatctgttttgttgctttgggtagtatgataattttaacaatattcttttgatccatgagcatggaattctttccatttatttgtgtcctcttcattttctttcatcagtagttttagttttcagtgtacaggtctttcaactttttggttaaatttattcctaagtattttgtttgttgatactaacacaaatggggctgttttcttaatttctttttcagcttggtcattatttgtgtatggaaatgccatgaattttttatgttgattttatatcctacaactttactaaattaatttattcaatctaacagtttttcatgtggtctttggagttttctatatatgggatcatgccatctgcaaatagagataattttctgatttggttaactttttttttcctttttcttgtctaaatactcttgatttagacaagctcttccagcacttctagtattctgttgaatagaagtggtcagagtgggcatctttgccttgtatcaggtcttagaggaaaagttctcagtttctcgccattgattatgtagttagctgtgggtttttcatagatggccttattatactgaggaacttgccttctatatttaaactgttaaagagtttttatcaagaaagtttaactttgttgaatgctttttctgtcagttgatatgattgtgtcatttttctttcagtctcttaatatatcacattgattcatttgtgtatgttaaaccagctttggatgcccatataaatcccacttgatcatgatgtgtaattttttgatatgttgaatttgacttgctaatactttattgaggatttttgtatcatcagtgttcatcagagatactggcctgtagttttattttcttgtaatgtttttgtctagcttaggtatcaaggtgatgctggccttatcaaatgtgttacagctctattatttggaagagtttaagtagtattggtattcttaaaaagtttggtagaattaagctgtgaagctgtctggtcctgaccttttcttttttgggaggattttaattaccactggttcaatctttttgtttgttatgggtctatttaggttttctattttttttctgattcaaccttggcaggttgtacttttttaggtattcttttcttctaagttatcacatttgttggcatgtaattgttcgtaacagtttctcagcgctgggtgtggtggctcacacctgtaatcccagcactttgggaggctgagacgggcagatcatgaggtcaggagttcgagaccagcctgaccaaaattagccgggtgtggtggtgcgcacctgtaatcccagccactcaggaggctgaggcaggagaatcgcttgaacccgggaggtggaagttgcagtaagccaagatcgtgccactgcactccagcctgggtgacagagtggaactctgtctcaaaaaaaaaaaaaaaaagtttctcatgacccttcttatttcttttctttctttctttctttttttttttagagacagggcctcactctgtgacccaagctgcactgcggtggtgcgatgtacattcactgcagtctcaattcctgggctccagtgatcctcccacctcagcctcctgagtggtactgcagatatgtgctacaacgcccagctaatttttgtattttttgtagagagagtgtttcaccatgttgcccaagctggtcttgaactcctagactcaagtgatctatccacctcggcctcccatagtgctgggattacagtcgtgagcagcggcacctggccaactgtttttatttctgaggcttctattgtaatgtctccactttcatttctcattttgtgtattttggactcctctctttttgtgtagtctaagggtttgttgattttatttttttaaaaaaccaactctttgttttattgtttttctccatgggttgtctattctctattttaagcaacttgtttgtatcttatttaattttcatcacagtcttctaaggcaaattgaatttataaatgaggaaactgacgcttacagagccttacacagccagcaagatgcatagccatacttcgaacctgttctcttaattgctccactgtatggcctttataatttggagagggaataagataattgcccgaaattggaagaattaaggttttttagggtacggcttaggaacttctactttttgtagtgttacttatggaggagaaagtaattgtcagtcatatataagtacagtttattttattttaaaataaaaattttaagagcaaataccttgcttttaaaactgatctggctagctataaaacagtggctgtgagagatgggtaagaagtgtagttgaaatccatattgcaaaatcagttttgagcctgagctcctatccttttcttttcttctttgagatggggtcttgctctcttgcccaggctgaagtgctgtggtgtggttgtggctcactgcagcctacctcctgggctcaggtgatactcttgcatcagcctttcaattagctaggactacaggcccgcaccaccatgccaggctttttttttttttttttttttttaattttttttgtagagatgaggtctccctatgttgtccaggctgggcttaagcaattcttctgccttgtcctctcagagtgcagggattatagatgtgagccacagagctcagtcaggatcactttaacagatgaaactgaatcttagagaagttaagtaactcttgccaaggtttcatagccagcaggtggcagagcctggattccaacccaggtaatttagtctagagctcttattcttaactactttactgtcctaagagtatgcgaatgtacagaaatggagccttgctacctctggtaacttgaaggcaccaattttgcgttcagttaacatatcatagtttgatccaaggcaagaacagaagtgttaacacttcacctgcctctgtccttattatttcccatggttattggtctcaataatttttgttgttgttttatattattcttgcactggaaaatcctttgtacttagttgtcatttaataaatgatgaatgttgaaaaataagccagaaattgaaaactttgaatcttaaatatagagaccaaattgtaatacatagttgattgtcatgtttttctttttaagaagttatttttatagtgtttgaagataattattattattatttttttgagactgagtcttgctcaggctgaagtgcagtggcatgatctcggctcatggcaacctctgcctctcaggttcaagcgattctcctgcctcagcctctcgagtagttgggattacaggcacgtaccaccatgccctgccaatttttgtgtttttggtagagactggttgttctctaccatggcgaactaccatggtgttcgctgtgttggccagagtggccttgaactcctgacttcaagtgatcctcctgcctcagcctcccaaggtgctgggattacaggcgtaagctaccgtgcccggccgaagataattaaatattaatagaaaaggttgatttatcattttcatgagttggagaaaattttctgtgcctctgaatcgaacttttcttagttgatgagtgttttaagtgtcagtttacagtgaaatcctgtgatgatacaaaatttagatatagtataattgactctcatcctctgccatgcctattgtctgcaggtgagggccaggctgacatttcatttttaatcattgacttaatattcagtaagtaaacctgatatgtgaacttttctctccctttcctttctattctgtaactggaagactaatggaaaaatacagttttctgttaactttgcaataagccctgtgtttcatgtattaatagttggattttttagaccccacttaaaagagtatatctgggttcttaatgtggtggcagtggtcccaaaccttcttggcaccaggactgcttttgtggaagacaatttttccatggacgagcgtggatggttttagcatgattcaagtgcattacatttaatgtgcactttattattattacattgtaatgtataatgaaatagttatacaactcatcatagtgtagaatcagtaggagccctgagcttgttttcctgtgactagacagtcccatctgggggtgctggggagacagtgatagattatcaggcagtagattctcataaggaatgtgcacctagatccctcgcatgtgtgctttacaatagagttcacgctcctatgagaatctgatgctgctgctgatcttacaggcggcagagctcaggtggtaatgtgagcaatgaggagctgctgtaaatacggatgaagccttgctcccttgcctgctgctcacctcctgctgtgcggtctggttcctaacaggccacagaaacccctgatatatagcgtttaatatataggatactcatataaccagaggtacttatttcctagctcaactatatggaattaagatgggaactagatagtagataaaaatgagcactgaggagctaaaagagtgatcacagaatctgaagtaaagttgttgtttttttatttaatcgatgtgaaagtcctttggccagtgtctcccacagtagttactctaaacgatgttgtgctgtaataaatagtctgtggccaaagtttaggaaatgctgcttatagcatccctcaaaggactgccattctttggatcagactttggaagctctgcattaggtgtatttagcccttgtttttatttgagaaggggaggaaacaaacatttaatgaggatttgctccactgggtctaagtggtatctccattttgcagacaaggagtctgagtccctgctggtgggtaaaggacacaaaactaataatgaggcagaactcggaatggatccctaatcttgccgattcctaagtttttgcccttttgaactacgttggcactgtgtggttataatgaactgctgtctattagactttactccttaggaacagcaatttggcaatatatacaaggaaaagactaagctgccctacttagggaagaagaaaaggataataagttgactattcaacacaggttccacatcatttgctttgtatattaatagatgaactctgtgtgtttctatagtgaaagatttcagacacgactgtaaagtgctttttgagtgagatttgataccatctttaggtattccatacatttttttttttttctgacattgtaggaccattttagcctcatctctgatcaggtatagtacagcttaggtaagatattaactttttatagcttcattgtccttatttggaagtggtgatagtaactacctttctgagtgattgtgaagcaggtaattatgcatgtaagatgcttagtacagtgtctggcacatagtaatcttagctgctgttattattagcagtaataacaataataaggcttaggttgggttgttacctttctatatcctctttttttttttatttaaactgttatatatacaggatgttttttttttttgttttttgttttgttttgttttgttttttgagatggagtcttgctctgttgccaggctggagtgcagtggtgcagtctcggctcactgcagcttccacctcctgggttcaagtgattcttctgcctcagcctcccaagtaggtgggactacaggcacacaccaccacacccagctaatttttgtatttttagtatagacggggtttcgccattttggccaggatggtctcgatctcttgacctcatgatccacccacctcagcctcacaaagtactgggattacaggcatgagccaccgcgcctggccttcccctatatttatttaaggaaaaatcatcaccagatgcatttttaaagtaatagaaattccatcttaaatatattttatagggcattaagaaagttgcagttgtcactagggatctggaagtttttaagtgagtattcgtaatcaggaaatgttggcctagtaggtacctttaaagttttgtttgtgtttaatgtgaaatgtgtattttcaatagatgtgatgtttgtatagttaatatttttagactgcattttttttttttttccaaaaactgttttcaggctagtctgtatgcactggcagtctggtttgtattgaccgttaggtattgagttttaataaaatgttcaaatatgatggacataccacattatggtgagatgtgaatgaagattgtcccccacacccccaactgggttgtccacagctgtattcagtagaattaacttaaatggtccaagatactcttcaaaaatttgaataactatttgggaccattcagtaccgtgaaggctattaactgtgaattgagttaagcagaatactgtatgttttagttttttctttagtttttattcttctcaattttgaggaagttggggagagaaacctttcccctactctgaaaaattttctcttccttcctttttatttcctactttgaaatagccaagatcattgggacctctaagaatatcagataatatttacagcttgaggtagagtgattttcatgggaaaccttttctcataaagtaaaatactcttcgtccctgtcaggacatttttagaggagcagcagacagcccctcactactttgtcattacccagggcaagggaagggaagaatgaatgacaggtttcttcttttttcttttccaccacttgttttcctttccctttccttcctttcttgttacccttaggtgtctgtgggttctgaatttggatttcagcagaatggagtaatttttattaaactttttagggaacctggtactcccaaacagatagtgaatacttttttccattttggggtttttatttttatgtcacttttaaattattaagaagttttcaattatataatactagatagactagtttaataaaccactgtatatccatcatcaatttctataattacatggtcaatcatactttttctataccgttactactaacccccaatttattattattattatttatttatttatttattttttgagacagggcctcactctgtcacccaggctggagtgcagtggcgtgatctcagctcactgcaacctccgcctcccggattcaagtgattcttctgcctcagcctcccgagtagctgggattacaggtgtgcgccaacatactggttaatttttggatttttagtagggacggggtttcaccatgttgcccaggcgggtcttgaactcctgacctcaagtgatctgccctctttgacctcccagagtgctgggaatacaggcatgagccaccgctcctggcgtcccaaatttttttttttttttttttttgagacggagtctcactctgttgcccaggctggagtgcagtggcgcgatctcagctcactgcaacctccgcctcccaggttcaaggtattctcctgcctcagcctcctgagtagctggtattacaggcgcgtgccaccatgtctggctaatttttgtatttttagtagagatgaagtttcaccatgttgatcaggctggtctcgaacacctgaccttgtgatccacccacctccgcctcccaaagtgctgagattacaggcatgagccaccgcgcctggccccaaatatttttaaagcaaatttcagacatcatatgatgtttttggaaaatactatttgtttctaaaagagaaggatgcataagacaaagaaatacaaatacagcaccatgattccgccttttaaaaattaaagaattctgtaacatcagatccagtcagtattcacatttctgtgattgtatctttttttcccttaaaaacatggtttgtttttttactaaagattcaaaacaaggtccgtagcatttggttgctatttttaactctcctttcaattggtaatattttttgtttagctcttccttgccatttatttattgaggaaatcagatcatttgttctttggaatttcccacattcaaggttttgctgattgcatccctgtggttatttaacaagctcctttatttcttgtatgttctgcaaatagttggttagactttttagtcagattactttattgcatggtactgaatacaggtgatcccgaattcatgatggttcaacttatgattttttgattttatgatactgcaaaagtgatatgcattcagtagaagctgtactttgaattttgatgcaaaattctttataataactttgttataaagttttatgtcagggctttgtgttaggtgattttgcccaactataggctaaattaagtgttggatcatgtttaaggtaggctaggctaagctgtgatgtttggtaggttaggtgtattaagtgcattttcaacttacaatatttccagtttacagtggatttagtgggcgtactccattttatgtcagggagcatttgtatttcctattgcataacatcaggtgtcatgtgatgcctagttacctctcttttttttttttttttgatgttacagttgattactgcgtctattcttttgaagagatcctttatgtattataaagttccccatcagtctttcacctaatctctttaacagtcatttatgatcattgtctagaaaagcttgtctaattcccgggccacatgtggcccaggacagctttgattgtggcccaacacaaatttgtaaactttcttaaaacattatgagattttttacaatttttttttcttcatcagctatcgttagtattcatgtatttgtgtggcccaagataatacttcttccagtgtcgcccaaggaacccaaaagaatagacaccgctggtctagatcaaggtttctgcaaactacagcttataggccaaatacgatccactgcttattttgtaaataaagttttattaggatgtagccacacttgttcctttatgtattatctgtagctgattttcacatacagtggcagagttaagtagttaagacagactgtgtgtcctgcaaagcctaaaatatttactatctggccctttatggaaagtgtgctgaccccaatctagatcattatttctttagagatagccaatggtcatattctgtcattcattttgtatttgtagctggaatttttctataaagaagaactttacctcaattttattattatcctaagaaaaagcagggtaagtcttgattctttagcatttgacagaaacagtgaggtgttttcttctttttaagtacgtatttatatgaacccccagttttgagtatatttgatgttttaatctattctctttaattgtcaaattgttccatttttgccaatgtgagccccgtcatgttggctctttttatctttccatataacctcagtagtctttggtaatcctgttttctagtatgacaagatgttccaggctcatcttgtacatcaactgtcttagatatctagaaccagtcattactctaaggggttctgattccttttagtggggagtagtatttagagagcacagtttgagtgtttgttgctctgggttggtggttgtttttaggatcttttagtggacagagctagggtgtggttgtatgtctgtgtgattttttaaaaaattgtgtgaaaatacatatagcgttgtattgactacttgaacgattttaaagtgtacaatcattagcattaagtaccttcaccacgttgtgcatctattaccaattagttccagagcttttccctaccctgcaaggaaaccctatattcattaaggagtcactccccatttcccccttgccccagacctggcattaatttgctttccaactctgtggatttgcttattctggacatttgatataaatggaatcatactttatgtgaccttttgtgtttggcttttttccacttagaataatgttttcaaggtttatccctgttttagcatgtatcagtacttcgttcctgtttatggctgaataatattccattgtatgcatatattacattttgtttattcagttgatggacattttcattttttatggcttttggctattgtgaatagtgttgctgtgaacatttgtgtacaagtttttgtttgaacacctgtcatttcttttaggtctgtacctaataatgaaattgcagggtcatatggtgtttctatgtttaatttactggactgccaaactgttttccataatggctgcaccatttcacatttcctccagcagtgtagtagggttcccatttctccgcatcttcttcaagccttatttctattttttgattattgctatcctggtgggtatgaactggtatttcatggtggttttgatttgcatttccttaatgactatcgatgtagagcatctttttaggtgcaaaccaccattgttggtggtttgtgtattttctttggagaaatgtttatgtaagttctttgtcttttaaaaactggatagtctttttgttgttgagttttaagtgttctttatatattcttgatacttcacctttgtcagatattataacttctccattctgtgggttttcttttcattctctttttagtgccctttgatgcacaaaagttttaaatttagatgaagttgaatgttcctgtttttttctcttcttgcttatgcttttggcatcatatctaaaaagagcattgtcaaatctaaggttatgatttacccatatgttttcttctaaggggtttgtagtcttatttcatatatttaggtctctgatccattttgagttaattttagtatatggagtaaggtaagggtctagttgtacaaactccatttgttgaagagcttatttttttccgcattgtgtggtgttggcactcttattactcatcagttgaccatagatacatgggtttatttttggatgcacagttcttattcccttgatctgtatgtttgtctttatgccagtactacactgtcttgattactgtagctttgtcacaaattttgaaatcagtaagtcctccaacttcgttctttttcaagattgctttaggtattcagggtcccttacaatttcatatatattttaggattgcctttttcatttctgcagaaaaggcctttgggattttggtagggattgagttgaatttgtagatcaatttggatattgacatcttgacagtattatgtcttcctatccatgaacacaggatttcttttcatttgtttaggttttctttaattttctttagcaatgttttgtagttttcaagttttgtaaatcttcagttaagtttattcctagttttttttatttttctttttggatactgctataacttgaattgcttattagtatccttttcagattgtttattgttagtgtataaaaattctgatttttgtgtgttgatcttatattctgcaactttgctaaattttggaatttttttaaatgggaaaacacactgtgattcatatcaatacttccaaatccgattttgtttcaagattaagggtttagggttataaggttttctctctttcttagctttatactttttttctcttatgctggaaattttgagaataatgaattgctttattatttatatttatacatatatattagtatttttgaagttattgtgaaaaatggttgttgaaaaacaatttaataattctttgcaattagtataaatactagggaggtacagttaaattgctttttcattaattaattatttatttaattttttttgagacagagtctcactctgttgcccaggctggagtgcagtggcacaatatcggctcactgtgacctctgtctcctgggttcaagtgattctcatgcctcagcctcctaagtagctgtgattacagatgtgcaccaccatgccagctaatttttgtatttttggtagaggtggggtttcgccacgttgaccaggctgggatcaagtgatctgcccaccttggcctcccaaagtgctgcgattttttttttttttttttttgagacagagtctcgctctgtcacccaggctgcagtgtaatggcacgatctcggctcactgcaacctctgcctcctgggttcaagcgattctcctgcctcagcctcccaagtagctggtattacaggtgcctgccaccgcatcctgctaattttttgtatttttagtagagacagggtttcaccatgttggccaggctggtctctacctcctgacctcaggtgatccgcccaccttggcttcccaaagtgctgggattacaggtgtgagccactgcacccggccagcactgatttttgtatgtaaacagttccatattgatggacattagggtattaaatagggaaagtttgatgggaccagatacttggttcttcttgtggcacttaaactttaaaatattttgttttcatttttatcataaagatattttaccacggatactcaaaacagtatagaagtgcacataataaaaagtttaaaatgttctctgattcccatctgcccacttttcctctattcatatctccagaagttactactggttagttcaagactttttattaaaggcatgtgcagcatttatacactgtgaatgcttttttaaaaaaattgggatcatatcatgaatattctgcaacttaattttttaaacatagcattattagttaaccttttaagtttggctaatgagaaaagagtcctgaatataaataagattttacaaaatgattttgggcctttttttctgtgtcattgatactacatacatattttttgcatgtttgtatgcttcctaaatcttacagtattgtaatctgctctaaagtccagaaagtagggtatatattcctctttactttctctgtaggctcaaacttatgtttcgaaagaaattgagaaaacttttattgttgtaatcaattatggtttaatactaaaagtaaatccagatacagttgagatgcctaccagcagagttgaatgtaagttccataaaagtaggggaaaagccttctgatcttgttcatcactgggtccctagcacctaggacagcgactacctcctcattcttcccattctctgtatttgttttacgttttactatataaattataactggatcacgccattcaaactgttctgagttcttttttcacttgtcagtatatcatagataggttttcatgtcaattcttaagtatctgttcttttaaatgagtgtattaatggtgatactatggattacataattaggcaattttaagacttaatttgaactcctttttttccccttagactttggtattagtgttttatctttaaatattagtgggagatactaagttcagagttaggatggagtgagagtgtataacttgctgtatatttttgaagcaaacatgtaatgaaaggtggtacatgtaacatactttaaacaagtctaatatgtgattattctgactatgaatattggagtagttcactttaaaatatttttggcaatgaattcacatgaagaggaatacaaacagtcaacatgtaaaagagtgtttagtatagttaaagaaacaaaaataaaataagataaaattatactctgtgttccccaaattgttaaaaattagaaatgcaatacttagttgctgacaagggtttcaggaggtatgttgctaaaggataataaattcatatatggccggaaatggtggcttatgcctgtaatcccaacactttgggaggccaaggcgggcagatcacctgaggtggggagttcgagaccaccgtgaccaacatggagaaaccccgtctctactaaaagtacaaaattagctgcgcgtggtagcacatgcctgtaatgccagctactggggaggctgaggcaggacagttgcttgaactcaggaccccgaggttgcggtgagccgagattgcaccattgcactccagcctgggcaacaagagtgaaactctgtaaaaataaataaataaataaataaataaattcatatattctttctggaggacagtttggaagagtttcagaaattctgcttctaggacttttaagtaaaataaattaagggtatgcccacaaacttagatatacctgactgtttatgaaaattttaatcctggaactaataacaataatagtgtctaaacagtgtttcttttgtgccagacattacatttatttaatcttcttaacactcctgtggtattcacacagtaagattacaaatgatgatgatgtagaacattttacattgaaataaattgataatagtgaaaatagcacattgcaaaggagtatgtgcagtgtattttcagaaattgactgcaacaaaatctagagaatttgtgagaatatgctctagaatatttagtaaattataatcccgttattcaaatagattttgctatctttattttcgaatttttctacagtaaacatgtattatctaacaagaaacctaagttattttttaaaatgtcctctgattttataaaataatttatttctggatgcctataaacagtaaattcctcacgcatctttaaaatagagatttaaagtgttactaatggaattctaagcaacgcttggtcagcacacaaaataggaattgaaatgtccttgtacagcctttgatttctgccaatttgatggtgtttttttgatgacagaaatggaaagagtagaaagttaaggttaagcatctaaagtttgaagcctgacaacagaaaagtgacaaaatcttttaaatgtttatttgtggttgctggcaatgaagcattaccataaataaacaaataaataaataggtggacagattgactgactgacttgctacattttcatggtgttgcaatcaggtattttctttttcccagttgaaacttagtaactcatatttattgtttaaccttcctaagaattgatttctttcaaatcatctgagtctttctgtcccaagattttctcttttttgtttttttgaagcagggtctcgctctgtcacccaggctggagtgaagtggcgtgatcagggatcactgcattctttatctcccaggcccaagcaatccttccacctcagccttccaagtagctgagaccaccagtgtgtgccaccatgcctggctaatttttttatttttgtagagacggggtctccctatgttgcccaagttgttcttgacctcctgggctccagtgattctcccgcctcggctccctaagtgctgagattacaggggtgagccactgtacctggccaagactttctgtcttgctcctacccttttcaagaattgaagaaatttgttcccagattgctgctaatgtatctggtctgtctaggtctaatacctgtgttagtttaatgtagtatcgtctttaagaccttgttaaaatgtttttcttaggccttcttttacacctttttaaaaaaatcggcagctgttgtccttgaccctgcactttccatttcccttctgtgctatattttttccttctaacacttatcaccttgtaatgtataatagattttgtttatctgtgtctattcttctcccctccctctttccgattttaagatataagcaccattgatgcaaggagttttgtttttgtgtggttcattgtgtattcccagagttcagggtaatgcctgtgtagttcattgtgtattcccagagttcagggtaatgcctggcatataacaagatttcagtaaaattttttttgaataaatgaatagtgggcactcagtatttgtggaatgattagatgttctatttggttgatgaaagtgaaatttattctaagtgtttacagaagaaggttagggttctatagctgttttgtagtatggctagttcattgtagctccagtaatagagttaaatttataggcaggttggaatttcagaactttgttccctgatacatggtaggtgcttattaagtattgctaaatgaatgtcaaaagaagctaataaagtgagtggggttgggggaaacctagcttgctgtgagattctggcctaactggcatagccttttgggtgaggaagttgaactaagatttcagagctctcttgctttctcactaagattcacctccagggactttgtgtttgtaagtatattcctttggaattcggaagttgtaaaaaagtgaattggatgaaacttgctgtaggattgcttttttggaatcaaaactgttgttgaatgttaattagcaccacagctttgtgtgaaggcttttagacttgagtgataatgagattcattattagagacagtgtgtgttaggtgttccaaggcttccacttttattcatccagcacttgagaccgactatatgctaggcattctgctagactcctgaagatacaaaagtcaaatagggcagtaacgaagagaagaagatataaatgtttgttccagagtgctgctttttgaggtttggggtttcccaaaaggcaggggaatttattttattgagtatttttggttttgatttaatttaaattaaaagtgaatatgagccattaattactgagaatatgtttaatcagaacataatttaatatcaatttgctattgagtcagtatgtatgtatgtatgtcttttttaaggaattagcatttattgagtattatatcttacactcttgacatgcattctttttttttttttttttgagacagagtcttgctctgccactcaggctggagtgcaatggcgtgatcttgactcactgcaacgtgtgcttcctgggttcaagcaatcctcctgcctcagcctctggagtagctgggactacaggcgcatgctgccatgcccggctaatttttgtatttttagtagagacagggttttgccatgttgcccagtctggtcttgaactcctgagctcaggtaatctgcccaccttggcctcccaaagtgctgggattacaggcatgagccaccatgcctagccacattattcttttgacacagtcctcataataatcctgagggagaagcattattacctgtattttacaaataagaaaattgagtttagagaagttaagtaatttgtttaaagtttacagctttataagttgtggatttcaaaccaggtttttttttttgatactaaagtacattaacagcaagacatgctaaagattactgcatcttgagatgtaagaatttctgtatatttactctcaggagactctataactccaccatccaaaattagcaactaattaccgctagtgacatgtggctgttgagcccctgaaagttggctagtccaaattgagactggctgtacgaataaaatacacaacagattttgaatacttagtttggaggggaaaaattcattaataagttttacattgattactgaaatggtatgatttatattatatttttacatagtagttaaataaaatatattatcagtataaaaaagtacattagctttttgtgatgcttctcaaaggaagttattatagtttttttctttgtctgtagataatgttaaataacaagatgtattgactttgaatactgtactgtaactgttccccccaaattgaatctttggctttttaaccagagtatttaaaagtacctttttccttactaaataaataaaatatttttgttgtagcaagcaatacaagattggtggaaaagaataaagagaaagaagaaaatatagctgggatagggagagagagattttgattgatcccttttaatcctttgatgtacaggtggtatttaacataatacagtttgggcttctgactctctgagagctggagtcaaatctgggctccactatttaatgggttaatgattggtgttttcatggttttggggtaagttacttattccggttttgcaaatgagagaactgaggtttagagatgtcagcctatgtcatagggttttgaagattaaatgtgacaaggcatgtaaaatgcttggaacatacctaaaatttctaggcacatcctagttctttgcacatagctatattatcttttactaaaatgagtcaacaaaaattgtaactgcttattttgaacttttaatatatatctatctttccacaggaatcaaaacatttaaaattatcagatgataactaatatatacaatggaatgctgtaatatattcagtatcctcttcttgctagacatttaattatttccagttttactttttgcagttaatgaaatgcataggtgaatattttcgtagcagaactttacctatgttcttatctgattaggatagagttggttggttgaagagtatgtatactttaaagatttttgacccattttgctgaattgccttctagaagttcctagtcacgttactaggatttacatgtaatcctagaatttatctggcgacctgtttttctaaattcttattaaatgagatattataatttaaaaaaaattgttgatttaaccacttttaatttttacattgctttgattactagtaagttcgaaggttctttttctttttcttttttcttttttgagacagaatcttgctctgttgcccaggcgggagtgcagtggcacgatcttggctcaccgcaacctctgcctcctgggttcaagcgattctcatgcctcagcctcctgagtagctgggaccacaggcatgagccaccacgcattgctaatttttgtatttttttagtagagacaaggttttgccattatggccaggctggtcttgagctactggcctccagtgacccgcctgcctcggcctcccaaagtgctgggattataggcatgagccactgtgcccagcttctgaaggttatttttctacctgtatattggccccttgtgtattatgctgttttcccattagtgtttcatgtcctcattcccattttgggggtatctgtcactcttctggctttggggtcatgctcagagaaacttgctcttcctcaaactataaattggcagtgtttaaacttcttgctctcaggactccttagtactcttaaaaactattgagaacctctggtcaggcacagtggctcatgtctgtataccagcactttgggaggccaaggtggggcaatcacttgaggtcaggagttcgagaccagcctggccaacatggtgaaaccccgtctctacgaaaaatacaaaaattagctgggtgtggtggtgcacgcctgtaatcccagctacgtgggaggctgaggcaggagaatcacttgaacctggaaggtggaggttgcagtcagccaagatcgtactagtgcatctccagcctggacaacagatcgagactgtgtctcaaaaaacaaaactattgagaacctcaaagacttttgtttactccatctcaaaaaacgaaactgttgagaacctcaaagacatctgtagatctttgccatcttagaaattaaaagagacatttaaaaaatactactttattaagatatagtaagccatttacatgttaacatttttaatgaaaattacattttccagaataaaaattaccaagaaatttttcatttttgccagtctgttgcttaatagaagatacctggattctgtatatgcctctgcatttaatctgttgcagtatcacatgatgtgtaccctctggaaaagttgattgtataatcaggagagaatgagaattaaaaaggcaaatgatgttattatgaaaatggttttggctttgtgggcttctgaatgggttcaaagatcccaagactacagtttgagaaccactgatgtaaataatcacttatatattttccttttgtgtttttatggctgaatttaaattccgaataaattctagtggaaattttctaatttctcccctctcctgcctaccctctccaattggctaatattctttgttgaaagatccattttcatgtaaataattgactgactaggatgaaacagtagacatatctgctttttcccagttgttactgagaagctcactgttcattgaaaacagtagtctttgcagtctttccttaatgttgtatctctattttggtatattcccttattttcttgatgtgttctcccctcctcactctaactcccagttttacaacaaattgccctaaaatcttaaagatttttcattcatactgattgtggcattttgctctaaatattgtttcctccctgatttagaaagtaatttaaaatgtacatagaaacataaaccaaaaaagaatattttgtcgctttgaatatgaaaagtgatattgatatactattattgctgtactgtgatggagtatatcacaggttatggaggttatttcaatattgaaaaaatgctagttgagtatacatacattcctgcaccattgttaataaattatattctttttgatgtgttgcttagcttcttgggaccctttactcactagacttttattgacagctgtgttccaaaatacagttagataaaaagaacaagacacatttttttttctaactttgcaggttggtagagaaatacacatgttctgtgataagtattattatagccacaaacaattaagaatattatgggagggcctaggcaagatatcgttaagaagtgagggaggaatgaaggaaagggaattgatttgaaaagattaaggttgtacaatcttcctttagtagttgatgatttctttaggtgtctgagtccaaaggagagagaaaggaggagtcaagaaagacacccagatatttgtctgggaagaccagatggataccagtgtttatcaaaatgtggaataagccctacttccaacaaaacaaaactcattacatgtgctacactttcaaagacactgtgttattgttttccaagatgagttcaattgtgggcatgttagtttgaggggcctagggtggagatttgtggaagccatttggagagctatcaggagacttgtatgagctgatgagcagtgtagcataagtaggtgaaggaaatgcttaagatcatcctttgagaatttttagtaggagaagagaagatggtcagagaccctctgataattgagggacaggcagagggaaataaattaagcaaaacataaaaaagaaaaaagggtcaggttgtgttgaaggcaagtgaccagtttaaagaaaaaaggttaggggagagagggatctgaaatgttaaatcctgcctaaaggacaaataaaatctgaaaagtgtttattagatttaggaataagaaggcattgatgactttggaaaaagcaactaaactgggaagttgggtgtagaagctggagaagtggaggatatagaggtgaggaagtggaagacagtgagtattgactaccgtttgaaaagtttgcctatgaagctggtggagtacaggttaaaagtgagcaagcagggatttgaaagtgttaatgttctagggaaggagagagaggagggaatgagagattgaactaaggataaaataccatcatcttgaactttctagagaaatgttttttatgcttcttgtttgctctttttgtaaaaattgttctaaggtattttttagataatgctgaatatcttttttgatacctttatgatatttcagctaccatgaatatcagttattgtactgccggtatcaaagaatatattaatagcaacagccctgttctaaaatacaacagttcagggctgtattagttcattttcatgctgctgataaagacatacgcgagactggatacttcatacaggaaagaggtttaatggactcacagttccacgtgactggaaggcctcgcaatcatggtggatagcaaggaggagcaagtcacggcttacatggatggcagcaggcaaagagagagcttgtgcagggaaactcccccttataaaaccatcagatctcataagacttattcactatcataagaacagtatgggaaagatgtgccccatgatacaatgtgggtccctcccacaacacgtaggaattttggcagctacaattcaaaatgagatttgggtgggacacagccaaaccattcaggggccgtttaatgtaattatatagtagctttttaatagccagttttatcttttccttgtgggtgcacatgtgttttgcccactaactcattgatgtggttggtcctagcaatagtagtccctattactacaaatgtgctaagtaactgtgaatgggaaatccacattaccaaattgttagaattgttctttgaatgaaattactgattttaatcattagattgtaaggaaggtctggggattacatctgtaaagtgtactctgtgatagatgtagttttctttttcttttgagccagtgtctcactctgtcgcccaggctggagtgtagtggcgcgatctgggctcaccacaacttccgcctcccagatttgagtgattcttctgcctcagcctcccgagtagctgggattacaggctcccgccaacacacccggctaatttttgtatttttagtagagaagaggtttcaccatgttggccaggctggtctcgaactcctgacctcaggtgatccacccacctcggcctcccaaagtgctgagattacaggcatgagcaccatgcccggcctatagttttctactgctgttttacttagaatgtagagctctatagtagagctctgtagttagactgctttagaatcccagctctaacacttacgaactctgaccttgaacaaatgacttacctaagagctttcttcatctttaagttggtgataataatagtacctatctcacacggctattgaggttaaatgatgtaatgcatgaaaaacatagcttggtacaaactaggtacttactcagtaaacagctgttaataattattttctccctgcctccaggattttgattttctaagcttgatgtttttggaagtcttcttgattagaaagatgttactatatttgaaaagggaatagagtcaactagagtcatattgcaaattttatgtgtctgagagtgtaatgagaatttgcagcatatgtaaacaagtagatttatttttctgaggtaggtccatggattgttaacagtggtttagggaaggttaatgtgtttaaaaatgagaggtaaaaaataacctagaattaccatatgggatattttacttggggtgcgttgggggttactgtggactagctattttaggacaggaaattaacataacctggtgaggctcatgaatctttcagccttagtatcagaactactcggtaacctgtaatttaacccatggcagtacttggagggcaggaagttagggtttgtgcctggtctgagaaagcctattttcacctttttatgagacttgtgctcttaaaagacagagaatgaaattatctaagacatttgacaaccatcaatttagtcattttcttccttctgtcaaataaattgtactattcttttaaacattttagagccctgttgaaaatttgcaccttttttatatagtaaaacatcaatggatggaaaatcctgttttctggggtagccctctttattgaacttctctaacacccacagatcccgtttataaagaagaggcacatttagtatgtttaaaaatagtgaactctttccttctgtgaatttctcaggatgtgttaatggtgtttaaagtttaatgctgctaaacccacacaggaattttcactggttttaaatgccacaaaaacagtttttacttttgttgatagctggtttagtaaaaagaccttgttgttaaattcaggaatgatttaacaaattattaaaggtttcattgcttttttcaaaatacttcctatttgcaagtctcttcatatctgcagtcataccaaattactcatatcttttttttttttttttgagatagagtctcactctgtcgcccgggctggagtgcagtggcatgatctctgctcactgtgacctccgcctcttgggttcaagtgattctcgtgcctcagcctcctgagtagctgggattacagatgcccaccaccacacctggctaatttttgtatttttcgtagagatggggttttgccatgttggccaggctggtctcaaagtcctgacctcaggtgatacacccgcctcagccttccaaattgctgggattgcaggcgtgagccaccgcgcctgacccagtatagtgctttttataattaattgtgtacttttaaaaaacctttattcatgataaagtaaatccagtctactgagtggctgtgctatatctttaacagctattttatctttatttccttcagtgttacagcttccctacatagtacagattatgttacctaattaaatagttgagctatgtcccattctagttaagaaaactgtcctgttgcctattttcatctgattaaaattttctgactttctttcaatatctgtttttttgtaaaccagcctattcagttttatgtcccaacgtcctgttctcttgcttgcccactgatttttttctccatcattaaaaaaaaaaaaaaagaaattgatagacagcatctggagtgtcctagaccctactagacctggtgtttggcccactgatttcttctcaaaatgattgtatcctgtagtttaatttagctacagtgacccataacacttcactcaaactgcttttctgcctctcagattgtttcctcagtcttgcctgcagagcacttctctacctctccagccacctaccccgtttaagtgtttatattttgacctcagccatttgttcatactctgcctaccttccttctaccctagtgatctggtttatgaaaaactagatattaattctactgctttgaccagatctagtcctctaacccagatcttttcagaatcctagacctgcaggaattaccacctacatgttccacagctactttaaactgaacatgttaaaactgaactcattttctctcctgaccttctctcctcccctaaacagaatgaaacctctatattcttcttcttcctctctatcttggtaaatggcaccattatccatagacgcccctggcttccttcccaggaaccaaagttgtttcctccttacacctagttctccaccagtagtcattcttcaagtctaccccctaaatatctttggaacctattcctcttcatttactagagccttcactttagtttaggaccttatcactctgcctagaccagaggtttgcaaactatagcccataaagccaaatacggctagccgtctgtctttgtaaatcaagttttattagaatgcaacttctgtggtttagatatggtttgtttttcagcattaaaacttgtttcgatcatcaatgtggcagtgttaagagatgaggcctggtgggagatgttttgtttcatggggggtggatccctcatgaatggctttgtacctttttgtagcagtgagtgagttcttgctctggtggtactagattagttctcatgggaatgggttaattctgagataatggattgttataaagccaggatgccgccagattttttgtcttcacatctgaccacttcccctttgagcttctttgtcattttgtgatgtagcatgaaagtcctcaccagaagccagggccatgccctttaaatttcctagcctgtagaactgtgaggtgaataaaccttttctttataaattacccagtctcatgtattctgtcatagcactacaaaatggattaaagcagctgccttgctcattagtttatgtattaattgtctattgctatttttatgctacgaaggcagaattttgtagttgtgaccgagactgtatggcttgcaaagcccgaaatgtttactgtcgggccttttacagaaaaggtttgcctacccttgggctgttacttattttctgtctccagccttgtctattcttctgtcctaccccaggtaaacattcagaagtctgatcatatatccccccatatgaatcttttatttactactcatttgttacagaataaagcttaagtcctttatcatggtgtaagatctttggctacctttctgggtttgtcttctgttcagaaggcatgtggatttgtctctagtatatgcttttacctctgccttaaatgttctctcttctctttctcgtcaacttacctgtctctcaacatctaatacagatgctaccttcccaggaagtcttctgacattccagatttttctttttcttgcttccttcataccctctaccgcttttgttgtatttaacattgtgcagcttttatccatttgaccgtatttcctttgtaaattttaaaaccacccaggccaggcctggagccacattcactgaatattccctcaagaagtgtctgttgactgaataaatggattgcttttgtgttagactcttcctaactagtcagtcagagcaaggattatgtctcatacaatttttacattctctgattgtatctagggacatgctagattcatagtaggtacagatactgattaaattgttgatttgaattgtcatgtagataaagtgaagagacagattcattttggagggttggacattcatcaaattgagactgtctagcaaaacccattaaaaaggaactagatttgccttagctactaacttttcccttgacatgtatagggattttttcccttttctgtacttgggaatttgattgactttcattgaatgttttgtgtcattggttaggcatcatatgatctgttttatgtagtgcaaatataagtgacaatttagatggaatgtgctaagttgatgtattaacatagggcagagtcatactaaaagtactgttattctgggtataaatgggagcagaaatggagtgttaccaatgacattagctgttaggatccataccttttcttaactggattaatttgcctgtttgtggtacctaggtgccaattctggtaggactcctagtagcagcagtagcagcagtaataaacacctatctagtgcattttttatgtgccaactactgttctaagcacagtacataaattaactcatttagtcttcataacaacccaatgagataagattctattatccctgcttaacagatgaggagcctgaggcacagggaactgaaataaataaacttggctaaagtaatacagttagtaagtggcagagccagtatttaacccaggctgtctggctccagagtttatgctcttagtgactatgctagactttgtatgagtcttggaacaatttgaatgcacagttcttgcattccttttgttcccctctaagagaaactctggagttttggaacagaggaggaaatgaggaaatactaaatggttaaaggtaggtttatttttaaaaggcacttatatcacattctatatttattatgagtagttgtaggactcctttttagcatcattctgtttttatcttttcctataagttgaggtaagagtttggttttcttttaccttatctgtagccagttgatgcttactaaatgattggctttaacggttctagacattgtaattgtatttacatagttctgtgaagaactgtgaagatacgtagttcatatatttggaagtgtattaacaattgtggtaatcaagaggaaatcatcattagagctggacagcattgatattctaggtgttgcttttaacaggcttaaaaaacacttggaggtagaacatatacagcagattgcataaagcctaagtgtactgctcagtgaatacaacacccagatcaagttaagtacagaacatcttcatcattattccagaaggctttcatgtgtcctttcccagtcaacaaataccctaccagttatgttgctttgtttttaaaatattgtatttattaccacttgacatatattagtttgtcttccctaatggagtttaaacttggttttgctcatcattctgttccattgctcagcatagtgcttgacatatagtaaatgttacacaactatttgttgaataaattaattggaaaacacttatttgttatgtaccatattctatagcacacagatgctattttatgtactagttgcttgaggatagatcataaatcttagttttctaggatgaggcttttgatatttagtgtgagacttacactacttttaagacaaggaaattgacttctcctggcattgggtttagtgtgtgttcgtctttggttacgtgaatgttagggaatatgagaaagttgctaccagtgagagatactggtagcctatctatctggattgccttcttgagagtatctgattccttatagttatctcagattcttaacttacactcttgatgatctttttgaatacttcaaacagttgcaaaagactaaatgctcttgatcttgtggagagaaaattgtgataggaaatacattacttatcattaatttacttataagacagtttggttatcaccttacattgaaaataatgaaaattctgtttgcctatcgtgaaaaggtttaagataaataaaggagtctgtgctttagaatttgttttagttcaaacttaggttcatgtagaaaagaaaataaattttgagctgaaagtgactctagtattctgattcatgttcctgtctgtcaaatttagctctctcattttacatttgaggaaactggagtgcaaagaagtaatgtgatggtctagtctaaaattaaaagtttaggctgggagcggtggctcacacccataatcccagcactttgggaggccgaggcgggcagatcacttgaggttaggagttcgagaacggcctggccaacaaggtgaaaccctgtctctactaaatatacaaaaaaattagctgggcatggtggcgcatgcctgtagtcctagctacatgggtggctgagacgtaagagttgcttgaacctaggaggcagaggttgcagtgagccaagattgtacccctgcactccagcctgggcagacagagtgagacgctgtctcaaaataaataaataaataaaattaaattaaaattttaataatgaataaactttccaagtgatacatactctagtggcttagaaccattgtgctgtcatgcctttttctttcttactttttaagaaaagagactgttctttaaagcagttttaggtttacataataagtgaacagaaagtagagtgagcctatataatcccttacccccacccatatttccccatttcccctgttattaacatcttgcattggtgtggtacatttttcccggttactaacatcttgcaatgctgtggtacatttgttacaattgatgaactaatattgatactaaattattaactgaagtatataatttaccttttataatttccattagggcccacactttttattgtatagttctgtgggtattgacaaatgcataatgtcatgtatccacgattacagtgtcatgcagaatagttttactgcctccagaatcccaacatccacctatttagccaccttctcatccccctcctcccatcccctcctcccatccacctcctcccatcccctggccaccactgaactttttactatctctatagttttgtctttttcagaatgtcctatagttggaatcatacagtatgtagcctttttagacgtggttctttcacttagcaatatatttaacattcctttatgtctttttctggctggatacctaatttctttttgtccctgaataatacctcattgtatggatataccacaatttttttagctgcttacctattgaaagacatcttggttgcctctacttttgggcaaatatgagtaaaactactgtaaacatttgtgttcacttttttgtgtagacatgttggaagtaaatgcttggtgctgcaaagtgaaaccagcactcaggcaaaagttttcttagcaaggcagtttatttttgcagaagggcgtgtctcgtggatggagcaatggcgagagcacagggaacaaaggagagaaggggtttttatttctgatgtgtagtccctacctcagtgtcactcccctgtgggctggggttgaaccgcacaatttaagctgacttgattggctacttgtgaatttttttttttaaataaggaagagggaaggggaaggtgagttacagtggtgggatgtgtggtttcgaagggtggacgtgtggtttcaaaaaggtggaatgggtgcagagtgggtaaccaagggaacagatgtgagttattgattagagctgatgggaaggttgtttacagtaactagaggcaaggaggcatggaggacaagaaagttgagtttgagaacaaagaacaaggaagttaacaggctgaacctttgaagagaaattcattatatcttacagttccctgcttttaatttttataatcttttctctttaaacctttttaacatgtctgggctttgctgtccgacttgatcctttaaaaggaaaagcttacttgcataaggtggaggagagctaagggagattttagtaagtgctgtttttataagcgtttgcactaacctacaatgcatggtatgacacagcacccaataagaatgagtacaagtattacaactgcaagcaaagtaagaattgaggccacgatttcttttcatttattgaaccacctttttagccatcttgaaaaagagttgttgattctagttaattcattggataaagctgtaatcccttgcaagaccttcactatgctctcattgggggcagtattgtttgggatgaaggtacaatattgagttttcattataatacaaactccacctttttcagctaatatcatgtctagggccattctgtttttccaagccattcttgctagtaggacctaattggtcagctattcctttgacagcatctctggtgtaattaataaatcgctgctgattataatagatgtaatctatccaatctacatttttgttgatacttacccatggaggcaatgattcaaatcctgcagctatttggtcttgggctttgaatttatcaggtactccctgtgagattccaattgcgtctaaataaacttaagagtcaaaagacccataaggggcttctcttacttttcagtgttgtgattttttttttttctctgcttgatgaaatgccagggtgaaagggatagccaactggacaagagagcaagtgctgctccagttacttggtggagtgcccagtaagggtccaccacaataccatcatatacctgctcaaggatggctaagggcagactgatgggtaagctcttggaagggcctaagctcactgtgttccattaagcttcccaggaacgccaagtttttcccattccgtgagagacacaaggtgaaattgacattgagagatggaagctggatggcccttgggggctgacccacagggtgttggactttgggatagagcagggagagagcttggcatgatttattactcctagctgtagaatcctggaagagagttaccatgcagcccatgcccggttgactggagcaccattcgagtggaaaggggacaatctgggcctctggccggccatgcacacaagcataacaattgcttttgtttaatgtgcggacagaatttttgatgcatttcaaccagacatttgtatcttgatatcctattttaattgctagagtttgccttagatcatctgcttctacaatgtttacttcagcattgttgtagggcagggtagaaggtttagatggagaaggaggagggaggttaataaaacgcattttaaagaagcatatagggttgtatccattggtctttactcttaagccgtaaaagtgctctgaagggggtgtagggttggtggaggtaggagtattaatggaaatagttactgggttacaatggtgagatggacaattagagggggtggttcctttggtaaaagtggaggtaagattttaggttggtacagccatctggggaggtccagcccggatctctggtagtccagatgatatctgcccattgggaacatatctctcaatttggggtgtcttggtacccctgccatgagcaagatgcagggtcagtggcttttctggaggggcagaggtatttttctaaagtagagatttgtctttgtcattgttcgtcctcacaaggtgtaacaagacaagcatcaaaggtaatgatttggggtgaatctgacctagttacattgataacaaggtggtcatcaatagaatgagaaaagaagaaagtaatagaatagatggaaagatgaaagagagttaaactttttgtagctttagtttgagaggtttttttctttggataatggcccatgactctggaggtggtggtgttttcttgactcgggtgtgatgggtccatcctttttttgctgtctggactgcggttttagtggttagaagccgcaggtaaggtccttcccaggccagctcaagtttttcctccttgatgaggacgtgatccccaggctgttattaatggactgagagctccagggttagcgcctgtgctaagagacctttggttttaagaaaaagagaaagtaaaggagagaccaagtatataattcttgaggaattggtcttttgttttaaatgtaggaatgtcagcagtggagtgtaaataaggcagtctgtagagccatctcataaagggaaaggcctgtatctttttgaggagcagtttggattttcatcagggcaattggaagacatttggtccatggcaactgagtctttataactaatttggttagatggttctttaagctctgatttgtctttttaaccttccttaatgacggtgggtgccaaggagtatgatattttcatttaatgtttagggcttgggatagctttttaatgatatgagctgtgaaatgggttccattgtctgagtcaatattttttattagcctgaacttgagcactatatttttaattaatgccttaactatattattggctgttgcatttgacccagtgagtgaggtgatctattgtcattaataagtattttagacaacccgttagaggcatctctgattaatttgggtactttggaatggtcttaagcctggactcctttccccaagggataatctcttttatagtttgtttatcagttttcatatatttacatataaagcaactatctataacccgttttggctagggtataagttccatgaagactgcatcacacatggcctggggtccttaatgagtcctttgatgcaggttggacaagacttggtctgtcaatatctatttttattctgagctctctttagtgcctgttcttactagcttttctttctcagtggaagagaaaacagagatcacagtaggaggaggaaggtaaggagttaagtggagaacaggcatttcagaagaaatggcagcctgtttggcttatctgatctgttaggttattttcctgactcttaaaagacaggcttttttggtgtctggggacatagacaatagcgattttctctggcaactgaaggttattcagtgctcgggtgattaattccttgtgaacaaggttttgaccttactgttaataagacctcgttcagcctaaaatctttccaaatgtatgagccaccccaaaggtgtacttagaattggtatagatggttcctcctggttctgcaagtactttaaggcttggctgagtgcaaacaattcacaagtttgggcagaccaattattgggctattttcttgactctatttctttaagagcgtctccatcaattactgaatacccattgtgtctttttccttcaatcacttgggaggagccatctataaagaactgtcgcccagtcctgaagtgagtttctcctaactttggccggacctttgtatggtaatcaattaaatctaaacatgtgtgctttctttttagatttagaactcttgttaagaaacctcctgggttaagcaaattatcagtggttaaatcatcctcttgttttaacagagtagcctcatactcatacacatgtaatacttttccagtggtatattttaatataagtgactctaaagaatcaaagttcttttctggtctggggtagccaacaatgatcacattgaggatttccttatagaagtcttttttgaaggtatgcatgatatgtgtttccatgggcttctttgtattcttgtagtatgggttccatcctgtgctcaccaccatcttatggacatctctacttccagcactggcccaactacaataaatgccggtggatatatcagctggaaggttatctaccacttactcaggaaaattagctgcagggatgcccagctgcttggagccgcagccaaatccctgcaccacttggccccagcagaagtaaggcagatgtcccataatgtagcctgttcggggtttggcccgtggcctggtctgcatgttttgcagagccaccctttacatggtgcctgaaccctggaccagcaagggggcaggagcgtgggctctgcctactgtggaggcacaccagggcccggacgatgccagtgacacagtgtggcagatccttgatgctgcccaccctacaaaagctgcctggcgacttaggtgcatggagtccctttaaggagggatacttaggctgggagggaataggttcatagggtgttgattcccaagagtgagaattgtaaggaggaggagtaacatgagtaggggaaggatatgaagcaggatctaggatggcaacagctgcctgagaggaaagggttggggtcactgagcaggggaagacagtgtaggggatcccatgtgatggtctttaggcatgggaactggcttttctaaccttttattatctttccaatattttaacatgagacctagggggctaggtcttttaacaagagattggggggactatctttgttgctgtttttatcctttttacttcttgtcttgcttggcgtatttcccatgttgggtcctagttaggctgaatcccttgtactaaagacttcttgcctattcttctctgggggcttgttaaggctcaatcctttgtatcagtgatttcttgcctattcttccttggggagcttgttaaggctcaataccttgtgttagagatttcttgcctatcctttagccccacctactggaggtttcttgcacccttttttcgctgcatctgctctggccacttccctcgcgggaatatttcaggtccctcttagcattgatggcgggtcagtataaactcctgacaggatccccaaagggccgccctaagctgtatgaggtgaccacggaactgccgataggacccactcactctgcacagcagtagtgcttagtaccattcacacaagcagcactgcaagcagtagtgcttgtgatcatttacacacttttaacctccagaatcccaaccaccaaggaaatactttgtcgcccctgcgaccttctgactttggtctgtgcacagagttacctagtcgctgtggtatgtgagcctcctttccccgagctgccggcctgtttctttcccatgttgctgagagtctgggtttatttgtcacactgggtgggcctcaattcctttatccctgaggctgctgcaacgaggcagtggggcgcacctcttcacgggagaggaccaagaccctttcccagaggagaatgggaatcccggatgagcccgcagatttgttggagataaatgctcagtgccaaaagttttctcagcaaggcaatttacttctgcagaagggtgtatcttgcagatggagcaatggcaaaagcacagggagcacaggagagaaggggtttttatctctaatgcatagtccctacctctgtgtcactcccgcatgggctgggttcggactacacaatctaagcagacccgattggctacttgtgaatattttttccaaataagaaaaggggaaggtgagttacagtgatgggatgtgtggtttcgaagggtgatgtgtggtttctaatgggtggaatgggtgcagagtgtttaaccaagggaacagttgtaagctattgataagagctgacaggaagattggttttttttttacagtaactagaggcaaggaggcatggaaaacaagaaagttgagtttgagaacaaaaaacgaggaagttaacaacctgaacctttgaagagaaattcagaaaaattcattgtatcttacagacataagttttcaactcacttgtgtaaatatctatgagtgtgattgctgggattgtgtggtaagactacatttagctttgcaggaaactgctgatgtgtcttccagaatggctgtactattttacatttctgcaaacagtgagtgagagttcctgttgcctcacattttcatcagcttttggtgttgtcagtgtttgggattttagccattctgttccagtggatgtataatggtatggcattgttttaatttacaattccctaatgacatatgatgttgagcatcttttcatatgcttatttgctacctgtatatcttcttttgtgaggtatcagttcagattttttgtacactttttaattcatttgttttattattgagttttaagagttctttgtatattttggatactagccctttatcagatatgtgtattgcaaatattttctcccagtctatgtcttgtcttttcatcctgtttcatagtgtctttctttcaaagagcagaagtttttaattttaacgaagcccacttttccattttttttttttttatggactgtgcttttggtgttgtatgtaaaaagtcctcgtcaaaccaaaggttacttggatttttcctgttatcttttagaagttttatagctttgcattttactttaggtctatgatgcagtttgagctaattttttaatttttgtgaatttttgtgcattttgagttaatttttgtgtaagttgatgtgtgtttatttgcatgtagatggtcgcttattctagtaccatttttcagaaaaagactgccatccctttttgatgtcctctttcaatgatgcacatgtttttttgagacagagtctcactcttgccttggctggagtgcagtggcttgatcacagctgactgtagcctcaacctcccaggctcaatcagtgctgccatctcagcctcccgcgtagttggtatcataggtgtggaccagggtttcaccatgttgcccaggttggtcttgaacgcttgggttcaaggagcacacccaccttggcctcccaaagcactgggattacaggcatgaaccaccatgcctggcctcagtggcacagttttgagggtgaattatgataactaaaattgaagaagaaacctgagattgttgtaattaataatttgacttcatattatgcctttaaaaatagcttaggaggaagtaacatgttgagtaagcgcctgggctctgggataaattccgaatttgctatttacttgctgtgacattggacaagttactgaactgtgcctcagtttccttgtatgtaaatgtatttttaggttaaatgaacaatatttaatataaattgtttagtacagtatcttgcatatgttatagctatggaagattattagtaataaagacttctttagatgttttatatattttttattcatcagttcctcttattttattctctatagtgtattacaggaaaatataatagtgttaacctatggactgtttgtgtagttattgatgtcatctggcagaagcagatttacataagggagtttaatgactgacctagaataaccatcataaagagcataaaatgtaaaaaatggagttctaaatgcatgcttatatgctcagtattttctgaaaccactaaataccattaaatacctcaagactaaaggtttgatgtgtaagatggtaaaaaaaaaaagagctgtagtgctttatttttattttttatttacttctttttttgaaatagggtctctccctgtcacccagtctggagttcactttataaaacatatttccttcctttgcatatgacgtattacacactaccggcttgcttaatatatttaggattgtttcttattttagattttgagttatctctccaataagaatacaagttcttcgggataatgtgtcttcagtttttcagtctgtcaccatttctggatggagaagctcatgtttcttaattgcttaagaagttaagcaatttggataattggataagctattacttttaaattattgctttattttatcttgccatttgattcttccttctagagtggatagagggaccatttacaatataatagcgaaatgcatatctgtttttaaagttagaactggaaggccagtaagataaacaagggtcctttagggcaataccttgagtaaaatagcaaagcaatttttttgtttgtttcaagtaattgctagtgaataaggagtatttctatacttctgtatattcagcatttttctttgtgttaggaaacaagatgataattgtttaatgcagtgataggaactgctgttctgctgagttatcacagactttgggttttgtcagttaaaacaaattttgagtgttaggttttgttttttaacaggtgttgacattaattttatttctatgtttgggaacttgtaaatttgtctgacattgattctaaaacttttattgtagcatattattttgatatctgtgtatccaagcttttttcctaaaactgctagatggcatagatctgctttttttttttttttttttttgagtcggagtcttactctgtcgcccaggctggagtgcagtggcgtgatcttggctcaccgcaacttctgcctcccgggttcaagcaattctcttgcctcagcctcctgatgagctgggatttcaggtgcccgccaccatgcccacctaatttttgtatttttagtagatacggggttttgccatgttggcccggctggtcttgaactcctgacctcaagtgatctgcccaccttggcttcccaaagtgctgagattacaggcatgagccactgcccagccagatttgctttttaaattaaatttttgtctctgagtcatttagtaattaagttagtcatggtacccccttctcttagatactttaattggcgatactctgggggtattgcaacaacaaaactgatgatcagaattagtttaacaacataaatattcttaatgaggatatcttctgaattatcataataaagtagtgtttatgtttttctgttacataaaaaaatcgaggactttctttggtaagttatacttcataccagttttagtcatttagcacattagagagtaccagtgagctatacattatgtagactatagatatgttaaaaagtacattctattcatgataaaactaataaactaggaatagaaggaaacttgctccatctgataacatctatgaaaaaacccggagctaatacatagttaatggcaaaatattggatgctttctgcctaatattaggagcaacataagggtgtcctctgctgccacttttttttaacattgtactagagtttctagccagagcaattaggcaggaaaagaagtaaaaggcatcagagtgataaggaagaagtatagctatctctatttgcagatatttatagaaacatcctaagaaatttgctaaaaatctattagagttaataaatgagttcagcaaagttgaactcattttatgtaggctcaatgtataaaaatcaactatgtttctgtacagtagaaatgagcaaactgaaactgaaattaaggaaacagttccacttataatgccatctaaaggaacaaaatacttatgaatacatttaacagaagtataaaacgtataatatggaaattacaaaacatcgttgaaagaaattaatgaatacctaaaaaaaaggatggacatcctatgttcatgaattggaagacttaatattgtttaaataatagtgctctcctagttgatctacagttccagtggtttatcaaaattccagttgccttttgcagaaggcaaagttccaattgccctttgcacaaattgataaactgatcctaaaattcatatggaaatacaagggatcctgtattagtttccgagggttgcagtaacaaagtaccaaaactaggtggcttaaaacaaccgaaatttttctcacagtttagaagctggaggtccaaaatcaaggtgttggctctgactgctctagggaattatccccgcttgctgcttctagtgtttgctagcaatccctgacatttcttggcttgtagatcactaaagtcacttagatttcctctacctgtgtgtcttcatattatttgctctctgtgtatgtttgtgtgtccaaaattctttttgtaaggacaccagtcatattggattagggcccatgggaatgacctcattttaattttaatttttttttttgagatggagtctcactctctcgcccaggctggagtgcagtggcacgatctcagctcactgcaacctccgcctcctgggttcaagcaattcttccaccttagccacccgagtagctgggactacaggtgcctgccaccacgcctagctaattttgtgtatttttagtagaggtgaggttttgccatgttgtggtcatgagctcctgacctcaagtgatctgccctacttggcctccccaaaatgctgggattacaggcatgagttacctcgacctctcctggccctcattttaatttgattacctttgtgaagaccctgtttctaaataaggtcacattctgagttactttgtgattaggagtttaacatatcttttttggggtacccagttcaacccataacagactctgaatagcccctcaaaaattgaaaaataaagaataaagttagaggaatcacatttctcaatttaaaaatttacatcaatccagacggtgtggcgcttggatatagattcatggagtatgatcattagtttaaaaataagcccctacctttatggtcaattggttttgacaaggataccaagataattaaatggtgaaataattttttatcccccacaaatggtctcagacagctggatatttgcatgcaaatgaatgaagtttaactcctacctcataccatctacaaaaattatgtcaaaatggattaaagacctaaatgctagagctaaaacaagtaggagctaaaactcctaggagaaaacataggagtaagtttttctgaccttgggtaggcagtggcttcttagatgtggtaccaagcacacaagcaacaaaagaaaaaatggataccttgaacattatcaaaaataaaaccatttgtgcttcaaaacaccatcaagaaagagacaacagaaagggaaaatacttgcaaattatatctgataaaggacttgtatccagaatacaagaagaactcttacagttcaataataaaataaagtccaattaaaaatttggcacaggttctgaatagacagaacctatgtatatctacttctacaaggaagatatacataatggttgataagtgcatgtattagtctattcttatactgctatacagaactacctgagactgggtaatttatgaagaaaagaggtttaattgactcacagttccaaaggctgtataggaagcatgggtgggaggcctcaggaaacttaaatcatggtggaaagtgaaaggggaagaaagcacatcttaccgtggtggagcaggagagagagagagagcgagcgagcaagcaaagggggaagtgccacacactttcaaacaactagatctcgtgagaactcactatcacaagaacagcaagggggaagtctgtccccatgattcattcaactcccaccaggcccctcctccaatacatgaggattacaatttgagatgagatttgggtggggacacagagcaaaaccatatcaatgcacgaaaagatgctcaacatcattagccattatggaaataaaaatcaaaaccatgagataccacttcagactcattaggatggctagaataaaaaagtcagattataaccaatgtttgttaggatatggagaaattggaaccctcgtacactgctgtgggaatgtaaaatggtacagctgttttggaaaacaatctggtagttcttaaaatacacacatagagttaccatataacctagcaattctatgcctagttatatatacccatatgttcatatatacacatgttcatagcattattcataatagctaaaagtagaaaaacaacccaaacatctatcagttgatgaatggagaaatcaaatacgggatttccataaatggattattactgaggaaccaaaagaaatggaccaaagcatactacaacatggatgaacctggaaaacattctaagtaaaagaagccaagcacaaaagaccaagtatcatatgattccacctatataaatgtccagaataggcaaatctgtagacagaaagtaggttactgtttccttagggctggaatggaaggattgggggatgacacctaagaggtacagtgtttctttttggggtaatgaaattgattggggtgatggttgcacaagcctgtgaaagtactaaaaatcgttcaatgtgcactttagttgggtgaattatttgttatgtgaattaaaacttaatgaagctggtttaaaaagtacattgaattattacaaggataagggaccctatgttctagggaaatgtacatattggaatatttaactaacatgagatgttataaagtagaatatatatgctgccaaatggatggtgtagatcagtgctgttcagaaagtatattgcaagccacgtatataattaaatttttctaatagccacatttttattttttagagaaggggtttcactattttgcccaggctggacttgaactcttgggcgccagtgatcctcctgcctcagtcttctgagtagctgggactacaggaacatgccactgctcccgccttataagagccactttttaaaaacttaaatggatgaaattaattttttttttttttccgagacggagtctcactctgttgcccaggctggagtgcagtggcacaatctcggctcactgcaacctccgcctcctgggttcaagcgattctcctgccttggcctcctgagtagctgggattacaggtgcctgccactacccttggctaatttttgtatttttagtagagacggggtttcaccatatcggccaggctgatattgaactcctaaccttgtgatccgcctgcctcggcctcccaaagtgttggggttacgggcatgagccaccacgcacaggctgaagttaattttaataatatattttatttaccccaatatataaaaatcttaacatttcaatatgtagtccttataaaaatcattattgagatattttacatccttcaaaaaaaattaaggccttaaaatctgttatgcatttcagaacgttcgattcaaactttgccactttcaggtgttcaatagctcatgtagtgactaggacaacataagtagagacattaagtgttaatccagagggaggagtaatcattgtagtctggaatgattaggaaaagtcacagaacttgagttattttgctcattcttttatttactgatttcagtcatttatccacaaatatttgagctcttactgtttataaaaaactagcaaaaaatgcagatatgtcttatctcctcatggacagtgcattaaacagctaattatatacttacttagggctggttgcttaagaagtagtggaaagagtataggcttagatttaaatattccctgtgaaacagaggatggtgataactgcattgtgggattggtgggagtaagtaaaattggtttgtcacatgcctaacactgtgcttgttgttaagtgttttctccttctcctacttcaccctctgtccctctacttccttgttgaatacgtgaggtgggccatgggaaaaaaaggcttaggaagtagcagagtcctgactacccaaggacttgagaatgagttcttaccctcatttcttatcctcagtcgcctttcacgggcctttccagggccctgcttcctggacccagactcaagtttgtattattagtactaaagcttattaaccagtattaaagggattttggcgggctaatttaggaatgtaattattgctttttctagagatttttatctacgtttcattccacacaactattttattaatgaaaatttgaaatgctatctgctaaattagtttgcctcttactcagctaagaatgtgttttagaaatatttcaagaagtgttgatataaaaattattttataccttttttgaaattctagacctaccattaaatacttagcactttggtccatgtaatttttgcattgtatgatatcttggaattttaacatactcttttacagtaagacttgttaggtctagttacattatgtttgagacaataatagatgagtttgttgggaacagttatagtgtattgaacagagtttgttaggagcagtttttatagtgtattgaatactggatttagattgggcagatcttggttttccatttgttagctatgtggttgtaagtacatttgttcactggtttttgattcatacagaagatatagcttgactaatatgctcaggacattattgtgtgtttacttatttaaaaattgtgattaagtgctttgaaggaaaagaactggttactgtgaatatagtaacaggtgaaaactattttagattggattgtcctagagaaggcatctctgaagaagtgaggtttaagctgagacataaaggataaaaaggaagggattagggatgggggtggtaaaagaatattgtaaggaaagggaacatcatgtgaaaaagtcctgaggtggcaaaatgctttgtgtattcaataaactgaaagaagcctagtgtgactgaaggattgtgagcaagggaataatggcatgatatgagaatgaaactagatcttacagggttttgtaaaagatgctgaaggctttaaagagttttaatcaggggaatgagacacaagtgtaactccaaagtggggagttgaatataggagtctctaaaggttgaaagagtagtctggtctacagaaatacatttgagaaccaacagctctcaaatggtaataatttggataaataccatgtatttaaagatctgggaatgcatgagataatctggggagagtatggagacagaagatagaagagaaagggagtctaggaccaagcctagaagaaccccaacgtttagaagctgatagggaaggaactagcataggaggacctcaagtgagcagagttttttggatgactgaggaaatataatggttctgaaatagcaatagggaacaaggaggatgtgattttttttttccaactagatataaacctttatttcacaactttgtcataattcaccttttaaaagacagactgggggacacagctgaaaacactgggaggccagatgctggtgtctttcaagccagagcatagctgtggtcactctagggcccatgtgttcctgtggctctcaggcatcatggaccagatacaccagtactgtccaatcccagttttacttagagccacctccttttttgggggccattagtcctcatttcgtgccagattttcactagaagctccctgttcttccaaaacagttaatgaccataagtaacataccatattccaaagagagctcccccaggatgcactgcatgataaaaaaatttccatcccatgaagatttctgctgtatccatggccataatggcttttagggcattctctgctatgaatgtgaacatctgaagggaaataatggcaagcctcccttctgggatcttagtgcagacagctgcaaagaccatcatgatggtgccagatgcaccaagtgatggtccctatcttcctgtggcaactttacacacataactgacacaattggaaataacacctgcagataataacactgctcttgatccagaatgttcactgtgctggaggcgaagctccacaaaacataagatatttgctgccatgtgaaataaggagaaatgactgaatgttgacagcaacattggaaaagaaagggcttttgaggctggattagatgcgaaatatctgatcattgtccgctgcagagaaggtactctctataaacagaatacaaagacattcatttgcagctgtgataacctgtcatagtctggtggccatcgcttaggttattctaccacttgttaatctcctttctgaagtctcttttttgtgatcttatgctatcccaaccgtcagcttttatatcatcaaaataactctggaccctggatttcagtgattcatattgccaaatagtagctgatccaaatgcacagcctgtaaacccaacagtaaaaaataaagattttatgagagtccctataggatacaaagaaggataaaaggccgtttcttccacaggaggaaccaaagcacttctcttgtatgcttcaccacttgtgcctgtgtctgatctttgaggttcagccttcctgggtgcttttctgaatccacatttttgcgaagaagccaaacctgcgtctgagcagccgtggcggggctagggcctcagtgagttccttgcagctgcggccgcccactggcggagaccacgcctggtcgctgccccaacctctctctgcccagcctcaccaggccatcttcccaacctctgccccaccatggcctgcaaggacatggttttgagacgtgtggaggaactgaataacatcagcctctgtgggatttattaaagtagcatatttgggggagttatgttttccataaggctagaaagtggagtttgctgatcacagaagaggtagagctgaggagctcattcagatgagagggatgtgtagagtagtaggggcatgagagttaacagtatttctggtggagacttacgtaaacaaggatatgaggtgttgatagatttaggccttttaatctcttgtaggctagagggatctatggccttagtggcatagtctggaaggtttcaaatagtttgttgggtaaaggactagtttctcgtgaggtaaatctgtctcagcaggttctagttatggtatgatgaggctccaggctggcctcttcatttcttttatctattaagtagtaataatacctcattaattgttatttaaatggacagtcacatagtgattaataaaagttaagactagataaacatacagccttttacgaaatcttttaaacacacatcccagtcagtcatgctctagccaagttattttcttgtcactcttttctgtgctgaatataacaatgcctaattgaagaataaacattgactatgaatgaggcactcaattcatctctttgctgattatactcaattttagaactaaggataaattattcaagaaatagttgctgattttttagtttgaagtgttctataagcatattggatttttttttagaacccagtgacagtaatacttctttggaaagaatataattgaatatctttttagaactttaagcaaatttatctttaaaacattttatatgctcagtgtagataatttggaaattacagagagatacaaaaaagaaaataaaaatttgcctgtattctcactatctagagaaaactgtttactttttggtgtgtttgtgtagcatttgttttcaaaattgggatcacaatatatagcaatttgtagctatatactctgctttttcatgtaatcttatgtcatgaatattttcctaaaccattaaatattcttggaaaatgtaattttaatgagtgcttaatagtctgttcattgtgagtgtatcatagtttacctaatgattctctgctattggacatctggattggctctaatttattgatatggtaaataatgccttaatgaatatctgtgtaaatcaattattttcttccagtttctttttactttcttttgccatattcctagaaagggaagtactgggtcaaagaatagggtttcatttttgttgctctgctttttttttttttttcctgagacagagtctcgctcagtcacccaggctggagtgctgtggcgtgatcttggctcactgcgacctccacctcctgggttcaagtgattctcctgcctcagcctcctgagtagctgggattacaggcacgcaccaccacacctggctaattttttggtatttttagtagagacagggcttcaccatgttggccacactggtcttgaactcctgacttcaagtggtctgcctgtcttggcctcccaaagtgctgggatcacaggcgtgagccaccgtgcctggccgttcctgctgctttttttttttttttggaagtcttgccctgtggctcagactggactgcaatggtgcgatctcagctcactgcaacctccgcctcctgggttcaagtgattctcctgcctcagcctcccaagtagctgggattacaggcgcatgacaccacgcctggctaattttttgtcctgctgcttttaaggaaaattatacttcaagatttcatatttaaaaaccaacgtgggtcgggctcggtggctcacgcctgtaatcctagcactttaggaggccgaggtgggtggatcactcgaggtcaggagttcaagaccagtgtgactaacatggtgaaggcccgtctctactaaaaatacaaaaattagccagatgtggtggtgcgcgcctgtaatcccagctacttgagaggctgaggcacgagaatcacttgaacctgggaggtggaggttgcagtgagccgagattgccctctagcctgggcaacagagtgagactccgtctcaaagaaaaaaaaaaagagtgtgtgtgtgtttgtgtgtgtgtgtgtgtgtgtgtgtgtgtgtgtgtgtgtgtatttggcggtatagcattgcttatttctctgtttccagcagtggtttatttttgtgtggatttgcagacccccttgcggtaataaatccttacaggttctctttggcttatatgttgagaacaactactatagattataaactacctcagttctctcccgtatcctttgtttcgctgctcttgctccagcagataccttctcattgcctctaacaaataactctttctcctttattcagtgattgttttttcctagcatcccttgcttgatatttggggaaagttttaagacttttagaaaatcgtgtttgagagcacctggttttattgtatcgcttgttagccttgagtgtttattttttaatatttgctgatttgtgagatgaaatatgataggtatatttgggggtaggaggaatgtctgaaatacattttctttcccaacttatgatttaataagagctggaaggagaaaagtaagctataaaacccagtatttgacattttatcctcttaatctggctaccgtccatcatactttcagagctgaaagggctttaaatttcatatttatccagtcctttcagtgtacagatagggaaactaaggcctaaaaaggacattgcttgacttcctcaaggtcataagccaaacagtggtagaagtttggcctgttttttcataacttagtgtgttggtttttctacaaacatcttctgcctctctgccgcatgtgtcccccaacctgccttttaggttgactaacttttaggtgactaatttttttctgtttggatgcctggtagactcttttttatccttgagtttaaccacagtactttgatcatcctgagtcagttcttgcatacattgtacctttttgtcctgcagatatagtgctctcaatttcaaggaaagtttcaacgaaattttctattcatgtgtggcatctacttatggaacaataattatccttatattggatcatctttgttcttcatatttgctatcttctccagaattacttcaatctcttaagtgattccctctgcattcactattatgatttcctttcttccctgccattaatttttcactgtgtttttttctgttctttgctgtttctagtttattagatctctgatgttttgttctgctaagaacaaactgagaactgttgtaggctaaaaaacagccctccagaagatatctgtcacccaggctcattgcaacttccgcctcctgagttcaagtgattctcatgcctcaacctcccaagtagctggggttacaggcacccaccaccatgcccagctaaatttttatatttttagtagagatggccaggctggtctcaaactactggtgtcaagtgatctgcccgcctccgtctcccaaagtactgggattacagacatgagccaccacacctggcccccagaagaatcttactatccattagcagtcactccccatctcctgacacacactgcctgctttagataaccaggtagattagtgagtctatcttctttctatagatttgcttattctgaacgtttcatgtgaacaggatcatacaatgtgcagtcttctgtgactgtccacttattttcagtttgttaaactccttcaagatctttctttttacatgccttctgccattaatgttatcgcttcccactccattgcttccctcctccccctaccctctcacacatttcagaatttcagctgaaataccactttttcggagggacctaccctgattggattctagatactccattatggacttaattgtaccccaccattctcttttgtacctcttacttcagttattatctaatgcagcagttatttgtataaatagtatctgcttttctcatgaggtagggagtttgttgttgttttgataaatacttagatgaacatattcaggcactttacccagtgggaggggaaggaggttttcagagaagagagatatgtgtacgtatgttaactattaatgtagcaatgaaaatgtaagctgaaaaaatagaaaattagtagttaactaggctgtatgtgtgtagactggacaggtaccataaagaatgtcaaagaatgtctattatactctcccagaaataccatgaatctggaattgttttttttgagacagggtcttgctgtgttgcccaggctggagtacagtagtggctccagtagtggagagcatacatggctcactgtggccttgacctcctgggctcaagcagttctcccacctcagccttccaagtagctgggaccactggtgtgtgccaccgtgcctggctaattaaaaaaaaattttttttgtagagacagtatctcactatattgcccaggctggtcttgaactcctgggctcaaacagcagtcctctcacttggcctcccaaagtgctgggattataggtgtgagccattaaaacctaagactgttaagtgccagcttaatttttggagtccatttatgttgttggttagaatatttggaacatttaccctacctgaaaagttgaataaaactgatatattctgttttaaatttggattcatcagttcagtttttcaagctcatttgtaaataatggggcatattgcctacgcgggtcacaatttgtttcaggaaggtgctgcatcatctgctttgatggagatcttaatgaaattggctgctcttgacctctgagattgaccatttcttcatcttttcatggtacctagattgtcaggtacactgttctcttctcccttagtgtttgctttactctttagtggatattggagatacctaggacaaaggtttgtgacgagggtgtgtgtatgagggtcttaagataagggaaatggtatttttcttcttgattaaatttaaggggcatcatgaaatttaagtaacaccttaagagtaataggtcagatggctttttgttgatattattgatctataaagttattgctagcactgtgtttaccatattaagtattaaaaccttatattctgtgggactcttcacatgggtatttagtttcagtttttataatggaaattggtgagaaaaaaggatgactctacaaatttttgttttgtagttaacttttattacaatttcatctaggatgcatgaaagtacatggaatttaggggcagtgttggacataaccttggagggacccagctcaggaaagtcttcagtggaacctttgtcttattatttgcatatattcacatcaaacccttttaaagtttccactgattttttttttttttaaatgagtatgtgttcaatgcttctgcaggttgtagaggtatggtataaaaaagaaattcaccagcagtcacagctagcagacttgggctagtcctatcctggtatatcattaagtcaggtagttaacagtaggagatgacaaagaccatttaaaaaagggcagagttaggcatgtacatatttcccaaggatgaaagttctgtttcaagggaaattgtttcctactttaatgtccttatctacttttgacactgcgaataatgagtaagcactgtgggagacactctgctttggcatcggatgtcctaagacattcttccatgacccaccaataagaggattagtctcattttatttctcaggcagtgataaaggaaccaagtcccagttgtagacccactgtgagtgcaggattattccagtgtcttttatttaatagagccagttagcatctcttctagtagagctttgtggggagaaagaaatgctaagtggactgctttgtgaattgacccacttaatctgacagtactcttccgccttgaagaggtggacttggacttgccctatttgaatggcattgccaggttctctaaaggccggtctttttttctttcatttctttcttttcctctctctctctcttttgtggggagaggattgggaggtgttaagcaagtttttagagggaggaagataaggcaaatacaaaaaccaatatggatttgattataagtaactgtaatcagttaggacaatgttatttaatatgtgcataccttgacaatattgtgggtttggttccagaccaccacagtaaagcacatcacacaatttttttggtttcttagtgcatataaaagttacgtttgcactatactgtagtttataaagtgcagtagcattatgtctaaacagccttagttaaaacatcctttattgtttaaaaatgctaataatctgagccttcagcgaatagtcatctttttgctgctcagtgttgatggctattgactgattagggtggtggttgctgaaggctagggtggctgtgccaattgtttaaaaggagacaaccatgaagtttgctggattaattgactcttcctttcacggaagatttctctgttgcatgagatgctgtttgatagcgttatatttgtttttagtttttaatagagatgggtgtctcactgtgttgtccaggcaggactcaaactcttagactcaagtgatcatcttacttctgcctcctgattagctgggactacagtagccagcaccccaggcattatagcattttacccatagtagaactttcaaaattggaatcagtcctctcaaaccctgctgctgctttatcagctgagattatatcctaaatcctttgttctcatttctgcagtagtcacagcatattcaccaggagtacattccatctcaagaaatcactttctttgctcattcataagaagcagctcctcatctgttcaagtttgatcacaagattgcagcagttcagtcacatcttcaggctctgtttctaattctggttctctttctatttctaccacatctgcagtgacttcctccactgaagttttgaactcctcagaatcatccaggattggtatcatcttccaaactcctgttcctgttcctatgaatcatgaatattctttttttttttttggagatggagtctcactctgttgcccaggctggagtgccgtggcgcaatctcggctcactgcaacctctgccctccgagttcaagtgattctcctgcctcagcctcccgagtagctgggattacaggtgcccgccaccatgcccggctaatttttttttttttttttgtatttttagtagagacggggtttcaccatcttggccaggctggtcttgaactcctgatctcgtgatccacctgcctcggcctcccaaagtgctgggattacaggcatgagccaccgcacccgctgaatcatgaatattcttaatggcatctagaagttgtttagtgtagttatcttcatcagtggtcttagctaaatcttctggataacttgctacagcttctaaatcagcacttactgctttaccttgtacttttatggtatggagagggcatctttcctctgctagcttcaaacttggcagccttccttgaaggcttctgtcggccttcagagaattgaagagggttccggccttgctctggtttaggctttggcttaaggaaatgttgtggttggtttggtcttctatccagaccactcagactttctccgtatcagcaataaggctgttttactttcttgtcattcatctgtttactggagtagcacttttaatttccttcaagaacttttcctttgtattcataacttggctgtttggcacaagaagcatagctttcagcctatctgggcttttgacatattttcctcactaagcttaattacttctagcttttgatttaaagtgagagataggtgactcttctctcacttgaacacttagaggccactgtaggcttattaattggcctaatttcaatatcgttgtgtctggaataaggaggctgaggagagggagggagatgaggaaacaactgattggtggagcagtcagggcatacacaatattaatctattaagtttgccttcattgatcacagatcaccataatgggtataataatagtgaaaagtttgaagtactgcaaaaattaccaaaatgtgacacagagacataaagtgagcatatgctgttggaaaagtgacactgatagacttggttaacatggggttactataaaccttcaatttgtaaaaaaacataatttctgcaaagtgcaataaaatgaggtatgcctatatatttttctcttaggcatgttccttattatattaactataaacagcatcattactaatgaagttgtaaactagaatttaatcagtatttgaaaatgtatggtgtcccttgcagaaatggaattaatgacaaaactcgtagttattacctgctattttattttccatagttaattttaagaggttcttataaagttgtttggaggtactctatcttcgcaacagttaaaattgtaatattttaggcatatctcctcctcctattttgttatacagaaaagctttagctgataaacagattgcattctaaaagttcataaactattttggaacttagaaagtattttttcatagtaagtttagtaaatatgtaaatgcgaataggttcccaggctatcctacaaaagcctagtgaagccatagttagctcaaatatctacatatttacagtgacacataatagaaaataatatggcaagaatagtagttaacaccagaacactgttaagaattagttttgaaaaattttcttctatgtgtgattgatacagggatttaatcagagggaggtcaggcgtagagacttgcgggagtctggcaggcctttctgtgtcctttcaggtactttataggttgctggcatttctttgttttgatattggtatacagcagaaatgctatctttttgcttaccggctatggttaagattgtattttaggttctgagtggaatgaaaatggtagaaaagaaattgtttcctcaggtaaaaaagataggaaagaacaatggaagacagagtaacctaggatgtgtgtttgaaaaagaaattgtttgagatagatttcattgacacacaaataatgcaggtttaagggaataggggtagagtataacatgcaaatgattcctgatggaggagagcaaaggtgttgataaagtgttttgggttagcaatccttatgctggtcagtaagtggttttggatatctttggaagctatttagaactgggaatggggagtagaatgctactatggtatttttatatgaactgtttcagtattttttttttcataacatggtacatcagctcaccaaaaatatttaaatagcttgatatacatttggagtgttttttgatgtattaaactattattaagaaaatatattttttgaatgtatgaaaagtaaaagtaaaatattgtactttagctaattcaacagatgcctaaaactattatgtgatgagagagatttggtttttacattccttttttttgtttgtttattgattagaaaaaccatgcttcctttttcaatttccaaaatgtttcatatttagaagaaagcagtccaaaaagaaaatcaggaaatgttccttctattcctacaaaagtagcacctgttgttaggctatgtattattacttagtattgtgataaatctgttgaagtgtttaaatgtttgctatctgttcactgatgtgatttgcctcgggaatttctggctctctttttactcgttttagatgatttgtctctcaggtagctttgtatatcagcaaaatgacacagttttatatttaaaggtatttttgcagtttatattagaagatgtaaagactcttactcaaaatgtgtggttatattggaccgagttatttttatggatttattttagaatgtaaattattcaattcactttcacttttataagtaatggaaagtatactaaacttaggaattggatgaggcaatttggatgagtactataagactatagtatagtttttaatactacacagtctaaactgtagtgttatactctagtttttaatactatagagtcattgctgttatacatggggcattggttccaggaccactgggcacatcaaaatctttgcatattcaagtcctgcagttggccctatggaaccaccgtatgtggaaagttggccctatggaaccaccgtatgtagaaagttggccctctgtatatgtgggttttacaccctgtgaatactgtacttttaatctgtgtataagtggacctgcgcagttcaaacccatattgttcaggggttacctgtatataaccttgagcaattttacttattttttgagtctcagttcccttctccctgatttagtactggttcttcctcctttgtaggactgttgcttggatcaaatgagataatgggtctaactgtgcttttaaactgtaaagtgcttcacaaatttatttgtgatgatgagtgatgatggtttggtgatcatgattggtcctagggaaaactgtcaaatatatgaattttaactgcattatcttctcaatctgttgaattgacttcactcttttaagagggtgattgcagggtccgaggtcatctctgaggaggaagtagaggtatttattttggtgactttcagactttagtttccaaagtatagtagaagcctgtagaaatttgtcacttataaatgaacaaatattttttcagactgctatctgcatagtcataaaatgggacgcccccagtgtgtgataactgtaagtgttgccctcatggattgggtattagttgttaaatcgtaattgataaagtatgtaggactgacttagagtaagcagtagtcttctgggagaaagcaaatcctgtcctctctcctcccctccctactgttttaattcaacagcaatttttgatttaataacctccactttaaagtgattaaaaatgtgtgtaaatttgtatgctgtgctatggaaaaagcagtttggattaaggagtgcagatatctgcatatttcaggtgagggcagggttggataagctgttatattgggggcatttgggttgtcagaatgcaatattgaggaccaaagggatgtctagtaggccaagaacagacctaattgatttaaaaaatatatttagagcactgatctggtattactgtcactaaagattacagtaaaatataccagctttttgagatctattttctggctttcttctggctgctcaaggttgatgtttctagaatgtgaccactgactggatcagtgagtgagtaattgtatttttaaaagtaattatttacttctttagtttaagtacttaccattttctcttttcatccatgtatagcgtactaattctttgataatctggtaaccttccttcttaagaaaaatttaaatatagtgtctacccttgccctccaaattgggcagcttgtaaatcaaacggataagcgtgtttttaagagttaaaaattctaatgtaaattcctaaaagccaaagatttttatgcacatgctgtaagccttttagacgtgtatggaaaataaacctgtttctaaattacgtcttgaaattatttcaagtgaggagttaagaccaagtgaaatatttattcactttgggtttggtttgattttattccagttcagtgaagttagagaaaaggaaaatacacttgattccagtttatggcttacaagtaaaataatatttaagttcagactgttgaaaattagcatgtttctgattttaggtttattgtttggtttaaagtaaaatttggttttgaggtaaaaccaagaagataaaaaattgagtcaattaaaattggctcagaagaagtatggatatttcaaagaaaaaattggagtggctaatagatattggcctcagcaccttttgtaagatatttggagttttaggttttgctgaacacaaaaagttggtttcagggcctaaatacccatctcttatatttatgacatggtccttagcaaagtaatgtagtaggacacgagtttttcagtaaatggacaggtctgaatcctgtggatatgagttgttgaagggattactttgagcaaattatcaaatctttttgaacaccgtaaattttcccatttgttaaaatgagtgttctgagtatgtcacaggctttttgtaggaattaaatggtatattgtggctcttcaagtgtggtagctggacctgcattaagaagcatttgaggtgcttattaaaaatacagtttctggagccccacttcagagctactgaccttttgggggatatattctagggtcttcattttagtaaactcccaggtgattcttatgcccactgtgagtggtgcttgcaaactgtaaacagttctcaaggaatatgcttacataaattgaacttaagatctcagttttgtctcctgtccagtttttgccaattgctgcatattgatgctctaaagaaaccagaattatttggaaaagggttaggaacagagtaaatttcccacccatacctaaatacacatgtgggacattgagtcatttgttttggctgcttgttgtcaccatgttttcagtaagccctccattctgttttactacttgtaaggcttttctatatatcttctccccaggaatttatatctgaccaatggcaagcataggaagacacaaaagcactacttaggtttaacatgaattagtcagttctcaaatcctaacaattctgcttgaataattcttacaaagtctgtggcaaacctttgctgagcatttgcattgtgctgaacctagtgcacatgggaaatgtaaatgattccaataatgtctagttttttttttttttttttttttttttttttgagacagagtctcaccctgtctcccaggctggagtgcaatggcgtgatcttggcttattgcaacctccacctcctgggttcaagcgattctcttgcctcagcctcccgagtagctgggattacaggcgtgcgccaccatgcccagttaattttttgtatctttggtagagatggggtttcaccatgttggccaggctggtctcgaactcctgacctcatgatccacccgcctcggcctcccagagtgctgggattacaggcgtgagccactgcgcctggccaatgtctagtttttctaagagctcttagtgaagtaaggggttcagacttttaattatagggcattgtgataagtaccgtgatagggcacatgtgagtggtaaaagagcatagtggagcagaataatatattctttcctatttccactgctaataatttgaagttcaaagaaactgtaatttattgagcacccactaagtggcatgcatttaaaaaacaacaactgtgtaacctgtgctattcaagtgtcacaaccctaagtagcagctatttccattttatagatattaagtaggttgaacttgtgatataaacctttgtatgacttcaaagcccatattcttttcattaggcctctaaggacaaaatttgatttttttctttttaaagaaggggatgttatgattcttcagaattgatatctgttaatttcagtagcatgatgtcttcctgtgattgaaaagaactctggagtctttctgtcatctcttgtcctctgccaccctatgttttccttttcatttctaaaagtgataaacaagtaaacatctccggaagccagcctatgtgacatctgtccattttgttttctctgcctctccttttatttactcttcctatccccaaaggaaatttagtaattgttttttttgttttttctaatctcttaggttagaatcatgtgaagatgaatggtatctaagggaaaagaccttgttagaaagatttttatcacatgaagtttacatagaaattgccctggaatttttatcctgctgttgatcattagggttgatttgattgattcaactgcttggttgatatttcctttctttcttcatagtttcatttgttccaacatatataagcagttgtgcactctggctagagcttccgcatcataaataaagcccctattttaatcacaagaataagttcatttttgaattttctaactctgatcccatggggctcagttttctactttttattccctttgctaattaatttttatgttgttttcttgtttctagcagtttgtttattttggggagaacatctttgctttttaaaattaaatttattttatttaaaaataaatagagacaaagtttcactatggtgcctgccatggcctcccaaagtgctgggattgcagatgtgagccactgtgtctagcccaacatctttgttgttgttgttgtttgttttgtttttgctttttttttttttttttgacagagtctcactctgtctcccaggctggagtgcagtggcatgatcttggctcactgcaacctccgcctcccgggttcaagcaattctcttgcctcagccttccaagtagctttttgtacttttagtagagacggggtttcgccatgttggccaggctggtctcgaactcctgacctcaggtgatccactcaccttggcctcccaaagtgctggaattataggcttgagccactgcgcccggccaacatctttgttttaatagttttttttttttttttaaacgtggtttctttggataaagagttgaacttcaacttaacctgaaaaatttacagtggccagagtttcctcttctgacagtcttatgtaaacatcctttaaaaatagctgtgtgttatttctctacatgatagagaaataattggcagtgcctttccctctgctaatactattgttagacagaaaatgtcatgaaaatgctttgtatcttctgtgtattcatgggcggtgtgctgatttctctaggcttttgttgcagacttttaattttctgttatttctacgtggagacttcattcttgaggtgaaggaaaatttttatagaggcagtcattgcagacttaactctccacagcaaggtcaccagagtatttattagactgtggcatgacttttctcagtttgtagtacaccttattcatattctctaaaagcacctcagttacaaagtgtctcagttgttgctttttctcagcactggattttaagaactagtctccttaaaacaagaaacatcaaattaaatgcctttgttaaatgaacaaagtatgttcagctttgagatttatcatttgaataaaattagagtgtcagtgccagacacctacctacgaaatatttaaacatccttgaagtggacttttggcttttggatactccctccataccttggatgggtccatgtcaccgtattcatcgtggagtggattggtctgaggagtgacttgagaagacttcaaaaaatatgactacaaaatgttgtttacaggtacaatcctgtttttaggagattgagaactaggctgtgtgtattattttgggatatgcctaaaccgccaaggcttttgtaaaaattgtattctagactggatacagtggttcacacctgtaatcccagcactttggggaggctgaggtaggatgattttagacttaaggccaggagttagagaccaacctgggcaacaaagtgagaccctgtctctccaaaaaataaaaaaataaaaaatattagtgaggcatggtggtttgtgcctctagtgttaactacttgggaagctgaggcaggaggattgcttgagcccaggagttcagtgttacagtgagtgagctatgatcacgtcattgcactctaacctggacgaccctgtctcttaaaaaagaaaaaaattattctgtatttaacagccttcaatagaaagtgattgttctgttgtataccgtcttttcattgcgtcatgaggggtctattctggtttatgtatttaagatttatctattatggtaaagagtagggatttaatgctgtgttttaattatgtagggaatttgtacaatgtgagtgaagtcctctgacgggagagagggcctgaagctgaactagttcctgctgctgtgctgttgtaccttcaaggcggtttcgaagaccaattttattataatcgctagtgccatataattagtttggaaattactgtttcatgtatgctaggaaatttagtactgggcaaagtggttattttttgtttaagcagatttgttacagtttctagtttttcttgaggattttggtctcttggaagcagtgctatggtctgaaatagcccaatttgttgtagcaaatattaagttgtttggatgtcacagaatcaactgttgtacagttttatttttgatatggttttgctttggtataaatttcaaaacctaatgttgccaaagtgtctctgggaatagatgttgatattaaggagtacatggttgaggccagaaaactataaaagcctgttgatttattctcttacttcctattactctggagctgatccttttacattatgttattggaaacattatcttctttttttaaggggaaatggagaaaagaatatgaagttagtggtagtgggaaggttttaaaatacagcagaggcagcacgaggcatgcgatgagttctgctacctaaagctttatgataaattaatctattataggtattaatgtagatgaaacaatgaatgggattgttctttgtaaacagtacaattttatacagttgttatcttattgccaagttggctaattccaatgccaaatggtaatgcgcttttccttggcacgctagttgctgaccgacccatggcactactaatgttcatgccaagtcatcccccatattgccctgccattcttttcagctctgctatttatcttttaattactgctaaggtagtaagtaccttgtctctagatgtgcaaaagtgtgaaagaattggtccttgtctacatcaaatctcagcttaaaattaagaggccttaagagtctctttcagctatagtatctgtaggacctgttttacctttccactctgcttttaatactttagcagaattgtcttatttgatgctttttcccatttgcaaattatgatgctgggatagacattcctaggctcagtggctggagtgaagagatgctaaatcccagggttctaatggatatctatcatagctagttttcaaacccttgaaatgttaccttttttaaaaagaagtagaacctgtggaggcaaatagggaacgtcatcacaaatgcttatattgcttaagttctcttactagagcaaggaaaatacaaaggtggggctcctgtttcctttattgaaatatggttatgcagtagaaaaacataatttttggttggaccccagacagtttttgttttccgtttttcagttaattaaaattgtggagtacagtaggctttttgtgttgctgttattggaaatggtcttgctgtgttgcccaggctggagtacagtggcatgatctcagctcactgcagcctcccgcctcctgggttcaagggattcttgtgcctcagcctcccttgtagctgggattacatgtgtgcatcactgtgtttggctaatttttgtatttttagtagagacaatgttttgccatcttggccaggctggtcttgaactgctggccttgagtgatccgcccaccttggcctcccaaagtgctgggattacaggtgtgaactaccatgcctggccatttatttatttatttatttatttatttagagacagggtctccctttgtcacccaggccaaaatgcagtggcgcagtcttggctcacttacaggcatgtgccaccacactcagctactgttcttaaaatctatattagtttattaatgatcctttgttttgtgagttagatgtgagattttgcaagcaacttgttcagtggaatttttaattcagagtcctcgaaatctttttttggtgttacagttttgacttggataacatagggaagagtcagactatgtttgaaataacgtatgctttttatgttctcttgagacaattggattgtaagaatgcattcaataccttggattttaacctttctggatctcttcatttagagtttaagttcagaagactgtgattctgtttttaggactgctaagatacaggtaaagaaaagttattttacaaatgagggagagaataacttatgataaggtttatagtcagcttgaaaagttgtagttaagattagcatagaattttggggggtggggcatgataatttagaactcagattaaactaggtttttagcaaatatacctaaggtttattttactgagggatgagacttgaagggagagtttgaaagctttgaaaactgctttttttatattggcttatgctggggtttgtgtgcatttaaagcatatataatactgctgtaaataaaaatggtggtgctgacaagatttgagttctttcttttcacaagtggttttggatttggggtgattaaatcatgccagtttgagtgttttaccttggacctaaagacataaatattcaagatggctggccatgataaatatagactgcctgtatttcacctggctatcttatgaaggaatccctcattgcttttgaatctgtcatgagtgccatctttttttttaattattattatactttaagttgtgggttacatgtacagaacgtgcagttttgttatataggtatacatgtgccatggtggtttgctgcacccatcaacccgtcatctacattaggtatttctcctaatgttatgtctcccctagccccccaccccccacagaccctggtgtgtgatgttcacctccctgtgtccatgtgttctcattgttcaactcccacttatgagtgagaacatgcggtgtttggttttctgagcttgtgatagtttgctgagaatgatggaatgatggtttccagcttcatccatgtccctgcaaaggacatgaactctccttttttatggcggcatagtattccatgatgtatatgtgccaaattttcttaatccagtctatcactgatggacatttgggttggttccaactctttgctattgtgaatagtgccgcagtaaatgtacgtgtgcatgtgtttcatagtagaatgatttataatcctttgggtatatgctgagtaatgggatggctgggtcaaacggtatttctagttctagatccttgaggaatcgctacactgtctttcacaatggttgaactaatttacactgccaccaacagtgtaaaagtgttcctatttttccacaacctctccagcatctgttgtttcctgactttttaatgatcgctattctaactggcatgagatggtatctcattgtggttctgatttgcatttctctaataaccagtgacgatgagcattttttcatatgtctgttggctgcataaatgtcttcttttgagacgtgtctgttcatatcctttgcccattttttgatggggtcgtttgcttttttcttgtgaatttgtgtaagttctttgtagattctttatattagccctttgtcagatggatagattgcaaaaatttttctcccattctgtttcttttgctgtgaagaagctctttagtttaattagatcctatttatcaattttggcttttgttgccattgcttttggtgttttagacatgaagtctttgcccatgcctatgtcctgaatggtattgcccaggttttcttctaggatttttatggacctaggtcttaacgtttaagtctttgatccatcttgagttgatttttctataaggtgtaaggaaggggtccagtttagttttctgcacatggctagccagtttttccagcaccatttattaaatagggaatcttttccccattgcctgtgcgtgtcagatttgtcagagatcagatggtggtagatgtgtggtggcatttctgaggcctctgttctatccctttggtctatatatctgttttggtaccagtaccatgctgtttggattactgtagccttgtagtaaagtttgaagtcaggttacatgatgcctccagctttgttcttcttgcccaggattgtcttgggtatgcaggctcttttgtgtttccatatgaaatttaaagtagttttttccaattctgtgaagaaagtcagtggtagctataaattactttgggcagtaaggccattttcacgatattgattcttcctatgcatgagcatggaatgtttttccatttgtttgtgtcctcttttatttccttgagcagtggtttgtagttctccttgaagaagtccttcacatcccttgtaagttgtattcctaggtattttattctcttagtagccattgtgaatgggagttcactcatgatttggctgtttgtctgttattggtgtataagaatgcttgtgatttttgcacattgattttgtgtcctgagactttgctgaagttgcttatcagcttaaggagtgtttggcctgagacgtcaaacagagacaatttgacttcctctcctcctatttgaatatgctttatgctttttcttgcctgattgccctggccagaacttccaatactatgttgactaggagggatgagagagggcatctttgtcttgtgccggttttcaaagggaatgcttccagcttttgcccattcagtatgatattggctgttggtttgtcataaatagctcttgttatgttgagatacgttccatcgataccaagtttattgaaagtttttagtatgaaaggctgttgaattttgttgaatgccttttctgcatctattgagataatcatgtggtttttgtcattggttctgtttatgtgatggattacatttattgatttgcatatgttgaaccagcctgtcatcccagggatgaagccgacttgatcgtggtggataagctttttgatgtgctgctggtttcgatttgccagtatttcattgaggattttcgcatcagtgttcatcagggatattggtctaaatttctttttttttgttgttgtgtctctgccagatatcatttggtatcaggatgatgctagcctcataaaatgagttagggaggattccccttttttctattgattggaacagtttcagaaggaatgataccagctcctctttgtacctctggtagaattcggctgtgaatttgtctggtcctggactttttttggttggtaggctattaattattgcctaaatttcagaacctgttattgatctattcagagattcgacttcctggtttagtcttgggagggtgtatgtgtccaggaatttatccatttcttgtagattttctagtttatttgcgtagaggtgtttatagtattctctgatagtagtttgtatttctgtgggatcagtggtgatcccctttatcattttttattgcatctatttgattcttctctcgtttcttctttattagtgttgctaatggtctattttgttgatctttcaaaaaatcagctcctggattcatttatttttttttttgaaggttttttttctgtctctatctctttcacttctgctctgatcttagttatttcttgtcttctgcgaggttttgaatttgtttgctcttgcttgtctagttcttttaattgtgatgttaggatgttgatttagatctctcctgctttctcttgtgggcatttagtgctctaagtttccctctaaacactgctttaaatgtgttccagagattctggtatgttgtgtctttgttctcattagtttcaaataacttatttctttctgtcttaatttcgttatttaccaagtagtcattcaggagcaggttgttcagtttccatgcagttgtgtggttttgaatgagattcttaatgctgagttctaatttgattgcactgtggtctgagagacagtttgttgtgatttctgttcttttacatttgctgaggagtgctttacttccaattatgtggtcaattttagaataagtgggatgtggtgctgagaagaatgtatattctgtttatttggggtgcagagttctgtaggtgtctattaggtccgcttggtccaaagctgagttcaagtcctgagtatccttgttaattttctgtctccttgatctgtctaatattgacagtggggtgttaaagtctcccattattattgtgtgggagtctcagtctctttgtaggtctccaaggacttgctttatgaatctgggtgctcttgtattgggtgcatatattttaggatagttagctcttcttgttgaattgatcactttaccattatgtaatggccctctttgtctcttttgatcttcgttggtttaaagtctattttatcagagaccaggatttttttttgctttccatttgcttggtagatctttctccatccctttattttgagcctatgtgtgtctttgcacgtgagatgggtctcctgaatacagcacattgatgggtcttgagtctttatccagtttgccagcctgtgtcttttaattggggaatttagcccatttacatttaaggttaatattgttatatgtgaatttgatcctgtcattatgatgctagctggttattttgcccattaattgatgcaatttcttcttagcatcgatggtctttataatttggcatgtttttgcaatggctgctaccggttgttgctatccatgtttagtgcttccttcaggagctcttgtaaggcaggcctggtggtgacaaaatctctcagcatttgcttgtccgtaaaggattttatttctccttcacttatgaagcttagtttggcgggatatgaaattctgggttgaaaattcttttctttaagaatgttgaatattttccccctactctcttctggcttgtagagtttctgccgagagatctgctgttagtctgatggccttccctttgtgggtaacccaacctttctctggctgcccttaacattttttccttcatttaaaccttggtgaatctgacaattatgtgtcttggggttgctcttcttgaggagtatctttgtggtggtctctgtatttcctgaatttgaatgttggcctgccttgctatgttggggaagttctcctggtgaatatccttaagagtggtttctaacttgattccattctccccatcactttcaggtacaccattcaaacgtagatttggtcttttcacataatgccgtatttcttgaaggctttgttcatttcttttcactcttttttttctaatcttgtcttctcgctttatttcattaatttgatcttcagtcactgatatcctttcttctgcttgattgaatcagctactgaagcttgtgtatgcttcaccatgttctcatactctggttttcagcttcatcaggtcatttaagctcttctctacactgtttattctagttagccattcgtctaaccttttgtcaaggtttttagcttccttgcaatgggttagaacatacttctttaacttggagaagtttgttattactgaccttctgaagcctacttctgtcatcttgtcaaactcattctgcatccagttttgttcccttgctggcgagtagttgtgtttctttggaggagaagaggcgttatggtttttggaattttcaacctttctgctgtggtttctccccatctttgtggttttatctatctttggtctttgatgttggtggcctacgtatggggttttggtgtggatgtcctttgtgttgatgttgatgctgttcctttctgtttgttagttttccttctaacagtcaggtaccttagctgcaggtctattggagtttgctagaggtccactccagaccctgtttgcctgggtatcaccagaggaggctgcagaacagcagatattgctgcctgatccttcctctggatgcttcgtcccagaggggcacccacctgtatgaggtgtctgttggccattactgggagatatctcccagtcaggctacacaggggtcagggaaccacttggataacagtctgtcccttatcgggagctgaaacgccatgctgggagaaccattgctgtcttcagagctgtcaggcagggacgtttaagtctgcagaagctgtctgctgccttttgttcagatatgccctgcccccagaggtggaatctagagaggcagtaggccttgctcagctgtggtggtctctgccgagtttgagcttccctgctgctttgtttacactgtgagcatagaactgcctactcaagcctcagcaatggcaaacgcccctccccccaccaagctcccacatcttaggttgatctcagactgctgtgctggcagcgaacaaggctgtgtgggcgtgggacccgccgagtcaggcatgggaggggatcttctggtctgcctgttgcgaagagcattgggaaagtgcagtatttgggcaggagtgtatcgctcctccaggtacagccactcatgccttcccttgggtaggaaagggaaatcccccaaccctttgcacttcccaggtgaggcaatgccccaccctgcttcggctcgccctccatgggctgcacctgctgtccaaccagtcccagtgagatgaaccaggtacctaagttggaaatgcagaaatcacctgtgttctgcaaagatcttcctgggagttgtagaccagaactgttcctattcagccatcttggaagcgaccatgaatgccatcttacaaatgaaagtgttaattggtcacagacattcactggcttttgtgtatattaatacttctgtgaatttaaatggatataaatattggttataatattccactagtgcatttggatattcatttatctagcaatttatatttcacagagaaaaatactcataaatattatattacttttcttttattaggttaattggaattcagttagtattttagaaaggaaattgtaatggaagaataacaggaaaaaataaaagtccaataagtgagcctaaggacttatattgtactgagaattggttttagatttgtcagggaagagagaagaaacagtagacatttttgtgtacgttgaagtcactaaaagaacaatgtaaagctcttttagatcagatactaggtagcataatacacagaatcgattaagaattcagaaaacaagatggtgactgtggactatccctagtgaagtaggaggtggctcttgtgcctgaattgaagagagccgttggattgctactggggtgagagaaggcaaaaaaggaaagaagaggtctgtgagtgaatagagaccctggagccagtgatatgcttatataattgggcctcagttcagaatgggttggattatttaaatttttatatctgttaaccccaaattaacattcttttctccacctgagctttctctgagaggtcttctactttgctaaaaagaagattcagggaagcaaacactcaaagccttactttcctaaatgcagaaggatctgaagagagaagttttaaacatttttatttgcacatattttattttcaagtaaaataaggtcttgaagaaagccctaaggccttttagaattggatggataaggtcagactcttatgggttcaccttttgctatttgaccatgggcaaatttaacattcttcctattacctataatatgggaacagtactgtctacctcatggggttgaaactaagcattgtgtctggtctatagaatgactgaatacatgggagtttaaaaaatgacttataattgaaacaagtaattgaagtaattacatgtaataaactaaaaataatttgtagtaaagaatatagtttactttgaccaggcgtggtggctgatgcctgtaatcccagcactttggaggccaaggagggcggatcacctgaggtcaggagttcaagaccagcctggccaacatggtgaaaccgcgtctctgctagaaatacaaaaattagccagacgtggtggcaggtgcctgtaatcctagctactcgggaggctgaggcaggagaattgattgaacccggcagacggaggttgcagtgagccgagattgtgtgactgcacaacagcctgggtgacaagagtgagactccatctcaaaaaaaaaaataaaataaaaaataaagaatatagtttaccttgtatattttggcagttttctctatttaattccttacagttctcgtgaaaaaaaaatttcctttcttcctttaggagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaaggtaaaatgtccagtatttcatatattttgttgctgtcgtaatacatataccataaatgattatttttttagttgcttctttttgcttcaaatttcttctattgtgcgcatttttgttcagctcaaccattgtagtgattttatttctagtcatgtattttgtagagataacattcactgaagattgcagtattacttttgtagtcattacaaatgacatatgtaatatatgtattttttaatcctgtgaaaggtaagttaaaatttcttaatttatactataaaaagtgacctgtcctcaccgttgtactgtgttgatgtagaccagaggaacactgcctgtagcatataagcattttatgtatttgactatgcttccttttttcaaaaaatttttttaatccatgttgcaacttgtgttagtctgtttttgcatctctataaaggaatacctgatattgagtaatttataaagaaaagaggtttaattggctcacaattctgcaaactgtacaggaagtgtggtgcagacatctgcttctagtaattgtctcaggaaacttctaatcatgggcagaaggtgaagggcgagtaggtgcatcacatggcaagagctgagcgagagcatgaagggggagatccagactttattattattattattgttattactattttgtagaaacggggttttgccatgttgtccaagctagtctcaatctcctgagctcaggcaagtcgcctgtctcagcctcccaaagtgctgggattacaggcgtgagccaccatgcccgtccccagactctcttaaataaccagatctcctgtgaactaactgagtgggaactcactcatcaacaaggggatggtgctaaccccattcatgagtggttgacccccttgatccaatacctcccaccaggctctaccccgtctctactaaaaatacaaaaaattagccgggtatggtggcgggcgcttgtagtcccagctacttgggaggctgaagtaggagaatggcgtgaacccgggaggcagagcttgtagtgagccgagatcgcgccactgcactccagcttgggggacagagtgagactctgtctcaaaaaaaaaaaaaaaaaaaaaaaaggagattacatttcaacatgaggtttggaaagggataaacatccaaaccatattacaacactatccatttttctcgtaactttttttttttttttttgctatcttcccttttttttcttgtccttctgcctttagacactagcaatcccaaaattcagttctcgtcatgtattttcattttctgtacctcattaggggagcctatgcatggaagctcagatgtcatctctatgttgtggtatagcagagagagtcagaagacaccgggtgtaattaagcctctgtcactatgcagcgttgctccctttactactcttccaacctctgcaacattttagtgggagcctccaatgtaccaagatctatggtaaacatgaagggatataacaagaagaagtgatcactgttctggtataacactagttataccacattctagtgtccatctgctgtaaatatctggaacattctaaagaatttttaaaaattcagttgtttttgttttcagttctcacctctcccccaagtactagtctcatgttgtcaaatgcccactggatcttgatatctgaatattgtactgttatctcaaattgtcaagccatcttatctaactatgacttaacttttaagactcaatggaaatctctcctgctagttcccagaactttggatttgccttttctttttaatggttttataattcactgttattcagactgttccatttgctcatccctaatatccagtcattgtttaacttgattttcttttttcctcatactgccttgtggctgtgtctcttctttttctttctcttcactactattctagtcagaacctgtcattatttggcttaaccacttataaacgtgtaccttagtcttctcctttctaatctcttctgtgtactaagactagaataatcccagagttctcattggtcatgttgtttctcttgcccataaatcttctatggttcttcatagtctagaaagtaaaacctgcctagtttacttgtgtacgcagaaacttccatagtcttccattttctccagatacttaactttccaaactttttatgttactctccttgaaatatgttcttctctatgaccactctgcttttattcagctcttctatcactcccattctcctgctgtctaaattctgtgcatttatgaaaactcaggtcaaattcttcctgtatgaaacttcccagatgattcaagtcagctatggagatcttgctattctaaattcctcgagcccatattgtccataacctaggaaattccatattttttatgacagggtctccattttttaaatatttgtctcctaaatacccacacattccattttttgggattggtagttaatgatgatagcagatacttactatatgtattttttgacatgagcagttttacataattcttaccattagtactgtgtgattgatattgttattatttggataaggaaacctaagtacaaagaaattttaaattaacttgtcagggtcacacagttggcaagtggatggatgagcaaggattcgaatctagacagactctccagtgccttaccacattttatcctcgctgtatcaagcccctctaccccttctctctttctctcacagtattcatcctctttctatatcatagtatcagattctcacagtgtcataacctctttctatcaaaatatttaaaccacaccctctttctccttctcaagagagattggttttatttcattctctaatggttgcattatattttattgtgtggatataccctaattagccattgttcaattgatagctattaaaatttttttccctgccaaacattgtatatatgtgctttatcagaatacagtttataaaagtagaattctagggtttaaactttaaaccttgatataggagagcactttaaaccttgatataggctgtcttccagttagattttatccatttaaatgtacgttatttgaatatgagtaatgcccatttcctgtgtagcctttagatattaggagtatttttatgtttttcagttctgtaggtgaaaaattatttttatattttgttactttcttttctgttttaaattttttgtagagacgcatctcactacatggcccaggttggtcttgagctcctggcctaaactggttctccctcttggcttcctaaagtggtgggattatggtctgaatcatcatgcctggccctgaaaaattattttaatttacaacaccattttacatgttgtgaccctttttaccccttttagagtgtgtgtcttccttactcatttgttaaaacccatttaaaatgtaatcttgtagagcaacaaagattttattatctcagtgcttctgtgcgttggaaattcaggcatggcttagctaggaacctctggcccagggcctctcaccagactgaatccaggcatcagctggagctgcagtctcatctgaaggtttgtctgaaggaggaaatatctgtccccaagtgtactatgtgattgttggcagactttggtcccttgccacatgggcctgttcactgggctttctgaggtcttaccagttagcttcctacatggcaagggagccactggaaaaacagtaagcccaagacagaaaccacagttgtttttctaacctaatattggtgagttgattaaacaagggtgtgaataccaggaggcaggtatcatttagggccatcctagaagctgcctccacaataatatgaaccatctgtcatcatctcaaagtgagaaaaaataatttcagtttgtgttgactttgcttgtgatatcttttgcttttcagatattgttgatttgggctttggtataatgctgagaaagggcctgttctatcttgcttataaataatgtcgttttctctaggaattttattttcacatatttctttaatttttctggggttatattttgggatgtagtgtaaagtagtaatataacttaacctttccccaagtagtaggtcattgtctcagaaaccatttttataaagtttatccttttcccactggtgggcaaatgccattttaaaaaccatgtattatgttaccatatatacattggattttcttctgggtttccttttcttttccgctgatttgtccattttggcttatgtaacacttctctgatgatgagaactaacagcatattttgagtgttccttgttttttctcttaaaaaaatctgtgtatcaaatatcatcagcctgtgatgtttctcctgcaaccccaacaaaacaaaaacaaactttagatttgattaaaattgtgtaaaatttgcagattagttgggcagaatgtgcagatttacaatattcagccttcccatccagaaatgtgataattctcagcatttaatccatttttgatataaaactttatagttatgtacgttttaaacttttcttacttttttaatctagatatgtgtagtaatttttctgttgctcttgggaatgggagtcttcctccatgtaaatacatcttccatgtaaatgcaagtcttcctccatgtaaatacatcttccatgtaaatgcaagtcttcctccatgtaaatatgtcttgttttaggcatttgaacactgcctcctccgttggttttgaacttgggcatcttctagatgcatttaatgtgctgaggccctttagtagggtactttactatgttgcttttctaaatattattacatgttcattataattttctagcttacctagaaacctgagtttaaaatgttactggtgaactagagtgtttttgttgttttgttttctgttgttagatgaacagtttttattatgattagacactgattttcatatagggtttatggtttttttccctatgatctaaatgtaatatataaacatgataatattcagtcggttttgctttctaataatcctgtttgctccaggtgttttaacattgtgattttttttggaggtattttatgtattagtgctttcttttattttttgtttttctcagtcagacttgctatatagctttgctttagttatcatctcttttcaaggaaccagctgttctacctttcttctaatcattgtatctttcatcactattcctttttaagaaggttctaaagaaagaatatgtcaaatactttgattaaagattgaaaatatgtccctatgtaaatgaactgtagaactctgaaaacaaacatttaaataaaactattaataaacagtgagaccctttctttttttattaaaaaaaaaaacactaaataaaactatttatattgggtagcatagggtaaaacacagtaatacttctgattgtcctcactgggaatttgaatacttttcatgtattacagagaggaacaagtaacctggctgaaaataaaaggatgtgagtcaggaaaataaaataataaagcaagagagcacccaaagccttggcttgcaagaaattgtgtattagatactatggtttttatatgtacttattgacagagaaagtggggatctattgcttaggtaacaaaatagtagatctaatgttttattatgatttttgtccttttttcatgttttgatggagaagatgtacagacagtggtttaagaataacaaacacttgaaacttgcaacttagcctatgctaggattgtgtgggtatttcaccaattaatttaataaatacagagttttgtctaatgagcggtttgaaagtaataatgtgcacacatagcattttgtagtttaaaaattagtttcatatacataaatcatttgatttttgctactttttattttgatataatttcaggcttaggccaggagtggtggctcacgcttgtaatcccagcacttcaggaggctgaggcaggaggatcacttgaggtcaggagtttgagaccagcctggccaacatggcaaaatcccgtctctactaaaaatacaaatattagcaaagtgtggtggcacgtgcctgtagtcccagctatttggaaggctgaggcaggagaattacttgaacctgggaggcagaggttgctgtgagccaagattgcgccactgcactctagcctgggggacagagtgagactctgtctcaaaaaaaaaaaaaaaaaattatttcaggcttaggccgggtgcggtggctcatgcctgtaatcccagcactttgggaggccgaggcgggtggatcacgatgtgaggagatcctgaccaatatggtgaaaccctgtctctactaaaaaatacagaaattagctgggcacggtgactgaggcaggagaatcacttgaacccgggaagcggaggttgcagtgagctgagatcgcgccactgcactccagcctggtgacagagtgagactccgtctcaaaaaaaaaaaataattattgtttcaggcttaacattgccattactgtaaagaacttccttaaaccttttatccagatttccaaatattaatatttatcacactcacttttgcagtttcttcctctctatatatattatttttatttttttcaggtgagatcttgccgtataacccaggctggagagcggtggtaaagtcacggctactgcagccttcaactcctaggctttagcgattctcctacctcagcctcccaagtagataaccataggtgtgcgccaccacacctggctagtttttaatttttttttttttttttgtagagacagtgtctccctgttttgcccaggctggtctcaaactcctgggcttaagtgatcctcctgccttggccttctaaagtactgggattatagacgtgtgtgccaggctacacatattatttcctgaaatgtttgagtgcaacttttaagtatgaggctcttttatccctaaattattcagtgtctgtttcccaaactcaaaagacattcgcttatgtaaccacataataattataaaaaaatcaagaaattaacaattatacaatactattatctaatatgcaggctttattgaaatatctccagttgttccaaaaatgtccattgtggcacaagaaaatcctgggtcatgtgtttagttgtgatatctctagtcttctataccaggaagagttcctcacttattgtctttcatgacattcatgtttttgaacagtataggacagctatttgggtttgtgtaatgtttcttcataattaggttcagcatattcatttttggaaagaatgtcattgaaatgatttttttcttcttaatgcattatatcaggagacccatgatgtctgtctagttaatgatgatattcacttctatcacttagggtagtgtttgctaggtttctttactgtagaattacggtttttctcttcataattataaggaatatgtaggaaaatacttttagactacataaataacctttttctcatccagcttttaccaactagttttagtatgcattaatgagtttttgttagaatcaattattataataatggctacaaatggtgattttctaattctttttttctttattaattagattctactctaaggattatgaggaaagcttttcccttctctcatttaattttatatatcatacggactcattgactctcatttaattgtatatatcatatggactcattgactctcattttattaaatgggttataatctttggtcatttattttgatactcagattgcttcaggccaggtgcagtggctcacacctgtaatcccagcacttagggcggccgaggcgggcggatcacgaggtcaggagattgagaccatcctggctaacatggtgaaaccccgtctctactaaaaaatagaaaaaaatagctgggcatggtggcgggtgcctgtaagggaggctgaggcaagagaatggcccaggaggtggagcttgcagtgagctgagattgcatcactgcactccagcctgggtgacagagcaagactctgtctcaaaaaaaattgcttcagatttggtcagcgaaagccctttcaagctggatctagatccccttagccagtagaactagagaataggtgtatgtgtgtgcacatgtgtgcaagcatattcacacgtttttttctgtatttctttctattaaaaattaatacttttaattgcagagcaacatcacagagttcactctagccttctttatttctatatttgtaactcctctttctgaactgagaaaacctagctgctgttatccacaatatatttacttatttgctcaatcctagaatttacaagctagtttcagagctgctaactcatatccctgggggcagagttggacttactaagagttcactgtttatgttttttatctttagcttgaaaggcatatggattttatattatattcaaaaagtatttgggtttttctttttttttaattttaattttttcctgtagcatagactattaatttgaagtagttaggttcatctgtttctgtatgtatcgcttcttagaacttcaccccctttctttttgattaaaaaaattatttatgttttcattctgtgaaacactaacatggttccaaaagtcagaaatctagtgtcatttcttttcgattctttctattttatttccactcactccctgtagatggccaacctcattatgttctggcttacttttcctcagttttgttttacacatacctgctcgtttgtgcattttctttttttttttttcttccttacacaggaggtagcatattggagacactcttttgtactttgctttttctttaagctatatttcctggcaatcattgcatatcagttcagagatttttttttttttcatcctttttacagctgctaaatgcccctccataaaggttataccaatttgcattctctctagttaagtatgagtgcctgttggtccatagttttggctataaaagttgccttactttttaaatttttgctaatctcataggtgagaaatggtatctcattatagttttaatttacaatcttcctattttgagtacactttaacatcttttcatgtattcaaggtccattttatttgtttttgtgagtctgtatgaattgtcctttttttggtccattttaaaaatgtgattttgatttttcccaatcaaccctgaagagtactttattatggaatttatattaggtgtgatatgtgtaatttttactttgtgtatggtagttttttttttctgatatgtaagagtccttccttccctctatgcagttgaatttattgctcttttatctggattttgtgttatggttataaagctttccagctatcctcaggttaaagaggaagtcatctatattttcctctagtagttatcagggttcagtttttgcatttagatccctgatccatttggagtttattcctattatggtatgaattattgatctaattttatcttttctaaatcattaactacttgtctgggtacaatttagtagaagactatctttgttccagttatttgagatgttactgttattatatgctaaatttctacatgtacttggctctatttctggactttccattttattccactaagctgtttgcatattcatgtgtcactactataccgttttgattatagagactttatagtatgttttaatgtctgatagggttaatatgcccttgtagcttttctttttggtgttttcctacctatttttacatgtttatttttctatatgaactttggcatcagtttatctaactttataaaaaaagtgttgatcttttaaattgggattttatttataaattaatataggaagaactgatatatttgtgagcttgaattatcttagccaggaataagaaatatttttccatttgttcaaatctacttttatgtctttcagaagttttggcttagtcaggctttagaaatttcttaagtttattcttaagtattttattgtttttgttgctattgtatagtttttcttgttttttattctacatgttaaaaagtgatggactgctttataccaagctcggtttttttccttgtatcacccttcctttgtggaatttaagtattctttgttgtggccaaaaaataaaatttgatatattgacctatttgtttctcttgattatcataaaataggaatttctctgacaacttaaaagtctgtagttttaaaatctgtgatttacatagctgctgtttgggattgccttaaattattaggtgtttccagcagaaggttgaaataggatcttctccttgttcaattaaataaaccaacctgcgaaatctcctaatggcagtgagaggctttttttttttttttttttttggtgaatagtaaagatgagagataatggtcaaagataaaaagaaaaccaagaataattatccctgtgtgccaggagtcacataaactgtacctaagacactgggctttttaaaagaatttctggtataatcttcatttcagtggtattttatttttttcccactgttgtccatcatactgtgacagagtacttaaactatcttaacttgggcagtttggaagtgcttgtgataggaaatatttgtgttgtggggataaagattgggtaggtacctaattaatgtaggagacctgtactgttgtaacagggagaacatcagtcaatcccaaacctccttatggtttgttgaggctgactgttccaacttgaaaatcagtaagttgcatgtttattagtgatcttggcttcatttactattcatttctcctttgaagtgagtcataggtttcaagtctcctgcctagtttcacttgttctgtaattgggatatagagggatagggatgtgaggtatcactattgttaggttgtgaatggagagcatagtctacttttaataccattttatttatttatttattggagacagaatttcactcttgttgcccaggctggagtgcaatggtgcaatcttgtctcactgcaacctccgcctcctgggttcaagcaattctcctgcctcagcttcccaagtagctgggcttgcaggcatgcgccactactcctggataattttgtatttttagtagagatggggtttcaccatgttggtcaggctggtctcgaactcctgacctcaggtgatccacccacctcagtctcccaaagtgctgggattacaattgtgggccaccgcgcttggcttaataccattttaatacctgcttttatagcattttggagaatgaggaaagatgatgtctgtttttgatccttaaggatatgagtattctctgaatatattctggagacagtgccagaggaagattttggtcttacttaataattttgaaagcttgagcatatctattgtaagagttaaagaaagaggaaagaaacacgaaatgtggctggcagttaaagacaagttcactttagacaaaacctgagaggtgctcctggctgattttgatcaggagcgctttctcttacagactaagagtatatattggttttagggtgagggggcttatcacaagattggaatgtttatgtgtgtggagaagtttatggtggggttagaatctctctgggaggaggggaggttatcttggggcagacatctttccggccaggaggggtgttatctcggggctagcatctttccaactggagcggggtttatttcggggctagcatgtctctggtcagggaggagtttggaatgtttctggttggagatgttatttgtggtttatggtcatgctgaccttagccattaggctgatgccctttgaatttaggcagttttttattaaggtgaattttagaatgaggggcttgtccaagatggcgatgctcctactctgtcaatccagaccctataatttgaggagggacggcgtgttcttctggctacttcctgctgactaggggatggagagttttctggtctcaggttgactgtaggagcaatgacatctgtagatgtttttgggtagttgtctgtgaaatggccatgatcctgtcagataaaaatctttgaaaaaggttaattgggcagggtaagaacattagtcccaggcatattattagaagggggctcaggaatgggatgacacatgctatgattttgtttccaaaccaagaatctgtttggttgtttagatatttccttagctttttatttccttttttttttttttggagtcagactcttgctccgtcacccagactggagtacagtggcgcaatctcagctcaatgcaacctccaactcccaggttcaagcagttctcctgtctcagcccccgagtagcttgggactacaggcgcctgcctgtacgcccggctaatttttgtattttttaagtagagatggggtttcaccttgttggtcagggtggtcttgaactcctggcttcaggtgatacatgtgcctcagcctcccaaaatgctgggattacaggcgtgagctacttcacctagcaagatttcttatttctttacattgatatttatgacttttgggggctgtcaggggttgcttccttagctttccaggctttgacttgagtgtgatgtatttaggagttgattcctgtaacaggtaccattttaattaccctgggttccgtggattcactagacatgggggtgaaggattctgggcaccctcagttattagttgtcagcaccagcagtgaagagatttccttctgtgatggtcggggagcttagaggcagcgtctgctgaaacatctagttttcagtttatagggctttaagaaagcccgacttattttggaaacttgtagccagaaaaattagaatttaatttaagcagtagaaaataataaaaactgaaaaatgttaggcaacactagaatttaacaacaggtgtgctatggttttttaaatataattttctttttccagtttcccatttttattaaaagacaaatcatggtaggaatggtttgctttattatacttggcttaattatttgcatacagtgcagcaagaataattatttgttacataggccttttaaattggctttgatggaactttgttctgtagaaggaatctgagagaaaaccgttttagccaagcccagccctggatttgtactatcaaatatctatgagttggtgaatttctttcctcttgaggtcccaagataacttggtattcctggtctgctagaaagtgacattctttacttaccatagaccagaaaccctgtacagggactgtgtacacaaaatatgaggccagttttccaagggcatttttgcctttgtaagtatggttccttaaaggaaagcatattattccagttaaagcctttgtaaaaataaccagtttttccaattctgtcctgttacaaaagaaaacagatttttagtgcacttatgcaagtactgtaacttaagaatactcacagatagtttcaaaattatggagaaaatcaggtagagagaaacaagtatgttccaaattttgttcatggtagtatagtaaattgttaaaagctgttaatagctcaaaagaaaaatttctttgactttgaaaagcaaaacaaaggattagcaatatgttaaaacattaaaaagattggtctcctgttagtttagttcatggaattaattcctgtcatgctcaatattaacattttatctcttcaaaagtcctgaacgtttttcctctattctgatgttacagtctccagtgttatcagaaacctgcatttaagagcacctgttagagctttatagctgattataaaaccaccttttaaagaggaccaaaacaaggcaacaattgtttatggatgacaaaaagttttagggtagccataaagacacaattgacaaggatgacacacaataattttaacataacgattgtaattattactgaaaatgtacactaatatatatcagaattataggagtcttccataactttggaacacatatgagtaacatacaaatatagaccaaagaaagccaaataccgttttatatttgacaatgcttcttgtatgattttataccagataagctaaatttcacctttctattagtgtgctattaatgttaaactcaattttaataagaccttgtaggcatatttatccaattttaatatctgaccataaggtaagatttttatagactttttttaatgctttataagttttgttaaagaacaggttagtgctttaagaaaaacccattgtgcttttgttttaatgcccaatttacagaaaaactggatgatacccctttaactttaccaatatgtttacacacagaatttcctttataattaacgtttcaaaacttgcttaaaccttcaaaacaattttttaactttttaatgtaggtaaaaatccacattcttatgcctccttataatccttttacgaaaagtatattttactttccttacacaccttgcatataaactgttttttcaatagctttaaatacatgttacaccattaacttttattattatactttaagttctagggtacatgtgcacaacgtacaggtttgatacataggtatacatgtgccatgttggtttgctgcacccatcaactcatcatttacattaggtatttctcctaatgctatccctccccgcagtccctcatccccttacagtccccagtgtgtgatgttccctgccctgtgtccaagtgatctcattgttcaattcccacctgtgagtgagaacatgcggtctttggttttaggtccttgtgatagtttgctgagaaggatggtttctagcttcatctgtgtccctgcaaaggacaggaactcatccttttttatggctgcatagtattccatgctgtatatgtgccacattttcttaatccagtctatcattgatggacatttgcgttggttccaagtcttgtgaatagtgccacaataaacaaacgtgtgcatgtgtctttatagtagcatgatttataatcctttgggtatatacccagtaatgggattgctgggtcaaatggtaattctagttctagatccttgaggaatccccatactgtcttccacaatggttaaaccaatttacactgccaccaacagtgtataagcattccagtttctccacatcctctccagcatctgttgtttcctgactttttaacgaccgccattctaactggcgtgagatggtatctcattgtggttttgatttgtgtttctctgatgaccagtgatgatgagcattttttcgtgtgtctgttggctgcatagatgtcttcttttgacaagtttctgttcatatctgttgcccactttatgatgggggttgtttttttttttgtaaatttgtttgagttatttgtagattctggatattagccctttgtcagatggatagattgcaaaaattttctcccattctgtaggttgcctgttcattctgatggtagtttctttcaccgtgcagaagttcttcagtttaattgatcccatttgtctattttggcttttgttgccattgcttttggtattttagtcatgaagtccttgcccataactatgtcctgaatgatattgcctaggttttcttctagggtttttatggttttaggtctaatatttaagtcttttatccatcatgaattaatttttctataaggtgtaaggaaggggtccagtttcagttttctgcatatggctagctagttttcccagcaccatttattaaatagggaatcctttccccatttcttgtttttctcaggtttgtcaaagatcagatggttgtagatgtgtggtattatttctgagggctctcttctgttccattggtctatatatctgttttggtaccagtactatgctgttttggtgactgtagccttgtagtatagtttgaagtcaggtagcatgatgcctctagctttgttctttttgcttaggattgtattggcaattcaggctcttttttgattcaatatgaagtttaaagtagttttttccaattctgtgaagaaagtcattggtagcttcatggggatgacattgaatctataaattactttgggcagtatggccgttttcaccatattgatttttcctatccatgagcatggaatattcttccatttgtttatgtcctcttttatttcgttgagcagtggtttgtagttattcttgaagaggtccttcacattctttgtaagttgtattcctaggtatttaattctctttgtagcaattgtgaatgggagttcactcatgatttggctctctgttggtctgttattggtgtctaggaatgcttgtgatttttgcacgttgattttgtatcctgagactttgctgaagttggttatcagcttgagattttgggctgagacggtggagttttccaaatatacaatcatgtcatctgcaaacagggacaatttgacttccttgtttcctaattgaatgccctttatttctttctcttgcctgattgccctgaccagaacttccaacactatgttgaataggactggtgagagagggcatccttgtcttgtgttggttttcaaagggaatgcttccagtttttgcccattcagtatgatattgattgtgagtttgtcataaatagctcttattttgagatacgttccatcaatacctcgtttattgagaattttagcatgaggggctgttgaattttgtcgaaggccttttctgcatctattgagataatcatgtggtttttgtctttggttctgtttatgtgatggattacgtttgttgatttgcatatgttgaaccagccttgcattccagggatgaagcctgctggatcgtggtggataagctttttgatgtgctgctggattcaatttgccaatattttattgaggatttttgcatcaatgttcatcagggatattggtctaaaattctcctttttttgttgtgtctctgccaggttttggtatcaggatgatgttggcctcataaaatgagtcagggtggatcccctctttttctattgattgaaatagtttcagaaggaatggtgccagctcctctttgtacctctggtcctggactttttttggttggtagactattaatcattgcctcaatttcagagcctgttattggtctattcagagattcaacttcttcctggtttattcttgggagggtgtatgtgtccaggaatttatctgtttcttcttgattttctagtttatttgcatagaggtgtttatagtattctctgatggtagtttgtatttctgtgggatcggtggtgatatcccctttatcattttttattgcgtatatttgattcttctctcttttcttctttattaatcttgctaccggtctatcaattttgttgatcttttcaaaaaaccagctcctggattcattgatttttttttttttttaaggtttttctgtgtctctatctccttcagttctgctctgatcttagttatttcttgccttttgctagcttttgaatttgtttgcttttgcttgtctagttcttttaattgtgatgttagggtgtcaattttagatctttgctgctttatcttgtggccatttagtgctataaatttccctctacacactgctttaaatgtgttccagagattctggtatgttgtgtctttgttctcattggtttcagagaatatctttatttctgccttcattttattatgtacccagtagtcattcaggagcaagttgttcagtttccatgcagttgtgcagttttgagtgagtttcttaatcctgagttctaattttattgcactgtggtctatgagacagtttgttgtgctttctgtccttttacatttgctgaggagtgctttacttccaattatgtggtcagttgtagaataagtgcagtgtggtgctgagaagaatgtatattctgttgattttgggtggagagttctgtagatgtctattaggtctgcttgttgcagagctgagttcaggtcctggatatgcttgttaaccttttgtagttgatctttctaatgttgacagtggggtgttaaagtctcccattattattgtgtaggagtctaagtctctttgtaggtctccaaggacttgctttatgaatctgggtgctcctgtattgggtgcatatatctttaggatagttagctcttcttgttgaattgactcctttaccatcatgtaatggctttctttgtctctttgatctttgttggtttaaagtctgttttatcagagactaggattgcaacccctgcttttttttttttctctccatttgcttggtaaatcttcctccatctctttattttgagtctgtgtgcgtctttgcatatgagatgggtctcctgaatacagcacactgatgggtcttgactctttatccaatttgccaatctgtgtcttttaattggggcatttagcctgtttacatttaaggttaatattgctatatatgaatttgttcctgtcattatgatgttcgctggttattttgcccattaattgatgcagtttcttcatagcatcagtggtctttacaatttggcatgtttttgcagtagctggtaccagttgttcctttccatgtttagttcctccttcaggagctcttgtaaggcaggcctggtggtgacaaagtctctcagcatttgcttgtctgtaaaggattttatttctccttcacttatgaagcttagtttggctggatatgagattctgggttgaaaattcttctttaagaattttgaatattggcctccactttcttctggcttgtagggtttctgccgagagatccactgttagtctgatggccttccgattgtgattaactcgacctttctctatggctgcccttaacagtttttccttcatttcaaccttggtgaatctgtcaattatgtgtcttggggttgctcttctcttggagtatctttgtggtgttctctctatttcctgaatttggatgttggcctgccttcctaggttagggaagttctcctggataatatcctgcagagtgttttccaacttggttcccttctccccatcactttctacaccaatcaaacgtagatttggtcttttcacatagtcccatatttcttggaggctttattcatttcttttcagtcttttttctctaaccttctcgctttatttcattaatatgatcttcagtgactgataccctttcttccacttgatcgaatcagctattgaagcttgtgcatatgtcatgacgttcttgtgccatgattttcagctccatcagtttatttaatgtcttctctacactgtttattctagttagccattagtctaatcttttttcaaggttattagcttccttgcgatgggtttgaacattctcctttaactcggagaagtctgttattaccgaccttatgaagcctacttctgtcaactcgtcaaagtcattctccttccagctttttttctgttgctggcgaggagctgccatcctttggaggtgaagaggtgctctgattttagaattttcagcttttctgctctggttctcctcatctttgtggttttatctaccttcgttctttgatgttggtgacctacagatggggttttggtgtagatgtcctttttgttgatgttgatgctattcctttctgtttgtttgttttccttctaacagatccctcagctgcaggtctgttggagtttgctggagttccactccagaccctggttgcctgtgtatcaccagcagaggctgcagaacaacaaatactgcagaacagcaaatattgctgcctgatcttcctctggaagcttcgtcacagcggggcagctgcctatatgaggtgtctgttggcccctactgggaggtatctcccagttaggctatacgggggtcagggacccacttgtggaggcagtctgtctgttctcagagctcaaatgccgtgctgggaggaaccactgctctcttcagagctgtcagatagggacgtttatgtctgcagaagttgtctgctgccttttattcagctatgccttgcccacagaggtggagtctagaggctgtaggtgttgttgagctgtggtgggctctgcccagttcgagcttcccggccactttttttacctactcaagcctcagcaatggcagacacccttccccagccaggctgccgcctgacagatcgatctcagactactgcgctagcagtgagcaaggctccgtgggcgtgggacccgccgagccaggcacgggagagaatcatcttgtcagcgggtgctaagaccttgggaaaagtgcagtatttgggtgggtagtttcccatttttccaggtagtctgtcatggcttcccttggctaggaaagggaaacccccgactccttgcgctttgtgggtgatgcaacgccccaccctgcttcagcttgccctccatgggctgcacccactgtccaaccattcccaatgagatgaaccaggtacctcagttggaaatacagaaattagccgtcttctgcgtcgatcatgctgggagctgcagacaggagctgttcctattcggccatcttggctacgctgttaacttttagtaaactttacttttgttgaaaaccttgtaagtttgggattttaattatgtgctaggtgtagagcccaggacctagaaagaagtgcagttaaatgttctagcatttaactccacgtgtcgtaggttttacctagctgcaaagcaggccagttgtccagctaagagttacagtgacattttataaagcattcaggaggcctaattacttttaaattatacaacatttcttgtataaattcccttttataacatttttcatgactttcacagacaatccttgacatgcctcaactttctgacttgtaaacatctctttctttaaacaaccagttaacttaggacaagaatttaccatataagattctttttccataacttctcctttctttaatgtcacagatgataaccattctttcccaaagcaaacttccttcatgtttgtggactagactgcctaagccacaagattagaagctaagatattttactaaatagtttaagatacagctatcttcattaaaccaatattaatatttcatttattaaaaaattacacaaggctgggtgcggtgtctcacgcctgtaatgccagcactttgggaggccaagacgggtgggttacctgaggttgggagtttgagactagcctgaccaacttggagaaaccccatctctactaaaaatacaaaaaattccccgggcgtggtggcacatgcctgtaatcccagctacttgggaggcagaggcaggagaatggcttgaacccatgaggcagaggttgtggtgagctgagattgtgctattgtacttcagcctgggcaacaagagcgaaactctgtcttaaaaaaaaaaatgacacaagcaaagatgattttgtttgggctgagttagttttgcagcctctatgcaaaatgttgacaccttacagtatttggccgagaaaagtatgaatttgcctgattaacaaatgcaaacaaaaaggtatgctggcaacttttaagatacttctaactttagtgtaccagtaagatttaaagattaaggtcacgtgaactgaaaggtcccacagcttttacttttcccttaaaaatatttgatttaagtgcttagttttcttaggccaattaattagagctcttttaatagacattgcacagacaacacatatatagccacacagacaaccagaagaagattcagtagctataacgttttttttttctctctgctaatttcccaattggattattgaccttcaggtgaggcccttgaagaacaggactaggaaaacagtttccagggtctaataaacaggtatagctggaagacaaatacaaagattttgagaggtacttatttacctctaattccacggtttccataaggaaaacagatttttccccaaaatgggatttgtggtgccttttctcttttccaaaggaatcccagggcaccagaagtcattttagggttttttacatatgcaccaagagttgcaagacacagtgaagaaaagtaattgagttaactgggggaaaaccttttccaggaaaataagattgatgaagagaaaactataaagaccttttgaatatactcatagttttgatctgtgacaagtcagacatggagaaagagacatgaacatgagcagtgcctttaactccagccacttcccagagagggagaatggcagaggtggtttttgaatgggtaacaggtggaggaggaggaggagtggggtttggggctgaaggcttggaggcaggtagggccaccagggtggagggtttggacaggctaggagcagatgcagggggataagaatacagagagggttcatctgcagggtcaaaaggagtttcggaggaaggagagacccggagagggtttttaataagaagaaagattttataagaggtacaagcttgacacaggtagggttgggatttaaggtagaagtagaagaaggcctgaatatagagaacctcttgccatttgccattcctggtcataaagttgtgaagttcctgagaatttgcaagttaaagatgccattttcggaccatgggctgccattatctaatgtgttttggggccaggctgtgttacagtaaaagactaaacacttaagccaactttggcgaggtcatgaaggagacagcacagtggagagaccgtaggaatgtggaattgtttggcacccatgtggactggtgagaagaagccgagggtgtctgtttttgttctaggcatccccagacaaaagacagaagtccggaatcgtctttctgaagaggacagtcatcaagctgagaagaaactgggcatccccaagatttcctctagcttagtcctgctggtcctctgaggaccgggatggctgacctgacttttcctgggtactgcaagaaagccaggggacggcagatcttaccagttggctggattagtgtccaatgttggatgttccggttggatttggcaaagggcctcctggactgcagccgcacgagggagagagagagaggaagaaaaaaaaagggaggatgagggaaaagtgaagtgagagaaaagatggggcatgtggccagagaccctcaggatccaggaattaactcaagatgagctgctgttgcccactgcttcctgggttgcaagagagcctctgcccccagcactcatcccgggtttgggcaccaaatgtaagagttaaagaaaggaaagaaacacaaaacgcggctggcagttaaagacaggtttattttagacaaaacctgagaggcactcctggccgatttcgttaagaagtgtttccttttacagactaagaatatatattggttttagggcgagggggcttatcacaagcttggaatatttatgtgtgtggagaattttatggcggggttggaatctctttgggaggaggggaggttatcttggggcagacatctttccggcccagagaggggtttattttgaggctagtatgtctctgatctgggaggagtttggaatgtttcaggttggtgatgttgtttgtggtttatggtcatgctgaccttagccattagggggatgccctttggatttaggcagtttcttattaaggtgaattttagaatgaggggcttgttcaagatggtgatgttcctgctctgtcatctgtgaaatttatgaccctcaatttcactgcctgtaaaatggggacggtactaatacagcaatgcccagcccagtgtttggtacataaaggaactcagtaaacattattgtgctgccttttttctcttcttcagtgttttgtgaatcaaatggaacaaagttttatgacaacattttctaagttttaaaactccttataactaaactctttgtttttattcttaaggttgtaagttactacacactgctatacgtcaaattttttccccctattggttttatactctcttaaatttggtaattctgttttcaggcacctaattattagtattttatatagccatttaagtcacttgtaaaaaatctatttgctgataaaaccgatagaaacttgaaagaaatagtaatctttgtgctatgtgtagcactgttctttaagtcatggaaatttgtagttttcaagtggaggctataattaggtggaagaaaagggaaggacagctacaagttggataggaaaagtgttacattgccagtacagctctaagaaagtaacagcatggcaatgggagtcctcaaaccaaagttaccaagtcagaggagttcagtttttcctgggaaaaggtctccattaagtatgcctgctgttctcagtcatataggctgggaatctttgcctgtccccagcaagagaatggatttgataggtcagcagttgggaccattgataatgatgttccttgcagtgggaaacctgagaggcaggttttcatacaaagtttcattaaaactttcatacagagtttcttaaggatacagactgttttatgctgcttttatagtttctgaagagcctaggtaggttaaatacatttttaaaattttaatggaattttcagggaattaacaaagattgatgtggaagaactgtattgagtttgctgttaaaattatatatagttttattgcctccttgattgttccactgagaaaaaatggaggcacgatatttgatatttgtggacttgaatttcttctttctggctttcagtgtcttgagtggtttgagagaaggagaaataaaacttgatataaggcataatgtgctttttaagttttctcactttttttgagtttactgttattttgtgtttatactgtttcaaattcccatatatatctgaggtaaggccaaaagcagtattgaatatcaggagttaaagttactcttttgactagtttcactaaaacctttctattttttttttttttttttttgtggggagtggtcagctgcttgatatatttatttataacaaccttattgagatataattaatgtgatgtatatttacccttttaaaggatataactcattggttttagtatagtcacagagttaggcaactatcactactatctaattccagaatattttcatctcaaaatgaaactgtgtacccattagcagtcactccctatcctttccttcccccaggccctggaaactactgatctactacctgtctatggatttgcccatcctggaatcacacaatgtacgatcttttgtgtctggcttctttcacttagaatagtatttttgaagttgattcctgttgtagcatgtctcagtacttcattccttttatggctgaataatattccattgtatggacatgccacatatggtttatctgttcatcagctgatgaacatttagattgtttatactttttggctgttatgaatgatgctactatgaatatttgtgtcaagtttttgtggggaaataggttttcatttctctttagtgtgttcctaggaatggaatcacaggcccataaagtgattaaatgattaacagtttgaagaaatattaacctattttctaaagcagctgtacctattttctaaagcagcaatcccccaacaatgtatgagcattgcaatttctctgcaactttgatagcacttgttattatctagttttttattatacccatcctcgtgggtgtgaagtggtatctcattgacatattaatttgcattttctgctgttctatatcttttcatgtgcttattggtggtttttatgtcttcttggggagaaatgtctatttgaacagttgtagttttacagaaaagtgagtggaaagtacagagaattgccatatatacactcattccaaccccatctctagagtttccaatattactggtgccttggtgtagtacatttgtgacaatcaatataccattatttatacattattattaactaaagtttagagtttgccttatggttcactctttgtgttgtacagtctgtgggttttgacaattgtatagggacatgtatctactattagagtatcatacaaaatactgcactaaaaatttcctgaataacacctactcatttttcccttcttcaagacacttgataactgttgatctttttactatttccatagttttgccttttcttggaatcatacagtatatagccttttcagattagctttttttctcagcaatatgcatttaaggtgcctgcataactttgtgatatgatagctcatatctttttttttgagttggagctttgctcttgtgccaaggctggagtgcagtggcacaatcttagctcactgcaacttccgcctcccaggttcaagcgattctcctgccccagcctcccaagtatttgagattacaggcgtgtaccaccacacccagctaatttttgtatttttagtagagactgggtttcaccatgttggccaggctggtctcgaactcctgacctcaggtgatccacctgcctcggccttccaaagttctgggattacaggcttcagccaccgtgcccagccgatagctcatttctttctagtgctgaataatattttatttatggatatatcacaatttgttcatccatttacctgttgcgggacatcttggtttgggtagctgaagtttagacaattatgaataaaggtgctgtaaacattcatgtgcaggtttttatgtggtcatattttcagtccatttgagtaaataccaggtagtacaattgctgtggtaagagtaagtttagttttgtaaacaagtgctaacctgtcttctaaagtggctgtgtaaaataatttcgtattcccaccagcaatgagtgagtgttcttgttgctccacatcctcaccgtcctttggtgatgtcagtgttttggattttagccattctaataggtgtgtagtagtagtatgccagtgtattaatttgcaattccctgatgacatatgatgttgaacatcttcttatatgctttattttcccatctgtttacctcttttgatgaggtatctgttcaggtcttgtcttagtccattgaagtagctaaaataccataaacatgggtgggttataagtaacagaaatttatttcttatggttatggagactagggaagtgtaagatcactggcagattcagtgtctggtgagggcttgttttctggttcatagatagtgccttcttgctgtgtccttccgtgggggcatcttttataagggtattaatctcagagtcctggggcttctggggcatcttttataagggtattaatctcagagtcctcatagcatgatcacctcccaacagccctacctcctaataccatcacattgatgcttaggtttcagtgtatgagtttgataggctagtctactaacattcagaccatagcaagccttttgtccaattttaattgagttgttggttttcttattgttgagttttaagaattctttgtatgttttgggtaacagtcatttatcagatacgtattttgcaaagacttttcctagagtgtgtgacttttcattcatgtcttcctcagagcagacatttctaattttaacgaagttcaacttaccaaattttttctttcatggatgtgttttttatgttgtattttaaaagtcaccaccaaacccaaggacacctagatttttttcctgttatcttctaggagtttaatagcttttcattttacagttaagtctatgatccatttcaagttaatttttgtgaaaggtataaggtctatgcttagattcatttctttaattgtgtctgtccagtttttccagcactacagttatttccccttactttcgggggtatgttccaagacccccagtggatgcctaaaaccatgaatagtaccgaatcctatatgtaccatttttttcttatacatgtgttctgtgataaaatttaatttacaaattaggcacagtaagtgattaaaacaaaataataaaatagaacaattatataattacagtatactgtaaaaaagttatatgaatgtggattatctccctctctctcaaaatatttattgtgctgtaaccacccttcttcttgtgatgatgtgagatgataaaatgcctacatgatgagatagtgaggtgagtgacataggccttgtgacatagccttaggctactactgaccttctaaccatatgccagtaggagaataatctgctgtgaatgatcctagatcagtgagccataataatgtccatggttagacgtcaagagcagacaatgccgatgactaacgggcaggtagtatatacagtgtggatgtgctggacaaatagaggatttatattcctagtgggatagagcaggacagcgtgagatttcatcacactactcagaacagtgtgcaatttaaaatttatgaatagtttattactggaattttacatttaatattttggaactgtggttgactgtaggtaactgaaactgtggaaagcgaaagtgcgggtaaggagagactaatgtagtagtagtagtagtaataatgcttactacttagggttaatataatgattaaatgtggtaatgaatgtgaaatgcttaatccaatgactggcacaaagtaaggtctcacttagtggcagcttttacgattagcagtagttcctcaatattggaaagtgtagaattctgaggagccaggcatccactgatctttttctaaattataccacagacaagcatcagtcaaattggaagcctaggaaaactattatttagacaaacaattgtaacaaacgtggctcttttaacttctatagaactgttttttgttattgttttttaataacaaaatattatggtacaaaacaattgaaagcttctagtttttcataggtcctacaattgcaaaaaggtataattgcttaaaaaatccatatcacagaacagtaaaaagaaaaaaataggtgggcgcggtggctcacacctgtaattccagcgctttgggaggccgaggcaggtggatcacgaggtcaggagatcgagaacatcccggctaacttggtgaaaccccatctctactaaaaatacaaaaaattagctgggcttcgtgacaggcacctgtagtcccagctactcgggaggctgaggcaggagggtggcgtgaacctgggaggcgaagcttgcagtgagccgagatcgcaccactgtaccccaggctggactacagagcgagactccgtctcaaaaaaacaaacaaacaaacaaacaaaatagataaaaattatcttttttcccctccccttaattctgtttttcattatagaatttgtaggtccttctaaatctttttctgtggataggaatcataagaaatgaagcccatcttgatttcttatgatgcctatcatataccttatttgaatctcatttattatgatgcctatcttataccttatttgaatctcattgtgttgtaccttgaaatttcttacataaagtttctgttagaggtaaaatgttattagctcttacctgtataacagctatctatatagacacctaaattggttaattattgctaagagaactaaacatttagtaaagttgttttataaaagtagtcttagactactaagtgacttgaccataagccagaaggagggtcatctcctttatttcttacagttctggaggctaggaagaaattagggaacttagttaagaaaataagtttaatttttaaagaaattagggaactttaattgccttataaaaattttattgcaggccgggcgcggtggctcacgcctgtaatcccagcactttgggaggccgaggcgggcggatcacgaggtcaggagatcgagaccatcccggctaaaacggtgaaaccctgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaaaaaaaaaaattattgcaaatagtttaagtatcaatatgttttatatcagactttttaggaagagatgtatacccaaatatataaggccagttactaaaactatggaagctataatgaaattctgttagcaagctgtggggcttttactaattctgccctaaaactcttccagtcaaaagcaatgatcaaattattactagagcataagtaaatgtttatggtataaatgaaactgtacctgtaggagttttaatacatttaaaatttaaaaattctacaaaatggttggtttcttttaaaaattctagttttgtgatgttttaatccagaaattgaatcttgactgaagttggtcttcccatacttgaaatttatgttttaaaatttggatggggatggggagaaactataagtaaaacctacagcattctgaggtccaaaacttcctagattgcttcaacttcaatccatttggggaattttggagacatttgtcatttatataagaaagttaatgattaaactttgagaagtcagtatatcatggaaacttttaccttaatgatattattctgaaaagaatataatcatacagtggaatctatgcagttattttaaaaatatgcttcttaatgtgggagatattctaattgtttaagaaaagtctacaaaacagtgaagtgatattttcatttagacaacaaaaaagaatatagcctatcagagtatttacagaaatagttttcatgatgctccagaacagtaagtgatgattgtttttgttttttattttttgttttttttgtttgtttgtttgtttttttgaaacggagtctcacactgtcgcccgggctggagtgcagtggcgcgatctcagctcactgcaacctctacctcccagattcaagcgattctcctgcctcagcctcctgagtagctgggattacaggcatctgccaccatgcccggctaattttttttttttttttttttttttaattttagtagagacggggtttcactgtgttggccaggctggtctcaaactcctgaccttgtgatccaccggccttggcctctcaaagtgttgggattacaggcgtgagccaccgcgcccggctgatcgtttttgttttttattctcaccagcagggtatcccaaggcttggtttagtcagtctctttaattttagctattctaataggtgtatagtggtatttcattgtagttttaatttgcatttttaaaatgactaatagtgttgaatatcttttcgcatttatttgccatccgtatattgtctttggtgaagtttctcttcaaatcttttacccattttaaaagattgaattgtttcccccgccccttatctgagaaggatacattccaagactcccagtgaattccagaaactgcagatggcactaaaacctgtatatactgcatttttgtatacatacatacctatagtaaagtttataaatcaacacagtaagagattaacaacaataatatgacagtgtcactactcttgtgctttggcaccattatgataagtaacacaagggttactggaacccctgcagttgatctgctaactgtgagagggctgctatgtgatgaatgggcaggtaacatgttcagtatggacaagctagacaaagggatgattcatgacctggtaggagggactgggatggcatgagatttcaccacgctactcagaacgatggcaatttaaaattcattcattattcctggaattttccatttattatttttggactgtggttaactgaaattgtggaaaatgtatccacggaaaatgaaaccgtggataaatgtttagactactctacttgttgaaaagactatcctttctccattgaaattgctttgttcctttgtgaaagtttagttgaccatatttgtttgctagttctgggccatctgttactgttccagtaatctcttctttcaccagtaccatgctgtctttattcctgtagctttatagaaagccttgaagtcaggtactgtcattcggctgactgttgttcaatactgtgatgactattctgaatcttttgcctttccatataaattttagaatcagtttgttgatatctgcaatgtaacttgctgggattctgattgatactgcattgaatttgcagatcaagttgggaagaactggcatcttaatattgagtgttcttatccatgtacatgatgtaagtttccatttatttagaccttccttgacttttttggtaacagttttatagttttcatcatatagatttttttttttttttgagatggagtcttgctttgtcacccaggctggagtacgttggcgcaatctcggctcactgtaacctccgcctcctgggttcaagcaattctcctggctcagtctcccaagtagctgggattacaggcgtgtgccaccacacctggctaatttttgtatttttagtagaaacggggtttcacggtgttggccaggtttgtcttgaactcctgacctcaggtgatccacccacctccgcctccccaagtgctgggattacaggcatgagccatcactcccggcctcatgatatagatcttgtacatattttgttagatttatacctaagcatttcatttttgcgggtgcttattatgttttaattttaatgccaattgttcctgatatatgagaatacagttgacttttgtacattaattttgtatcctgcaactttactataattgcttattagttctaactgtttatttgtggttgattccttgaggttttcttcacaggcagtaatgttattggcgagcaaagacagttttattttttctgtaccaatctgtatacttgctattcacatcccttatttttttttggcatattgcatcttacacaattttgctttgtagcatctattttctcaccattaaatgtgatgttagctgtagagttttttattgtactttatcaagctgaagaagtaaatttgaattttaccaaatgctttttcagcatctatatatatgatcagtttgtttttcttccttagcttgttgatgtgatggattacattaattgacaaatgttaaaccagtcttgcatacctggaataaatgtcacttgctcgtgatgtataagtatttttatacattgttggatcagtttgctaatgttttgttgcaaatttttgaatctgtgtttgtaagagattggtcagtagttttccttttttataatgtttttgtccagttttgggttaatttctttgtgtgatgtaaagaagtggtccaggctgggtgtggtggctcacacttgtaatcccagcactttggccaaggcaggtggatcacttgatgccaggagtttgagaccaccctgggcaacatagggagcccctatctttatgaaacattaaaaaataaaaaatgggctggacgtggtggctcacgcctgtaatccagcactttgggaggcccaggtgggaggatcacagggtcaagagatggagaccatcctggctaacacggtgaaaccccgtccctactaaaaatacaaaaaattagccgggtgtggtggcacgtgcctgtaatcccagctactcaggaggctgaggcaggagaattgcttgaatccgggaggcagaggttctagtgagtggagattgtgccactgcactccagcctgggtgagagcgagactctttcaagaaagaaaggagagagagagagagagagagagagagagagaaagagaaagagagaaagggagggagggagagagggagagagagagagagaaaagaaagagaaagaaaagaaaaggaggggaggggaggggacgggacgggagaaaggaaaagaaaagaagtggtacagcttcagtcctttacagctgaatatccagttgtccaaagcttcatttgttgaaaaatctattcttttgttatagaattgtgttgacaaccttgtcaaaatctgttggctataaacatttttggactctgaattctctttcattgattgataacatatgtccttatgccagtaccacactgtcttgattactttgtctttgtaaaaagatatgaaatatgagtcctccaacttcttctttacaagattattttgactatctgagtgcacacaaatttccatatgaattttaggattatcttatcaatatcttttttttaaaaaaaagatagctggccaggcgcagtggctcacgccggtaatcccagcaccttgggaggccgaggcggacatatcatgagatcaggagtttgagaccagcctggccaacatagtgaaactccgtctctactaaaaatacagaaattagccaggcatggtggcacacgcctgtagtcccagctactcagaggctgaggcaggagaatcgcttgaacccgggaggtggaggttgcagtgagctgagttcaggccactgcactccagcctgggcaacagagcaagactccgtctaaaaaaaaaaaaggtagctgtaatttcaacaggattgtagtggatctgtagatcaattttgggaatattgccatcttaacattattaagtcttccaatccatgattatagattgtctttatttatatctttctatgagattttagagttttcaatgtataagtcttacattcatttgttaaatttattcctgaatgttctttttgatgttattatatttggattttttttcctcttaagttcttttgcagtgtgttctttgctagtacttcattattttgacatcttttcagtatgtaatgttaaactctgtcctgagtttattttaatattaagataggttacattttataaagatcaccctgagataaaaaatctatctagatagggaatggtggtgaagtaatagctattcctgaactctagggtttaatttctatttttctgtaaattattggaaaaggactttgttgttaacccccagccagtctctctgattccttaatgtggttggcgattataatacctgaaggtacttgtacttgcctgtgcctcttttccatcagtactagggtttcttattttcctaatcctcccaagttactgaatagagagccctttaagaaattggagaagtcattttacctctctaaatttgtttcttcttggtaagatgtaggatgaaatatttaataatcattttaagttttttcatacttaagtgagagtgctcacataaatagcttctttaatctgtaaagaaatattcaaatattagggttcctttctcttcaaattagaattttcctttcttagcttgggataatctatcaagatttatctgaataagctacttcacctcacctttctgttaaaaaacctccactgagtttttgcattacaagatcaagtgtgaactaaggctggccaactcaggttgtatatactgtgtgcaaaggactacattggatgttgtgtaggttacaaggagacatatgaacagtctgtgcaatcaaggagtttatagtctatgtgaagtgttaagtgaattgtctagtttggtgccagctgtgggagttcatgatttatatgatagactatcttcctgtagattccccggaaaattgtgctcacatgaaatcttagagaacctacaagatttataaacatagtaactgtttatgatagataacaaccaaactgaatttctagggctttattgaggagagtgtttatctaggcactgaccatgttactacccaactcttggtaatgcctttaaaatttttttaaattatttttggcagacactctgtgtgaggtacagattttcagtcctttgaacacaataaatgaatgaaaattgatctgaagagaatattaaaacattttcacttgaatcttcacaatctaccagtactagggtttcttatttcctaggtagatggaaagtgttttcgatgaatttggaccagaattggtacaaaaatcaaagaattcaaaatattaaattattttgtctctctttaagattgttataatttgattaagtattcttttttagtgtcatcagaaaaataaagcataggctgggcatggtggctgatgcctgtagtcccagcactttgggaggccaaagcgggaggattgcttgagctcaggagttcaagactagcctgggcaacctagtctcttttatagagaccttgtctctataaaaataaaaaaaaaaaatggctgggcacggtggctaacacctgtagtctcagcacttcgggaggctgaggcaggtggatcatttgaggtcaggagttcaagaccagcctggccaacatggtgaaaccccgtctctactaaaaaaaaaaaaaaaatacaaaaattagtcggacttggtggcaggtgcctgtaagcccagctacttgggaggctgaggcaggagaatcacttaaacccaggaggtggaggttgcagtgagctgaggtcacaccactgcactccagcctgggcaacagagcaagactttctgtctcaaaaaaaaattaaaaaaaaaaaaaaaaaaagaaaacataatattacaaaaacctataattcaattattttgcttttgatttgtttgctgtgttaaaaatgagcttttagatcggccttccctagctttattgagatataattggtatatttaaaaactgcatataattaatgtgtgcaatttaatgcatttggacaaatatgtgtgctcatcttaccatcaccacagccaagataataaacatatatctattgcctccaaaagtttcattatgcccctttcttactgtttttcatttttttgagagggtaggtaagaatatttgacatgacatttaccctcttagcaaattttcaaaagttcaccttgttaattataggccctatgttggaaagcatatctctgtaacttacttatcttgtataactaactttataccgttgaacaacaactctccatatcttcctcccctgtctcctagtaaccaccattctattgtctacttatataggtttgattccttgttatgaaaaataatgcagtatttctccttctgtgactaacttattttgcttagcatagcgtattttccaggttcatctgtgttgtcacaagtggtaagatttccttcttattaaaaaaacttgtaatatttattctattgtatgtatataccacatttttcttatcaattcatctgggttttcccatctttgctattgtgaataatgctgcaatgaatatgggagtgcaggtatctttttgagaccctaattgcaattcttttggatgtttacccagaagtgggattgttggatcatatggtctttttgcatatgcctgttggtcgtatgtatgtcttctttgtctattccagtcctttgcttattttttattcatctcattttttgctgttgagttataggagtttgttatattttctgaaattaactccttgtcagacatttggtttgcaaatattttcttccattccataggctgccttcttactctgttgatggtttcttttgctgtgtagaagctttttagtttgatatagtcccacttgtctatttttggcttttgttgcctgttatccaagaaattattgacaagaacagtgtcaagaaggttttctctgatgatttcttcaaagggttctacgttttcagatcttatgtttaaatctttaatccattttaagttaatttttgtgtatggtgtaagataaggttccaatttcattcttttggatatgggtatccaggttttccaacaccatttattgaaaaaactgtcctttctgcattgtaaattattggcacctttgttgaagatccattgactttatttctggattttctgctctgttccatttgtccatgtgttttttttctttttctttcttttttctttttttctttctttttttttttttttttttgtggggaagaagacagtctctactctgttgcctaggctggagtgccgtggcataatttcagctcactgcaatctctgcctcccaggttcaggcgatctcccacctcagccttccaagtagctgggattcatatgtctgtttttatatgcttgtatcatactgttttgattactgtagttttataaaatattttgaaatgaggagtgtgatgcctccaacattgtcatacttgctcaaaattgctttgaatatttgaggtcttttgtgattctatgtaaattttaagattaaaataaattatgtaaaaaatgtctttaggattttgataggggttgtgctgaatctatagatggctttggataatatggacattttaacaatattaatttaatccatgaacacaggatgtttttccatctatttgtatctgccttaatttctttcattagcgttttataatttttagtgtacaagtctttcacctccttggttaagttaattcctaagtattctgtttgttgctgttgtaaatgtgattgctttcttaaacttttttttggatagtttgttgttggtgtataacaggggcccccaaccctcaggctgcggaccagtgtggtgcatggcctgttaggaaccaggccacacagcaacagatgagcaccaggccagtgagcattactgcctgagctccacctcctgtcagatcagcagcagcattagattcccataggaacatgaaccctattgtgacatccgcatgcgagggatttaggttgcacactccttatgaggtagaacagtttcatcctgaaaccttcccaccagcccctcaccggtccgtggaaaaatagtcttctatggaactggtcccttgtgccaaaaaggttgaggactgctggtatatagaattgcaaccgattttttaaatgttgattttgtgtcctgcaacttgactgaatttattctaacagttttgtgtgtgtgcatggtctaggattttctacctatttcatatgcaaacaaataattttcttttttttttccaatttagatgccttttatttctttttcttgcctaattactctggctaggacttacagtactatgttgaatagatggcaagagtgggcattcttgcctaattgttcataatcttagaggaaaagttttctgtttttcactgttgtgtatgatgtgttagttataggctttttttgtttttttgtttttgtttttttttttttttgagacagagtttcactcttgttgcccaggctggagtgcaatggcacaatcttggctcattgcaacctctgcctcccaggttcaagcgattctcctgcctcagcctcctgagtagctgggattacaggtatgcaccaccacgcttggctaattttgtatttttactagagacagggtttctccatgttggtcaggctggtctcgaactcccgacctcaggtgatccacctgcctcagcctcccaaagtgctgagattacaggggtgggccaccatgcccagcagttatgggcttttcatatacacatgctccatgacctaaaatgaagttacatccagataaactcattgtaagttaaaaatgtcattaagtcaaaaatacatttaatgcacctcatctgtagtacattatagcttagcctagcttaccttaaacgtgctcagaacacctatgttggcctgcaggtggacaaagtcatgtatcgcatagcctattctataataaagtgttgaatatctcatgtaatttattgaatactatatgggtactcatcattaatgtacacagctgaaaacaccattgtaaggttgaaaatttttgaggtgaaccatcaagtcagtgactgattttataacttttattttgttgaggtaagttccttctatatctgatttgttgatggtttttgtaatgaaaggggggtgaattttgtcaagtgctttttccttttttttttttttttttgtttgtgagacagagtcttgctctgtcacccaggctgagtgcagtggcccaatctcagctcactgcaacttctgccttttgggttcaagcgattcttgtgcctcagcctcccgagtaactgggactataggtgtgtgccaccacccctggctaatttttgtatttttagtagagacggagtttcaccatgttggccaggctggtcttgaactcctggccttaagtgatctgcctgccttggcctcccaaagtgctgggattacaggtgtgagccactgcgcccagcctgtttttctcatcacttttaatgcaccaacaatgaggaagagattagtgctgttgttatatatacacaatctttggtgagccagagcttggctgaagaatggtattttcaagcctggccctgtgaggatagggctcttgatcagtttttaacctttggagagattagctggagagggattagagagagtggtacatcacagctctattctcaggaatgggaagtagtcctttacatgtgttttgaggggtgggggctcgtgacttgggaataacttgtttttctaactcctttatttttctgactggtagtgacacttcttacaatgaaatgtattgatatgtattaacgtatctctttttgtcagtaggggtgttattttatatcatcagtgtaaatacatgttcgaccatagattattgagttgatatgggctgggcattgttctagattctagacatacaacaataaacaaaacagacaacaaaactatgtcattttaaagcttatattatagtggggagggatggacaataatgaataaatatgcaaaatagatataatgtattagatgattgttactctggagaagattgaggtaatgaaggggtttaaggattgctgagagtaaatgttgaggttgcaattttaaatagtgtgagaaagtatgtagcatttgaggaaatacctgaagtatgtgaggggggctgatattcagatatttatgcagttgtaaagcaaagtagagaaatcccacatgggtaaaactctttacccactttccctcaatgattacatcttgcaaaattatagtacattatcacaattaggatattggcattgatagaatccactgattttattcaaatttcctcagattctcttgtactactttggagagctgtatgtgtatatatttctttctattttttttttattacatgcataggtttgtgtatagaccatgatagtaaagatacagaacatcacaagtatcctcttactgcccttttataaccacattgacttctctccctgccattactccacccctaacacccagtgtaacccctggcagtaaccactaatctgtttaccatttccaaactttttttctttcaagaaggttataaaaatggaatcatatccagtatgtaaccttttgggctagctttttttttttttttttttttttgtctcagcataggtttctggggatgcaaccatgttgttgcatggatcaataattgtttcttttttattgctgagtagtattccatggtatggatatactacagtttgtttattcacccagtgagtgacatctgggggctattctaaataaagctgctttgaacattcatatgccggttttaatgtgaacgtaaatttctatttctataaaacatatgcccaagagtgcaattgctgggatcctgggtaagttttgaaggttgagccaacaggatttcttgacagattaatgtgaggtcaagccgggcgcagtgtctcatgcctgtaatcctagcactttgggaacaataattgttattgctaataatagcacttagggaggctgaggttggaggatcatttgaggtcacgagttcaagaccagcctggccaacatggtgaaaccccatctctactaaaaatacaaaaaaattagctgggcatggtggtacacgcgtgtaatcccagctactcaggaggctgaggcaggagaatcacttgaacctgggagacagaggttgcagtgagctgagatcatgccactgcactccagcctgggcgacagagtgaggctctgtctttaaaaaaaaaaaaaaaaaaaaaaaaagtcagagagaaagaggaatcaagacttcatggtttttgacctgggcaactagaggagtggaattgccaacagctgacatggggaaagctatgggtagagcatgtttaaaggagatgaaaaattttacttataacatgtcaagttgatgtatattagacacagaaaaattgtcacataagcagttggttaaaagattttggaattcacaggggtggtctggtctagagatatacaattgggagtctgatcagcctttaaagccctgagactggaagaggtgatagaggcagctgaaagaggtgagagactaagttctggctacttcagtgtttaaaaaatagggagattatgaagaactagcaaagaaaactgcaaatttggccaatgaggaaggattaaaaagcaagataatttagtgtcctggaaagtaattggagaaaacatttcaaggagagagggagtcaaccaatcatttttgtgtaaaccttagttgtagcacaaaggcatattctgcacagtaaccagaggttttagttgtgttgtattccaggggatatataggctgatgcccccgtttctagtttctcctgagtaaggagaattttcgagttttagtttctgtattcagaaagcaaatggtctaagagacaagttgaattctacctgttaagctccaacttaacactcttttcatgaaaaaagtgaacactcttttcatgaaaaaagtgaacactcttttcattaaaaatctttttaatcttcctagttggaagtattctttctgtttttagaattctccatcctttttttttcttccagggtcgagggagagacagggtctcaccctgttacccaggctgaagtgcaatgtcacaatcatagcgcactgtagccttgacctccctggttaaagtgatcctcccacctcagtctcccgagtagctaggaacacaggtgtgcatcaccatgcccagctaatttatttttattttttgtagagatgcaatctccctgtgttgtcaggctggtcttgaactcctgggcccaagtgatcctccttccttagcctcccaaagtgctgggattacaggcatgagctgccacacccacccacccacccacccctttttcgataactctagtggcacttaacattttctgttgtatggcatctgtgttagtccattctcacactgctgtaaggatactccttgagacggggtaatttataaaggaaagatgtttaattggttcacagttccacatggctggggaggccttaggaaatttacagtcatggcgaaagggaaagaaagcaggcaccttcttcacaaggtggtgggagagagagtcatcgtgcaggagaaactaacatttataaaaccatcaaatcttgtgagaattcactcactattaggagagcagcatgggggaaaccatctctcccagtcacttccctcccttgacacttgggggggtataggtcctcgcctcagcacgtggggattacagttcgagatgagatttgggtggggacgtggagccaaaccgtatcagtatctttatttgcctacttgtatacttgtcttattttgaattataagctctttcaatactgaagtcctcctttattgtcattgtattttcaagcacatagtaataaatggaattgagttacatttcattgtgatagagcatggaatctgacacacctggtttaaatcttgcctgtgctgtttgctaccaaatttcttaacctctctatgcctacacccgctcttaagtggggatcataatgttgcttacttgataggtttgtggagaggattgaagttgtgaagatacagctcttaacagagtggtcagctcatagtaaattctttgtgttctgtagttgttggtgccatcattgctattgttaataataatatgctgttgtgttattggctctcaaagatacagggttaaaggaatacacacttaagctggcattgacttacatgctagggcttccgaaaactaagtattcaagaacctggtttatatctactttagtagtcatattacctgtcaggatataaaagtccgttattcccttagttcatgcaggcatcctttgtgttgtttctcttccatttccctttctgtgctgggcaagtagtctgtgtaactgattgggaaaagtagcatacatgagtcctttttattgtttctataaaattggggttttgttacagaacatgttaagcctacctaggcagtatgttattttataactataatttaaaattttaacttacacactaattatagttatgtaaaaggaaaatatcataaatttccagaacattagtccctaaatcaatatagaaagtccactatgtatatgtactaagtacaaaattgagattgacggtaaggaactttcctttcttgccttgtggtgcagggattgggaaacagtgaaccagccacctgtgtttgtaaataaaatttataacaaacagccacgttaattcttacacatattgttgtaaacagacagccacattcattcttataaatattgtctgactgcttttcccctacaactacagagttgaataaacctgaagaatttagtgtctggtcctttatagaaaatgtttatttgcccctgttcaagggtattatttttgtggctcatctcttagagcttttctcactctgtctcttgtattgattccttttcttctgtctgcttaaatgatgatattctccagaatcttttcttgaatagctcacctttcctaaggcttacgttactccactgtctaggttctactttctgactaatttctttttagtgtcctttatagactttctgctgctggggagtttgatcactttttttcttattttataggttgagcatcttgaaaccaaaaatccaaaatttgaaatgctctgaaatttaaaactttttgagcaccaacacgatgtttaaagggagtgctcattggagcattttgaattttggattttcagattaggaaggcttaatgggtaaagataatactaaatccaaaaaaagtctgaagtctgaaacacttttggtcccaagcatttcggataatgtgtattcaacctgtataggcaataatatatactctcctatggcttctgaaccacccacacctccttgaggataagaagaacatacggtattgctttctggaccttttcatctgcttcagtgtccctgagtcgtttcaaaatgaatgtgtcccaaattgaactcacttctttctgcttccaaaacagcttccccggccactgaactgtaccgtgttcatagtcatgtgagtgagaagcctgatagctgtcacctccctccttctatttactaattcaatagcagtaggtgatcactactattgtagacccattttcttccatttgtttttctctgtttttactagtgtagatcctagttttatccgccgccacctttgtaacattttttttcatagcaatcttcctaaaacaaatatcttgtgtaaaacacattagtgggctgggtgaggtggctcacacctgtaatcccagcactttgggaggccgaggtaggcggatcaccagaggtcaggagttcgagaccagcctggccaacatggtgaaaacccatctctactaaaaatacaaaattaggcaggcgtggtggcaggcgcctgtaatctcagctattcaggagggtgaggcaggagaatcgcttgaacccaggaggcggaggttgcagtgatttgagatcatgccactgcactccagcttgggtgacagagtgagactctgtctcaaaacaacaacaacaacaacaaaaacaaaagcaaaaaaaaccaatcagtggttctttatgccttgtaatatttagcgctgtatttaagactttccacctgaccccattccagaattttacaaattaataagcaaatttttcttagctcctgtccccaagtatctatagcactttttaaatgagctcaacttaatcacattgtgttatagttgttggtttgctcacccattagactgtaaaacatttaaagattgtaaatttttgtattctcagggcccccagttctgacacatagttggcttcaaaatatgatcattacatgttctatgcatggaacaaaatatccatgtacctcataaatagtaaaatatcatgtatcaattaaaaatatacttgtgcaaatggggttaagtttttttgtgtttttttttttttttagctgttggttcttttagggcaactgaatttactgtgaaatcaaagatttagagatgaattaaatgctttttgagtcttatgatgactaaggatggataatgcaactttggataaccttcattttttttgcagaaggtgtactttgggaaaatattctttaagcaaaacattggaaatgagtcctattttactagattttatgaaagtagaactttctatcatgggggaagatctcccaacagaacaaactttgatgaagaagaaaagatgataaccacatgctgcccatcatacacccacaatattaagcttgctggttgtttcttcacctaaaagtttacttttcctttattaaactttatgtaatggtacaaaaatatagccttctggaactttttcagtctgtgcttttagcttccagaagtttttctttctcttttctctctctttttaaagcatgagatgtcagatattgtaaaaatgaataaaagcctccatgtctactctagaggtggatatatgttaatgagtattggttaaaacaaaagaatcagttttcctttaaatgtgccatttagactttttaaatactgatattatgcttattccatcttgggtagtcattttgttttataaataagttgagaatgattagtaagaatcttgtttacatgtattcttttgtcttttctatttgaaaaaagatgcctttacaggcaagtcagttaccacaatttaaaatttctaactatgaccttaaagtaagacatgcattttaacattgcagcccgatgcacagaaaaataattaacaagtttcatgaaaaaaacccttactgtatgtaaagtgatattctattccattctattctattcttattcctccattctattttatgctatgctgttctgttctgttctgtttcttttctattctattctattctattctattctattctattctattctattctattctattctattctattctattctattctattctccttccacacttccttctcagtgctgattggctggtgcccatagtttggaaaataacaccaagttggaaagtagaagaaattacagaagctgggaaattactttgttaaagacaaagtaacctgctctgagaactctgagagtaatacagaaagatgttcttaaaacacttatagtatgttggttgggcagggtttgtgagtgtgtgtctgtgtctgtgtctgtatagtgactataatatctgatttctataagctacttttgaaaagtatccttttatataacatcaatgaactgtagttgtaaaaataaaaccaagagaacatatagtaaatttcaaaatatttgtaagtgctgcagggagagattggtgatggcctcgttaaaaaggtaaaagttaaataggaacttaaaggatgggcttagaatgtgtgtaggttggagagagagtcagcacattccactgatggtaatgtgatggtaaatgtaatagaattgggaatataattggtttagaattgggaatgaaacaatatattcaggaagagtgagaagtagcatctagcgataatatagaattcacattggtcatgagtggaagactaaactgaaaaggagaggccagattataaagtgatttagactttatcttgtaggcagtgggtctctctctctgtctgtctctgccaacatgtgccgctccatctctcttggttactgtttgtattgtatatcatttttcatccttttacttttaagttgtttgtgtctttgaatctaaagtattaattcctagggatagattatagttgcagctttctttttatatagccttttgattgtatgcttagctctttcacattttatgtagtgtgtgatgtagctggaattacatgtgccattttgctctttgttttctatatctctcatgttatatttgttcttccgttcttcgttactatcttctgtgttgaacaaatattttttagtgtatcgtcttaagtctgctgttgatttttaacattaaattattttcttagtggttgctctaagaattacagtttacattccaatttatcaccattaactttaggttaatactgacctaatttctgtaaaatatagcacttgtgctccattataactgttttctccccctcctttttgctgttattgccttatatattacatttatttacgttataaacccaactgttatcatattctgtcttttaatgaaattaagaggaaataaaaatatatctatagtcttttatattttctcatttatatttaatatatctggtggtcctcacttgtttttgtgaattccagttaacatgtggtgtcattttcttttagctcaaggtacttgttcaagtatttcaacttattctccatctttttttttaagtgtgaggatgtctttattcttcctttcttgcttgaagaatacttttgcagtaaatagaattcttagttgatagttttttttaattttttgttactgccttctagcttccgttttttcctgatgattagtcggctattaattgtattgtttttcccatgtatgtgatgagtgatttctctgttgctcctctcatgtttttttttttggtggcttcaaactgtttaaatgtgaaatgcttagatgttgatccctgtgtttatcatattttgggttcattgataatttttttttcttttctttcttttcttttttttttttttttgagacagagtctctctctgtcagccaggctggagtgcagtggcacgatctcgactcactgcaacctctgcctcccgggctcaagcaattctcctgcctccgcctcccgagtcgctgggattacaggggtgtgccaccacacccggctaatttttgtatttttagtagagatggggtttcaccatgttggccaggctggtcttgaactcctgacctcaggtaatccacccgcctcggcctcccaaagtgctgggattatagacgtgagccaccgtgcctggccggttcattgatcttcatggtttgatagattagtggttttcagcaaatttggggaatgttcagccattatttcttctaacatttttttctcctttctctttttccagttctgcttttacacatatgttgatatgtgtgatgttatcccagaagtatctgaggctctgtttatttttctgcagtattttttccctctttgttctttcttttggacaatctctatttcttcaagttcactgggtttttttttttaatgccatctcaaatattttcttgaggacatagtgaatctcatttttgttataatgtgtttcaactctagttaattctcatgttttaaaagttagcatttctttattgagattctctgtgttgtgcaattgtcatttttctttaatttttaaaatatggtttcccttaattttttgaacttaattataaattataatagctgctttgtattctttatctgctaaatccaacagctttgcccaccaggagttaatttctgttgactgcttttttttaacctgattataggtcaacttttcctgtagtttgcatgtctcacaatgtttttttccaattgaacatattatataatatattgtagcaatgctaggttctgaatttttttgagacagattctggcttgttttctaggctggagtgcactggcatgatctcagcttactgcaggctccacctcctgggttcaagcagttctcctgcttcagcctcccgagtatctgggattacaggcatgcgccaccaagcccggctaattttatatttttagtagagactgggtttcaccatgctggtcaggctggtcttgaactcctgtcctcaggtgatctgcccgcctgaacctcccaaagtggtgggattacaggcgtgagccactgcaccctgccactttcagcattttttgatgatcctatatgtgagttgggaagtatattcaaaattctgacatttttcacattagccttggcttttactttctccggggctctgttgagcctccctccacccccatatgtgcactgcctgtcagacagttatgtgtggggagctaatttaggccctctgtgcctctctcatttccagtatccccttattacatttctgggtgtccttctagtctgtcattttctctaactgggactacattctcattttagcagagctatgggcctttcccctttctgtccttccaaatttgctatttttattgccagtgctactaggcatgggtttttgctctctatcctaaatgaccatagccctctggttttcactgccaacactgccctgggaaaactgcctcactgattgagttgggtggttgtagggggatggtagcatcgcttggcaaagaaggccattttcctctactgttctgtcatatctagcagcttttcaagaataaatgcttctcaatttgttgttgcctttgctcagtttccagagccctgaagtgattgttttcttcagtgtacttcagttttataagtattgtgggggagtagatttgctactgtcatcattctgctgtacctggaaatctctcctcaaactttctaaccccttaggaccctgtgtcccctacacccactttaattcaatctagtcagtttttttcaccattgttctactgaaaccattcttgtaagagtcaccaacatcttgcattttcatagcaattgttattttcctgtgtttatctagccattatccttctgcctatttcttgaatatgccaagcccaccactgagtttttacatttgcagttccctgtaaccatcttccctagatcttcacatggttcttacctttatatcatccaggttgatgttcatatcttaacctccttcataaggttttctccggcttccttatgtaaaatgtagatatttctaaagtaacgcaataggaacagaagagtaaacatagatgaatatttaggtccttatacttgcttcacttaatgcatctttcagtagcttttgacataaatcatcactcagtttttctaaaaattttttaacgtggcttctaagataacattctttcttatttttcttcctgctttattaactactccactctgtcctctatttttcatttcatgaattttttttctgtatgaacttcttccgtaggtgatatcattaggtacccttgctttaaatcttctttatctagatgactcccaaatgcatttttccagccttagcttctaccctgacctcacatgaatgtcgtataaggatttcaaacttaatagataaaaacttaataggttaaaaaaaacttaatagataaaaaacgaaactcttgatctactttttccttcaaatttattctcttaaatttttagaatgctttttttttttttttttttttgagacggagtcttgctctgttgcccagtgctgtggcgtgatcacagctcactgcagcctccacctcctgggttcaaatgatagaatgcttttctgaaaggtaaatctgattgtatcactttcctggtcaaaacctccattagcttccctttacatttagagtgaaattcagtttcttaccatgtttgatgaaggtttatatgatcttgtgcctgcctgcctctctgaacataactcctacctctctgaacttaactcccacctttctctccttactcagaatgatgcagccgtattatctttctggctatttcttgaatatgccaagcccaccactactaggtttttacatttgcagttccctataaccacagtgctgtttcctcagatcttcacatggttcttaccttcatatcattcaggcttgtgttcatatcttacctgtttcatgagattttctctaacttccttatctaaaataagacatttttaaagcaacacaataggaagagtaaatgtagatgaatacttagatccttgttcttgcttcacttatgcattctttttgatcaaaagtattatgcacaatcttatcaacttctgttatctgggaatttaatactctgttaccttggttagtgagtctggaaaaataaataacttattttaacatagttggctatgttaaaaaaatcagtcttaatagctgtatgttatgagaggaatgagtattgatttactagcagttctccctcaaagccagctacctaaatgtgaattaacaaagtatagaggctgggcgctgtggctcacgcctgtaatcccagcactttgggaggctgaggcaggtggattgtctgaggtcaggagttcgagaccagcctggccaacatggtgaaaccctgtctctactaaaaatattaaaataattagctgggcgtggtggcaggcgcctgtaatcccagctactcaggaggctgaggcaggagaatcgcttgaacccaggaggcaaaggttgcaatgagccgagatcacgccattgcactccagcctgggtaacaagagtgaaactctgtctcaaacaaacaaacaaaaaaacaaagtatagaaatacttccagaaatagcttcaatttggaatggaaatggacacaattttgaagttggtagttttctatgttcctctctctgtactagtcaacacttaagagtcttttgttagttatttgttaaagtaattgtactgtactttctgacttattttctgtttactttcggactttctgtttccgttacttttactaacaagtaatggaatgaaagattgatatcttataaaaggattttaaataccattgtacacataagcccaactccgaaaggtcagtttaaaggaacattcacttctgctctcatcttctaagaactagaagccatcctacccttcctattctaggatagctatctttatctggttctttggtcccaacgcatcctcatccccatccttcttggcctcttttctgaccgtaacaccatttcctttccagattctcaggtgtatactttatgttttctagctcttctaaatcattactttaactaaagctttatttttttggctagaaagctggcattttggtattgcttctgtgtgttatgtaatttgtccttcattaagtggatttctgtcagtcaagtatattgttggactataataacctctttgttaatgtacagtctagtcagatttactgctgcgaagtctgtggtattaagaaatcatcatattatttagtgatttctttttttgaagaatgaacataactttgaaacttcccaaagggaaaatagttttgtgtggtctacaaatctatcaggactgtataatttcttttcttttctttctttttttttttttttgagatggagtttcgctctgcattcactttttctaaaacttagcaactgagctcttttcatggagttgatgctcagttttttttttcactgcatcttagaaaacaataaggtcaagctatttgtggtaaagtaacagttcttacagttttgaattcatgggagactgttgtttttgtgaaatatacaaatgaattgctagaaagatgaaatttttaaaaatataaaatatggatcctaattcttattaccatattcttagatatcaatttattctctcattgttataaaagctctaaacactctcaatttctgtatttgtcttattggagagcagtaacagatagttcatggactagtaacaaatattttgcagatcagaaccagtctacagatcacactttttagtagcatatctatagcttgctaattttagtgcttaatgttatttcagaagcatcttgttcaatatgatgaatagctgttatcctttaaaaatatatttttatgtgtttccttttcagaatcgagagctccagatcatgagaaagctagatcactgtaacatagtccgattgcgttatttcttctactccagtggtgagaaggtaggtttgacacaaagtgtgtactgaatattgtgataaatttgttatttttctagaaattaatctccccaccgctcatagacagttttgctaatggaatggaaaaattccgaaacacttgccccagcaatactcagcccaaggtatggtcattgatatcattagttatatcaggaaagcattagtaagataaagggaatgcacttttaagaattttcttctttgttaaagtatgtcttcacttctcaagaaaatctatatactttttttttttttaaagacatctatgttccttatgtgcttacgcagccctctgatagtttacctatttattgcagggaggaaaattaagtcattttgttaagtgaagttgataagaggtaaagaggtagcaagaggactgataggaacagaatgagagttaaacatttggaagcaagttgagccatccttggccagtgaaaaagttggaaaagatgtaatattgcagagcttactttaagtgatccctatgtctgaccacattgaattctgcgaaaccaaaggataaagaagactttgtgctgttaataactccttccaacctctttctcttcagaagacacaataatcaatgaagttggagcaaagaaaataaatttcttctgtttttccttggtgtctttatttttctgcctgtcgctatcctttctttcttttcactggtttggagtgcatacgcagggcaggtaacaaatcacttgattcttttggctaaataaatatgactttggataacttcagttggtattttggacttcagatttcatgcatataatatctaatgttcttagatccataatacttgattcctagctcagtgttcttgttactttatgtaactgttttttagtataacagtttttagccattttctattaagctttttttttacagcaaatattgcctgacaacattttctattaaactttatttagattatttccatagaaagtctttttacattacagtgcctattatataggaagatgtcaatgtattaagcccgaattttgatataaatacctacagtcatataattgttgtgtatatacacatgtattacacaatacgttttaacaactatagttagcaaattggccctttattgacacttgcttttcagttgctttttttttttttcagtttaaatgatacctctggtttactagggattgtgaaccctcataagtattagagctttttaaaacctaccaaatggcctgttattatctttattctgatctgttttggcatgtgtttgctttttcctgtctcttcctaagtaaaattgtacagcctctgttaggttcacaagtacactctttgtaatgaaattttgcatcttcattttgttgttgttgttgttgttactagatacatcatgatactcctgggtaagaattatttgttccataaactgctggattacaatagattcatttgaaaaaaggcctcctcccagagttcttaaacattttctctttgattacttactttaatgtatatagcattccttatatgctaggcactgttttaatgctttataaatattaactcatttaactcttaagaagttgaagagcagaaaagaaagactatcaaaccgtttgaatgcaaggcatctgatggcttacttgtctggttttggtttggtggaaagctcctgtcatagtcagctcttgccctgacttaatcctgcattgggggtgacaactgactgcctcttatctgtatgccccagaggataatttcctctctgctgctgttctcagtttcttacttatcttaaaggttcttacagataaagagcaattagaagtcattaacagagattaacctctattataaaaagtaatagacctaatattttctttatgtcagaactttctcttgagctttgtaaattttccaaaaatttttttaaaaaaagatacctcctaattttgggatagcttcattaaaaacaaaaaggaagaagagaagaaaagttgtgatatattgctgaaaataatggtccttctttgtcaataaaggaaatttttctaagatgtgcagaaatacataggttcaagacttcccgtaatctcccagccaccaaagagagaatttctttttgccccattcatgatctccacttctgaattgttctggcaggactaagatatccttcaggagatcaagtagtaaactatttcttcccataggagaagatggattcttttctagggttgagtgctcaggttagttattttcagttctgactttcttggaataggttactgagagtatccactgctgtttagaaaggaacctaaatgtctgtgaaattcttgcaaatccttgaagatgatttatcttggaatgtggcagtcctatatttggtaggcatttttctagcagaagcattatgtattgcttatgtatattatatatgagaagagttttggaggaggaaataataaattaatcatttgagatgcttatgacacatgcaagtagagattcccagtagatggatagatgtgtgacttcgtagatggtggtggtggctctaggtggagaagtgacattgacaaagtaggataaagagggcgtgtagccatgtggagcttggtggggaggagacatggagtgctgtttgaggccaggacagtgaaaaatcagcggtttggcaatatttattgccttctttttctagaatttgtcaggaggtgaccagtcttcatttgaaagaactgcaaggaaaccagtgcctttaggtgagattcaggttttctctagagcaaaacggtaaaaagaaccctgcatgaagaaattgaggtcattagaaattttcttcttgaggtttcagttttctttttgaggtttcttttgacgtttaggtgagatccaggttttctctagggtaagacagtaaaaagaaaccctccatgaagagattgaggttattagaaattttctttttgaggtttttgagagaatgttatgggagggagatcagtgataagaacatgagcctacaggaagtttgaggaggcaattagggagattagttgaggacattgtgaagatgagaaagaataactcattgtttcaggattagagagattaaaggcttggttaggttctagaatagatgacttaaaatgcatttgggaatttaaaaaaattctctcattctctataacatctctgactttctttccctcttttttttctatcagtctctttctctagcccttttttcgcctttgtctttagtcttcacctttctggattcctcaacctttcactgtaggctccaagtctctatctttctgtctctgggcttttgtcccccctctcttggtctgtgtcaccccctttttggatttcttttccctccatataactctttctcttttctctgtctctttctttcttgtctctctcttctcccccacaccctttttctctctgtctctctccctctcttatttcctttttatgggtctccatcaacctctttctctgggtttctatacctgtctttcaatgggattttacccatcccactctctttctgattgcttctaaatctgtatatgggttctcatctgtcttaaatcttcttcgttgtctctcttagtgtctcttctctctgggcctctattactccctttttggaaaatgttaaggaaaacaactaaattattcactactactgtcctgacctttgacttcagttctgtgtatcttattccatacgtacatgtttatataggtatatgcataattatttatgatctcataaagatttttttctcttgcctcataatatgtataggcattttggttatctaatattccattgttatactatataatattttatctattcccacattgttatttttaattttagaattgtagctaagctaccctgtggccttgtcataatttctgtctgtaagtacttttgcagctctgggagaattaaagtaggatttctgtaataacaggcatgttttacatttcattgaccacagtttgactgaatgatatgtctattttggtaaccttctttttattgtattctgccttttgcattttaaagactttctttagggatagtggataatcagcttttatcctgtaacaggggaaaggggtagccaatgtaactagaagaaagaggaattaagttaggacagagattagagatgtgtactaattagccttagttttctggttggaattattaatgtttgctgtagtaactagagcatcatgtagtcaattcgtatactctatgttacccagtagtaactaactatgatttagtttagagtaagtagtacctcctctcttaccttcccattagagaaaaatgagatatttgagtgttttgaaaccctgggcttatagttttgtgagttgcaagtatcttacagaatagtctttctctttttttaatatttggcagtagaaactttctacatccccaatgaaattttctgaagacctctgatagaacatatgaaaagcagagttgccctagttgaagctaaggtgtgggatcagatgattttgccttttctgagttatagcttaccccaaagaactctaggactctgaagatcatatgataaaaaccatgttatgagctgggtgtggtggctcacgcctgtaatcccagcattttgggaggccgtggcggttgaatcgagaccagcttggccaacgtagtgaaaccccatctctactaaaaatacaaaaaattatctgggtgtggtggtgggtgcctgtaatcccagctactcgagaggctgaggcaggagaatcgcttgaacccaggaggaggaggttgcagtgagccgagattgccccattgtactccagcctgggcaacaacagtgaaactccgtctcaaaaaataaaaaataaaaataaaaactgtgttatgtcttcacaagatccttaaattttatttcaatcaattaatagttttctgtaagaaaaacttctctaagtaacttttcatttaaaagtttttttccttgtggttcatagattactttaataataaagcgaaaaacctctgccttctactttgtgagagactgttcaaataacaaagatgacagttgattttatttctcatgccacctttgttctattgatagtaatgggggcctgtatccagaaggggaggattgtaccactgcatctgggatcagggagcagaatgctgtgcttaattttgctggtctaccactagctctggagaggaaagggtaggggtgacatatatgccaaggattcattctgaaaagtaaagagcactggctttgtagctagaaatatttgagtttgcattccatttctgccacttgatagtagtatgactctgggcaagatatttacttttataagcctcctccccgccatctgcacagtaggggttttgtaaggaacaaatgtaataaggttatcacgtgctcattatagtacctggcatataaattaacacttaataaacattaatttactattttacctattattgtgtttattaatagcaaatatatattgcgctttttatatgtcaaacattgtcctgagctgtttacatattttataactcacttaaacctcatagcaaccaattaaagaatgagttttatagaagcactattttatagaagaaactgatacaacagccgttaagtaagttaccatacaactactaaatggcaatgttgggcgtcagctccaagtagtctgactccagagtctgtatagtatgctatagtgtttagcataggacgtacattcctccagaagcagtgtgctagaaatagaacattaaaaaatatcttagtaaattatcaaacaaggggagattataatattaatagtagtattacgccagtcaaggtggtttacgcctgtaatcccagcacttttgagaggccgaggcgggtgaatctcctggggtcaggaatttgagaccagcctggctaacatggtgaaaccccatttctactaaaaataaaaaaaattagccaggtgtgatggcaagtgcctgtaatcccagctactcaggaggctgaggcaggagaatcgcttgaacccaggaggcagaggttgcagtgagccgagatcgagccattgcactccagcttgggcaacaagagcgaagctccatctcaaaaaaaaaaaaaaaaaattagtagtaatagttgcagtactagaagtagtggtattgataataatagctaaaaggtatgagatactatatgccaggcattaatctaaacacattacttatatttactcatttaatcttcacaacggttctatgaagaggagatggagagaagttaagtgacgtgacctgcccatagagtgatttaaaccagtattttaaatttctagtttgcaggaagtatgtgactcttctaaaataaagattgtgcattttttcgagggagctagcttgggtgttttattaggttttcaaagaaatctctgacccccaaaagataaaacagaaccatcaagccattcacccaagagccaaggagatataattatagcaagctatgtaaatatttataaatatttattagaaatgttttgatgagatttgagcccccacagtctagctctggattccattaaccttccctaataccaccactcttaatttcagtatatgtttttcttttaataaagaacttttatcagaggtttctaaaacttgtatgtatgtatgtatatgtactcatttctaattcagttttaactttttcgagagcaggtatgtgtgcatatggttatattaagggatgaaagaaagaaagaaaaggccaagaacagttccttcttaattttgaagcagtatagtataaagaatcaatttgagtatgaatggtgactctgctgttcatgtgtctggccatgaggactggctatgtgtcctcaagcaaattgcttaatttttctccttatcgctaaaatgagggtaatatatgtatgtataatatatatacttgtgttttatatatatttttatatatatacttgtgttatatacaccatgtgtatacacacacacacacacacacacaaacacacacacacacttttgttactgtgaaaattgtggccatgtaagtaaaatgtttatagtatcatgtattagtttttctttcattacctctaaccattagttcttggtgttccctagggttttattctccatttttctttactttcatacttatcctgggccatattctcaatccttgtgacttcagtgactctctaatgtttatcttcagttgaggtctccctgatgaactctagagcctcaaattttagtaccaaccagaaatctctacctggaagcctgagccgtcaacatttagcatgttagaatttacgactccccttatccttgctatatatcaattattaaaataagaaacctggatgttatgctagacctttttgttgacttatttgaatctgttctttctatttcactgataatgctgccttagttcaggcccacattatttcttatctaaactcttggtgatttctcttcttctagtacatacctttacttcctccccacccctagcctaaacagtattttaaaacagggataaatcatagattattctaaacaattagaagagacaatatgggtcattgtcttaagaatttattgtctgggtttaccaaaagctttatatgatcaattagcattggagatgttggagatgggagttgtgaccatccctaatataacatggcagcaggcaatccttctgattgtatcacttcttcagttacctaaaaggtaaattccagatttcttagcatgacatacaaggtctttgaacatctatatcgtgaccttacttgactcatctctcctttctcacaggctatctatgttctagccttgcccaaagtatgtgcaattgtcagaatatgttgtattatccctgttaatgcaaataccttctggaatgcttctttcctaccctccacccctgctctatttttgttgagttctttttcattgtattgtctctgctcaaaagtagctccatctgatatctcactattttctctccccccagccccccttttagccccaaaagcttctctctccttggaatatcttgcacatacctctttcatgacatttttcttgtattttaattattttttttttaactagatctttttactaggttgtgtaattctcagcagcatggtgagattttattaattttgtttctttaatgcttagcccagtgcctgagacaatgtgaagttggctatgtagatttattgaaagccttctagatagtcgtagaaaactgagagctaaaataggctcagcccagatattaacttttgttttttgaagtatattaagatttattataggtattacaataactatttctatagattgtgttattgtaaaggaattattttcttaattttacttttgggatattcattgctgaatatcaatatataattgtatagaattcaagagcattaattacgtatatgatataatgcagctatcactgttatttttaaaacattttccgttttatcacgaaatggaattggagtttatcagatgcccttttggtgataattgaaatgatcgtgtggggttttttccatcattttattaacatgatatattacattgattttcttatgttgaaccactgttgtattcctgagataaaggattataccccaatcttggtcatggggtataatccttttaatatgctattagatttcatattctagtattttcatgaggatttttgcgtttatatttataagggatattggttggtaatttttcttttttgtgatgtgtttatctggctttggtatagggtaatgcttgccttgtagaatgaggtaagaagtgctccttccttttctattttttttagaagagtcttagaaagattattgttaatttcttctttagtaaactggcagaagttaccactgaaatcatcaggtcctggactttactttgttgggaggtttttttctttattactaattaaatctctacttgtgtgtatgtttgttaagatcgtctgtttcattttgagtcagtttgataatctgtgtatttgtaggaatttgcccatttcatccaggttgtctaatatgttaacatacgattatttgtagtatgctgttttcatcctttttgtctctgtgaggttagtagtaatggccccactttcatttctgattttaattctttttctttttatttgtcagtctatccctacatctaaactcttttaagggtttgtcagttttgttgattgaaaagaaaaaaaacttttagttttgttgattctgttttttttctgttctctgttttattcatctctattcatatatttcttatttccttccttctgttagctttgggttcagtttcttctttgtagttctccggttgtagagttagattaatgatttgagatcttctttcttaatgtaagcatttacagctgtaaatttccttctgagcactgtctttgctgcatcccgtaagtcttggtattatgtcttaactatgtaaaaatttttcttgtttaagagtattatcttagtcagtttagtgctgctataaaggaatacctgaggccaagtaatttgtaaaggaaagaggtttatgtggctcacaattctgctagctagaaagtgaaagtctcaggctgcctccacttgtggtggaaggtgaaggggagccaacatatgcagtggttacgtggccagatagcagagaggtgtcaggctctttttaccaaccagttgtagtgggagggaactcatagaatgagaacacattcattaccaagaggaaggcacaattccattcatgagggatccacctctgtggtccaaataccttccattagaccccatctccaacactgaggatcaagttttaccatgaggtttagggggacaaacagtccaaactatagcaagtgtgttgtctgttatcttctttaaaccattggttgtctacagtgaattgttcaatttccacacatttgtgaattttctggtttcccttcattactaatttttagtttcatttcattgtggtcagaaaagatatttcatatgatttcagtcttttaaaatttattaacattgttttgtggcctaatatatggtctatcctgcatagacctatgctcaggtgcaaaataatgtgttctctgctatttgggggtggctgtattctgtatatgtatgttgggtccagttggtttttagtgttgttcaagttatctgtttccttattgatctgtcttgatgttctgttcattattgagagtggtatattgaagtctccaactattgtacagctgcccatttctcccttctattctgttagtctttgtttcatgtattgggggctgtgttgattggtgcatttgtgtatataattgttatagctccttgatgacttgatttttttaatcaacatacgatacccaaggctaggtgcattggctcacacctgtaatcccagcactttgggaggccaaggcaggtggatcacctgaggtcaggagttcgagaccagcctggccaacatggcaaaaccccatctctactaaaaaatacaaaaattagccaggtatggtggtgtgcgcctgtaatcccagctactcaggaggccgaggcaggagaatcacttgaacctgggaggcagaggttgaagtgagctgagatcctaccactgcactccagcctgggtgacagagcgagactttgtctcaaaaaaaaaatatatatatatatatattttttatatatatttatatttttatatatatttatgttatttatatatataacctgaagccttttatttttttcttctggacaccgttctggctagagtctctagtacagaagagataaatgcagataaatgcttactagaagagataaatgcagacattcttgttcttgctcctgattttagtggcaaagcattcagtctttcatcttcaagtgtgatgttagtcttgggtttttcatagatatagtgttatcagttgaggaagttctcttctatttccagcttgttgaagcatttttatgaaagggtgttggattttggcaagtgctttttctctattgattatgtggtttttgtttttattaaaatggtgttttatataagttgatttttgaatgttaaacctaccttacatttctgagattaatctcacttgggtcatagtgtgtaatcatttttatatgttgctaaacagtactttgttaagagtttttgcatctatattcataagggatatcgatcagtagttttattttgtttttgttttccttgtcatgtctttggttttagtattgggtaatactggcttcatagaatgacattgagaagtattccctctgcttctgttttttgaaagagtctgtgaagaattggttttaataccaattaataggaactatttagataaacagtgtagaactgatgtatgccaaggagtacagagtattttaaagacttggtgatgtttaatagcacagtgtgtgtagaggagatacaagtagtttgaaattgctggtatataaagttggggtagtggggattgtagtggagatgaggctaggaaagtaaaagtgggttcatatatgattaggtcaaggtcagcctcctagtataaaaataattttgttgtcgttgttctttttcaattagatgaaaaccccaaattacagaaatggtaccttgatcaggttgacaatatgcaattctcccctcttttcctctctctttatcttactatattatgaaaattactgatagaagtgaattttattcagtgaaataaagtgattacctatctaagttcgcttggtctatcttccttaaagttttatgttgatgtcttattaaagtggtttattgattatctggatgaaacttgactgatgaaatgctaacattttcaatggagtatgacctaggaagtgaatatataagtaataataaagctcttgagtttgaattatcaggacctcctcataaagtataaatgttactgacagcaaatgaggtggtaattatcatctgctcaaaaataatctaggagtttcttttgaatatcagttgtcatctgtttttatgttattttaatatttcagcaaaactaaaatgttattacatgcatactatataaaaaattgaagtaatacagaatggagtaaattaaaatgaaatattttttaccctaatccctaaattccctaatcccactcttaacagatataaatttcttatatgcatgtatgagcacatatattatatgtaaattagtaataacatatgtattttggaacatgttccctttttaccaacaatacagtaatagggcttttatgtctatatggacagaccctgcaaagtttcccctattgttaagatattaatatggtacatttgttacagttagttaattattatagtttattcagatgtccttggtttttgtctaaggtcttttgtttatgatcccatttgagataccacattacatttacaaagtataagaaagaataccacattgaggatccctcattacaacataatgagatgttagtggtcttatcttacttacctgcatggcagtttcttagactttccttggttttgatgatctcgacagttttttggagtactggtcagatgttttgtaggatgcctcactattggagtttgtctgcttttttaatgataaaactggggttacacgtttttggcaagaagacaacagagaggtaaagtgtcattttcatcacatcatatcaaggacatatactgtcgacatgctttatgagtgctgttaactgactttcaacacctgactgatgtagtgtttagttttcttgactgtaaaattactcttttccccactctttccatactttattccttggaaggaagtcactatgcagagactacacctaaggtgggaattatgcccctatacttcttttccttaatggtggagtagctacacaaattatttcaatttattttgcgtaagagacatgtctcttttcccccatgtattaatctatttagtcatttatttctatcagtgtggatttgtggatatttattttattggccacattgtcccaggtttggccactgggagttctgtcaactacctcctgtgcttcttgtttgtttttttgttttatttattattatggttattatttttaagaaggagtctcgctctgcctatcaggctggagtgcagtggcttgatctctgctcactgcaacctctgcctcccaggctcaagtgattctcctgccttggcctcccaaatagctgggattacaggcgtctgccaccatgcctggctaatttttttattttcacaagagacggggtttcaccatgttggccaggcttggctcgaactcctgacctcaggtgatccacccacctcggcctcccaaagtgctgggattacaggcgtgagccaccgcgcttggcggctcctgtgcttctttaacatactatcatgtgggctttttgttttattttaaagcatgtttttgttttctggcattataagataatggcatgcagatgtgtttttaatttcttgctcttcacaaattttatgcatgcacgagcgtgtacacacacagaattgttttcatagatgtaatccctaaagtgtcattgctgagtctatttgggggggctctatgtcttcacttctgaattaataacccattgcctgcccaatagattgcatgagtttggactttcacaagaattgttgtagttcatattggatggtgatctatttcaatacttagttttctcagactgaattttcttttctagtttgatagatgaaaaattgtggcttattgctatctttattacatttctttaattatgagtgaggctgagcatttttctacagttttgttggccatttttatgaccatctgttaatgcgtatttccatatttcctttcttgtttttgtttgtttgttttgcaggggttggggtgggcaacggcatgatttcgatcactgcaacctccacctccggggttcaagcaattctcctgcctcagcctccagggtagctaagattacaggtgtctgccaccaaatccagctaatttttgtattttcagtagaggcagggtttcatcatgttggccaagctggtctcaaacacctgaactcagcctcagcctcccaaagtgctgggattacaagcgtgagccaccgtacccagcctatatttccatgttttctgtttgaatactggttttttcttgttgacttccaagtactatttgtgttttaaagaaaaacaaatcaggcttttatcatgtgtagcagaagtttatttatttgtttatttgccaaattgtcatggtttttttttactgggtttgtgtgagttttttttcttaaggtagtatagatgttttacagttctgtgtaatcaaattggcattatttttctccatggcttagctcttttgtcttccaagatttgcagaaatacccatgttttcttctggcacatttattatttttctaaaaaaaaaaaaaaaccctttggtaattttggtgtttattttgttttaaggtgtgagatagggatgaaatttgccttaatcgttgtatgctaggtgcctaccctttcccatttccttcccacagcctctcaagcaaaaaggtattctcttacctaatccacacactctttactattagtactatattctcatctctagccagcctggcgtacccatcagagaaaagatagctattttggagatttttgccagttggaatgcccagtccaattctgatgagaatcctgggttactgtgatgctgcatttgatgtcttattgcggggatctctaatatgttgtattgcattacatcagatttttaaaattatatcatgattctattaaaaatgtaacctcaagtcattttatttccttgacctcagaagttatctggagataacacatcatactaatgtgttttccaaaggtaggatggagttatctcagctgttgttgagttgttgttacatgtgatcaaaattttatcacttaaatttcttactcagtgtgtggttttaatgtggagttcatagaaaaatggttatcagtgattagagaatagtttattaataaaattaataactaggaaaactgagtcatagagagaaatactccctaaattatacattatgtaatttttttggaagtttctaagtagatccactttgtataatagcagattcctttcaaaggacttctggcattgactcttttgccagttttttggaaacctagtatcagttatttgaggttagcagactcagcgttttcagctcctgcctcccaaatgatactgtgtgttctatttaggttttttaaatttttattttatttttttatttctttttgttttgttttgtttttttgagacagtcttgctctgtcacctaggctggagtgcagtggcacagtcttgactactgcaacctccgcctcctgggttgaagcgattctcatgcctcagcctcctgagtagctgggactacaggtgcatgccaccacgcctggctaacttttgtatttttggtagagacagggtttcgccatgttgcccaggctggtgttaaacttccgggctcaagagatccacccacttcggcctcccaaggtgctgggactgctggcgtgagccactgtgcccggcctatttagtttttaaagtttacaatttgtgagtattctagcattgtgtctgagaatccaaaggagatagattacattaaggaaatagcaaacctgagatgtttcagaaaattacgagtgctagaaatgctgaatcctggtcttctcaaaatattagcatctttagccttcttaattatgttgtgcaagaagcttcaaacaaaaccgtactttggaactgcaaaaatactactgttttccctacatatggtgtttcctagggatgggcactgtcccctcggggttattccctgtggctatttattttacattagaacatttgtaaatttaacaagagcattttttggtgccttcatatttctcatagtgtagtaaattagattgttcagcctatagcagacatgctttcattagccttcctttgatgtattctgctgtgttaatattatgaataaaatatgtggccaataaagggatatcactacttgcaatgcagcatgagctatacaaagtagagtggaattatcttcattgatttgaacacaatacttttgtagcctaatgttgcatgatgttttttattggttgcatctcagtgttttttctttttgagttctgttcaattacacttctttaatttccagatgtattactgtcaagtttgatctggagttcctctgttctctcattctcaataatacattgacactttgattttagtcatagggcctttgttaaatttcatattgcaggtttggtctatcataccagcccaagttctttaaatttttattcaatgtttcattttaagccattcttctaggtctgtgtcaatcttaggtggctgtgttgaaatcagaggttataaattgacagccaaatctatcccatgatgtgttttatttgacaagcacatttttttttttaaactagaatgcttgtagttagttaagttttgtactgtgtgatttaccaactgttgccccttattgcgttacacaatttggcttcacacttttacatatgttgcttagctgttgatggcatttgcaacctgtaataaatgaaatcatttataaaaatattgagacaagggaaggacagtttatagtcctgtaactagaaatctctgttgaacttggcattcatcttttaatcttacatataatttaaatatataaaattaaataaatgtgtgtgtgcatgcgcacgtgtgtgtgtgtgtgtgtgtgtgtgtgtgtgtgtgtgtgtgtgtaggagacagaatctcactgtgtcgtccaggctgggttgcagtggtgccatcacggctcactagccttgacttttcgggctcaagtgatcctcccaccttagcctcccgagtcgctgtgaccacaggcgtgtgctgccaggcctggctaattattattattattttttttttttttttttgaggcagagtttcgctcttgttgcccagactggggtgcaatggcgtgatctcagctcagagcaacctctgcctcctggttcaagccattctcctgcctcagcgtccagagtagctgggattacaggcatgcgccaccatgcctggctaattttgtatttttagtagagacgggatttctccatgttggtcaggctggtctcgaactccagacctcaggtgatccgcccacctcggcctgccaaagtgctgggattacaggcgtgagccaccatgcctggccaattttttgtttttttgtagaaatggtgcctccctgtgttgcccaggctggttgtgaactcctggactcaagtgatccttctgccttggcctcctacagtgctgggattataggcatgagccactgcgcctggttcagcctttatattcttaactgttagtatgattgtttagcaaatgaagtaaattggaatgtagtggtatacacatctccaggttgttcataattgtataatgattaattttgtaaaatggatcattaaaatcaagaaactttgtgattgagtaatagttaaaaaaatggttttgacatctgtgttaatgacctttctaatacctgagactcaccattctttgaattctatttaatacagcaatccttttcacaccaattgagcattctgttaatttgttctctttattagacctcatttacaatgggaactgattgtttctgaatattgatctctgcatttttttcttttagccttcagcattactcttgttacccctagttttgcatatcatcttatctcttactttacaagaaataggatcatcacacataactcattcagcttacatttctttccttttccctaatacctaatcacttattagatttaaagaaaaaaagataactaattttttaaggatgaaaatagctaaaactcgagtctttattgtgtgttaggtaatgtatttaaagaatttcacattgatgatttcttataatatcatgacagttctttaaaatagatactgtcattagctctatattgcagatgaggaaactgatgcttagagaggttaaataacttgctcatagttacataactcctagtaacagaattgggacttctatatagttttatgtgactcatgctaatcttacttcttaattactatgctatatagtttgtctttgtccttgtttcttccttctcctgattttctagaatcttgcttcttatcccttttccctctttttttttttttttttttttttatttttttgatatggagtctcgctctatctcccaggctggagtgcagtggtgtgatctcagcttactgcaacctctgcctcctgggttcaagcgattcttctgcctcagcctcccaagtagctaggactacaggcacctgccaccatgcccggctaattttttgtatttttagtagagacggggtttcaccgtgttagccaggatggtctcgatctcctgacctcatgatccgcccgcctctgcctcccaaagtgctgggattacaggcgtgagccaccaagcccggcctttcctccttttttataactttattctacctatgtctacttagcttacaaataaatttgggtctcttttagccagtaaaaagtgcaaacaaaagcccagcaacaatgtttctttgacttttgctccccttgagttaccaactcccagttctctccttacttcatcaccagagactttgtgagaataaactgaattaactacctcctattcctcatctgccacctgctcacttgtgaacccaataaagcttgattgctactggcaccatggtctgaaacctctttttccaagagcatcactgaaccaagatacaccaagatattgggtatagatcgaataacctctttttagtcctcatctcgtctgctattgaaatgcttcacagtttcacctctcacccttatagaaatttccactttgaattgtcgatgctaatctttccttgctttcttttgcctcttagttttttcttcatgtttgctaagtcccctgtgttgtctgcttcttaaatttagtgtttttcaggattccatactcagtcctttccttactctccaactctgggtggtcttcttaggtgaacaagtctattctcatggcttcagctattgcctgaatctcaagctgtactccactcttgcttcctgagttttgccttgacctctgtttgcaaccacctatgtaatataactactcaaagtgttctgagattcattaagtaaaagttcaaataatttttccttcaaacctttctactgtttttgctttctgtgtctcatcacctatttggttgcccaagcttaaagacagttaaattaatcccaatctctttgtttcttggattctctattagtctcatgttcaactagccggtatacctaccctgccttcccgctgctttaaaaaaagtccctattgccttatgccctgattcagcctttggactcttgtagaaggtttccagctgctcaccctgcattcattctcactttcccaagtctcctctccatacccattttccataatactgtttgagttaaaacagacatgtcagtttgtaatctaatttttttcttcaagctttattgatataattgaaaaataaaaatgtatatatttacagtgtacatgatttttatatgtgtatacattgtgaaatatgtaatactacattttttaaaatgcccattgcccttacacttaatttagtcagcaaatatttactgagtacctactgtgtgctaggcactattctacatggagatagagcagtgaataaaacagacaaaagcctttgccatcatggagccttgtctagtcagaggaagcggataaaataaaataaactatgtggttatacaatggtaagagtcatttataaagtggggaaggcagatggggaatgtgaaggtggaggaatgggttgtaattttaaatggaatgattaaggaaagccatactaagaaggtaacacttttaccaaagattttgaaggagttgagacaaatgagctttgagcatgtcttgggggagaacattctaggttaaagaattagaagccctgaagtacagaatccctgaatctgaaaatgtaccatatgtgttcaagaaatagcagggaagtagcttggctagagaaaagtaagcagtgaggacagcagtggagtagaagaatagaggacagaaataatgagatgggtgtgtagtaccttatagaccaatgaggtagagtagaagagatacagtctaacttgacttgattttagttgatagtagactggaaaggaataagggtagaagcagaccatttaggaagatattacagtaatccacgtgaaaggtgatagtagcttggacgagggttctaccagtgaagatgatgagtggttggatccagataaattttaaagatggaaccattcatatttcctggcatttggatatgagggtgtatgtgtgggtataggggtgtgtgtgtgtgtgtgtgtgtgtgtggaagtgtaagagagaaggggctgggagttgattaaggatgattctaaggttttcagccagagcaataccagatacacagttggatatactagtgtggagttaagggcagaggtctagtatatgtgtatgtatttatatataatcagtgtatagttgtattgagtcattagactggatgagattatcaagggggagtgagtttaaagaacagaagagatctgaggactaagtccagggatcttccaatttttaagagatcaaggagaataggaggaagcagaaatggagaaaagaagagttgtctgtagggtaggatgaaaaccaaaaaagtatagtgtcctatgaagtgaagaaggtgcttcaatgaaaggaacgtgattgacttttccaaatggtagtgccaagtcaaatagatgagaagggtaacatgatgactgaaaattggctttggattcagcaacaaggagattgttcatctggaataattttggcatagtggtagtgaaattctgttggagaaagttttagaataggaggagagaggaactggagaaagccagcttaaggagaggaggcagaaatatggtggtgattagtgtggttagagatagttttttttttttaagatgggagaaataatagcaagtttatgtgctgattgtaatgtttccacacggaataattaatgcagaaagacaaaaaaaattctggagcaatgaccttgagtacaggaccatttttgtttttgctttaacatgtagtgatgtttgccttaggaaggagctgggttagcttatctctagttacagaagtaaaataagagtatatggagatagatgctggtaggttagtgcttgtagtgatgcaagcaaggtcatctgttgaggctgaggttagagaaggagtgttagaggttgaagaggaggaaagaagaaattaagtggtctggaaaagtgaatggaccaggaaagtgtataattattgctggacaaattgattttgagacaaggtgtggctctgtcacccagactggagtgcatggcatgatcttggcttattgcaacctttgcctcccaggctgaagccattctcccacctccttctcccaagtaactgggactacaggtgcacaccaccacgcctggctaatttttgtattttttgtagagatggggttttgccatgttgcccaggctggtctcaaactcatgaactcaagcgatctgcctgcctcagcctcccaaaatgctgggattacaggcgtaagccaccacgcctggctaacaaattaaattttgatgatgacttgaacatgacaccagtcagcataattagaggtttctttgcagccatgttgagctctttggtcgtagacatggactgggcagaaagctgaactcaaccagggttgtggaaaaaccacaagctttttcttagatatttttggctttgtgggctatctagtctctgtcacaattactcagttctgtcactgtagtccaaaagcagccatagacaatacttaactgaataggcatagctctgttccattaaaagtttatttttaaaaacaggcagcagactattcctgttgtttgttaactctcgtgttagagaggataatgatggaccgtggaatttaagcaggctaagaagggaagagaggagaacataagtgcgtgaagtgtagagagaaggtagtagggtcaatggcatgatgtttcactatttcctaatctctgctctactcttacatgaccttatgttttgaatatagttttctctctctgcgtgaaatgcttcttttatacacagtttagatgtcatctcttttatacttttatttcctctgctgtggccactgataatatggaaaatgctcagtaaatgctgaaaattgaaattctccacttcagtttgataggtaagattatttcaaattgggagattgtgaaagtttgtttatccacagtatttctcactcagtagccaagcctatgcttgaacatggtgattctcaactttgttttctgcttcaatttacctgatactggccacatgaacaaggtaagggcagtgccctgctcttccttcctacttggtgcttaagttagaatgcctttctttttctccactggtgttgttgagttggtgtctttatgctgcagagcatggatatttaagcataggacctgtaatgagcttgtgagtatggtaacacgttgccttataggttattttttaaatttcagttaattatagaagacaagtgattgttctatcagttatatactatcaggtaactataagatctttttgtgtgtgtgtaggttagagacattttggactgtttagtctgacttttcatattgtagttgaagaaactgaagcctagagatgcaagtgacttgcacacaccaactagcagagctagggctagaaatccatgtttcttagtttcagattcagtactctccctgccaccacgatacttatcttaataggatatgaggacattgattatcagcctagaaaatattatattttatgacaggtgatatcctctaaaatcatggtaaccaaaataatcgtttaaatgtaattgggaatcttaacaatattgaaatgactattttttccccattgaattattagtaaaaaggtgtggttgacttgttttattattatggttatattctgaagtaattttatataattaaaagtgacacattaatctttaaaaatccgaataagtaaatatctagagaatttactctaatttgaatatttaaagaccagtttctaatctgttttgttttttgtttttttaaataaactctttcagaaagatgaggtctatcttaatctggtgctggactatgttccggaaacagtatacagagttgccagacactatagtcgagccaaacagacgctccctgtgatttatgtcaaggtatgtaaaaacataacaattttccattttagaaaacattttttatgaaccaaggagagcctcttttatgttttaactatattattgtatttaatactcagggagaaaaactgtaaagtaggtattgttggaactttgttaaatccttttacagttttaaaatgtgaattgtgcaccttttgaagttttataactcagatagtaaagatattctggtaaattacagtgaatatttttcacagacatttatagattttatttctgctagccacagtacattttaatttaacttctgatatctttattgtgccacaaaccaaaataggagcgtatcatataaaattgtaaaatatagttataaatatgttcaaattcatttgttcacagatgccatttatatattttttagcatctgtagctgcagcttaaactgttggcaattttaaatatgcattgggtgttgtttattttttggtatctaatatcaatgccatcttgtttatttcctagaactcatggcagaatggctaggtatttatctagcataatttgtattaaagttgtggttagttattagtaacaaattattttattaactatggcattgtgaaaactagtagttaagtttgaaagttcacatatctaaatacaatttttaaatttaaccctttatggaaataactgtagaaaataaaatgtcattgactctgaatgtcttatggttagagtttttgaagtcttgttaaaaatttaagcttcacaataataaaagggttatcaaagtagaaccattaataatagaagttctttggatggattttgcataagctaataataataataataataatagaagttcttcaagcacttttgttagctagattactttggcaaggagtcatatatatatatagtatatataatatacactatatattgtatatataatatataaactatatatactatagtgtatattacatatttatatataaactatactatatgtagtgtatatcatatatttatatataaagtatatatatactttatgtatagataaggatgttcttttttattatttttccctcctcccttccttccttccttccttccttccttccttccttccttccttccttccctccttccttccttccttccctccttccctacctacctacctacctacctacctaacctacctccttacctacctacctccttccttccttccttccttcatttcactctgttgcccaggctggagtgcagtggcacaatctcggctcactgcaacctccaccctctgggttcaagcgattctcctgcctcagcctcccaagtagctgggattacaggcacacgcgaccatgcccagctaatttttgtgtttttagtggagatgtggtttcatcatgttggctgggctggtcttgaactcctgacctcaattgatccaccagctgggactccccaagtactgagattacaggcgcaagccaccacgcctggccaggatattattttttaaaatgtaattaaattaccattgtcatacctatgaaaataaatggtatttcattagtgtcattagatacccagtttatatttgcctatgacataatttttttaagtttatatttttgaattggcattcagttaaggtttatatactgtgattggctgatgtgcctctaatgtttcttttaatccataggttctctttttctctctcttgaactttgttgctaaagaaaccggatcatttgacctatactttcttggtctggattttgctgaatttctgtagtgtaattagtatgttctttggtcatctatatttcctgcaaattggtggttagataactgataattaaatatatttttggtaggagtcaggaccacttcatttgtagtattgagtgctttctgaaggaagaacataatgtctttcttttcctgacgttagtagccattgatgatcactgcctaaattcattaattcattaaaggttacaaaatgttgatagtctactgttattattccttcttaatttataagctggaatagtttcataaagagagattttcccatatcaactatttgtgccctgagatatagtttatataaaaagacaggaaatgtatttgatcccttttctttatttacgaaaatttataatagtctattccatagcatcctccataggtaccaatgaatgaggtgttttgttgttttaaagtatcttatgtgagtcttaataatatttgaccgtttccttgctttctaacatgacaaagtattccaagttcatcttacacatttcttcctcagacttagaatcaaccttttcttcaagaagaccttgttcttttagtgggaggtggtatttagagaccacagtctggatgctagaggtgctaattgtcacgggattggttattgtttctgggtctttttagcgaacagagctaggaaatatgtgggtaattttctcagatgtaatgcatcttctttttttccaaaaataaatacaccatgagttcatactgatactttcaaataaaatcgatgagtttttacttaactgtaataatcttatatttgtatttcctttctcttacattgaaaatttcatttctcaatgatagtagtataattaataacttattctacccagcaccctacataaccatcttagaataacaattccaacactaggaccaccagctaaaaataatttaagatctaagcacagctcagttttacccattagatcccattagggatgaacaatcaaatttctctgttctaaagaaacactgaggtcaggcacattggctcatgcctgtaatccagcattttgggaggctgatgtgtgcagatcaccagaggtcgtcaggagttcaagaccagcctggccaacatggcgagaccccacctctactaaaaaatataaaaattggctgggcatcctggagcacacctgtaatcctagctactcaggaggctgaggtaggagaatcacttgtacctgagaagcagaggttgcagtaagccaagatcatgccactgcacttcagcctgggtgacagagcaagacttcatctcaaaaaaattgaataattcctctctgtgtgatgatgttccattaagcatttgtttgtttcagtttacttttattatagattgtggttttctaaattttcaaataaaatttactttaaaattcatataaaatatttatacatagttcaaagcaagattgacaaaacaaggtatgttcaaagaagttcagcttttatctctgttccctctgctctgttcctttcctctaccagtgttaagtattttttaagtgttcataatttatcctaccatttttaaaataagcaaacatgcagttattcttactctttttatataaatggttacataaggtacacatttttctcttttttttcagttaaaaatatgtcctagagatcagtccatattttcgtgtgtgtgtgtgtgtgtgtcaccttctttatggctaccatattacttcatcatgtggctgtattctagttaattcacccagtgaccaataatgcacatattgttttcctcttgttttttgttgtggtttttttggtttttgctatagtgaacaaccctgtgcacttacggtatttttgccagcaaatagttgagctagatatctggcatcctgtattttaaaaaagaaaataaaattattgtacatactgaagttggcatacagagagaaatctagcactcctttctttctcccccttgtgggaaaagggatgatttttcttttttttttttttcttttttttttttcttatgtaaccacagcgctttcagaggccagatctattggctctgggcattattctttggggacatcgatcccttttatctgaaaagataaaaacagtgtctggaattgatgagttatgagttgatctccttcacgtagtcattctacatagttttattgtaatcctattgtatgaaaaatgttagattgtgagagcgatataaaagtagtaaatgcaagtaactcttgccttcatgaacctcatcatctgataaggacttgtttggcatacatgttaccagaatggcaactgcatttctttataattgcctttcactttttctgggagagttgagtattttctatgagtaatacacaggttaaaaactgagcacctttgcttttctgatgagtatatttcagaacttaagtaggttttaaaatgcaactctaaaacagatgactggcaaatcattgttgtgaacttaatttttttttaattgaaaatacatgtttaaggacagggtctgatcacacaagagagttggatgcattttttgaatcatgaacaaaattgttattacttgttttaacatgtattctattgttttattattttattacttcagatattatggcacagagaagagggcattggatttgagttaaatataactaactgttcaaattgtgctcaactgcaaaatacattcctttctgtgatattttgtcctgtaactgaagcatagtaattgttttatggaaatgttaacagttttgtaccaactttgaataaaatgaaaaataaaaaaaattgttttcattgaacctcaatatcttctgtaaaaacagggataatgtgtatctcagagtggttgcaagaattaactaaacaaataggcaaataggcagaaatgcttttcttggtttggcacttagtatatgttaaatctgttatattttaaagtgtgttaattatatttgagtttctaagaattaatcattctagtcttttatttagccaccaaactgctttttttttgttttgttttgtttttgtttttttgagagagagagaattccgctctgtggcccaggctggagtgcagtggtgcattctgggttcactgcaacctctatctcccgggtccaagcaattctcctgccccagccttccgagctgggattacaggcgtgcaccaccaagaccggctaattttttgcattttagtagagacggggtttcaccatgttgcccccagggtggtctcgaactcctgagctcaggcaacccgcctgccttggtctcccaaagtgctagggttataggtgtgagccaccgcgcccggcctccaccaagctatttaatgattatattttacacctttatttaataaaatatggcaaaatacataaatagtaattgaaagagaagcacttactcaaaccactacagaatattgtgtgtgtgtgatattgagcaagttgcttaatctctctgggcctcagttattttatatttaaaatggtaataatattagtacttattacataaggttattataaggagtaagtgaaataatacacataaagcagttagaacagtgcccagcccatgataagttgtttttctgttcttgtttttgctatcattctagtctagaaagcctctcagctttcttataaatacacttttacaaagtgaaataacaacaaatatataatattgcatatttcattctttatcttgacttttctagataaacttttcaaaacaatgaagatagagtagatctttcagatgaaagaatacagtaaaaagacatcttaaaattaacctcatttaaacgattaagctttttttttttaaaaaaaaaaaagaaaactcgttttcactgattcttgcagacctagacatcaataccctgacacagtaaaatctctttttttagtaaatttatgcctatgaactcagtttcttatgggaggttatctttttagtggtaatgaattaaactcctacagtaagctttaggacttagaaatggattctgtgaatgatggtacaataatagaactatgaagtttttaaaaaatgttacatggatacctaaatgccttaattttaactttgttggcaaaatgctgaccttataaaagatcttatctgtacagtttacatcttgcttcattttaaagaaaacataaacttttccagctaatagttcacattaacttcaaggatgtactaaaattcagctgtctgcagttttcaccatgcttcttagaatgaaagtaggtttcatgttaactttaattaacctaagccttagcagtagcttttaaagatggtgccatatgtcaatgcttcattagacttttaagtatctttttttttaattgtttttttaccaagatatttattatctttggtttgactcatttcaaaaattctgtccatggtgtaactttggggtagaggtcagaaggtaaacagccagaaaagtttaaaagtgaatcagaacactgaccttgagataaacagaaggtttaaattatactctcttcgtccatgaagtttctctctcctccagtcaagagtctgattctttactccatgcagacccagtttctcagtgtatctttttggtaagaaaactgagaaagtcttctcttttgaaagatgatgtcaatagtcaggcatctgtttttgaatgtcattttgaattatactttttctgaaggatattaacaggaagaatggtgatgctactaacagggttattgatgtcttgatagagcagtgattacagattttctcagtccacatgagttttcctagaccactgtatcaatcaccttggaggctgacatctcagaagaatattctaaagatcacactttggaaatacgtttacattcagcagcatgctctactagaaagaactataaacttaaaaatcaggagacctggtttctggcccaaagttactgattattcatgatttggggtgcctctctgagcttttcatttgtgttttaaataaaagcccatcttagcaggcttgtaatgtgagatgttagtaaaattaaaagttataaagcacattgtaaaattttcttcttttgagttattaaagaatgaagcgaatagtacatatggttaatgacagtttagctaattcatatagaaatagctaattaatacagaagtgacacaaaacttgtgaaaggaagattgagtgccagtaatccatagcatttcacctgttcttttctgagaatctgttactttattgactgcttaaggattctgatttgtttagaataggaagtatttaattaatttctactatctattccagttgtatatgtatcagctgttccgaagtttagcctatatccattcctttggaatctgccatcgggatattaaaccgcagaacctcttgttggatcctgatactgctgtattaaaactctgtgactttggaaggtaagctactgtccctctccctgccccttcccttttcccctcctccccctactatcttcttttaaatatattccttttgcaatatcagcctaaacacagtaattcttattcaagaagtataaactagtgcagctgtgagagtagttttgttagtttctgtcaaaattataaatgtatatactccttcatttaacagtttaacttttaggaattatttccttaggaatacatatttacaatacagtgtatatatagggatttttattatagcattgtttataagcatccattagtaggaaaatggctaaataaattacggtacatgcagagtaatacatgcaaccatgaaaaagaatgaggcaactctctgtgaactgaaagggaatgatgtctaggaaacattaaatgaagaaagcaagatcaagagtatatgtattgtatgccaccttttatgttttaaaaacatacagaatgtgactatatatatatatatgtacacacacacatatatacacaagtacatgtgtggcttatttctgaaatggtacgtaatccataatatttgattgcctgagaaaggatctttgggaatataatgtagttgggaaactcactttttcattgattgtattttagtattgtttgaatttacattaggtatatatactcctttttcagtaagaactaaattttttcaaaatattgattattatacattcctgtgtatctttttaacttcacaattaacctcaattcagggatgagataaaaaacaatgcagtgttttcttcattttcctctataatttaaccttttattccaccctttttcctgcaaatttcatcaaactttctgcttttaaaagctcatttcttggtaatatactgtgaatttgtaatacaactttattctttccattttcagttttcagtactttgtgcctctctgatctaaccttttaacatgtagtcaagcctacattcccatgtaactcaggcagttaccccgctacttcctcactggtgtaatcacctaatctctctcctcatggtccctaagacatctttacataatttggattctctccaatttaattttttcacccttgttacagattcttttcaccatagctctatagataacatattgccccacacttgttaatacctgtgttcctatccctttgtaatacttttacatcaattcacagtttaaatttggggtttaacataggatatgttttccacaggcttctcttgagacagatcctttaaaaaataagtaaatctgttctgtcatgagttaatttttcttagtctcacaatgtgtcctctttcagactgtcctcattttactgttttctaatttcagatgacctatcactttgcaggccatgactctgttccctgaactatttttaaaaagggtgaaagatagtaaaactcttacctggtgtttgaaacaaagcactactacttgtctataggaaaaactgcccaaatcagatatgttttattgttaatggggtgtcttcaccattgagagtgactgacttggtagggacagactgagacaccaaggttagaagagcttctgttctcatatagtgtcaaaacacttggtacagcccattctcttgggacataaaatagctttattctgataaatttggaagagggatttcgcttgtttgatagctcatttttataaaccctctgatttttttgtttttaacaaaattggtttggtcagatggagtttgggaaacactttggactgtacattatcagattggtaatttgaacaaaatactattactttcttcttatttttttactagatgaaaaggggccaaaaagtagctaaagaactagttgggctatttaggaacaaaaagctagatgtgatcacaatattgggaagtagttaaaaccagtctcctctgtaagagaggctgctccgtttttgtagctccatcaggtagtgaaaaaacttcaggctacaaagcttgggtattaacgttttgtctcgtaatgaattttctgaacataattagcatctatcttgttaattaaaaagcatcctttagaagatataacagtctcctatcaattatgctgaaaactaaatgtcataattttcatgcagcctaaaaacagggattagtatttattaatttatgaaatggttattgtatgtcagtacttggggagagggttaaaaaacaaacttcaaaactagtgatgaaggaaaaagaagttaaggttagagtcatcattgactagaaataaatggaagaaaagaaatcccagtcattgaaatccttgtgactactttcatctgtctttaccctggttttccagtacctttactttttttttttggcacttactgttttactgttttgattttgttgcattttgtccctttatttatctttattattaatgtatgtcagaatggctggcttctacagcagtttattttattctgtctttggattatcttgggcgacatctcaaaagaaattaattgtaatttattatacttcatatttaaaatacatgcaatgaagtattttagtaggaaaattggaataagaaaacatgcaattatacttactatagcagagttagaatttataggcagtgaatcatttctgccctgaagttgaagggcctgtttataatttattgggtaataaatgtttatgtggttaataaattgaccaagatatggaaaaatagattgagtctataagccttgaaatattcgccgtgagtcagaagtcaaaatgcaagacagtggccagtttctcaccctttttaaagaaatacattgggtgaggagtattcattcttagaatccattgtcatgctttttggtgggggaggtacctcttttctgcccttgcttctttgtattttagaggtggaatttggggaacagaggatcagaagaaattcaacacttatccttttttgcttttggtgccttcttaggtgacaaacgctttctttaaaaattgctattcttttttgcagagcaaggtgataaaattgtaaatttcttttatyagagatagttacagtttacattttctttttcatatatgattcaaatgatgtgaatcttataaaatcatttgatactgtgaaaggatagcagtctaagttctttaaggtttagatttaaagaatttctgctttttatttttattttttttctgttagtgcaaagcagctggtccgaggagaacccaatgtttcgtatatctgttctcggtactatagggcaccagagttgatctttggagccactgattatacctctagtataggtaagtacaaatctgaatataatgctcataattattaattgcttcttgattttggtaatttagtactactaatctgtacttttatttccatggaaagatggatatatcccataatgttttttttcttaaagcttgagatgcaaacatgtataggcatacctcattttattatactttccaggcactgcttttttttttttaacctctcaaaggtttgtgtcaaccctgagtcaagcaagtctgcccacactagttttataagagcatgtgctcacttcatgtctttgtgtcatcatgtctttgtgtcacattttggtaattattgcaacatttcaaacattctcattgtatctgctatggtgatctatgatcagtggtctttgatgttactattgtaattgttttggtttgccacaaaccacaacaatataagacagcaaacttaatcgatcggtgttgtgtatgttatgaatattccaccagctggccattccccatctctcttcctatcctcaggcctccctattccctgagagacaacaatattgaaattaggccaattaataactctgcagttgcctctaagtgttcaagtgaaaggtacatttttcacttttaatcaaaagctagaaatgattaagctgagcaaggaaggcatatcgaaattgtgccacacagttagccaagttgtgaccagaaaggaaaagttcttaaaggaaattaaagtgctactccaatgaacagatgaatgataagaaagtgaaacagccttattgctgttacagggaaagttttagtggtttggatagatcaaaccaaccacaacattcacttgagccaaagcctaacccagcaaggcctgaactcccttcaattctgtgaagacagagaggtgaggaagctacacaagaaaaatctgaaggtagcagaggttatttcatgaggattaagggtaaaaagctgcaccatgccataaaagtgcaaggcaagcagcaagtgctgatgtagaagctgcagcaagttatccagaagatctaactaagatcatcgatgaaggtggctacattaaacatcagagttttaatggagaccaaatagccttctactgaattggaagaagataccgtttaggactttcatctctagattttctagagatggaagacttcttctagagaggagaagtgtcattgcctggcttcaaagcttctaaggacaggttgactctcttgtgagggcctaatgtagctggtgacttttaagtgcaagccactgctcatttaccattccagaaatcctagggcccttaagaatgatgctgaatgtattctgcctgtgctgcataaatggagcaacaatgagtggatgatagcacatctgtttatagcatggttcactgaatatttgaagcccactgctgagacctactactccagagaagagtatttctttcaaaatattactgcttatggacagtgcaactggtcacccaaaggttgagaaatacatttgtaaggctatagctgccctagatagtgattagtctgatggatctgggcaaagtcaattttaaatcttctggaaaggattcaccattctaggtgtcactaagaacatttgtgactcaatcacatgaacaacattttggaagaagttgattccaagcctcatgggtgaccttgaggcattctagacttcagtgaaggaagtctctgcagatgttgtggaactagcaagagaactagaattagaagtggagcctgaagatgtgactgaactgctgcaatctcatgatgaaacataaacaagttgcttcttttatggatgagcaaagaaagtggtttcttgaggtggaatctactagtggttaagacgctgtgaatattgttgaaatgacaacaaaggatttagaatattacataattgtagttgataaagcagcagcaggggttgagaggactgactcccattttgaaagtggttctaatgtgggtaaaatgctatcaaagagcatctcctactagagaaaaatctttcatgaagggaagagtcagttgatgtggcaaacttcattgtcactttaagaaattactccagccacatcgtatagccttcagcaactgccaccctgatcagtcagcagccatcgacatcaaggcaagaccctctgccagcaaaaagattacaactcactgaaggcttagatgatcattagcatttttttttagtaataaagtgtgtttaagttaatgtattacatttatgtttttatacaattctattatacaagtaatagactacagtatagagtgaacataccttttatatgcactgggataccagaaaattggtgagacttgctttattgtgtcttattgttatggtctggaaccagttctgcaacatctctgacgtatgcctgtatagaatttttcaaattctactttttagaatttaaggtatacctactattctatttttcttccttggactgttagaatcataaagctaatagcttttaccttaagaatttaaattaattctaagtaacttttttttactttcttatttaaaatcctcctgaaatacttctgagcatgattatactagttggataatttattaattaatgttatttcattaagttaataatgaaagtgatagtatggccatattgctaacatttccttcatgccatctccattatttaatataaaatcagcttaaatcactagtagttttgctagatgcactgattctgcagaagtatgcacccaagatgtttagtcatagaaggaactgaatagagaccattagctttaatcatcattgaccttctcaagtcttcaccccagatgtttagtgataggaggaactgaatagagaccattagctttaatcatcattgaccttctcaagtcttcaccccagatgtttagtgataggaggaactgaatagagaccattagctttaatcatcattgaccttctcaagtcttcacccaagatgtttagtgatagaaggaactgaatagagaccattagctttaatcatcattgaccttctcaagtcgtcacccaagatgtttagtgatagaaggaactgaatagagaccattagctttaatcattattgaccttctcttttactacatcttagcccactagccaggagtgaacagagagagcatgatcagctggacatgaataggaagctaacaaacaaggagcaactttaagctggatgtgcaagtggaaaccaaaattattttactcttacatattgtttctgaagagaattcctttagtccagaaacaaacaaaagcatgcaaataataaaccaaaaacatccccacagtaacagaaaaccaaaacaaacaaaaaacagacctggcctggtgcagtggctcacacctgtaatcccagcactttgggaggctgaggtgggcagatcacttgaactcaggagttcgagaccagcctgggcaacatggtgaaaacctgtcactaccaaaaattagccgggcgtggtgacatgcccctgtagtccccgctacttgggaggatgaggtgggaggatggcttgaggccaggaggcagaggttgcagtgagctgagactccaccactgcactccagctcggtgacagaatgagaccccccctctcaaaacaacaacagcaacaacaaaaaatagaacagaccttgtgacagtttttttgtctttttgatataaatagaaattctctgatacaagcaaacaccctaatcagaaacacattatcattttttattttgccaaatggcaaaatgccaaagcacattttgatttctgccaagaggccagcagttttacccaccatttcctttgtaccctcagtacaaatgttaacccagtgaaggtgtattcttatgaaaagagttttgccctctgggacatctagaaatgtctagaggacccaggtgcaaagagtacaaagtgtcctctgtagaccacactttgacatctgctttaccagagggtagagagcataataatgtgtatatgtatgtgtatgtaattctcagagtcttagtatggctgtggtacatggcatatatgaatggacagatgaaaaatagtacaaagaatattttgcttgtggtcgacctgggtttgagtgtcaggtcacttgaagtaaagtggagattactgacagttcttttctgagtggctgtgaggcttaaataaaatgttagtgaaaatactgtagaaatgaaaggtggtattcttttgtggcatttttagacttgactctccccaagagcaaatttcagccccttgtctttgggtatgtcattggattttccagctgctttacattttattcaaatatatgatactgattatctgtatataagatactggtgtgagctgtgccagaaacacctaaaataaaggcagtttgattgttctcaaaaagttaacacatcatctcacacattaaggaacagttatataaacaggagtcttttgaaccctagcaagcctaggtttggcttcccattataaacttaggatacttgcctcttaagactgtaatgtttttaaacagaccctcccaaaaagtatactaatattgttacatttttaaatgtttttaacagtaatgtttgaaatgaaagagtaactgtttctgacaagattattgtcatatttagatacagccagaggtaaagctgatgtctctgattatattatcttactgacaaatggcagaatgagaacaaaaggcttccttagaacaaggaattgaacttacagacagacccttgaagaagctgttattctgtgacagtgagacagttattcaagagagtggtgttttgattgggaattgagaatgattccaggcctagctctactattcagtacctttaccttgggcaagtcgtttaactttatcatgtcttaatttccttatctatgaaataagtgtgttaaactagatggtcttgaaagatcactttaacatgaacccgagttaaatatgaaacaaaatgctattctggctttaaccccaactaactaaaattcagtgtgccaaagcactatttctatttttccggtaccttctatttagttgaagttttattctatctggagaatttcatccatttgtcctttgaagtaagagcagaagagattttagtgtcctccagaaagtgattgataagtaccttgtttttactaacttgactgatgtaacatgaattgagggctgctagtatagttgtcacctgaatcttttgagtgtcagtcttctgtgataacttctaggcaagagtattccttgatgttttactttttgtcttccatatttgtagctaaatcttacctaagaaagtgtttcctattgttttttatgaaggcataattattcccttacaattttttcagccttgtatctgagatgaatcattccgtattagagaggaaaaaagttgagtgcatattaccttccatcttattaactaggtaaagggatgttttaaaacttagattaaaataagcatttacataaataaggagatacatcttttccgcttgactataacagttagtattactattggctttgagtaaagattccctaacaagttttcatatgtgcttaatttagcaatttcctaatatattaaataggactcttggtctatatttgcagaatagatgtcttggttattaagttaggactggacaaaatcaactcgagtttgttaaatgatactatgtcatttttctaggtcaataaaaagtagtcactggagcaacgatttccagtctctcttgatagtacatctcaccacatcagacctttaaaattttttgttgtcaaccatttactgttccttcttctccttccgtactggtaggtggtaaagtccagcaatgattcatattcgtctccctaaagcacttgagtgttggtacattaaaaacctaggaatctcagaaatctggaagctaaaaagcagccttttcttgtaggaagtcttaatagcgggctttaaaatagagtgaccgtacggtttatcttctaatccgataatacttttgagagagaaagaggtgttactaataattatcccaggacaacatataaaccataactatcctgggtaaaccaggatgtatactttaaagtatttttaaagtgtattaattgagttaatttttagagttttctctccggattgaggttttcattttgacttaaaagtggattatttttataatttatgaagtaaaatgtattcctccatcattgctattccattttatttaaaaatgatatatttaagcacatttttctgtcagtcatcgtagaacataaaaatcaaccatttaaagtcttaaaagtgttcctcagaatttgcactgacttaaagcccagtgtctgggagagattgaacctttccttgctgctcctctgtaagtacctactaatatgcagacgttgtgtcactttgacttatttttaagtaagataaatagatagtcctgccatagctttggtactgtaatcctctgtctagttatttgaagacttgaatacctgaaatttgataataatagactttaccctttattagaatattcttattggtctggtttcctctttggtaagtttaacatttaattaaaaagtacattaaggaagtttgatgactttttctctgttttggattgtataatcttttcctgtgatatttactctgtgatcaaagtaaaattagaaaacgtatacaaacacttttcagctcaagctatgaagtattaataaggcttcgttttcttttctctttagatgtatggtctgctggctgtgtgttggctgagctgttactaggacaaccaatatttccaggggatagtggtgtggatcagttggtagaaataatcaaggtgagaaggttgaatcagaatatttgaactggaaggaatttttgtgctactttaataatatatatcacacatgtacacatacgagaaacttaaaaaatggaggaagcaatccaaggtcacgtagcaagttcagctataaagtgggtataagaacgcccccccagccttttggtttttatttgagttactcttctcttgtatgatcctagaaaagctattatgtatatctgatgcaagaaaggtatatatctgttatttaaagtacttaaaagcaaaattagactataagctcactaaaatgtatgtaagctgaataaaagcagaagctgtgtctgcttttgctcactgtatactccctgctcctaatactctgtggagcacataggagatacttgtatttggtcactatttatatttattcagtatttataaaatgaaagaatgaatgcctacctgtctggctagatcattgatagaaacaaaattttgacttatggatttaatataattttatttattgggtctatatgaatttaatataattttatttattttaaattttatttaaagctgaataaagatatgtctggttttgctcagacacagcttctgctttcattcagctttgcatacatacacctttattaacaaatatttaaatattatcttagaatatatgttttcttttaagaacagtagagctgagattttaattgagtttttaaatgtaatttttttaagttttaaatatttgagatcttaaaggaagtccctttacttaaaaaaaaaaaaaaaacctagtgtctagatttatatcactagattgtttcattctaaggtttcagtttggacaaagcaacaacacctactgtctctggggctttgtttgtgatatcccaaattttgcatagaattgtaaaattgaaaaatctccttagaggtcatgttgtccaatgtacaataaaataaccttttagttacattcctaacaaatggttttctagtattttcttgaacacttccagcatcagattactacttagggatgcagcttattctgtattggacagcttgaatttagtttttattgtactgagctgtaactcctcagtagcttctaccagttgaacataggactgtgatcaagttcttttcaaatccttgtacttcattctataaaaataggaattttaaaaatcaaaggtggttttgctagatgtcagtgtgtgcaagtatggttgaaaagactagtgtgtaataagcgatcttctttattatccatataatttcttacaatttgcagcttagaccactgtggtatttgcatcttctttttggtaccatttttgagtttcctatctaatcttttacacttgttaacagaactattttctttttcatttctgtgctacacttagatgtaaatttgaatagtttgctttctttattttctaaaagcctaccccatcatgtaaattaatgcttgaatacggagcttggccaactttttctgtaaaggactagataaaatattttaggctttgtgggccaaaaggcaaaatcgaggatatcatataggtacttcagtaacaaaagagaaaacacattttcacaaatttttttattgatgaagttagaggtataataattgagtcttttttttatttttatttttaaatacagctctactaaccagaagaattgagttctttttggggaaatataacctttctcttaattgatgttcaaagttagtactttctaatcatcaaattgatggcaaatctgttaattcttagcttatgggcttcacacagacaatgggctgtagtttcctggtctctgctcttacacaaccattcagcatttctcctgtccctaggcttttatttgacgtacttgagctcaccatcaccatgctacaattctgtgggcatattagctattctatttgtcatatatgtttagctcattacaattgtatcatttctctactgattatattttagaattttgtggcgatttgtcattaattttaattcatacataatgcaagtagaatttctgaggaagctaagtgagcatctgtgataattccctatctcatgcttgccgtaataccttataaatctttaagtctagagcttgtttgtacaactttttgttagtccttgtaattcattttctgctaatactattttatgctacctgctattaatatattttgttactgataatttcagacttcattttcattaagtgcttattttttccaaaatggtccgtgatcaatatgtagtagcaggccaactctgaaataaaacactcatcatttatgtgctatgctgtcatcttgcatttatacttaagaaatgtacttatcaccttactatagtaagtaagttccaaaatcactgcttatagaaagttttgataatgatataagataagagaactgtaagtatttccaagcatccagggcattttgggatatctggtttctaggtgccttctatgttcttgaccgtggctgcccaggatagatggaattaccagtagtagtttaccatccatttattgactggaagttaccacacattgcacggaggctctaggctttgtattttgtgcaagtgtgtatatgtttagtatagttctaagtcttattgcatgaaggttaataaacttcataagagtgttgtgtgtgtgtgtttaatgtagccccaaatcttactggtccggccaggggccgtagttcacgcctgtaaccccagcactttgggaggccgaggtgggtggattgcttgagcccaggagtttgagactagcctagggaacatggcgaaacccagtctctacaaaaaatagaaaaattagccaggcatagtggcacatgcctgtagccccagctacttgggagcctgaagtgggagggtagcttcagcccgggagacaaatgttgcagtaagccaagattatgcccctgtactccacctgtgtgacagagcgagactgtgtcaacgagtccatcaataaaataataaataataaatatcacagccaagcgtggtggctaacacctgtaatcccaacactttgggaggccgaggtgcgcagatcacttgaggtcaagagttcaagaccagcctgaccaacatggtgaaaccctgtctctactaaaaatataaaaactagccaggtgccatggcgcacacctgtagtcctgggtactccagaggctgaggcaggagaattgcttgaacctgggaagcagatgttgcagtaagccaagtaccactgcactctagcctgggcggcagagtcagactctgtcacaaataaataaataaataataaatatcaaatctttttctttctccttccttcctgcttgcctccctccctccctccttcctattattgctaacatctgttcttcaaatgttaaatatatatgaggtttcatttgaataaaaattcctaagaaatgctgatctagctattctagttttcctgatttaagttggaaaagagaagttatatgtgtttgttttcaaagttaaatgttattaagacgttctgtattttaaacaaatactttcatgtactgataaacatgactttaagaatagatttgaaattttcttaaaggtggtctcataaatgcccagttcagttctcacgttttttaaggtaatagtcatgcacccttagtgcttatgataagtagggaactcgtattaaactgttcttttctgctttcttttcattttctttctagaaaacctcataagagagtatctgggggaaactaaccctggaaaaatggataactcttctctgaatttaagtttaagcaaaaaaaagaaagtggataatcatataataagctttatgaagtttttttatgtggtagacttgcctcattattttataaacttttgcctcattattttataaactttctgagcagggacttttttttattcttatgttttaatatctgagggcaccaaaataattttcaattaaggtttgtattgaattaacttgttaaaccattgtccattcatgaatttgaaataagctgggaattaataagaaaagttaatgtggttgttgtgtatgttgtttattgactcagatgtccatgccagtaatttacagcatgagtaaaacagtttaattaaacgaactgtatctcattctagctaacatttttaattttcctcactgccctctaataaggtttttgtaaaatatttagaataaaatccaaatacattatctttagaataagttaaaagtataagaacagatttgaaagtcatggaaaatcatttctaagcatgaaaaacacaagtatttaactttctgaagttaatttcatgcatagaattgtcagatcagcatttcattatgccatttgacctctacatatcaaaaatgcttggattttcacggcatttgtaaaaagacaaattccattaggggcttctcagacaatgtgctttttttcctttaaattcttccttaatataaaaacctcaagaaatatatgatatagttccagatacatgggaaaaaataaaatatttttggaaaatctctaccaggaattttgccaaatgtaatgttcactgacatacaacattgtcctttggcacctctgcagttggccatagcagtggagctttgtagaagagagtcctgtgcaggcttgtataatgctggtgaaggtgataatttttaatgacagttactttttttataactacatagtcttagattttatatgctcaatgcagaagactgagaaagcacagacaaatagacaaaaatcacttactgttctagcacatcatgaaaaatacttctataaatattttcatatactatattttctttttatttaaataaaaataccatcttactgctttgtagacttttaaaaactgaacaattaagtatattttttaatgactacatagattccactgcatgattgtattttgaattacataattgatgctgaaaataacagcattaaggtaaagtgatacagcattgtgtttcaaaaagtttacatgatgtattggttttcgtaagtttgattattaactttcttacataaatgacaggtgtcccccagaaattccatataagcaatctaggaagttttggacctgggaaagctgtaagttttactaataatttctcccctctactcttcaaactttaaaacataaatttcaaataaggtaagatgaatttcagtccagagtttaatttctggattaaactatcacaaaactaacaatttaaaaataaaccaaccatttgtagaattttttaattttaaatattaaaattcctacagagatagtttcatttatatcataaacatttacttatgctatagtaataatttgactattgcttttgaatagaagagttttaggacagaattagtcaatgctacacctcttgataatagaatttcagtatagctttcatagtttttgtaactggcctttagaatttgggtatatcttctctaaaaagcttgacattaaatatattgatacttaaattatttgcaataataaaattattttataaggcaattaaattttcttaatttcttttgtaatacagctttactaaactactgtttagttttctcttaacattaattaaccaacctagatatctatgtagatagcttttacacagggaagagctagtaatttcagttatctgtaactgaaattttatgtaagaaatttgaaagtacaacacagatgcaaataaacttttgattgtgctatgggagccaagtagatgggcgggcttcatttcttatgatgcctatccgtagaaaaagatttagaaggacctacaaattctacaatggagaatagaattaagggaaggggaaaaggagagaatttttattttttgctttttactactatgtgatgttgattttttctaagaaatcatatctttttacagttgcaggacttatgaaaaagtagttgctttcaattttttgtttttgttagtaaacaaaaacagttctacagaagttgagagttacatttttgttacaaatgtttgtctaaataattgttttcctaggctttcaatttgatgcttgtttgtggtataatttagacaaaaagatcagtggatccctggctcctcagagttctacgttttccaatattgggaaatgactgctcatcataaaagctgccactcagtgagaccttactttgtgccaggcactgggttaagcactttacattcattaccacacttaattattatattaaccctaagaagtaagcatcactactactactattactgttactacagtagtctcctgatatccacaggtatcactttctactgttttagttacccagagccaaccacagttcagaaacattaagtggaaaattttagtaataaacaatttatgagttttaaattgcatgctgttctgagtagtatgctgaaatctcatgctgtcctgcttcattcctcctgggacgtgaatcctccctttgtccagtatatccatgctgtatacactacctgcccgttagtcatcgacatcatttgctcctgacatccagccatggacattattatggctcactgatccaggatcattccaagcagatgatcctcctcctggtgtacagtcagaagatcattagtagactaaggctatgtcacaaggcctgtgtaattcacctcacttcatcttaccacataggcattttatcatctcacatcatcacaacaagaaggataagtgtagtacaagaaaatattttgagagagagactacattcacagaactttacagtatatcattttaattgttgttgcttttaatctcttactgtgcctaatttataaattaaactttatcataggtatgtatgtataggaaaaacatagtatatagtttggtactacgtatgttttcaggcatccactgggagtcttggaacatatcctccacagttaaggggaaactactgtattaccgtatatacaggaagagtagggtggtggaaaaaccatctgattcatctgtagctcaatttatagcctctattcatggaattttgtaccttcgggaaaaactatatgatccttaatgtcaacttgatacccagaacccattgagttgtgtttattacttattatatgtatattgtaaaaatttgtgtctaccagcaaattagtgatgcaacccctaaatcttacagtagtgaaatcaatttgaggtatctttaggtttaattctgtttcaaccattgtaatggtcagttcctttgctttgctttcaattacacatggtccccacaattcctttgctttgctttcagttacagacagtccccgacttaggatggcttaatgattttttgactttccaatactgggaaagcagcacagattcagtggaaactgtattttgagtacctgtacaaccattctgtttttcatttttaagacaatatttaataggttacatgagatgtttgacactttatcatgaaatagtctttgtgtttgattattttgcccaactgtaggctaatgtaattgtgctgagcacatttaagggtagtctaggctgagctgtgatttcagtagattggacatattgtttgcattttcagcttaggatattttcagcttactatgtatgggtttgccatgatatgaccccattttaaatcaaggagcttctgtacagttcaaccttattttcaccatcttcatttagcaaatattttggattttaattatccctgtcctgtcctatagtttgttcagtgcccatgtgtacctacactcttgtttcctgacttaacattttcattgtcagacaatctttctttttctttttcttttttttttttttttttgagacagagtcttgctctgtcacccaggctggaagttcagtggcgctgtcttgactgactgattgtgttagttggtacttaatttgttcttgtattatcagttaaattttttcatttattgctgatcagagcagcagctcttaatagtttttaaatcatggactactttgaaaatctattagtaatagtggacacttccccccagaaaaatgaataaataccaaacagtttgtatgcaactttaggtgattcttggccttcccgtagctctcccaagaactccagggtaaacatctttcccctattatagtacaaggaaataagactacagttaataataaccacctgtagaaagtgggtattacacatacgatatgatgccaacatttttaaaataagaaaaacggaggcattaaaagaattattctaatgcaataactgctcaacattagtataatgttttccatagaccaaaagaaacattaattttaactaatattatgaaattgtatttggtctacagtaacttacccatacttagttaaatccctgtatacttaaaagtatgtgtgcctaggcatggaaaaataactcctggcgatgtagctcagtttgtcttttgacttgcagataatggcatccatttgaaatcgttacttttcccagaatatgcagtgtagttatttactaatttgccagtgatgttttatttatacaaagaaacttttttccagacaaactgaagtttcatgacattgtcacttcaaaactaatttatttatagttgtattttaggacctgagtcaaaggaccatccatatgattacttttcctgggatataataccctgtagtcatcaactgcagattcttgtgcagggttgccttttgagttattttgtgtgttgacctaatttcagggcctcaatttaatacattttctttggagaaatctataaagtatatgaaggggcaggtgagagatgggacagggacagaaatctgtcaggttgatagcatgcatatgctagacccctgacttcaggatctatgttttactaaggaattcttgcttccttttttgcaataaaaggttaaaatgtttgacaccaatgattcttaatagttggctagtaggaaaagtataagaaactagcaatcttgtagaaaggggcatttctagtttttaaaagtatactgaatgctatttcatatttataaagcaatattttattgtttatttgatgtataagatattaagacagtatgaaggaaatcttggcttgtgatgctacaaagaactatagtgttagtgattctcgaaggaaagtaaaactggttgatgtgtatacagatttttacatgtatttttttttaatccaagagattcatatacccccctttctattttatactgcttccttgagaataccactttagctgaaattcacctagaaaaaaaggtcttttgactcctatgaatatgtatcaaccaaaaatgaatttttcactttatgttcaatagtttacgtaatcaaaactttttggcggggaggaagatgatttgtttccaagtataaatttttatttagtaagaaaagaatgaagtccaaaacagtcttgaaaaagaataaagttggaggagccacttttttttttttttttttgagacagagtctcgctctgtcacccaggctggagtgcagtggtacaatcatggcttactgtagcctgcaccttcttggctcaagctgtcctcccaccttagcctcctgagtatctgggactacaggcacacaccaccatgcctggcaaatttttgtattctttgttatagataggttttcaccatgttgcccaggctgatctcaaactcctgggctcaagaagtgatccacctaccttggcctcccgaagtgctgggattacaggcatgatccaccacgcccagccacacttcttgttttcaaaacttactacacaactgcagtaatcaagacaatgtattactggcgtgaatgtagccatacagatcaattaagtagaatggagagtctagaaataaacctttatatttattgtcaattgatttttgacaaggattccaagataattcaatggggaaacaatagtctttttgacaaatggtgcttggacaactggatccatatgcaaaagaatgaagttggacccccacctcacaccatatacaaaaattatatagcttgtggagaaaagggaatccttgtacactgttgttaggaatgtaaattagtacaaccattatggaaaactatatggaggttcctcaaaaactgaaaataaaattacagtatgatccagcagttccacttctaggtaatctatcctgaagacttgaagtcagtatgtcaaagagatatctgtactctcaagttctgttcataaaagccaagatatggaaacaacgtaagtacctatcagtggatgaacggaaggaaaaatatggtatatatatatccactacagtggaatactacttagccttaagaaagaaggaaattctgtcgtttgtgacaacatgggtgaacctggaggacattatgctaagtagagtaagtcaggcatagaaagagaaatattgcatggtcttactaatatgtggattctgaaacgattgaattccagaaggagagagaagaatagtgtttaccagaaggtggagaggatgggaaatgttggtcaaagggtacaaaattatagttagacaggaggaacaaatgataagtatttaatgtaatcggtatgttaattagtctgattctgttattccacattgtatacccataatatcactgtttacctcaaaactatatacacttataattcgtcaattaattaaaaacaaatgaattcagaatgaatcaaagacctaaatgttagagccaaaacgataaaactcttagaaggaaacagatataaatctttgtgaacttcaattagtcaatattttcatacatgtaacacaaaaagcacacattataaaagaaaatagaagtaaattggactttgtcaaatttaaaaacttgtgctttagaagacagcatcaagaaacttaaaagacaattggagaaaatttttacaaattatgtctggtaagggatttgtatctacagtatgtaaggaattcttacaactcaataataaaaagaaagcccacttgaaaaatggacaaagaatcggcatagacacttctctaaagaagatacacaaatggccagtaagcatacaaaaagatgctcaacatcattattcatcaaggaaatgcaagtcagtaccataatgaatactattatcacactctccataatgactaaaataaaaagacagtaactaatgttggtgaggatatggggaaactggaaccttcatgcattgctggtgtggttgtaaaatgatgcaaaccttgaacaagtttggcacttcctcaaactattaaacattgagttatatgacccagcaattcagcttgtaggtatatatcctaataactgaaaacttacatccacacaaaacatgtacgcaaagtttatagcaatgttatatagccaaaaaaggaacaaataaaatttctagcaactgatgaatagataagaaaatgtcacacacgaaaggccacatattatataattccattctatttatattaaatgtccagaagagacagattgtggagacaaaaagattagtggttctggggagctgggggaaatgagaatgatggctcatggatacaggctttcattttgaggtaatgaacatgttctaaaattagacagcagtgatgattgcacacctctgtaaatgtagtaaaaatcactcagttgtataaggggtgggtttcatgatacatgaagtacagtaaagcttttttttttttttaaggcaaaagtgagcacatctatctgtaaaagaatgtctttgtcatttccactgcatcttaaggaaggcaataaaccctgactatagttttgcaaaggactctaaagggtttctcagagttgtaccatccaccccgattgccccctaaaagggaaggagagctgatcaacttgagttaaatgacaggcaagaaagagtgaatgaaatagagtataggcacacggggggaccctcctttagtagttcattctgtctagtttaaataaatactttacccaaattgtttacttgtgctcaaataatgtgacagcttgctgctctgaaaaatgatgaaaatcactgaagttcttaatggggctttaagactcatattgctgcagctatacctttttgctctttgccttttggaaattagaaggaaaccctgagagagaaatattgcaccccaagaaaatggttgcttataccaacttgtaatcatatagtataggggagaggtataaactgggtaaaatctgcattattgagcttattactttagctacctacaactagtagatttaaaaagtaaaacctgctgggtgcggtggttcatgcagtaaatcctagcactttgggaggctgagatgggtggatcacttgaggccaggagctcaagaccaacaggaggaagcttcatctctattaaaaatataaaaattagctgggtgtggtggagcacacctgtaatcccagctactcaggaggctgaggcacaagaatcacttgaacctgggaggtagaggttgcagtgagccaagattgcaccaccgcacaccaaccaggctgacagagcaagactctgtctcaaaaaataaatagtaaaatctgtaaattgtaggaaaatgccagaggagatgacatttcatatcagtcctctataagtgaaaatgatagtgattctattgtcattagactgatattttgaaacagaatacatgttttctatttagaaaaatttgtccttttgaggatatagcagccaatctaacacaacccaacactccaaattccatatgcagaaaataaatgaagatgtttaggtattcaaccatttatacaatgattatgttgtgttatataatttcatttttaaaacctttattgaattcattatctataagcctagccatacatccctttagtctttcaaggtgcataccacattaactacatgcgttaatttagtgaacagtttttactaggtgctgggcaagaacttagaggtttcttgccttcaggggcttcagagactgatggatgggaggagaggaactgagggcaatagggcaagcccagtattattccagatgtctgagcagaatacaaaggcagaagcatttcactcagagtagtgctgtctggaaagccttcttaggagagctaaccattgagctgaatattgaaacatgaaaaagtatttaccagatagggagcggaagggagtggataaatatttagtaaaactaggtttggtgtgtttctttatggttgtcgcttaaggttctgcctggtggggttgatagaatatgagtttaccaggaaggagaagactggagctgttgacagtggccagatcacattagcttgtaagcagttggaagcaattgcaggattttaagcggagtagcgacatttctaataggttttcttttacttcgctcttaaaggctacacatgcacctctattgctgaagtttggcgacctctggtgttgaactataaaataaacaacgttgtggaagaaatactagcgtttcagagcttgtcagttgctttttgtgaggtgcttcttgggccgtagtacttctttgaagacagtaattatcaactcataattcctgtgttgatatttttcagtgtttggataaatttctaactaggtagggccaggaacagataaattgattgatttaatagtaaggggatgaacaacatccccaaaggggtggggcaggctgatcttgatgctttttgcaacaaatgaaaagatcatgagttctagtaatagataactgagaatatgttcccttcccattggtgtgataatttttaaaattcccccctcttttgtatgaaatacacttacagaagagtgcataaagcatttccatctagtttaatttcctttaagtcaaaaaaggactcagttttcaggaaatccaggaataagtattaattactcaaaatatatgtctttagtctaaacgttttggaggaaaaaaaatttccatctcttaaaattcaagtgaaccctaagacttgttttgtgtatgaaatgataatttgtcaaacatcttttttatctataaaattattcaattattctgtttcaaaagttccaaatgtagaattcagctggagtaaatcaggtagaaatatactctgaacctaataaaaacttaatataaaacctaaagatatactcagaggttgaaataggatagttgttaaaatgtcttatgtttgtggtgttaaagatagtcatgcgttctgaatctttgctcttcatatctatcacttgcttaccttttagcctttaattatatcatccatctataatccagtccttattctcttttagaatgctgtttttcacctacacatctagtgtgcatagaatgtactttttactcatttgcctgctacattccttaagtaaaatcagctcttgactccccttccagaacctttcctgaatcccttcctctttccagtgcagtgtagcactttcacctcacacattacctatacttactttgctatactattgtcacctgattgttgttgatttacgtacaagtctctctgccctagagcttcttgaggaataggctctaagtaaacgttttgaaatgtgaataacttcagaggctgaaaagttttttaaatgcctcttgattatgtctcatctttgctttcttaaaaaatatgcatattgtatataaaatagttctgattttagggtttgacagattctgatacttttaatgctagccttataaatagcaatttaagaaggttaaacttctgtttgcatttagtaaaggcaacacaaattacataatggtcccttttttttttttttttactttttaaatcaagttaatagaaatataatttatatgtaataaaatataggcattttaaatgacagtttagtaaattccaacaaacatgcccatataaccactaccctggtcaagatttagaacatttctatcaccctaaaaatgtttctttttatagcctggaatgaccaaatgtctcagtttgcctggactgaggggtttgccaagatggtcccaggcaaactgggatggttggtcaccctgttcattctcctttgtggtcagtttcccatcttctgtctcaggtgatcattgatctattttctgtcactacagatgcctattctagaatttcataaacgaaatcatatatttcttttgtatctggctttttttgctcagtaccatgtttttgagattcgtcagtgttgctgcctgtatcacctgtttattcctttttatgctaagttgtattctgttttatgagtataccacaatttctttatccattcacctatgaatggacatttgacttgttttggggttttggttattatgagtaaagctcctatgaatgtttgtttacaggtgtttttgcagacatatgtttatatttctcttgaataaaaaagtagggctgaaattgctgatcatatggtaagtgtatggttaccaaactattttccaaagcagttttactattttacactcccaccaacaacacatcagaattccagtgcttcacattctcatcaacacatggtattctcaataattttagatttagtcttaaaatgacttttatcattaggaaatgcccctctttatttttgtttaattttcttatcctgaagtcttcattttctaatattaatataggcactctatctttcttatgattttgcatggtataacttcttgcattcttttacttttactctatgtgttttaatttaaagtgcatttcttacagacatatagttaggtgttttttaattgagtttgacaatttctgcctttgtaattgaagtacttagactatttacattcagtgtaattatcactgtggttgagtttaagttttgcaccttcgtatttgttttcctttcatcccatctttcctttgctctgtttttccccctcttttactgccacctatggattaaatcagtggtttttattttttctattggcttttaagctatacctccttgttgcatttttagaggttggtctaggatttaaaatatgcatcaatatattacagtcagtcttcaagtagtaatgtaccatgtcatatagaaaacaagaaccgtgtgacagtatttccattccccttcctgttctttgtgctatagttttcatactttttaattctacatgttataatccttacaatatttgttgtttatagggggaacccgcccctaatatttcaacataggttttttctattttccatgagtgtcggctggctgagaaataaagagaaagagtacaaagagaggaattttacagctcggcctccgggggtgacatcacatatcagtagaaccgtgatgcccacctgagctgcaaaaccagcaagttttattaaggatttcaaaaggggaggggatgcaagaacagggagtaggtcccaagatcacatgtttcatagggcaaaaggcagaacaaagatcacatgcttgtgaggaaacaggacaaaggacaaaaggcagaacttttgataagggtctatgttctgcagtgcacgtattgtcttgataaacatcttaacagaaagcagggtttgagagcagagaactggtctgacctcaaatttaccagggcgggatttttccccaccctgctaagcctgagggtactgcaggagaccagggcggatttcagtccttatctctatggcataagacagacactcccagagcagccgtttatagacctcccccaaggaatgcatttctttcccagagtattaatccttgctaggaaaagaatttagtgatatcttccctatttgcacatccatttataggctctctgcaagaagaaaaatatggctctattttgcccaaccctgcaggcagtcagaccttatggttgtcttccctagttccctgaaaattgctgttattctgttctttttcaaggtgcaccgatttcatattgttcaaacacacgtgctttacagtcaatttgtacagttaacacaatagtgggcctgaggtgacgtacatcctcagcttatgaagataacaggattaagagattaaagtaaaaaaaagacaggcataagaaattacagaactgttaatttggggaactgataaatgtccatattaaaatgaaatcttcacaatttatgttcagagattgaagtaaagacaggcttaagaaattataagagtattatttgggaactgataaatgtccatattaaaatgaaatcttcacaatttatgttcctctgccgtggctccagctggtccctgtgttcagggtccctgacttcccgcaacagttgttactttgctttaaacaatttcttcaagaggaattttttaatgagggggaaatatcatacatttaaccacatatttaccatttttggtgctctctattcctttgtgtagatcagaatgcccatttcagaagtctttctttaatcctgaactacttttttgtggtgaagatctgctggtgacaaacttggttagcttttgtttgcctaaaaatgtattttgaaggatattttcacttgatgcagaaattggtttcccttggtttccagggtttatttttccttcagtttttggagagatctctattttcttctggctttcattgtttctgttaagaagtcaactgttgataccctgtatataccctgtctataatgtgtttctggcattcttgaagattttcactttgtgagtggcttttatcagttgattatgagtttttatagtattctttgtatttgtcctcattaaggttcattgaacttcttgtgtttgtgggataatcatggtttttataaaatttgggtaagttttggtaatgtttcttcaaatagtttttctgcatctgccctacttcccaccccatacttcccaccatcagactcaagtcacatgtgtacatgttttttccctccatgcttcagctttgatagtttctattgctatttttttaattcagtgattgagtttcttccctgtgtctaacctgttaatcctctcctgtgaagttttaatttcacatactgtattttttaactgtacaatttccatttggttcttttttatatcttctatttctctccttgttataatagctattttaaagtccttatgtgccaattctgtcttctctgtcatttctggatctgtgtctgttgactgacttttgattttttctcttcttatattttcctacttctttaaaaatgttgaattttgtttgtcagccagtttcttagttgtttacacctggagggcagtttcagtgttcgttatttcatcacagctagatgcagaagtctataatggccttttaaaaaaataataataccatgcaagcctttgcatggcattctattaagtatatttcacaaagcattttaaaataagttttagattttttatactcaattttatcttgtttagccagttagatgactgtttcttcatagtattcttctgttgctaaagacctctgttattaacagtttaaaatatttttttccaacttttattttagattcaggggtacatgtgcaagtttgttacctgggtgtattgggtgatgctgaggtttgaggtatgaatgatcttttcacccaggtactgagcatagtacccagtggttagttttgcagccttctttcctgctctggttgtcctcagtttgtattattgccgtctttatgtccatgattacccagtgtttagctcccacttataagtgagaacatgcagtatttgcttttctattcttgcagtaatttgcttaagataatggcttccagcagcatctatgttgttgcaaaggacgtaattttttttatggctgcatggtattctatggtgtatatgtaccacattttctttatctagtccactgttgatgggcatctaggttgattccatgtctttgttattgtgaagaatgctgtggtgaacatgcaaatggttgtatctttttggtagaacaatttttcttttggatatatacccagtaatggcattgctgggtcaagcaagttatttgagaaatctctaaactgctttctacagtggctggactaatttacattcccaccaatagcatataagtgttcccttttctctgcagcctcaccagcatctgttgtttttttactttttaattagccattgtgactgatgtgagatgatatctcattgtggttttgacttgcatttctctgatgattagtgatgtgaagcatttttttcatgtttgttggccacttacatctcttctttagagaagtttctgttcacgtgttctgcccattttttaatggggttatttgttttttgcttgttgatttgttttaaatttcttatagattctggatatgagacctctgtcagttgtgtagtttgtgtctgttttctccaattctgtaagttgtctgtttattctgctgatagttgtttttgctatgcagaaactctttaattagttctcaccaatttttatttttattgcagttgcttttgaggacttagtcatagattatttctcaaggccaatggccagaatagtgtttcctaggttttcttgtagaatttttatagtttgaggtcttatatttaaatctttagtccatcttgagtcactttttgtatatggtgaaagataggggtccagtttcattctttagcatgtggttagccagttatcccagcaccatttattgaatagggaatcctttccccattgcttatttttgtcgactgtgacgaagatcagatggctgtgtgtggctttatttctgggttctctgttctgttccattggtctatgaatctgtttttgtgccaatatgatgctgttttgattaatgtagccttatagtacagtttgacgtcaggtaatgtgatgcctctggctttgtcctttttgcttaggattgctttggctatatgggctcttttttggttccatatgaattttagagtagtttcttctagttctgtggaaaatgacattggtagttcgataggaatagcatgaaaaatgacattggtagtttgataggaatagcattgaatctgtagaatgctttgggcagtatggccgttttaacagtactgattcttccaatccatgagcatggaatgcttttccatttgtgtcatctgtgatttctcttagcagtgttttgtagttctccctgtagagatctttcaccttctcagttagaggtattcctaggtattttattttattttattttattttattttattttattttatttgtggctattgtaaataggattgtgttgttgattgggctctcagcttgaacattattggtgtatagaaatgctacttattcatttgttgattttttatcctgaaactactgaaggcatttatcagttccaggaaccttttggcaggaacctttgtctttagggctttctaggtatagaatcatatcatctgcaaagaaaatagtttaactacttatttttctatttggatgccatttatttttttctcctgcctgattgctctggctagcacttctggtactatgttgaataggagtggtgagtgggcatccttgtcttgttccacttctcaaggagaatgcttccagtttttgcccattcggtataatgttgatgttcattttattaagttgaattaccatgataggggtagattttatttaaatcagggttttttttaagttgccaaaaagtctatttaacttttgaaacttgtttataacctcgtgatcttacaaaacctttatatattttatacaactcaaaaggattcatgtttcttttttatggtataatcactgtagtttaagaaaatgactttttaaatgttattttgtaaaaactcttccttgttttctgcccttaacattggatagaccattttagggagtcagtgacacatgcatttgtatccctatctaccactgacaaattgtaaccttgagacttctgtcagccttaggtttttcacatgtaaaagaggataatattgtatctgtaaaattcaaatgagaaaagaaaattgatgtgtctagcacactgctcttacatgctatgtgcttaataaatatttgttctttgttctctcctctctctctcactggcagtactttgaaaggaaaacttaattccactaaactcatgtcaggtgattatggtcggcttgctcttcccgtgacctcagcaagatcagaaaggaaaccttagtggccacctgcagtgaacaccttttgggctgtattttttgcctgggttcattgtgtcataagatacttctctgtcgtttaagtgttcgccttcctttgcccccatggtgtcactgtcttatgggacctcacccaactccttgtacttctaactcagccttctgtttcaatttctcttttttctgtatatttgtatgcccatctttagcactcttctttgtctgctttgcctgttatacactgtcacagccacctagcacttataccgcaggtactaccaaagcagttcttttagcctcaagctctcttctaagtaatatatctaatgttcaccacttggtggagatctccttctggttcttcctgaatgtctggtctcaaatattattttctcttctgatctactccttcatgcctaataccatcaagatttttcacaactactgaaattgtttaccttccagccacattcaataaccagattctttgatgccacctcctaaaatttgtttaccactgataagatttaaattactttctagctctgtttctagttatgacctctgaaaaggtactccttctgggtctgtttttctcatgtttaaaaatgtttgtactagatcaagggtcaacacagtagggtccaggggccaaacacagtcttctacccatttttgtaaataaacttttgtttgtggttgttttcatgttgcagagttgagtagtgacagagactttgtgtcccacaagccttaaatatttactgtctggccctttccagaaaacatttgctgacccctgaactagaaaatctctcatctagtttcctcttttttttaataatttttttggggaccattcatgatcacgtctaaggatagtaggcatttttaaattgctccctaatgttttgttttaattggtttatcattagaagtcaccaacagaaataagtgctttttttcctatattgtgaggaccttgtcataataaaatattcttcagtgtttcaaatttgagagctttttgaatttttagtattttttaatatgttgtaaatcccactgtcagccaccctaaggagaacctggtgttaagatttaaaatacagtaagaaaaagactagaaaaatgatagtagttgaattaaacgtggaacatactgtaatgttaggatctctgtttcccatgtaatagccttcgtgattcagtcatgtgatggtaaatggatgaataggattttctttgccattttgtgctgacagacctttttttagttaattgctttttaaaataatttatagtgaatacaccaacaattctatttacaggttggtagttaaagcctcttaacagtcactgataactatattttgaaggtaaagaatatagctagttaaaactctgaccactgattgtaaatataataaagaaacctttttttaaccagtggggaacccaggtgaaataaatgcaaatcaacagcaaaactgatggtaaaagtgtattaatctttataaaggaatgagttgcagaagaaaaaaaatactttttaaatatcatcactattcatcaaagcagattatttcccattgtggtaaagattttctacgtgcattttctggtatatattaaaggaggattcaaggtatgtaaaaaacatacttacataaaaagttgtaaatatcaaaccttaattatatgcatgctgatgtgtactgatacctgcaacttattttgaaatgcatcaaaaaataggtggattgatgtgtggatagagggttgtgtatgtgatgaaccaaatatagtaaaatgttagttggagaatatggatagtggatatagtgggttggtgttcactgtaaaactttcacaacttttttgtttgaaattttccttaacataatattaggtaaaatgtttagatctaagtggctgtgagacagtgaatttagggagaaggatgtgagagtgagtacacacacatggtataccttaaataaattattaaaagtttatatgttaagcataccgtgtatataagcatatgtttctcaactgttttattgtgtatttcacagatttgcttctattaggaatattcttaggttataagtaaaaaaaaaaaacaagaattgtaaaactttcatttcagggaacatcctgacttttacattatacattgtaactgtattagtccgttttcacactgctgataaagatataccccagactgcataatttaataaggaaagaggtttaattgacttacagttccacatgcctgggaaggcctcacaatcatggcagaaggcaaggaggagcaaagtcatatattacatggatggcggcagtcaagagacgagagcttgtcttgtgcagggaaactcccctttataaaaccatcagatcttgtgagacttattcactataatgagaacagcatgggacaaactactgccgtgattcagttatctcccactgggtccctcccatgacatgagggaaataggggagctacaattcaaaatgagatttgggtgggggcatagccaaaccatatcagtaactcttgaattatcccccataacctctatgaaagttagccgaatgcttaagaagagaagtccatgtgatttctgttcacacagtaaatggatacatttactaatggacatcacaagagttagtactgtgtcttatgttagcaaagtactgaatatgtagaggatggctgtacaacaaatactgattcaagagtgggcatttctgaagtgtcattttggttatttcgaaatatatcaaaatgaaaaagtatatgcaaatatattgttttaagtacaatagtaaatattcatgtaactacaactcatattaagaattagagctagaagaattacttttgccccatcccagtcacagcccccttttccacatcctctttgttaaactggataatctgtacttgattttaatgatgaatatacttatgttttttatttatttttacacagttttactatgtaaactatagtgttgtctgattttgaactttaaggcagtcatgttgtgtgcattcattgttgagtaaaagctcaccactgtgtgagattcatccataaaggtatgtgtagctctggtttagtcatcttcattgctagttagtatgctattgtatgactatgccacaatttatttgttaattctactgttggatttttgcttttatcggcagtattttattattatgaacaatgttgctttagaggtataagaatttctctgttagggtatatagcttgaaatagaaatcagtaagttgtagggtcactgaaggtatacacttataaacactcatttattcatgtcattgccgctgttgtttgttttcagctctcaaaagaatccaatagagttgcatgtgacggaattgttgataagcttattagtactactccctcattcttcactcctcaccctctcaccaggtacatgtatacacatacacatgcacatatccccagaacaatgttagaaataggggttagaataagtgttaataatttagattatttttacagtcacctttttattttctttccaataacattaggagtttgctcagaaattaccttatacacagttaaaggtcggtttatttttaactagattcctttttgatttatatcagtgaataattctgtagctcagaatatccttccatctatcttacccaaactttatgtattctaatagttaaataatttctgaaagtttttattacaggggtaattggcatacaagaaactacacatagggaccacatcaagataaaaagcttctgcacagcaaaggaaacagttaacagactgaagagacaattcacaaaatgggagaaaatatttgacaagagcttaataaccagaatatataagaagctccaacaactttataggaaaaatatctaataatctgattttgaaatggacaaaagatctgaatagacattattcaaaaggatacatacagatggcaaacaggtatatgagaggtgctcaacatcattgatcatcagagaaatgcaaatcaaaactacaatgagatatcatctcaccccagataaaatggcttttatctaaatgacaggcagtatcaaatgctgacaaggatgtggagaaaacagaatccttgttatgttgctggtggcgatgtaagttagtactgcttccatggagaacagtttggaggctcctaaaaaaactaaaaatagagctgcctaccatctagcaatcacactgctaggtatatacccaaaagaaaggaaatcagtatattgaacaggtatctgcactcctatgtttattgcagcactcttcacaatagccaagatttggaagcaacctaagtgttcatcagcatggacacttcatgaatggataaagaaaatgtgatatatatactcataaagaatgagatcctgttatatgcaacaacatagatggcaccggaagtcgttgtgttaagccagacacagacaaacttcgcatattctcagttatttgtgagagctaaaaattaaaacaattgaactcatggagatagggagtagaatgatggttagcagaggctgggaagggcagttgctggggaagtagggatggttaatgggtacaaaacaatagaaagaatgaataatatctagtatttcatagcacaacagggtgactatactcaataataatttaattaaatatcgaaaaatacctaaaagagtataattggattgtttataaaacaaaagataggctgtgcgcaatggctcacacctgtaatcccaacactttgggaggctgaagtgggtaaatcacgaggtcaggagatcaagaccatcctggctaccactgtgaaaccccatctctactaaaaaatagaaaaaattagccaggcgtggtggtgggcacctgtagtcccagctactcgggaggctgaggcaggagaatggtgtgagcccaggagccggagcttgcagtgagctgagatcacgccactgcactccagcctgggcaacagtgtgagactccgtctctaaataaataaatataaataaacaaaggacaaatgcttgaggtgatggataccacatttaccctgatgtgattattacacattgtatgcctatatcagagtgtctcatatattccacagatacatacacctactatgtagccacaaaaataaatattttttaaaactgcacatatttaaagtattcaacttgataagtttttccatacgcttacacccgtgaaaccaagataatgaacattcataatcccagtagtttcctagtgtccctttgtaatcttgcttctcaaaactatagatcttctgtcactatagatagaaaatttactagaattccatatgaatgtgattatactttaggtactctttttggtctggcttctctcactcagcttaattatttttagattcatccatgttgttctttttaaattactgagtaatgttttactgtatggctatgtaccatttatttacttgatgatgccattttggtttgtttctactgtttagcagttaaaaaaaatatgctatgcatatttgtatacaagtctttgtatgaacatgtgcattcatttctcttgggtatgtacctaggagtgagaatgatgggttgtatgatatgtatatgtttaacttttaagaaacttccaaactgttttccaaggtgctatataccatttcacatttccaccatcaatggtgagagcttcagttgctgtacatccttgccaagatttgttatacatagtctttaattttagccttcttattgggtatatggtggtatcttgttttaattagcatttccctaatgattaatgatgccaagcatcttttcttgtgcttatttgccatctctctgtcatccttggtgaagtgtctgtacatatcttttactgatttgtattggtttgtcttatttttgaattataagagttctttatatattgtaaatacaagccctttgcagaatatattttgcaaatatattctgcaaagttcgtgactagccttttcattttcttaagagtgtcttttaaagaacaaactttttaaaatttgaatgaagttctgaaagttttagcagtgtctagagaattagaaccaacaacaaaaacataacaaattctcactttgccctgaatggttacagaattatgcctgattattcactggttgccaccacaaatctagtacttaccattacattcttctacttctgtgcagttattcttgaactgtaactggtttaatattttatttcaataaatttgatttatacacggtcttataaagatgagtaatatgtggtatttctcttcaaagtcctcaatttttagcatttaatgtttcttcctgtcatccatcttgtacagccaggtccatgtactttcttttgttcagtacctagtatagctctagagacaatgaagtgctcaatacatttttgttgtgtactgtaattatacttttttgacatacaaatatgtaagtttatggtagcaggaaatctcttgttctttgtatcctccaaaggtcctaacatagttccttgtatacaagggacactattcaacgactgaatgaattgttgacggttttaaaataaactttgcctgatatatcgataatacaattggccctccatatctacaggttccgcatctgcagaatcaaccgactgcagatcaaaaatattcagaaaataacaatttaaaaatacattaatgaagaataatatacaacttaaaaacaatacaactatttatatagcatttatgttaagtattataataacctaaagatgatttaaagtataggaggatgtaaggttatatgcaactattatgccattttatataagggactcgaacatctcttgattttggtatcctcgggggctcctgaaacaatctgaataccaagggacagctgtataatctttggctatactgaactaagtgaagaaaaaatactctcagatgtgattattgcaatagatagtttttctagataaagatatctgatttttacttgaatatttttgagtatggtaatcttttttatgacccaaatttaacattattgttatcatagaagtctgtctatggtaatacagcatggaatctgtagtcagctacagataaaaggctttcacaaagtttgtttgcctttgtctgttttgtgctactataacagaataccaaagactggataatatataaacaatagaaatttacttggttcactgttctagagactggaaagtccaatgtcaaagggcccacatctggtgatggccttcgtgctgtatcatctcatattggaaggcagaaagtcaagacaaggcaagagtgagagggagccaaacttacatttctaaccagcctgctcttgaaataacaatcctgcttccactttaatgacatgagtccattcacaagggcagagccattacctaattaccatttaaacatcccacctctcaacactgttgaattgggtattaagtttccaacacatgagctttgggggacatattcaaatcatggcactgctctatttaatgagttacgtattttatttaacttgggaattccttttttcagtgaatccaatgcctgaaaacatggagactaaatttgtaaatatataggaaaagatggcaataacttgctttctttctaggtcctgggaactccaacaagggagcaaatcagagaaatgaacccaaactacacagaatttaaattccctcaaattaaggcacatccttggactaaggtgagttagatttttttttaagtatattaatcagtagttaactaaaacaggtttctcattgttcttaaactattttgagatcagatagttgcatgaatacaagcagttctgcatgtaaagatagttgtatagatgtacttgagtgtttcattgactttatatagtttcatcctcattgatttcctgtagatagtttctagcctggttttttatttgcaaggtaagggcagtgtacttatatcattaaagtacagaatttgatttaggagtaggatcttatataaaattttgacaacctaaaattcagaagtttatttacatagaggtgtatagtttaatatcatatagcatatcatacatttcaaacattttctttattcttttaaactgttaaaacatatatcttattatagatggcattttgtgtttttcctctcttgtttactttcctactggcattttggaaacaagattagaaagcaaatgtaatcttgctgggagtatacattaaaaacaattgatatgttaactggatgtcactagcctttggaaaaacccaaccttaacattagatagaaccagttctaaacagcatagttgctaatgtaccagggaacttttttttttcctttggtatttaattggaatgattttaaaagagattttattaaaaagtgttttcaccttagtttgatttgtgagattaagtatgaacagaaaaaaaatagaatttctcagtagggtttataagtagagtgaatctctagcttcttcattttacaagttcagaaactctgaccaaagattcaaagtgacttacccaagattacatggatggcttgtgacaaagccgggattaaacttctgtgtgtgtgtgtgtgtgtgtgtgtgtgtgtgtgtgtgtgtgtgtgtgtcacgggttaccaatgatcgactttataatacgtattttaaggagagacttactgtattttaaaatgtacatataacaattcattttatatacaattaaattcatttagttatagatcttttataattcgaggtgaagtcatttagggctataggtgaaataatttaccctctgtagaatcaaagtatgtttctttctctctctctctcttaatcagatatattatgattccagttactttttcttcaagttcttataatctcattatctatcttcagaaaattctcgttactaattatttatttcctatttagaatcaaagacctcaatagtattttagtgccagaattataggttgtaacttttactacaacctggccaacagaccacgatcttgcctcccttttttaaaagtttatggtaagcactaagtagtctgagattcaaaagtgttctttgatctttgtattcaaggcttattttaaatctgggtcagaagatatttagggactctcacctggctctaaacccagaagatctcaggtggcctacttggatttagaggctgcattacagtttgagtccattatagttattgcaagttaaattgtttatagtaagtgatttgtagtatactatgacttcagccatgttaagagaaaaacaacaaagagaattctgtgtattaaataaatacagggaaatattccaaaatgccaacagtagtttgggtaagaagaaagtgcatgattttattttctttgttctaccttttcaaactttcatattttctttaataataatgttatttttatgaggaaagggaaaaattctttttttggtgtaatctaagtattcatcacatattatcctccttatagagtttctgacaaatataaagctgaaattgagtccatgagatcctatttaacagtccccatcctccattattattattattattttatcaagactgccactagtcctcctttatctaaggtttcagttacatttggtcagcctctgtctgaaatcggtgatgtagtacaataagatattttaagagagatactacattcacattattttattatagatattattctaattattctattttattattagttattgttgttaacctcttactgtgcctaatttatgaagtaagcttttatcactgtatgtatagagaaaaacagtatatgtagggtttagtactacttgcagtctcagcatccacatgagggtcttggggacatatcccctgtagataaaagggaggggacactgtatttagtgtgtgtcaaattctgcattttccccctgaattatatctctctaacgcaagtattagctagagtagaatgtgaggaatggttgattataattaaaatgcagctaatgaaagagctgtactaaatatgtatgctgtaaatcttcctttgccctcttcaattcttgatgtttttatatcttatactaatgcttttcaaacttgaatcgtctatgtaaagcacatttatttaggtttccattcctgtaccatgtgtagaataatgactcagttctcccatcttttttgtctattaaaaggactgtgaacctttcattcgtctggtgttttttgacataatttggttttcacttcatacacatgcattatgtaataagtgcacttatgttggttcctcatttgtgcatactagagtgggtgttactgggattgtaaacaaaaaagtaaattcagatgcagaaattttattgtttttctctgtcatctagatgattgagaatatgcttgtgttggtttttttaaataggttattataagaattaaaatgcaaaacttaaaattcctagagggaactcagactcctttccctaccactgtgatagtggacagcaacttgagaaatgctcttagggtaagcagccaattgagtcatttaaaaaccatatagaatgtcctccaaagagaagaccattttgcccttggagggcaggtaaacaggagtctggaaacaggaaccactttacctttgttgctgtcttctgttagtcctatctcctgaagctgtggacatgtgaaccctggtttcctgtgctgcagtgttgtgcagctatatccccaagattgggacctggtgttctaaaaaaaaaaaaagtagacagaaataaagaccaaaagagaaaatgtcttttgtggaaacattgaggtaaggggatacaggtgtgtaaaagaaatagcagaaaaataacactgagatgatcccagtgtggtgtatggcagaaatcttgagaattaaaaactccaataagttgccctgggctattacataaatagcaatagccagtgcaacataattcattaataaagcacttgtggctttagtaggaaaggaaataggtgctaatattctgaaacaaggattattattggaggtgaggggatgaagacctatgccttggtgtacgctgtacactgcaaagaagaaaaacacatggaaagaaaaggaagtgatgcaatagtagattgagaattaagcagagacagagattggttggaagaagagaagaaatttaaattctgcatttagtatgtgcaggtgtggttaaatgatgggctattatatacagagtgagaatctagtttaaacactgctcctaatgtttatgcctcagcttttttcttttgtaagatagcgattatctgctgtgtcttttctcaggattattttgaaaatacagccataatatacatataaaggattactttagttttgaataaatgtgaaggatgtagcactccaatggagttttctgatttgtcttctctgtctccactgccctgaccttagtttaggccaccatcatatcatagaattattaaaaacaacctcctacctcctgatttccatcctttggtttcacatcattttaatccatcctcctcatttctgtcaaagttggcttttttaaaaagcacatgtgatgctgtcactcctatttaaaattcttctctgattcctcatagcctttaggaaaatttagcgttaaatacatataatgacaagggtgatcttatgtgccagtttttgcttgtccagtgtaattattaattgcttgcctcttcactctcaggaaggtcccaatttgggtggcagattatttagtcactctgttcttgatcttttctgcctttcttttccagaaaaatagagtatttagtatttgtcctattcccaaccttcttgaggtatctagttcttgaacccaggctctttcccaagttttacagcttcctgtatccttaaatgcatgttgttaggcaatgaatttgaaagtttagtgtctagttagggttgattagggatttttaataaggttatatcatagtaggtgtatgaggtgccttcccgcccactttcgagtgcctgatcctgctaccttgtttaaatttctttatgacaagcatttctcctctatcctagtaataaacaatcctgtgaggtcagttaggtaacacagatgtctgttttattctttcagagtaacaaaagaataaagtaataactggttagtagaaggaagtagattacaatcctaaggcctgctgatctcttaacagttacattagtgctatttaatatgttggctacgtattggagtgttgtccagtttgtagctcactttgaagggaccatgcagccatgctggattttctggtttagtcagattgtctcctagttgttccccagagaagccacattcatttctgcctcttgctagtgtctcccttccctctctctactcccccttccactgtcttagaatgttctttttttcttctctgcttaccaactcttaacctttcttaagttctcaaatctcacttctcccatgaaatattttctcattgtctaagttctttatgattatcctaccacataactcctatagtacctaccaaattataccatatatattatagcaattcctcattgcttttttataataatagtattaatatgaatgccaattacttcaaaatatttgttgccaagttgttggcatatatggtagtttaatacttaatagtaaagtgtctgtagcttttgtatgaccataggtcatttggtaagtctcccagaaagcattattcacaaaaattaatccttgcatacttgatttcctaaaacactgtcaatatgcttcatagtgaatcaatactagtgtttctaactggctaacagagtaagcagaaaattgaattctcaggtcatttcagctttcaaaggaattatttttgactgaaaaagttacttattctctatgcttacagctgaaccaaaaagccccactgtacttatttctttttaagactttgtccataatgctacagtttttcccctgagccattcaatagaattttcagaacttggtaaacctacctgttttaaccgttgttcaatgtattattaaaatgccttcagagaggcctagagggcacaaaacaaaaattgctgccaaagttttctttcacatccattctatctaccttttctggtctctctgcattactcatttcattcacatgtattccagaaagcataaaaccatgttggggatttctattggctcctaagcttgccacttgtctgttttgttctattgcttatggtaaatcaatttttatattattattcaacccccctctgccctgaacattctacctagaccattctcacttgcctcacaaatacatatagtatgttctctactgaaagttctacccacatccttattattgctataataatcacttcctctccctttctaaagccactttcctatgttagtagacctcacttttttatttctactggctatatatttttatttgtttgctggaaacccacattaaaatccagcttttagggtggctaatccaaatgtgaaaggaataatgtgaaaaaggaagaatactaagtaagacataattttcagattgcttgtgggaggcaaaggatgaggttggggtggatttgaaaagaagacaaaattctatttttaatttatcatttattctttaaatcttgtatcttctatgtaaccagtgctgtgctgggtaccataaagcatgcaaaagaagtaaaatatatagtctttgtccttgaaaagctgacagttgaaaagcttagttgaagcaagtattgaataatgtgagatactatgtaaccgtatacttaattctgtgctgtaagtgttcagaggtgttccacatggctgtaggtgttacaaggcagacttccagtgaggatggtggagggaaaaaggaacccccacttagggataatacatgaggtgggatgttgaaggggaggtttaagatttgcctaggtgtagaaaaggttaggaattgttatagcctgggtgaatatctctcaaggtggagagagctgaatgaaaagaaaatatttctatattcataggagagccaacctgactgtaataattgttttgtgaacaatgaaagattaaattggataagcggacatttggcaaatcccactcctgtgtaggcatgtagtactatttcatcagcacatgatgtgagggagagaaattatgttacatagttaaatgctgatatctacatatatacagccagtcatttaacatcgttagtaggtgcttagaaactgcaactttaagtgaagttatgcacaataggtcctcaaataatgttggttcattcagcgttgatgagggaaaacatttggttttattatacgtttcactaaaagtcacagttttcaagaacctatcaatgacattaaatgaagatttactgtataacttttcttacctatagtagaacttgggaaaactcgataaggcttttaattagtacagctgaaattcactggatgttagatttgtcatggctaaggaaaggattctttgtgtttcttatgctcaaccttctttacctgaactggtatcttaacgaaaaggcttagaaaattatgcagtagacttgagctgaattcagtttgtgtatcctaccctgttagatgccaacttcatagatagcaattttacttgggaggaggactctgaaagttgttttcagtagcctctggtaatcatttttgatagtacttaaagtgaattggattaccttgtatgtcagtgcatcattagatcaatgtagcaaagcatgtgaaagactgccaaagaatatatgtttttttttttttttttttttttgagatggaatcttgatctgttgcccaggctggagtgcagtggcacgacctcggctcactgcagcctccacctcccgggttcaagcaattctcctgcctcagcctcccgagtagctgggattacaggcacccaccactacacctggctaatttttgtatttttagtaaagatagggagaatattttaaaaaatagtatagactaatttgttaagtaattattgaattatgtgctacatatgagcacttaagtagacttgatttctttcttagttaacataattacctattcaacaatccatggcattgtctcacttttaatcctatgtttaggtttacaaattgcatataatttcagatgattaatagattacttgagagtcaatattggagttgctctttaggagtattcatggtttattagtttttaaaactggatgcttatattttgtatatgaccaggatataggatgagtgttgtacatttatagtaataaaaactaatgaggaatagctatttggaacagacatggctcttgttctgtgttctgttagattaattatcactgccaacagccatttaaaatggacatttttacattgctctgattggctacataatacaatagagttgctaagagctgatgctgtgagatccatgttaaccataggtcttagtatggtgtatgtgattgtttaaaaatggcctttttaaagtgcttgagattcagtcacaacacttagctccaggttgaaatttgaaggacaaagtctattctaggacatgtagggcagaaattgcctctgtcattttttagttacccaaatgcaaatttattttgaataattcttcatttaaggacttctttgcatttgttcaatactgcagaaactttctttttgaaaaaatgatttttttctgttagtttcttctatgggctagtatttgtgatgggagttgatatttgtttcaatttaagaaaatgagttgctgcataggtgaggtagtatttgaatcactttaaaagttcatgtcagtgttttggttcttttcagtatttcctttctcttttctttcttttttccttcctctttttcttttctttcttcttcttcttattttttgctttgttttattttgttctgttttatttatttacttttggttttgtttttaaaggggtgttttgggatctctgtggccttgctgcatacttcgctggccatatttagtctgcgtactaattgttagttggcccagttgagtcttattttgtgctctcagttctttcagtctggttttctatttttgtccacctggttgtgttttagttcatgagagaatttattctgatgggccagattttaatctgtgtactaaattttagtctgcagactaaaagtaatccgcggattatttatttttgaaaaatatttaattcccccactggaaactatcctaaaagaatgttaaattctcttagaatagccaggggagttcacagatactggtgagttctgccattttttgttctcaatacctagacataatagaccctgctacctcccttgcatatttggctccgttgtgcctaggtgttggtatacattactatgcctgtcacctgtaactgtgactgtcccttagttgtattgttaattttatatattttgtttagcttttggtgggtgaattgggcatatttgcagatggaactgtgtttaatttgtagtgctggaagatgtaggatttattttttcagagagtttttccaaagaggaaaaaatgttctctatttatttttcctaagtaccgggtgcaagggtatgagcgggcaaagcccacaatagaaaaagtaagttaatacccgcactcctgaggtgatatgtcaatttcctgatgagtcctaagataatgagaatgtcagcaaagaaacatcttttagcagagaagtcaatccaaaatggagaataatccgcggtggcctgatccttcttagctcacacaaattgaaagtatctggcttgaaatctatagtaatataacttaaatatatttattactttcctctcatcactaataacttagcaaaaaaatgtttatactgtgtaactggaacacattaatagagaaaaaagtagcatattgggaacttgaaactttaaaagcaggctaatatgccccctcccagccatgtctgtggatgatgaatccaggtcatagaagaccattttactttgggaactttacttctagatttcattttgattttaactttttttggttttgtatttttttaataaacgcactggcattggaacataataaactaaaattaaaatgtttctttactgtgcctcaccattgcacaatcaaaaaaaatttttttgcatacactgtggtgaaataatttttaaaacttggacttgctactgtgaagactgatgtttaaagtttataactgtataaacattttctatgtccccccccacaccatggatttcttataacattggatatatttttccttgctggactattctcttttggattgacttcccctgtttggattatttcactgcattatcatctttaggattcgtcaggaacaggacatttcacctcaggagtgcgggtatgtacttaatttttttttcccaaaatttcagggcaggtctataaaacttgggggaaatcttctttgagaggctaaataattactcattcttgaaatgtaatagtaaacaatgtaagtagctgtgatttctgtttttattcactgtatggcataggtttcacttatagtatatttagattgtcagcctcttgggtgattcattgacatctaggctgaagtttaggaattgtggggttttaaaattattatttctattacaagaataaatattgatgaagatccatgaaaccaaaggtctgagtgttaagcaatttcagtgagtaaggaaagaagtgacagcggaagcttctttaattaaaagacaaatttcagttcttggtggtttccttttctttagaagcaaaaataattcagatgagctagtttctagtttgccctataatttttagaagttacatgctaactcaccctgtattatggtcagaaatctgaactgtgggagatactgggttttgaccccacgtaattttccacttaacctttattcacagagtactgaacctaggcttttctcatcaagaatctctcaagggtttaaaatgacagtgtatagtttttgtaaaggcaggttaaatcttgattttaatgtaggcttttgacatgtattattttcttcattgtttttaactcttgaactttatgagttaggattccctgacaaatatacgctaataaatgtcttagtaccgatatggatgatcacttgtaggcagtaaaacaccaaaagatatgggtgacttaaaaaattactggaaataggccgggcacggtggctcacgcctgtaatcccagcactttgggaggccgaggcgggcggatcatgaggtcaggagatggagaccatcctggctaacacggtgaaaccccatctctactaaaaatacaaaaaattagctgggtgtggtggcgggcgcctgtagtcccagctactcgggaggctgaggcaggagaatggcatgaacccgggaggcagagcttgcagtgagccaagatcgggccactgcattccagcctgggtgacaggaagactccatctcaaaaaaaaaaaaaaaaaaaaaaatatatatatatatatatatatatatatatatcggaaataagcttataatactggtttattaatagctttaacacaaaatctttgaaaataagaaatttttttatttaaaataactcagcatccagccatcctacatttgaatgttagtgggagatatttatttctacttttggtgctacttttaattccacttatgtgctgccatattggtgcaaaaacattttggattagctagcaaaataaatccatatactatcaaacagaagcaaacttgccttcagggataaaatatgccaatactgtttaatatttttacgaaaattttctgagttttcccactgattattttctttttacattctgtatatgattatggaccttgggagcaagagaggggaccagtaacagtggaagagctaaacatttcaaattcgacagtaactctaaggtttccttgactgaggcgagtcagagtattcaaaagaataaagataaggcaggttttgaccttacaaaaaggtttctttgtaacataaccaagaaattgactaaggcctttctttatgttccgaaataatttcctaagagcctactttacctaatttctcaattttcatggaaaatgtctttttcaataataaattaaaaacaaatacaatcctcttttcactgtagacagcactcataaataatctctggaaagtctaatgagctctgtagttgtaactagttctactaactgtcagcataagtgaacaaccaaatgttacatgagccattcttacgaggaaaccttagccttccaggctgctccatgccctgtcacaatctgcgtgggcttttctcacttgtgggtccgtttccctccatttataagtgtctacttctctgcccacctccatcgtcacaccttttatttcgttgtatgttcttcctgtcgagggatatatgtttcatttttctttaatttcaaaggcttctcgtctgtttatttctatccttacctttttatttttattctgtaggtggccataatttagaatgaagaatatttttatagtttttgtatttattagctttatttaagacacaaattagtagaaagctatctattcctttaagtagattaatttaattgaatttttcttcaatatagccttcagttgactttttctttgtcttaatcattcttaatgaataatacatttttttctccttgattaaaaccctggaatattcattcaagtgggggaaatctggccacattcatggtaacacctacttgagaaaaaaaatgcaggacattttatcaaattctaggtattcaagaatttcttcaaataatggtaggctctgtttattggaaataataccaaatcagtagcatttatagacctagagtaaatagctcttctttctcttctgtttgattgccaagttttcgaacagtgctgctcatcattaacaattgttcggctaaaaaaacaatgcaaaagaggcaatcttttatttactgccatagcagtgaaaactttaaaatttaaaaaatatttttctagttgtgcttgccaccagaactcagcctttcccatgtgcctgtcatattttgtatgcatgtctgattgttattactttatgattagcatatatagatttttctctgagcatttctcatataatctttaattagcaattcacccctgtgaatcatatgcatttaactctttccattctagaaggtcctttcccaaatgcaaccatgtttctgtacatggtgggaattaagtcattattcacagggtatttgcttagattcttaaaataaaacattaattgttctttccctttttttgtactgcaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaaggtaactccaaacacttagcctcttcaccagttctaggaaagctacaccctgggtgtgaaatgacattctattaattaaaatctgtggaggacaaaaacggacatattaagtacttaggtaggtgaaaagagcatcctttctaacttactggccactacttacatggttttgaacagaagagtagcacgtaaaagttttccaatagacatagcttctttaatttttagcagaatataatgttggcacatgagaagttacagagagttcagtgagtgaagtttatcactaattggacaaatgtacaatttaacttttaggtttggatctgaaacaaaatagtagcatttcttgcttccaaagagtttgtatcaagtttttttaaacctggaatgaattggctatatttgtgtaaattcagttgtcttccaaacaggaggaaaagtagagccctataaaccctttctaagaacttaaacaatagacacaaggaaatttccatcccctctgagactggcaccattgctgctgctgagataatactgaaagggaaacgaactttgtgcattgtggtttctgggagaccttgggatagtcactatttcttcactatttctgtgttcagttttcttttctccttgattaagacctaagtgattcattcattcagtcacaccgcctctgctgatgtgactttcttattacactgagcataaaaagtgtacaattcattacaaaaaaaaaaaaaaaaaaacaagaaatagttgatatattgattagtaagcatactcaggctagaaaacaaaaaagggagatgtagagttttatgtgaccaagaaagaaatatatttcctttattcatatgtatcatatctttctttttttttttttttttttattatactctaagttttagggtacatgtgcacattgtgcaggttagttacatatgtatacatgtgccatactggtgtgctgcacccactaatgtgtcatctagcattaggtatatctcccaatgctatccctcccccctcccccgaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggatatgaactcatcattttttatggctgcatagtattccatggtgtatatgtgccacattttcttaatccagtctatcattgttggacatttgggttggttccaagtttcttttaaaaaaaaaaaaagaagacttttattttggtattttaaaatcttatgtttgcccagcacattagatataaatatgtgtgtcttgaacatttatgtttttgagggtttggagaatgcatttgtatttggatgtaatgtgtatagatacagtgtgtggtctctgtggttatcagagtgcttatgtgattctgtgagtatcagtgaaaaagtggaccctgtaaacacagtcatgtattgcttaatgatgcagatatgttctgagaaatgtattattaggcaattttcatccttgtgcagacgttacagagtgtgcttacgcatactaagtatattattgaaagtgctatcattttgaggtatggccccttgttaaaaagtgttcaaatgctctgtcttagcacccattctgaaactgggctgtgatcacaaagtagtactttctcacggtctttgtaaatcgctattgttactgtttttgaacagtgtttctatgcaataaagacacagatgtccagggaagaggtggggtttttttgttgttgttttttggtttttttgtttgtttgtttgtttgttttgttttttttttttttgagacggagtctcactctgtcactcaggctggagtgcagtggcatgataccggctcgctgcaacctccacctcccagattcaaacaattcttgtgcctcagcctccttagtagctgggattacaggctcctgccaccatacctggctaatttttgtattttcagtagagacggggtttcgccatgttggccaggctggttttaaactcctgacctcaggtgatctgcctgccttggcctcccaaagtgctgggattacaggcatgagccaccatgcctggccttgatgatgccattctatttgatatgatactctgtattctaattttggagcagtgtttccaaatggtgatcttgttttctaaggaagctgtaactgtgtacagatgcagctttaaagcacatactagttagtatgtggatgactagttagttaatggatgaccagaataaaactgggtaatccaaggggttgaagaaggagaatcaattttccacttggttgtatagctaaaggaaatctagagatagaaacattgtgggtctggtgaagggtgaggggtattagtgaggttcttctaaaggcagcataaagccctcctccatcctcataacaaattgtcagtgacatctgcagcactacttcccccaaataaaataaagtacatatagtaggtgctcagtgaatgtttgagtgccgtgtaccactttaagtaaacataatattcattaattcatacaaccagtatttattgggcatttctaatgttccctctggttgttattactattgttgttttaattagtaaagtaaacctgctatatgtacagaactataaagattaaacacttgagtaaaatatagtgttttattaaggtaaagcttacaataaagctaactgcattttgatgtttgttactgtaccatgaggtaattagccataaacagtaattagtattactttcatgtaatcacagtatttccatattcagatagaggaaggagagagatgaacagatatgagatggacaactttaagatctttctgattgcctaaatccataactcaggagaatatcagacctctcatttttccagcactgaagcttcagtgttccaggaccccatgactgacaatattatctgcattcagatgttggaaattggaataaacctttggcacacccccttctttctatttcttcctgtcattagaatttcaaattcagccatagctgaatcagaccttaggaaacaggtagaatcaggaaagcccaataaggatcagtattttcttatgaactaatttgttgcctattccaagactggaatatattagggaatcaattgttttgtatgtttaaaggtgagtctccccctaactctatcacacacatccccaatctcagttttttacctaatttgtatgaaattttaccaattgaagttatttagtaaacattgtcctcctgagagtgtagggtaggatgggggtggtaaagatattatccatttactttagtatgagctaatgtgaacattctattcctctaccacttggctagattatgtgagaatggtagctttgtagggagataagtaagagtggcagcatgttggctaggccagaagaagcagtgatttagtaactctggggaactgtttaattgagagttgaaacaggatatttggggcagccagtgagcaatatgcagactcagacagactgaatattttggggcggggggtaagagtatctggacacgagaaaagcaggtgatgagatagaaaaggtgttccagaaagcaagtaaagaaaagaacagtaaaaggaagattgagatataaaaaacaaaggaaaataaggaaagggttagaaaatattttttgaaagagtataagaatgtgttacagaataggaattgatgaaaacatggaattgaaaatgagtaggagatgggcaacaaaagcaagtgttctagaatccaagctcatcaaaagtttgtatttctttcatttgaaagcagtaggctacttatttataaaatctattgtaattaattatgataactagcagattccatatttgtaacaatgatttttaaacttattttcctgttataaaataacaatagacataaataaggtcagtgcatctatttatctgcccatgtgctaggcattttcttctaagttgccttatcctcagacacatctgaagtgaatatagtattacaactacctgagattaattaaagtatggacatacattgttttattgagcttcactttattgcatttcacaggtaatgcttttttttttttttatatacacaaataaaacgttcgtgtcaactctgtgttgactaagcctgtttttccaacaggctatgcttactttggtgccatttttccaacagcttatgtttattttgtgtctctgtgccacatttggtaattcttgcaatatttagagctttttcattgttattacatccattatggtgatctgtgatcagtaatttttttgatgttactattgtgattgttttggaagtttgtattgaactaattgatcaatgtgtgtgtcccaactgctccaccaatcagccaatccccatctctctccctcttcttggatcttcctattccctgagacacaacaatattgaaattaggccatttaataaccctacaagagcctctaagtgttcaagtgaaaggcagagtcacacatactctccctttaaatcaaaagttagaatgattgagcttagtgaggaaagtgtgtcaacagctgagataggccaaaagctaggcccattgtgctaaacagtttgccaagttgtgaatgcaaagaaaaagttgtcaaaggaaatttaaagtgctactccagtgaacacacaaagaagtaaatagagatcaaaacagccacagtattttcttaaggcaaagcctaatccagagcaaggccctaatgtcactcagttctgtaagggctgagagaggtgaggaagctacaaggaaaagtttgtagctagcagaggttggttcatgaggcttaaggaaggaagccatttccataccattcaagtgcaaggtgaagcagcaagtgctgatatagaagctgcagcaaattatccagaagacctctagctaagatcattggtgaacatagctacactaaccaacagattttcagtggagacagaacagccttctattggaagaagatgccatctagaatcttcttagctagagaggagaagtcgatggcctggcttcaaagcttcaaaggataggttgactcatttgttatgggctaagacagctggtgattttaagtggatgccagtgctgctttaccgttccaaaaatcctagggcccttaagaattatgctaagtctactcttcttgtgctctataaatggaacaaaaaagtctgcatgacagcacatctgtttacagcgtggtttactgaatatttgaagcccactgttgagacctactgctcaggaaaaaaagattctttcaaaatgttactgcttaatgacaatgtatctagtcatccaagagctctgatggagaggtacaaggagatgaatgttgttttcatgcctactgacacagcatccatttctgcagcccatggatcaaggaatgatttctacttttatgtcctgttatttaagaaatgcatttcataaggctgtaactgccattacatagtgattcctttgatggatctgggaaaagtaaactgaaaaccttctggaaaggattcaccattctagatgtcattcggaacatttgtgatttgtgaaagaggtccaaatatcaacattaacaagattttggaagaagttaatcccaagcctcatggataactctgaggggctcaagacttcagtggaggaagtcactgcagatgtggtgaaaatagtaagagaactagaattaaaagtgaaacctgaaaagtgactgatttgctgcagtctttatgataaaacatgaatgaatgaggagttacttcttatggatgagcaaaaaaagtggtttcttgagaaggaatctgcttctggtgaagctgctatgttgagatgacaacagaggatttagaatattccataatcttagttgaaagcggtgacagggtttgagagggttgactccaattttgaaagaaattctaccagggtaaaatgctatcaaacagccttaaatgctacagagaaatcttttgtgaaaggaagagtcaggcattgtgacaaacttcatggttgttttgttttaagaaattgccatagccaccctaaccttcagcaaccaatatcctggtcaatcagcagccatcgacataaaggtataagaccctccacccacaaaagatgactcgcagaaggctcaggtgatctttaggattttttagtgataaagtatttttaaattaaggtatatatgtggttttttttagatgtaatgcttattgcccacttaatagacgatagtgtagggtaaactcatcacttacatgcagtggaaaaccaaaacatttgcgtgactcactttattgtagtggtctaaaactgaatctgtagtatctccgaggtattcctgtatcttctaaggttattctcaaattactatgaactctgaggtgagtcataatggtttttagacttattatttacaatagccatgataaatttacataggataggaactcttgatgtgtgtaggacacatcaatagaattgatgtcagaattattcttcagtactttctaagtaatctagaagagtaaaatttctatgtaatcaggtatactcttctgggcaatgaaatgctaggtaggccagtggagacacaccacagggagaacttgagaaaatttatgagtggactaaaagggggcaggtaagatttagttcaggtaactacagcagtaatgacaaataatgagtgactgtattcaagattttaggctctaaattatctattttatataagaatttttattgttttgcctgagattatttggtccaccctgctactttggatgtactctactggggaaaatgtagagtggtgtaggaacaaacaatcaaacactagtatcttgaatgaaagttctgtgtgtagtttggacagatatacttacagaagaccagaacactggtggtatatgatatataaccaagacagtaaagaaatgcaacattgcaggagcaaagagtaagtcagacctaaagcctagttccctttaactacacaggtgtagaccttggtcaacacactatccctacctgagtttccccatataaataaatgagattgttggattaaaggatattcaaggacatatgcatcttcaaaattttaattataaaaatgaattgactccagaaagataaagaaaaaatgacaaagctaaaaatgtagaccacaataggattgagatacaaaatcatgatataaataaacatagaaaaatgtagactggttctcccagaatcaaaatattagcagtattattatactttaagttctagggtacatgtgcacaacgtgcaggtttgttacatgtgtatacatgtgccatgttggtgtgttgcacccattaactcgtcatttacattaggtatttctcctactgctacccctccccctccccccaccgcacgacaggccccggtgtgtgatgttccccaccctgtgtccaagtgttctcattggtcaattcccatctgtgagtgagaacatgcagtgtttggttttctgtccttgcgatagtttgctcagaatgatagtttccagcttcatccatgtccctacagaggacatgaactcatccttttttatggctgcatagtattccatcgtgtatatgtgccacattttcttaatccagtctatcattgatggacatttgggttggttccaagtcttcgctattgtgaatagtgccgcaataaacttacatgtgcatgtgtctttatagcagcatgatttgtaatcctttgggtatacacccagtaatgggatggctgggtcaaatggtatttctagttctagatccttgaggaatcgccacactgtcttccacaatggttgaactagtttacagtcccaccaacagtgtaaaagtgttcctatttttccacatcctctccagcacctgttgtttcctgactttttaatgatcaccattctaactggtgtgagatggtatctccttgtggttttgatttgcatttctctggtggccagtgatgatgagcattttttcatgtgtctgttggctgcataaatgtcttccttatgactactggatacataacaaaatgaaggcagaaataaagatgttctttgaaaccaatgagaacaaagacacaacataccagaatctctgggacacatttaaagcagcatatagaggaaaatttatagcactaaatgcccacaagagaaagaaggaaagatctaaaattgacaccctaacatcacaattaaaagaactagagaagcaagagcaaacacgttcaaaagatagcagaaggcaagaaataactaaggtcagagcagaactgaaggagatagagacgcaaaaaacccttcaaaaagtcagtgaatccaggagctggttttttgaaaagatcaacaaaattgatagaccgccagcaaggctaataaagaagaaaagagagaagaatcgaatagacgcaataaaaaatgataaagggggtatcaccaccgatcccacagaaatacaaactgccatcagagaatactatgaacacttctacgcaaataaactagaaaatctagaagaaatggataaattcctggaaacatacaccctcccaagactaaaccaggaagaagttgaatccctgaatagaccaataacaggctctgaagttgaggcaataattaatagcctcccaaccaaaaaaagtccaggaccagacagattcacagccaaaatctaccagaggtacacagaggagctggtaccattccttctgaaactattccagtcaatagaaaaagagggaattctccctaactcattttatgaggccagcatcatcctgataccaaagcctggcagagacacaacaaaaaaagagaattttagaccaatatccctgatgaatatggatgcaaaaatcttcaataaaatactggcaaactgaatccagtagcacatcaaaaagcttatccaccacaatcaggtgggcttcatccctgggatgcaaagctggttcatcatacgcaaatcaataaacataatccatcatataaacaaaaccaaagacaaaaaccacgtgattatctcaatagatgcagaaaaagcctttgacaaaattcaacagaccttcatgctaaaaactctcaataaactaggtattgatgggacgtatctgaaattaataacatgtttatgacaaacccacagccaatatcatactgaatgggcaaaaactggaagcattcctctttgaaaactggcacaagacaggggttccctttctcaccagtcctattcaacatagcgttggaagttctggccagggtaatcaggcaggagaaagaaataaaggaaaagaattaggaaaacagggggtcaaaatgtccctgtttgcagatgagatgattgtatatttagtaaaccccatcgtctcagcccaaaatctccttaagctgataagcaatttcagcaaagtctcaggatacaaaatcaatgtcaaaaatcacaagcattcttatacaccaataacagacaaacagagagccaaatgatgagtgaactcccattcacaattgcttcaaagagaataaaatacctgggaatccaacttacaagggacgtgaaggacctcgtcaaggagaactacaaaccactgctcaacgaaataaaagaggacacaaacaaatgaaagaacattccatgctcatggataggaggaatcaatgtcgtgaaaatggccctactgcccaaggtaatttatagattcagtgccatcctcatcaagctaccaatgactttcttcacagaaatggaaagaactactttaaagttcatatggaaccaaaaaagagcccgtatagccaagacaatcttaagcaaaaagaacaaagctggaggcagcacgacttgacttcgaactatactacaaggctacagtaaccaaaacagcatggtactggtaccaaaacagagatatagaccaatggaacagaacagagccctcagaaataatatcacaatctacaaccatctgatctttgacaaacctgacaaaaacaagaaatggggaaaggattccctatgtgataaatggtgccgggaaaactggctagccatatgtagaaagctgaagctggatcccttccttacacctgatacaaaaattaattcaagatggattaaagacttaaatgttagaccgaaaaccatgaaaaccctagaaggaaacctaggcaataccattcaggacataggcatgggcaaggacttcatgactaaaacaccaaaagcaatggcaacaaaagccaaaatagacaaatgggatctaattaagctaaagagcttctgcacaggaaaagaagctaccatcagagtgaacaggcaacctacagaatgggagaaaatttttgcaatctacccatctgacaaagggctaatatccagaatctacaaagaacttaaacaaatttacaagaaaaaatcaaccccatcaaaaagtgggtgaagagcagtattatttttaaatgcccaataagtttattttttaaattggatttgaaaaacttggtaatgcctcatttattgcatttcattaagaatagagttctgggctgggtgcagtggcttacacttctaatcccagaactttgggaggcgggggtgagcagatcacatgaggtaaagagttcgagaccagcctggccaacatggcgaaaacccgtctctactaaaaatacaaaaattagccaggcgtggtggtgggtgcctgtaatcccactactcaggaggctgaggcaggagaatcgcctgaacccaggaggcagaggtcacggtgagctgagatcacaccactgcactccagcctaggcagcagagcgagactctgtctttaaaaaaaaaaaaatagagttctgaatataagattttcaagtacatcactagaactttaaaaatattaacactttgctagataacttctaaaaacacctgtactataatgtagttcaataaaagcattcagacattttttggtgagtggtataaattggaaggatcccaaagtcatcatggctttcagcaaatatcatattatatagtcctcacatgaaacatcaccagtattaccaaaacagtatcagtaagccttgtaatttgcttactatttgtctgattttctaattagtttagcattaggaacatggtcaggaatttcctgggcactttggaattgttaccaaggagagaagcagttatgatgtggtttattcattcatctacaaggaaatatatacaagtaagtaagcaccattagaattcagaaatgtgtgagagcaagggacatcatcttagcatttgaattattgcctaaaaaaatacacaaatgtaatgggggcactccattattaagtcatttaaacctttttgtgaatagagagaaatagcacttttaactgtatagacaaccactgacaattattatatgtaataagaggcctggtacacacatgaggaggtcagacttcttgtagaagaaaaagaatgtctgcttattttagtaactcctttttggagatctgcagggtgagctccttcaaagctagtagaggaggaagattcccttttatataatgactgctaagtattttattacatcactgtaagaggagatctagtcagtagaggggttgattttcttccctctgattcacacattgattgctgctctcaaatttgtcctcatctttcacacaacagtttgtacaaacacgttaccactagcacctacctgccccctcagaaaactaggaaagttcttacctgtcccattttatcaatgaatttggggatcttgtacttggaagaatagaaacttttaaggcaaatttcagattttaccaactagatgtaggtttcatttttccattggaaaagactgctttatcaggcatatttgagtttattgagtttgtcttttcatgggaatttttcttctgtgttttgcagtataaaacaatgcagcaaaatttagttaaatggattttgttaactttttggttgattcatatctatatttaaactcattttgcttttgtttaaattgtgcattaaaatgtttaattatggaacacataattattgatgcaccatcaaaacatttgtagttttccctaataaactacgtggctttttaatgaatagttttcatgacttcttcagttggctgaagaaatttttatcacattaatttttctaaagttttctggaatttcttgccgttttgttttaaaccatgtctctaggtaacacagtctatcttttatccttcctatttttcattttcctctcacttctccagagctgtcccacttcattatggacctttttgttcttatccccctactctcaagcttagcacacatatctagtaaataaatattaccttacatttcaatttcagtctcttcaacactcctctgccttcaagaatacatacactgtgtctgatgctaacaggtgctgttactttgctgtttctatcctccaaaataccaccattttttctattctttcactgcagtttatattcaccctcactgactccatttccttatcactcattgccatgggtagacaaagtgattagaagtaggtagtaatggttgggattaaaaaaaaacattttgaactggttgacatagccctatagaaatgcccatagtctgtccccataaaaagtgtggacttacagtggcaagaataggactatagcaagagtgcaaggaggcttcctcacagggaaaagttgtaagtaacagttaacaacctctttgaagtagagaagactgaacagatgaaagcaatggattttaaggattgcctgtaattaaggaagagaagaagaacaatggggtgtagaagttggaaggatgtagaagtagtagtgattgttcattttagaattcttaacttttaggttcttgctagaaaaaggttcacaaagtaccccccttcacatcttggaccccaccctcacccgcactccgtgtctttgggtgtggcaaagcctccgagatcccaaatcttttctatttcttgtcttcttaatttcttcactttttttccccttacccttcattagttacccctctctccttatctaaggccaaaatgacagtctttttacatacatacatacatacatacatctatagatttacattacatacatctatagatttacatctataaaactatttacatagtatttacattgtattcggtattataagtaatctagagatgatttaaagtatatgagaggctcatgcacgagaatcgcgtgaacccaggaggcagaggttgcagtgagccaagatcacatcattgtactccagcctgggcaacagagtgagactgtctcaaaaaataaaataaaaaataaaaaaataaagtatatgagaggctctgtgtaggttacctgtaaatactatgccattttatgcaagggacttaagcatctttggatttgggtatctgaggagggtcttggaacaaatcccccaggataccaagggatagctgactatacattacatattcttgtccagactaagacacaaaactgctgtggccttcaactggcattcagatacacagaaggataggtgatgtggtaaaagagagtaaatatatgggagaaaatggacagcatgggccataaggagagggaccacagaccactgatactcagtttattttaaagattatttttaacagctttgtcaaaatgtcttattaaaaactccggtcctcttttcatttttaggtttattttaggtatcgagcacatggttattgagatgttttcatttgacagtgcccctgtcctaagtggttgcctttcttgccagattgttttttcagttgttcttgtcaatttctgttgatcagacaaaggacagttgtctttttatctccctatcctgggaccttacacatttcatgttcaatattgttggatggatatgtgggtgggttgatggatggttggattttaaagaagcctttctattttagatgcttttaattgagaatggtaaatctgaatttctttagaataaaaaccacatattaatgcttttttttgtacctggagccataaagttttcagccatcacaattgctagcaattattttgctttcctgcatataagattatgggttaaaattgaactgacttgaagtaaaatggcataggagctttctgttccttagtgagaacccagatagctaccaagtctgattgtgagaaaacagtatctcctagaacctgataattagggcagtatcttcaactgtcttcccaggcaagactatttgatgagctggactaaattttctcatttaggggaaaggaaaatgaatagttttctcagttttaagcttttcctttcattcatttttaataaatcttagcatcaatttattgcttttctttaccacaatctgatatcatttagtatgtaatttagtacaaaatttagatgtttaggtactgatttgaataaatattattggcaggggctttgcaccagaaaacattttaaaactactacattaaaagtacaatttcccttgctagggtcttaggggaagggattaatgtagagtgactgcttgcgagtttggtgtttccctttggggtgatgaaaatgttttggaactagaggagatggttataaaccttgcgaatgtactagatgctgaattgtgctgaattgtacacttttaaatggtttatgttaacataaattataccttaatttaaaaaaaaaagcttttcttgaactttttaaaattcatgctgtatctgtgaattgccaaagtgtgtgctgtagctcagaggataatgtcctcctgtagcttgttatggttatgattgttccttcaaactgtgacacatattagagtgaaaattagtacttacaggctaaatacttaagtaacaagcataaaccagggttatcccaggtaactgggagaacagtcacattacctaaaactaacaaaaaatgcttttgtgtacaagctcgctaccactacttgctacagaacttgattcttcaaagaagtaagtacagttgacagagacagcttcccccatgcagataaaaagcctatttcaaacttgtaaaaagtaacaaaaataatcttactgagtaaagaattggataaagacattttcagttttaataaaagcagtgacactttaaacagatgtttctcaaatcctttttccactagtgtctagataagtcttatctgccattggctcataaacagttggataggttggtcagtacatccagttgtacatacatacacatacagacacacattctcattcatattctctttgctatgacaccaccaccttttaaaaacataaagaatatgtttgtacagtatttcctgtagtcaaatattgacatagtaggacaggaaaacctaatatatttaaatcaatgaaagatgcagatatgtcttattaatatacctactccaaaactttggccacatctttccccgagatatttttagttctcagacataattttagtagataatgcctttgttatatatcacagtacttctgtgggctttttctgtaagttgattactaactcagttagctagtttgagatataggtttgtcatgtgggtttttttttcccctgttaaactggtagggtttgttttgttttgttttgtttttgagacggagtttcgctcttgtcgcccaggctgaagtgcaatggcacggtcttggctcactgcaacctctgccttgcgggttcaagcaattctcctgcctcagcctcccaagtacctgggatcacaggtgtgtgccaccacgccaggctaatttttgtatttttttttaaacttattctttttttatgattattattatactttaagttctgggatacatgtggagaaggtgcaggtttgttacataggtatacatgtgccatagtggtttgctgcacccatcaatccatcatctacattaggtatttctcctaatgctatccctcccccttcccccctaccccgcaacaggccctggtatgtgatgttcccctccctgtgcccatatgttctcattgttcaactcccacttacgagtgagaacatgccatgtttggttttctgttcctgtgttagtttgctgagaatgatggtttccagcttcatccatgtccctgcaaaagacatgaactcattctttttttatggctgcataatattctgtgatgtatatgggccacattttctttatccagtctatcattggtgggcatttgggttggttccaagtctttgctattgtgaatagtactgcaataaatatatgtatgcatgtgtctttatagtagaatgatttataatcctttgggtatatacccagtaatgggatggctgggtcaaatggtatttcaaattctagatccttgaggaattgccacactatcttccacaattgaactaatttacactcccaccaacagtgtaaaagcattcttgtttctccacatcctctccagcatctgttgtttcttgactttttaatgatcaccattctaactagtgtgagatggtatctcattgtggttttgatttgcatttctctaataatcagcgatgatgagctttttttcatatgtttgttggccacataaatatcttcttttgaaaaatgtctgttcatatctttcgcccactttttgatgggattgtttgtttttttcttgtaaatttgtttaagttccttgtagattctggatattagccctttgtcagatggatggcaaaaatttttcttattctgtaggttgcctgttctgatgatagtttcttttgctgtgcagaagctctttagtttaatttgaccatttgtcaattttggcttttgttgccattgcttttggtgttttagtcatgaattctttgcccatgcctatatcctgaatgatattgcctaggtttccttctagggtttttatggttttaggtcttacgtttaagtctctaatccatcttgagttaatttttgtataaggtataaggaaggggtccagtttcagtcttctgcatatgcctagctagttttcccaacaccatttattaaatagggaatcctttccccattgcttgtttttgataggtttgtcaaagatcagatggttgtagatgtgtggtggtatttctgagggctctgttctgttccattggtctgtatatcttttttggtaccagtaccatgctgttttggtgactgtagccttgtagcctttgctgatgccgccagctttgttctttttgcttaggattgtcttggctatacgggctcttttttggttccatatgaaatttaaagtagttttttctaattctttgaagaaagtcagtggtagttcgatgggaatagcattgaataatctataaattactttgggcagtatggccattttcaccatattaattcttcctatccattagcatgtaatgtttttccatttgtttgtgtcctctcttatttccttgagcagtggtttgtagtcctccttgaagagatccttcacatcccttgtaacttgtattcccaggtattttattctttttgtagcaattgtgaatgggagtttgctcatgatttggctctctgtctgttattggtgtataagaatgtttgtgatttttgagattgattttgtatcctgcgactttgctgaagttgcttatcagcttaaggagattttgggctgaggcgatggggttttctaaatatacagtcatgtcatctgcaaacagataatttgacttcctcttttcctatttgaataccctttatttctttctcttgcctgattgccctggccagaacttccaatactatgttgaataggagtggtgagagagggcacccttgttttgtgctggttttcaaagcgaatgcttccagcttttgcctgaaacgttcagtaccgtattggctgtgggtttgtcataaatagctcttactattttgagatatgttccatcaatacctagtttattgagtgtttttagcatgaagggctgttgaattttattaaaggccttcgccacatctattgagataatcatgtggtttttgtcattagttctgtttatgtgatgaattacgtttattgatttgcatatgttgaaccagcctttcatcccagggatgaagccgacttgatcatggtggatacgctttttaaggtgctgctagattcggtttgccagtattttattgaggatttttgcatctatgttcatcagatatattggcctgaaattttctttctttcttgtgtctctgccaggttttgctatcaggatgatgctggctgcataaaatgagttagggaggattccctctttttctgttgattggaatagtttcagaaggaatggtaccagctcctctttgtacctctggtagaattcaattgtgaatccatctggtcttgggctttttttgttggtaggctattaattactgccattggtctattcagagattcaacttcttcttggtttagtcttgggagggtgtatgtgtccaggaatttatccatttcttctagattttctagtttatttgcgtagagctgtttatagtattctctgatggtagtttgtatttctgtgggatcagtggtgatctcccctttatcattttttattgtgtctatttgattcttctctctttcctttattaatctggctagcagtctatctattttgttaatcttttcaaaaaaccagctcctggattcattgattttttttttttaagagttttttgtgtctctgtctccttctgttctgctctgatcttagttatttcttgtcttgtgctagcttttgaatgtgtttgctcttgcttctctggttcttttaattgtgatgttagggtgtcaattttacatctttcctgctttctcctgtgggcatttatttagtgctataaatttccctttaaacactgctttagctgtgtcccagagattctggtatgttgtgtctttgttctcattggtttcgaagaacttatttatttctgccttaatttcgttatttacccagtagtcattcaggagcaggttgtttagtttccatgtagttgtgcggttttgagtgagtttcttaatcctgagttgtaatttgattcactgtagtctgagagactgtttgttatgatttctgttctttggcatttgctgaggactgttttacttccaattatgtggtcgattttagagtaagtgcagtgtggtgctgagaagaatgtatgttctgttgatttggggtggagagttctgtagatgtctatctacagaacttggtccagagctgagttcaagtcccaaatatccttgttacttttctgtttcgttgatctgtgtaatactgacaatgtggtgttaaagtctcccactgttattgtgtgggagtccaagtctctttgtaggtctctaagaacttcctttatgaatccggatgctcctgtattgggtgcatatatatttatgatagttagctcttcttgttgcattgatcccctaatttttgtatttttagtagagacggggtttcaccatgttggccaggctgatctcgaactcctgacctcaggtgattcacccaccttggcctcccaaagtgctaggattacaggcgtgagccactgcgcccggcataaactggtagtttaatgcagaatcttattttcactcttaccaataaaatagttttgcatacaaagttaagagtggactctaacatttgtgtcttaaccttctatagaactgtcaagtaatccacctctggctaccatccttattcctcctcatgctcggattcaagcagctgcttcaacccccacaaatgccacagcagcgtcaggtaagaacgttctaagtctcttctataaaaacatattctgggctgggcatgatggctcacacctgtaatcccagcactttgggaggccgaggtgggtggatcacctgaggtcaggagtttgagaccagcctgaccaacaaggtgaaaccttgtctctactaaaaataaaaacttagccaggtgtggtggcaggtgcctgtaatcccagcttctggggaggctgagacaggagaattgcttgaaaccgggaggcggaggttgcattgagccgagattgcgccactgcactccagcctgggcaacagagcaagactccatctcaaaaaaaaaaaattctgtttttcaaaaagatatgattacctttcatttcatttgatgagtatgaacatttaatatcttttatatatcaaaaacagcacagccaaatgagtaaacctgaataaaattttttgctttatttccaaaaagcataaaacaaaagcacccacccaggtaccttatatataagatataaatatcttctgataatgttaaattctgtcatcttagtgagaggatcactcgaggccaggagttcaagatcatcctggacaacatagtgagaccccatgtaaaaaataaaaataaacttaaaaaatctttgaagttacggtttttacataatttcaataggaaatcaaatcaaatgagatgtttatagctgaaaacatataaatatgttgttactgaaaataatacacttttgtttttttcccctcgaatgtaaattgcagtagtatactagagaggcaaatattttcaagaagtataaattataagtttatacttatatttaatcatttttttaatcatttgaaagggctaaaactgcaatgcaaatatgtttaactgagatttagatcagaatgtgacaagactcttatataccaaaaaaaaattattcacgggttagagctagggtgtgctgcagggcctgaagccagctaatggtggggcctgatttagtgagaaccggtgtccttgcaaatattctgaaaatatccaacccttaagagtgaagtgaaattcttccaaataatgactggatgcttttaaaacttaaaagttttaagttagaaacataataggtagcacagttaagggctcagaatttggcaccttttttttctctctctctccaaaaagctaatggcaggagctgacttcattttctttaaatagtcttgaactgagttggatcagatccatttttgcctaagggtatttatgctgcaggagataaaatctttcattactagttcttatcattaaacagtaaggtcaaaatccatgtcttgttgtttgatggtgttgtttctgtctaatcttaaaacttttaaaatgttgctaataaagatttaaaacatttcattctagttcatgttcaggaaaagtctggaaagaaagatctcttttcttgttccaagtttatcagcacatgctaccttgagaaagtaatttgaactttattagtttaattagtgtcttattcattttcccagtctgtaaaatggagtggcaattactttaggacaacatttgtctttacttgtctttatttgtctgtactttaggacagcattgccactccattttacagattgagaaagtgtgctgaaatctgctttaaaatttttaaggtaaaaagattttttaaaagtatgacctgtttagaatcaagacagaattgctataagccattaacaaatttaatactggcccaggttacgttaattgttcacaacatgaaaaaggccctaactctgaactaacacatttacttactgagctcatttctacagttttactagaatggctttacactgtgtataaaaatgagtaggttttgagcagtttaaagtaactttcttaatgcaagatagcgaactaattaatgattgtgacttatgtttttgagcaaattgtctgcttagtaggagcctagtataacctgattttcatgttgttttgatacaggttacagcaaactgaaaaataataataatttacatgtgtatatttttacagctatagaaatcagatttcaacataaaaatagaaactgacattgaactttgactctgagagtttactccaaagttgaattggggtatagttaatagaggaatagtaagctgaggtcagagctaaaattagtttagagaaatgcatgccttgagtaccttaatagttaccgaagaggatatttcagatattggtctgagctaaagtaaataaagaagtgttataattctgttaaaatttcccattgttcattagattgaaagcacactggtggcttagaaaaaacggtgtttctggccctgctgtttaagaaatatttctcctttagggcctcagatttggtactgaaagagatacagtaaatgaacttaacatcatcattctaacaaaaaatgtgagtttcaaatagatacttttttttaaagttccccttattgtaacccaaaaacagtttaaccacagtatagttaaacagttttctatggcgctgcagtagtgaaggcccaagtatgtcaccaaacatacttcaggaaatgctggtctagaccaagtgattgattgcttggccttccggctgcctgtagtttaagattcatgtatgttttaaataagaatttgtcattgtgagttacattcttttgtaagaccttaaaatatatatgtcctcggccaggcgcggtggctcacacctgtaatcccagcactttgggaggctgagacaggcagatcacctgaggttgggagttcaagacgagcctgaccaacgtggagataccctgtctctactaaaaatacaaaattagccggacgtggtgatgcatgcctgtaatcccagctactcaggaggttaaggcaagagaattgcttgaacctgggaggcggaggttgcaatgagccgagattgtgccattgcactccagcctgggcaacaagagcaaaactttgtctcaaaaaaaaaaaaaaaaattctcggcatcagacatgttttttttaaaaagccatgtggagggcaggattgacatccatcctcttatgacaaaagaacctatgacttttctttcctggataaatggacatgcatctctccacacagacagagtgagggactgctaagctacagacatagccgaagctttcctcacaaagcatttcaggatctgctccagagtacaaaataaataaataaactattaaactataaagctctagaccttgaatatctgaacctggaaatatttcaaatttaaattttcaaagaatttctaatcacccatctaagctacatcaccacagtaatatacccccacataccctggggcgaattaattataatatgaacccctaagtaactaaactagttgtcatacttgatttaaatcagtgtaatggagcacataacatcaaaatgattcgtatcttagaggtggagccacttttagtttatgacagtcatatcaacaagaagcaaaataatacttaaataatacttaacctgttttcagtagcctgagaatgtgcctccaaatcatctaaaaatgaccttacatcactaaaatacaaattcaaaagactaccataactgaattatcatctgatctgttaaaataccattcctgttgttgctcagcatcattgcaattcaggtactgtcaagtatatttgtgtctagagcaagatcatcagaataccgaggctcctagataaactgcttgggacactttttatgtttatagctggcttaaacaagaatggagttttctgacatttgtgtagacctgggaacaaagctcaatcctaaagtatgtggaataaaagcagaaaaacaaagcatattgacaaagatgccatcaaccattatcatactgactatattatggtgaaataagctttggattttagtcagttactgttgttagccaaagctgttaaatggctttcagatatttaagtcttattaaattttaaatacattataggcagaaatctaattctctatattgctgcttctgaaaaagcaaaagaaaatagacattttcatacccagattgtggcctaaaatcatactgtgctaattctgggaatatgtaattgttctctgtcatcaccaacaaactttgactagtacaacaaaatatcacaattctttctcacctttgaaaatggtaaagatggtgcaaatattaagtttgaggacattaacactgagggaactgcttttgcagatgtttcaactttccttttgttgtgttctgcaactgtatgtgagctgataaaaacttgtgggaaagcattatagtataaaacaagagctgtcagaattcagaatttttagttcagtaattccactttagggattttaacttcaggataaatctattggaaagaaaaaggctatcatatacagagaggggaatcacataatatatataatcataaatcatataataaaacagtatgtattaatatataacaaaaaggagagccaatgtaacctgaatggtcaacagtaggaaattgttttaatacattgtggaatctccatttttatgtagccataaaaaacaattttcatgaagcttgtggacattcatggaaatactcatcctaatttatgtagagaaaattttaaaatctcatgaaccttgttggttactattgtcaaaatgtgtattttcatgtggatagggactagacagaaatatacaaaactgtaaactgttgtgctaaattagtgggtatggatgttgatttttttcctaaaaaacattctttataagttgtttttaaaatgctttagtatctagagacattattgttaatcatggctctgattgacatgagaatctcttgaaaattacccatactactatcaattcctaaagtataatttctttgtttctttttttgagacagagtcttactgttgcccagactggagtacagtgtgtgatctcagctcatgtaacctccaactcctgggttcaagtgattctcatgcctcagccttccaagtagctcggattacaggcgcgcactaccataccctgctaattttttgcaattttagtacagacggggtttcactttgttggccaggctggtctcgaactcctggcctcaagtgatctgcctgccttggcctcccaaagtgctgggattacaggtgtgagccactgtggccagccaaaattcatagtataatttcttatgtcgactgaaatatatatatatatatatatatatatatgtgaatggtcaacgtaggaaatgttcaacatttatatatataaaatatatatttatatataaaatataaatatatattatatatgtcttattttctttgtacacccagtcatctcattcactccatttaggagaccattcatctggaacatctaaagtaataaatctcaagacattatcattttcctaatcaaatacaaaccattttagagagcatctgctcagatgagttgaatgtgagatgttgtagggaatttcaactagagcagttttctggttttatctgtttggtatattggtcatcagcataaagtttctttcaatgcaatggttctgctgttaaaaaccatggatttcagttcacctttcattatctaccgagaagtggtcatctatcccatggttatgtatattcagtaatagacattattgcaagaatgagcacatttttatctttgcatcactctattagaaaagtttggccaggtgtggtggctcacgcctgtagtcccagcactttgggaggccgaggcgggtggatcacgaggtcaagagatcgagaccatcctggccaacatggtgaaaccccgtctctactaaatatacaaaaaaaaaaaaaaaaattagccgggcgtagtagcgggtgcctatagtcccagctattcgggaggctgaggcaggagaatggcgtgaacccgggaggcggagcttgcagtaagccgagatcacgccactgcactccagcatgggcaacagagcaagactgctctcaaaaaaagaaagaaaagtttatccttttttcaccctgaaatttatttctgtatgccttcttccagtcagtgattctactatgaaattataacagaaccatccagatccttccatgtaaaagtccttcaaatgttaatgatagctaacttgccctccatactaatgacagctttaaatcctaatcctggatttgttctaaagtattagggtattggattttctacccaattgtatgtcatctgtacatttcacaaacatgtcagttttttacctccatccaagcaactaataaaacatatcaaagaggacaaggcagagcctactagaaatgttttgcctttattcaagttactgataaattcattggaactgggctgggtttcctagagattactcttctggtggtctcttagtgattggtcattcctcttcgtgactggtcattcctcttgtttgcttaactacagaccaacctttaaaatgcccatttataggaggaggttataaaaagcactttgtaaaatgcttcattgttttaagaaaactttcagcctttttccattaaaaaaaaagtaaccatatcaaagatggaaatcaaaccgttgtttttaagacggggaaatttctagtgcctaaatatctgtaagctgtgagtttctgtaatccgttttagatttgtttcttggactatagattgtgagtttaaagacatttaaaatttttgattgccctcaccttccttggggcacattttatttattaggatttttattatatcttttcaagttgaaaacccttctaacagaagggggagtcatgataacatttggatgaggtctgtcattttacgtggtctgcaggcttaacttgtcttcatcttggtttgaaaagctcttactgttactttatttgcattgagattatttgcaattacttgatcaaaattttgtttatgcttttctatttcttgttagttattagcctcctttgaagactttagccacaaaatcatacctatttgttcttcaaacttctaaaaattcatttggctatagtctcgttcagattttgctatggctagtggcccctttgtcttacaattacaattattttcaccccagaattcctgttacctacataccagccaattctttttagttgggcagaattaaacacagaataatttctctcattgtttgtttctactgtttttgagaggtgaaatgttaagcaaattgtcagtttattagatgcttgcttttctaaaatgacagttaccctccctcatcctttccccaggtactactaatattattcctggtctttgggccagttttacagtgtacattgggaaagctgtgttcatattctgtctagtgtagaagctgcagtgcatgccctgttgttcctctttgtaaacttgttcttcctgttttctctttatcttaacccaaatagattttaaatttcataaaagtctgcaactttttaaaaaaaaacaaaaaagcaggcatttttgtctatcaagaacatatagtgtatagttattttaggaagtaaaaattgcctctattctactttcaaagataatcacacataacattatgttttagtctatatatgaatgggcagagagacatgttttttaaaaaatgggcttaggctatttagagggtgaaggtatagactgtagtattcaaccaatatcctaaggctataccatagttaatttcttattcttggacttttggtttgtgtcgaagatggggttttttgtttttgttgttctgaaaaatgctttgaagatatctttgcataaagctgtacttattcttctaaatttttagaagtagagtcaaaaagtataaagaattttaaagtttaatgtcaaattgctttctgaaagtttgtcccgctgtcagttcatactccccactgtcagtacaagtacttttctatttccccattgccccatgtcctcatagagtgggagtaggggaagtacagtgtgcatgtgtgcacatacacatttttaggtgttaccaacttggtagccaattaatatgtgcatcttttttaggtacagtgttgcatccatttcctcctgttggctctgcatttgaaatacagacttccattttgaactataccattttgacaaattcactgacaccaatgagattgtatctaccccatgttagggtttcaggttcactttgtgagtttgtatatagatacctaaaatcaaaccagcttagtcattattctcaccagagcagtcctagacatcacttctagaagttcttgctttctgtgcaaaacatgttctctcctatcaagtcaaaaattttatctcggtttttcccctcctctaaaagtaatttaaaatctggattaagttggaattccctatcagacatttttccgtgtgtccctgaagtgttcctcagttccttgcctgaagtcacctacttttatttatatgtccttttttttctttattcctaaattaagcattttaacttaaaggaacagtgaaaatgttacctgtgtgtccccatgaccttcagttttctaccctgaacagccaaacttcttaaatacaatgtgccctttccctgagctcacagggaactgagacctctcagctgccagcagatcaaatataaacagtcttattgacaggtcttccaggtatcctggtggatggggttggctcacaggcatccgaattttactgctatttttataatcactgaaggctaccttagtgttctgtgccacatcttttccttgcaggtgtactttgatttcatgagtgtaaattataatttcaaattaaatataagtttagggtatactttgattctctgtgagtaattatcttgtttgttaatgtgccagttaataacattaatatctaagacatagttttacagtagaagcatttccacttggaacagcttgagtaggaacatcctgagttaggtacacagtataaataatatctcccaggctgttaattttatcttctagagagattgacctgtcataagacatttctaactattatagaaagaggatacctgataagtagaaacacgtaaaatgtgcttggaagagattgttattgggcaagagcgtagtaaaggaaatacgggaataaaaatatacctggcggggtgcagtgactcacacctacaatcccagcactttgggaggtggaggcggtcagattacttgaagccaggagttcgagaccagcctggccaacatggcgaaaccccatctctactaaaaatacaaaaaaaaatttagccaggcatggtggtgcgtgcctgtttaatcccagctactcgggaagccaggagaatcccttgaacccaggaagcagaggttgcagtgagctgagattgcgccactgcactccagcctaggtgacagagtgagaccctgtctaaaaaaaaaaaaaaaaaaaaaaaacaacctaatgtttccaagtgtacttataaagtgcatgtaaaacacaaaaaggctcatttgatttgtctaaaatatgttattttactaatatgccaacaatataaatagtaaaatgtttaataattttgtagctgagaacttgaaatgtgttgagagcaaataaggaactgaattttaatctttaacgtcaattactttaaataaatagccttatgtagttagcagctactatattagagaatataaatagaacatttctgtggttgctgctgggccagatagtgagcatttcaataaatcagaagaatttaattttttatagacacacagatctagaatactaattcatggtggcagggcctttggaaccctgtctcctatgtttattttctgtcttctcctcccaacccatttccttgttttctactctctttttatttaaatcctttggcagtaggtgtaaatggagaccttccattcactctccttcctgttcctcatttgcacctttgatcaaccacatttgggacagggagtatggacaggaccatttgtgggcggggggtggggggatgcagacagcaaggaatcaaggcaagaaattctgcgtttatgacttgaacatttgggtggggcagtttcctcagtattgagacagttgaccattaatccatgactccattatctagccaactcttacagatctcttgagaatctgaaaaaaacaaacaaacaaaacaaaaaatgctgtagatcctgtcctataaaaagaaattatgataccagatatagaattttacatatgaaaatttactccaggacaaaaatcacagcctcatggactcttttgggaacctgatacttaagggttctacccttggtttcactgtgggactgcctaagttgtgttttacctcttatgtcacacagaataaaattggttttaatacatgtgccaatttgctttgcagcatctggtacaaagccatagagccatagacttttcagaaaacaattgattcctgagaaaacaattctggaaaaaagaattaaatctaatataatccttttttgtcttgtacagtttcttagaagcacacctaattgtgtagggagattttgtcagcgattccctcattggcttgtgagtgcaacccctaaatggcatttaagttcttcgacatatacatgtgtgttatctatttctgcatattagtttacccccccaacatttagctatttaaaacaagagtaaacattatctcacatagtttctatgggtcaggaatttagaagcagcttagctaggtggttctgggtcagagtctcatgacatagtcaaaacattagccagggatgcagtcgtctgatgacttgcctggaggcagaagctctgcttccacagtggctctcattcatgtggttatcaaatatgtgctgcctgtatgcaggaagcctcagtcactcaataagtggccctctccatagtgctacttaagtatctcacatcattgcagctggcttcccctagactaagtaattcaagaaagaaggcaatgcagaagccacagtatcttttatgtcctagccttggaagtcacacaccattatttctggaatatcctattagttacacaggtcagtcctattcagtgtgggaatggactgcattaggcatggataccaagaggcatgaatcattggggtcatcttggaaagtagctaccacagtgtgtagggatgattttgtaccttcctttccttccccacaaagctgattgttctatttcctcttaagtccatccttggtttgggggcatttttttgtttacttgtttttccatagtagcattagacaggtgataatgggcattggtttataggaagcttcaaagtcagtgaaatgtcagttctgtagttgtttgcagagcacttatatctctgtacaccccatactgaaaataaagtaactgtgctcttaatcttaatttttcttatcataaactcactcttctcttatttttttttttttttgagacggagtctcgctctgttgcccaggctggagtgcagtggcatgatttcggctcattgcaacctctgcctcccaggttcacgccattctcctgcctcagcctcccgaatagctgggactacaggcgcccgccactatgcccagctaatttttggtatttttagtagagtcagggtttcaccgtgttagccaggatggtctctatctcctgactttgtgatccgcccacctcggcctcccaaagtgctgggattacagtcatgagccaccgcgcccggcccactcttctcttttaatgtaaagtctcttacagataatagaaatgtgcctgtagaaatcacagtgattttattgaggttagttctaaatgaaagtcatcttaatctcttttcttcttttgctaggaaatcttttctgctgccacctactgcccataagatggattattctcctcccttgtgctctgtgttccactcccctacttgttagctatatggtcttgggcacattccttaatctatttgtgcttcctttctcctatttgcaaaacagaggtaacagttgtacctatttctcagatttgctgtgtgagctaaatgacatgactgcaaaatacttagtgtggtgtctggcatattggtaagcagtatgctgtcactagtatttactttggtgatgatgttggttattgcagtcacactagattataaatatatgtatttttagatgtctcttttaccttcccatctattgaacaattgctgtatgccagatactgtgctaagcacttcataggcctcatttcataggtgggtggtattactaccattttaccaatgaggaaatgagagatagattaaataacgtgttccaaaatcacacagctattaagttgtacaagtaggcttcatactcaataaatgtttatagagttggtttagttttaagaaagatgtatcagtgtgaagttcagaagagcatctctggttgaaaacaagagactagagtgggttaatgatccatagtcagatcatttataatgaaaatgatcttagcctctcttttaaaccataatgccatttagcgtcactttaccagtgcatatgtaacgggaagggaatgtaaggagaagtaaaatgaactttgctgtttttatgttgtggagtcactatttaaatgtttgagaagaaaagttacagaaattacactaaactaaatccaatgacaatttatcggaattttaaattaataaacattttaatagaaagctcattaattcagattccattgtgtaaaataaatttacagagtgatggtgggaacaaaataagcatgtgccaagatttgtttcttgagttatttctaaaaaggtggtaatctgtgttgtcttgtttcctgatcttgaactagtttcttcccagccaccaagaggtcagctcctcctactttacctggcccaggggccagtccatctttatgatgggattgtagagattatgaaatgtaactttactcagtcagtaagctgggtggtcttttaaacctactattgagtattgaatttatagaactttgcctttttttgttttactttttccctctctgtactttgtcttcctctgaaaaagagaactctgttcttctcagcaaggacatgtgtgatttgattttaagtggagctcattgcatcaggcaaaagtttttttgatgaatgtctgtaggactgtgactttcgatatcaaagatatttataatcaaaataattagcgttaaaattatgttcattaagtgatttgaaaataattcaagagaaaaatatgagatcattacaggtctgaagagcatgggtgtctatcaaattttattgttcagaaagtgaatttgtttctggttttatgctcacttaaaaggcttgtgctttttactgtgatcatgcaagcagtcgttgaatttatccaagaaagaggagactggttttggcaatgtcacaagctcacaggctgtagtcagtgaaaggacatctgcacattaatgccaaataggattttgctaaagagcaaattaacataaatgggctggtggccatgtagatgaggtaaaactgatgtcagccagtgtagttaatggctgccgtggccagagctgctctctccatgttggcatgggtgctgatgtagtggtcaaacagatagataaatttctgatgatcagtgcctcccataagtcctggggaagaccagaatgacagttctacctgttatcttcccatactgaaaatgagaaggaaatgctgttggatgatggtcatgcataggttctgagtgtcatcagagcatgtgacatgctgcaataagcatgggtttgtggtgttccaggttttctctggattttttttttaatgcagcctgtgcatgaatcatgaaaatgaacattttatctgagaaaggcttggaggaatctgtggtctagacaggaattgatgttaggttattgatgcacagaatgtggatgatcattaagggtaaaacttctttaaacagggcatatctaaggaaggtcaggagtcacgaatgaagtgttagcatagttcctcagtgattgttagaaaaggtggtcagcctggctttggagagtaggttctgtgtcatataccctttcctgccacctgttcatttgccatactgatgaattaatatgaccccattacatgggctatatgaacctaatgtgtggattctgatgccataatatcttcacttgtgtacatgacaacaagattagaagcagctttagctattttgaaggaaattaaatgaattaaacctaatcattcacattaagagatattcacattccttccccaaaggctgtgctaaatgtatataagggcagcttatcgggaaattaggaagtataatttttatagattggctttagaaatgataaactgaacagctaatccatcatagagttccctatgtgtccttggatattctgtgtctccctctaattcagaaattcaaagtagaaagatccaggaccaaacgttgcattcaaccatgacattggttattctactcaccatagcacatggtagatctacaaatcccttcagtcctagagagaaaaaaacttctgagtctgcctttcggtgtcaccaaccttgatggcattgtggcttctagagagatggcttttcaaagttgcccatcccagtcaagctacctctcttgctcagggcttagtgtgaaatctttgggagaaaatttggccagcagtaccttctctgtcattaagagagatgacaaggatgcaacttgatagccactaaaacagcagtctactaaaccaatttggacacagttagatgcccacttaaatgttaatggatgctgtgaaataataatagaccttaaaaggggggaggagataatggttagttgtagagttgtacgtatttctaaaagattagggcaaagtaagctctaagtaagttctacgtcaagatttgactacaagatgctttccttttcttaatttttaatttttgtggggacatagtaggtgtatatatttatgggttacctgagatattttgatatagacatggaatgtgtaataatcatatcagggtaaatgggatattcatcacctcaagcatttatcctttatgttacaaacaatccagtcatactttgttattttaaaatgtacaattaaattatttttgtactgtagtcaccctgttttgctagcaaatactaggtcttatacatttttttttttgtacccattaaccatccccacttcccccacccccgctacccttcccaacctctggtaacaatccttctctctaactccgtgagttcagttgttttaaattttagctcctacaaataagtgagaacatgcaaagtttgtctttctctgtctggcttatttcacttcacataatgacttcagttccatccatgttgttgcaaatggcaggatctcattctttttctttttcttttctcttttctttcttctctctctctctcttttttttttttttttttttttttaaagacggtatcttactgtgtctcccaggtggtcttggctcacttgcagcctcaacctctctggctcaagcagtcctcccacttagcctcccatctttctctcttatggctacatattaccccattgtgtatatataccacattttcttttatccattcatctgttgatacaagatgctttattaaattgactaagtgaattggtactgcagttgctgtttcttttttgctggttgtctgtcttccatctctatgaataatgaagagggtctgttctgcccatcaacactgacccacgttactttcattgcctccacctatttacttacttaagcagatggcatattgtaacatggttcaaatttttagcaagttttcccagctgtgcactgttcatttattttcaagtgaactagactccagctgagtgttcatatacagctgatcccagctgtagcaagagctttgactgaaatggtctctggctccctgagtctgcaacttattcaaagacactgtcttcccggtaccaggaccatgctgaggcatgaacttaaatccaagaatagagaaattcagatttcatagtgttggccttatttaaaacaacgttccaaataacagcttggaaggccatacagttcacagcctgcagttgaaacagtgtacttgttctcttttgttgttatagcattgctcatgctctctcttgggactttttgacttgcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatatt

SEQ ID NO: 2 - DNA, 1639 bp, Homo sapiens
  CDS (233)..(1534)
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 3 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 4 - DNA, 1600 bp, Homo sapiens
  CDS (233)..(1495)
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 5 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 6 - DNA, 1540 bp, Homo sapiens
  CDS (233)..(1435)
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 7 - protein, 400 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQDANTGDRGQTNNAASASASNST

SEQ ID NO: 8 - DNA, 1501 bp, Homo sapiens
  CDS (233)..(1396)
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 9 - protein, 387 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQDANTGDRGQTNNAASASASNST

SEQ ID NO: 10 - DNA, 4356 bp, Homo sapiens
catttatatttaaatatgttattgacgagttattctaatttgtcttcctttccaatcttgatccaaatgtacttatattttacagattggaaatatggtagatatacaacctgaagttctatatttttcaattagtattgtttcctaaacatatttctgagttacttttggtggttgtatttatttttactggctgcctaatatggagttaatacagcatagtttactttactcctctattatcctgtgatattttcctttattttctcttggcccacatcttagacattacataatttcaaactatctcaaggtctccaaataccttgttaaacatttcagccattgcataaccttttctctttgcctggaatgttctttccttccttatccatctgtacaactgtacaactattctttgtctaaactagtggttcataaacttttttttttttttttttgagacggagttttgctcttgttgccttggctggagtgcaatggtgcgatctcagctcactgcaacgtccacctcctgggttcaagcaattatcctgcctcagcctcccaagtaactggtattatagacgcccgccaccacgcccagctaagttttaaagttttagtagagacggggtttcaccatgatgaccaggctagtcttgaactcctaacctcaggcgatccacctgcctcggcctcccaaattgctgggattacagtcataagccaccactcctggcttgtccataaactcttgatcttgagctaacatgatatacacattataagatacacacagaaataaaaaaaggattaaaggtgcactattttaaagataaatttcaaccggcccggtgtggtggcttacgcctgtaatcctgacactctgtgaggctgaggcaggcagatcacctgaggtcgggagttcgagaccagcctgaccaacatgaagaaaccccgtctctactaaaaatacaaaagtagctggcatggtggcacatgcctgtgatgggaactacttgggaggctgaggcaggagaatcacttcaatcggggaggcggaggttgcggtgagccgagatcgcgccactgcactccagcctgggcaacaagagtgaaactccgtctcaaaaataaataaataaataaagatacatttcaattatttaaggtcccaaaccattttgttttcaccctcctactgctatcggggcccctaacaattattaacactttttccaatgcctcattggccaagggatgtttttaggttggcctgcagggtttgaaaatttggttaccaacatttaaaaatgcagagatttcacacaaaaatccaattttggggtttcttttgggaggaaaaaaattgaacatctgagaacactggacccacattcctgagggcaacaatctgctagagttgagcagctgttccttttagttatggcatggatattccaatttgccattttccctatcattccccatttgtcttacacaaggccagtctcactcatttgctttacttgtctggcctctgtagccatttgaatctgcaaacactatctagacctttcctgtcttcccatcaaagcttactttgttctgtcccaagtccttcatcagtgtttcaaagcaagagccaggtaatctgatcaaatataggtcctttwacatgtgctttctggagacagcatcttaacagaaagagatgactctgcaggtagaaattacaggcaccactggattacaatcaaggatggcaactgtctgttctactttctctgttatcttgacgtagccatacctcactgttctaaagcagaccaaaggaactggttctatggtagaacaatggacgctggtcttatgtatgaaatcttctcaagctgcacttttatagatcaccctagttccaaaagatccaaagctaccacgggctcctctgacccccccatttcctcggaggcttcaggatatccggcctgacggcatttcccctcacctactttcggggcccgttttgcgctgggcagttgcgcccaaggagcgcgcgcatccagcatgagctcatttctcatgggcgtttccagaggcccggccgggcgcgcacaccaagcgcagcaagcctctgtatcaatggcccccgcggctgggcggggcgcggccgcccaaaggcgctgcctttctggaagctttgttcccattttagcgtctgagagcttgcagccggctgggaaggcccccttggtccgtctggccctttcggggaagaggccaacactcggcacacgcgatccacggcagaggggagccttgggcgcgcagaattggctgcgccccgccgagagcctcctgtgggtggggagagcccctccacccctctgctcgcttgagcgctcagagcccagggccgccgaccgcagcactttccgatttgctgcaccgagggcccgcggtccctgtgtgcggtttccaccgttgttggaggcggccgcaggcgaaccctcgggtcgtcagccacgacccgagtcaggcatctccccgctcctgggaccggggccgaaggccaatcacactgcagctaggtctttgcggattggacggcagtgagagccgattggccgccgccgcgagtttcgggctccctccctctcccttttgccccaactccagcgcctaagctttaggccaatgagacagcgctttatagacgccctcccttcgctttcttctccccaccttggagagggaggggaagtcctgactggccagactgtcctcggaagcccccttctcttcaccaatcaccgaaggaggtacgctcccagcggagttttccagccaatcacaaagcggcggcggcgcccagccgtgcagtttcaccagcgtctctgggtttcaccgtcctcaactcttcaagcctcttcgtaagggccggcgactctgattggccactcttgccattgtcaaatgtctcctccagccagccaccgaacaaggcgaattcaccctttccgttcggctaccttcggcattttccgctctttgggcgtggcttgccagcgtcactttctaattggtttctcaggctgatcggctttttccgggaggagccgcaaacaaacgacgtccgtgattggctccgttcgggcttcggctcccagccgaagcgggcgagcgtggggctcggccggcgattcccagacgcctgttacgcgggcggcggggcgctgggcggtgtaaggctgggtgggggaggaaggaggaggaggacgagtaggaggggggaggaggagtggggaagtgcaaggcggctgcgcagacagcgctcctcacacagagcagcycctgacccgggcgaatgcgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatc

SEQ ID NO: 11 - DNA, 2946 bp, Homo sapiens
  CDS (372)..(749)
aatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgatt

SEQ ID NO: 12 - protein, 125 aa, Homo sapiens
MEGQGTQGRPTVPLSPTCTLACSCQPPRQPSPLLSSTCIVSALHTRQRGTHPLGNGSFPPVPAGSHAVCSAGAAEHIHRCCPALPICTLLSCSWICLFMLGFTRVTMIVKRKKKKKKDACILKCL

SEQ ID NO: 13 - DNA, 1804 bp, Homo sapiens
ccagcaactcagaaggctgaggtgggaagattgcttgagccccaggagttcaagcttgccgtgagctacgattgtgccactgcactccagcctgagcaagaccttgtctccaaaaaaaggtcaattccactgacttttctaaggtgtacaccatcaaggggcagctccatctccaggccattggctcatgagacattctgtagtcagaaggctagggcagattgctttgagcaagcccccatggtggttctcactcctacttctttgggtatatgcccctctgtttaaaaataaagttaatatgcatttaaaaaaaaaaaggagaaaaaggtcagttccagaaactgtgtgaataaagcattttacttgctttttctattaatctataacatatgttgattttttaaaaagaatataagagctatgcaaattggagcttcaagacaacttcccatctccctaggaggagatggctgccctaaacccccctacatagaaatcatcccactgcttgggcttaaacttgatgttggggaaatgaaaaatccaagctaaggccgaagcctggggcctgggcgaccagcagaatgaggaccactggtcagtttcaggctgaggtgcgtcttccaggggacaatctctagctggcccttaaacattcagacttcaagctctatttacagcataaaggtgtttcaaaagacgtgatacaaataactgcaaatgctctgcgatgtgttaagcactgtttgaaattcgtctaatttaagatttttttttctgacgtaacggttagattcacgtttctttttttttaagtacagttctactgtattgtaactgagttagcttgctttaagccgatttgttaaggaaaggattcaccttggtcagtaacaaaaaaggtgggaaaaaagcaaggagaaaggaagcagcctgggggaaagagaccttagccaggggggcggtttcgggactacgaagggtcggggcggacggactcgagggccggccacgtggaaggccgctcaggacttctgtaggagaggacaccgccccaggctgactgaaagtaaagggcagcggacccagcggcggagccactggccttgccccgaccccgcatggcccgaaggaggacacccacccccgcaacgacacaaagactccaactacaggaggtggagaaagcgcgtgcgccacggaacgcgcgtgcgcgctgcggtcagcgccgcggcctgaggcgtagcgggagggggaccgcgaaagggcagcgccgagaggaacgagccgggagacgccggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgctagagcaagg

SEQ ID NO: 14 - DNA, 1809 bp, Homo sapiens
ccagcaactcagaaggctgaggtgggaagattgcttgagccccaggagttcaagcttgccgtgagctacgattgtgccactgcactccagcctgagcaagaccttgtctccaaaaaaaggtcaattccactgacttttctaaggtgtacaccatcaaggggcagctccatctccaggccattggctcatgagacattctgtagtcagaaggctagggcagattgctttgagcaagcccccatggtggttctcactcctacttctttgggtatatgcccctctgtttaaaaataaagttaatatgcatttaaaaaaaaaaaggagaaaaaggtcagttccagaaactgtgtgaataaagcattttacttgctttttctattaatctataacatatgttgattttttaaaaagaatataagagctatgcaaattggagcttcaagacaacttcccatctccctaggaggagatggctgccctaaacccccctacatagaaatcatcccactgcttgggcttaaacttgatgttggggaaatgaaaaatccaagctaaggccgaagcctggggcctgggcgaccagcagaatgaggaccactggtcagtttcaggctgaggtgcgtcttccaggggacaatctctagctggcccttaaacattcagacttcaagctctatttacagcataaaggtgtttcaaaagacgtgatacaaataactgcaaatgctctgcgatgtgttaagcactgtttgaaattcgtctaagatttttttttctgacgtaacggttagatacatcatagattcacgtttctttttttttaagtacagttctactgtattgtaactgagttagcttgctttaagccgatttgttaaggaaaggattcaccttggtcagtaacaaaaaaggtgggaaaaaagcaaggagaaaggaagcagcctgggggaaagagaccttagccaggggggcggtttcgggactacgaagggtcggggcggacggactcgagggccgcccacgtggaaggccgctcaggacctctgtaggagaggacaccgccccaggctgactgaaagtaaagggcagcggacccagcggcggagccactggccttgccccgaccccgcatggcccgaaggaggacacccacccccgcaacgagacaaagactccaactacaggaggtggagaaagcgcgtgcgccacggaacgcgcgtgcgcgctgcggtcagcgccgcggcctgaggcgtagcgggagggggaccgcgaaagggcagcgccgagaggaacgagccgggagacgccggacggccgagcggcagggcgctcgcgcgcgcccactggtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccatcagctccggcaccaacagcagcgccgctgccaccgcccaccttctgccgccgccaccacagccaccttctcctcctccgctgtcctctccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgctagagcaagg

SEQ ID NO: 15 - DNA, 24 bp, artificial sequence
  oligonucleotide for amplifying GSK 3 beta promoter
tttatagacgccctcccttcgctt

SEQ ID NO: 16 - DNA, 24 bp, artificial sequence
  oligonucleotide for amplifying GSK 3 beta promoter
tccttccttcctttgtcacttggc

SEQ ID NO: 17 - DNA, 24 bp, artificial sequence
  oligonucleotide for amplifying GSK 3 beta exon 3 SNP
tataaaagctctaaacactctcaa

SEQ ID NO: 18 - DNA, 24 bp, artificial sequence
  oligonucleotide for amplifying GSK 3 beta exon 3 SNP
aatgctttcctgatataactaatg

SEQ ID NO: 19 - DNA, 24 bp, artificial sequence
  oligonucleotide for amplifying GSK 3 beta exon 5 SNP
gcttttggtgccttcttaggtgac

SEQ ID NO: 20 - DNA, 21 bp, artificial sequence
  oligonucleotide for amplifying GSK 3 beta exon 5 SNP
cgaaacattgggttctcctcg

SEQ ID NO: 21 - DNA, 23 bp, artificial sequence
  oligonucleotide for amplifying GSK 3 beta exon 5 SNP
gccatgtctgtggatgatgaatc

SEQ ID NO: 22 - DNA, 22 bp, artificial sequence
  oligonucleotide for amplifying GSK 3 beta exon 5 SNP
caatgaatcacccaagaggctg

SEQ ID NO: 23 - DNA, 21 bp, artificial sequence
  oligonucleotide for amplifying MAPT promoter
actgagttagcttgctttaag

SEQ ID NO: 24 - DNA, 26 bp, artificial sequence
  oligonucleotide for amplifying MAPT promoter
tagttggagtctttgtgtcgttgcga

SEQ ID NO: 25 - DNA, 22 bp, artificial sequence
  oligonucleotide for amplifying GSK 3 beta using RT-PCR
tgttggagttcccaggaccttg

SEQ ID NO: 26 - DNA, 24 bp, artificial sequence
  oligonucleotide for amplifying GSK 3 beta using RT-PCR
agtaactggtggtttttcctgtgc

SEQ ID NO: 27 - DNA, 24 bp, artificial sequence
  oligonucleotide for amplifying GSK 3 beta transcript using RT-PCR
cgagggacactaaatacagttcaa

SEQ ID NO: 28 - DNA, 24 bp, artificial sequence
  oligonucleotide for amplifying GSK 3 beta transcript using RT-PCR
agtaactggtggtttttcctgtgc

SEQ ID NO: 29 - DNA, 23 bp, artificial sequence
  oligonucleotide for amplifying GSK 3 beta promoter
aaaagatccaaagctaccacggc

SEQ ID NO: 30 - DNA, 24 bp, artificial sequence
  oligonucleotide for amplifying GSK 3 beta promoter
tccttccttcctttgtcacttggc

SEQ ID NO: 31 - DNA, 27 bp, artificial sequence
  oligonucleotide for amplifying GSK 3 beta SNP rs 3755557
gccgccatcctgattgtaatccagtgg

SEQ ID NO: 32 - DNA, 25 bp, artificial sequence
  oligonucleotide for amplifying GSK 3 beta SNP rs 3755557
gcttactttgttctgtcccaagtcc

SEQ ID NO: 33 - DNA, 24 bp, artificial sequence
  oligonucleotide for amplifying GSK 3 beta SNP rs 334558
tttatagacgccctcccttcgctt

SEQ ID NO: 34 - DNA, 25 bp, artificial sequence
  oligonucleotide for amplifying GSK 3 beta SNP rs 334558
ttccttccttcctttgtcacttggc

SEQ ID NO: 35 - DNA, 24 bp, artificial sequence
  oligonucleotide for amplifying GSK 3 beta SNP rs 6438552
gcttttggtgccttcttaggtgac

SEQ ID NO: 36 - DNA, 21 bp, artificial sequence
  oligonucleotide for amplifying GSK 3 beta SNP rs 6438552
cgaaacattgggttctcctcg

SEQ ID NO: 37 - DNA, 24 bp, artificial sequence
  oligonucleotide for amplifying MAPT promoter
actgagttagcttgctttaagccg

SEQ ID NO: 38 - DNA, 24 bp, artificial sequence
  oligonucleotide for amplifying MAPT promoter
tagttggagtctttgtgtcgttgc

(19) United States
(12) Patent Application Publication
(10) Pub. No.: US 2008/0152589 A1
(43) Pub. Date: Jun. 26, 2008

(54) Diagnostics and Therapeutics of Neurological Disease

(76) Inventors: Peter Schofield, Marsfield (AU); John Kwok, Potts Point (AU); Clement Loy, Darlinghurst (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/852,912

(22) Filed: Sep. 10, 2007

Related U.S. Application Data
(60) Provisional application No. 60/843,214, filed on Sep. 8, 2006.

(51) Int. Cl. A61K 45/00; C12Q 1/68

(52) U.S. Cl. 424/9.1; 435/6; 514/789

(57) ABSTRACT

The present invention provides methods for diagnosing a neurological disease and/or for determining the predisposition of a subject to a neurological disease and/or for determining a subject at risk of developing a neurological disease, the method comprising detecting a marker in a glycogen synthase kinase 3β gene or expression product thereof and a microtubule-associated protein tau (MAPT) gene or expression product thereof. The present invention also provides pharmacogenetic methods, e.g., for identifying a subject that will respond to treatment with a therapeutic compound.

### RELATED APPLICATION DATA

This application claims priority from U.S. Patent Application No. 60/843,214 filed on Sep. 8, 2006, the contents of which are incorporated herein in their entirety.

### FIELD OF THE INVENTION

The present invention relates to a method for diagnosing a neurological disease and/or for determining the predisposition of a subject to a neurological disease and/or for predicting the response of a subject to treatment.

### BACKGROUND OF INVENTION

### General

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:
(i) Sambrook, Fritsch & Maniatis, Molecular Cloning: A Laboratory Manual, Cold Spring Harbor Laboratories, New York, Second Edition (1989), whole of Vols I, II, and III;
(ii) DNA Cloning: A Practical Approach, Vols. I and II (D. N. Glover, ed., 1985), IRL Press, Oxford, whole of text;
(iii) Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
(iv) Nucleic Acid Hybridization: A Practical Approach (B. D. Hames & S. J. Higgins, eds., 1985) IRL Press, Oxford, whole of text;
(v) Perbal, B., A Practical Guide to Molecular Cloning (1984);
(vi) Methods In Enzymology (S. Colowick and N. Kaplan, eds., Academic Press, Inc.), whole of series;
(vii) J. F. Ramalho Ortigão, “The Chemistry of Peptide Synthesis” In: Knowledge database of Access to Virtual Laboratory website (Interactiva, Germany);
(viii) Sakakibara, D., Teichman, J., Lien, E. Land Fenichel, R. L. (1976). Biochem. Biophys. Res. Commun. 73 336-342
(ix) Merrifield, R. B. (1963). J. Am. Chem. Soc. 85, 2149-2154.
(x) Handbook of Experimental Immunology, Vols. I-IV (D. M. Weir and C. C. Blackwell, eds., 1986, Blackwell Scientific Publications).

### DESCRIPTION OF THE RELATED ART

Neurological disorders are a large group of disorders characterized by changes in normal neuronal function, leading in the majority of cases to neuronal dysfunction and even cell death. Generally, Neurological disorders affect the central nervous system (e.g., brain, brainstem and cerebellum), the peripheral nervous system (peripheral nerves—cranial nerves included) and/or the autonomic nervous system (parts of which are located in both central and peripheral nervous system). Neurological disorders include, for example, neurodegenerative disorders (e.g., Parkinson's disease or Alzheimer's disease), behavioral disorders or neuro-psychiatric disorders (e.g., bipolar affective disorder or unipolar affective disorder or schizophrenia) and myelin-related disorders (e.g., multiple sclerosis).

### Neurodegenerative Disorders

Currently, it is estimated that there are in excess of one hundred neurological disorders. However, there is still little understanding of the etiological cause of these diseases. The most consistent risk factor for the development of a neurological disorder, such as, for example, Alzheimer's disease or Parkinson's disease, is age (Tanner, Neurol. Clin. 10: 317-329, 1992). For example, such diseases are more prevalent in aged or aging persons, with a doubling of risk every five years after the age of 65.

Over the past century, the growth rate of the population aged 65 and beyond in industrialized countries has far exceeded that of the population as a whole. Accordingly, it is anticipated that, over the next generations, the proportion of elderly citizens will double, and, with this, the proportion of persons suffering from a neurological disorder.

Two of the most common and most studied forms of age-related neurological disorder are Alzheimer's disease and Parkinson's disease. Currently, it is estimated that there are 4.5 million cases of Alzheimer's disease and 1.2 million cases of Parkinson's disease in the US alone. It is estimated that in the period from 2001 to 2010 an additional 1.5 million Alzheimer's disease cases will be diagnosed in the US, while currently there are approximately 480 new cases of Parkinson's disease per million people per year diagnosed. Alzheimer's disease alone is the third most expensive disease in the United States, costing approximately US$100 billion each year for therapy and/or care of sufferers.

Alzheimer's Disease

Alzheimer's disease is a complex multigenic neurological disorder characterized by progressive impairments in memory, behavior, language, and visuo-spatial skills, ending ultimately in death. Hallmark pathologies of Alzheimer's disease include granulovascular neuronal degeneration, extracellular neuritic plaques with β-amyloid deposits, intracellular neurofibrillary tangles and neurofibrillary degeneration, synaptic loss, and extensive neuronal cell death. It is now known that these histopathologic lesions of Alzheimer's disease correlate with the dementia observed in many elderly people.

Alzheimer's disease is commonly diagnosed using clinical evaluation including, physical and psychological assessment, an electroencephalography (EEG) scan, a computerized tomography (CT) scan and/or an electrocardiogram. These forms of testing are performed to eliminate some possible causes of dementia other than Alzheimer's disease, such as, for example, a stroke. Following elimination of other possible causes of dementia, Alzheimer's disease is diagnosed. Accordingly, current diagnostic approaches for Alzheimer's disease are not only unreliable and subjective, they do not predict the onset of the disease. Rather, these methods merely diagnose the onset of dementia of unknown cause, following onset.

Furthermore, not all causes of dementia are easily detectable by methods currently used for the diagnosis of Alzheimer's disease. Accordingly, a subject that has suffered an ischemic, metabolic, toxic, infectious or traumatic insult to the brain may also present with dementia, and, as a consequence, be incorrectly diagnosed with Alzheimer's disease. In fact, the NIH estimates that up to 45% of subjects diagnosed with Alzheimer's disease actually suffer from another form of dementia, not necessarily caused by a neurological disorder.

Genetic studies of subjects with a family history of Alzheimer's disease indicate that mutations in genes, such as, for example, amyloid precursor protein gene, presenillin-1 and/or presenillin-2 cause some forms of this disease. However, these forms of Alzheimer's disease represent less than 5% of total cases of the disease.

Studies to identify polymorphisms and alleles that confer susceptibility to Alzheimer's disease have identified a large number of polymorphisms and mutations (reviewed in Rocchi et al., Brain Res. Bull., 61: 1-24, 2003). The most widely studied of these is the 4 isoform of the apolipoprotein E gene. A number studies have shown an association between apolipoprotein E ε4 (ApoE-ε4) and late onset familial and sporadic forms of Alzheimer's disease (for example, Corder et al., Science 261: 261-263, 1993). However, less than 50% of non-familial Alzheimer's disease sufferers are carriers of the ApoE-ε4 isoform (Corder et al., Science 261: 261-263, 1993).

Parkinson's Disease

Parkinson's disease is a progressive disease with a mean age at onset of 55, and the incidence increases markedly with age, from 20/100,000 overall to 120/100,000 at age 70. This disease is characterized by resting tremor, rigidity, slowness or absence of voluntary movement (i.e. bradykinesia, hypokinesia and/or akinesia), postural instability, and freezing. Over time, symptoms worsen, and prior to the introduction of levodopa, the mortality rate among Parkinson's disease patients was three times that of the normal age-matched subjects. The majority of Parkinson's disease patients suffer considerable motor disability after 5-10 years of disease onset, even when expertly treated with available symptomatic medications.

Parkinson's disease is diagnosed by clinical evaluation of a subject. Subjects that have two or more of the principal symptoms, one of which is resting tremor or bradykinesia, are diagnosed as suffering from Parkinson's disease. Positron-emission tomography (PET-scan) using radio-labeled dopa is helpful in confirming a diagnosis in difficult cases. However, this test is not widely available. A SPECT-scan is a simpler test using a variety of different isotopes and is widely available but is less reliable in confirming PD. Magnetic resonance imaging (MRI) is useful in excluding other conditions such as tumors, strokes, and hydrocephalus. However, MRI cannot confirm PD.

As with Alzheimer's disease, diagnosis of Parkinson's disease is only accurate, or even possible, following onset of the disease. Accordingly, methods currently in use for diagnosis of these diseases are of no use in determining a predisposition to a neurological disorder, or in a prophylactic method of treatment for a neurological disorder.

The observation that a family history of Parkinson's disease is associated with an increased risk of developing the disease has provoked a considerable research effort to determine genes, alleles of genes or mutations that cause neurological disorder or are associated with a susceptibility to a neurological disorder.

Several mutations that cause Parkinson's disease have been identified, e.g., Ala⁵³→Thr (A53T) and Ala³⁰→Pro (A30P) in α-synuclein are associated with dominantly inherited PD (Polymeropoulos et al., Science 276, 2045-2047, 1997 and Kruger et al., Nat. Genet. 18, 107-108, 1998), loss-of-function mutations in the gene encoding parkin cause recessively inherited parkinsonism (Kitada et al., Nature 392, 605-608, 1998) and a dominant mutation (193M) in UCH-L1 was identified in one family with inherited PD (Leroy et al., Nature 395, 451-452, 1998). Notwithstanding the discovery of genes responsible for several forms of Parkinson's disease, these mutations are relatively rare and the number of subjects suffering from these forms of the disease represents a small proportion of Parkinson's disease patients.

Researchers have also attempted to use linkage and association studies to identify genes and/or alleles of genes that are associated with a susceptibility to Parkinson's disease. However, these studies have met with limited success. For example, Persad et al., Neuroepidemiology, 22: 357-361, 2003 showed that there was no significant association between polymorphisms in the CYP2D6 gene and Parkinson's disease in Asian populations, while Oliveira et al, Arch. Neurol., 60: 975-980, 2003, showed that there is no association between polymorphisms in the Parkin gene and Parkinson's disease in Caucasian subjects.

### Behavioral Disorders

Approximately 26.2% of people in USA aged 18 or over suffer from a form of diagnosable behavioral disorder in a year (Kessler et al., Archives of General Psychiatry, 62: 617-27, 2005). For example, approximately 5.7 million American adults suffer from bipolar affective disorder and approximately 2.4 million Americans suffer from a form of schizophrenia.

Bipolar Affective Disorder

Bipolar affective disorder generally commences in late adolescence or early adulthood and is characterized by periods of elevated mood (mania) and/or periods of depression (Goodwin, et al., 1990, Manic Depressive Illness, Oxford University Press, New York).

The most severe and clinically distinct forms of bipolar affective disorder are bipolar I disorder (severe bipolar affective (mood) disorder) and schizoaffective disorder (manic type). These disorders are characterized by at least one full episode of mania, with or without episodes of major depression (defined by lowered mood, or depression, with associated disturbances in rhythmic behaviors such as sleeping, eating, and sexual activity). Other forms of bipolar affective disorder include bipolar II disorder (characterized by at least one major depressive episode and at least one hypomanic episode), and unipolar disorder (characterized by recurrent major depressive episodes).

Currently, bipolar affective disorder is only diagnosed by clinical assessment. Diagnosis is based upon two main schemes, the International Classification of Diseases of the World Health Organization (10th Edition) and the Diagnostic and Statistic Manual (4th Edition). However, these schemes only detect bipolar affective disorder following onset of the disease. Furthermore, delays in accurate diagnosis using these schemes may extend many years and be associated with instability of presentation. For example, in an adult cohort diagnosed with their first psychotic episode, only 75% of patients were diagnosed with bipolar affective disorder after six months (Fennig et al., Am. J. Psychiatry 1994).

Additionally, the subjective classification systems currently in use for the diagnosis of bipolar affective disorder often lead to incorrect diagnoses as schizophrenia, shizoaffective disorder or psychotic depression.

As a consequence of the current inadequacies in diagnostics for bipolar affective disorder, several groups have attempted to identify a marker useful in the early diagnosis, and determination of a predisposition to the disorder.

Segregation analyses and twin studies have suggested that there is a genetic component to bipolar affective disorder (Bertelson, et al., Br. J. Psychiat. 130: 330-351, 1977; Freimer and Reus, in The Molecular and Genetic Basis of Neurological Disease, Rosenberg et al., eds., Butterworths, New York, 1992 pp. 951-965; Pauls et al., Arch. Gen. Psychiat. 49: 703-708, 1992). However, efforts to identify the chromosomal location of genes that might be linked to bipolar affective disorder have been disappointing. For example, reports of linkage between bipolar affective disorder and markers on the X chromosome and chromosome 11 could not be independently replicated nor confirmed in the re-analyses of the original pedigrees (Baron et al., Nature 326: 289 292, 1987; Egeland et al. Nature 325: 783-787, 1987; Kelsoe, et al., Nature 342: 238-243, 1989; Baron et al., 1993, Nature Genet. 3: 49-55, 1993).

Recent investigations have suggested possible localization of gene that are linked to bipolar affective disorder on chromosomes 18p and 21q (Berrettini, et al. Proc. Natl. Acad. Sci. USA 91, 5918-5921, 1991; Murray et al., Science 265: 2049-2054, 1994; Pauls et al., Am. J. Hum. Genet. 57: 636-643, 1995; Maier et al., Psych. Res. 59: 7-15, 1995; Straub et al., Nature Genet. 8: 291-296, 1994). However, such linkage studies have failed to sufficiently define or validate a locus that is closely linked to the disorder or a predisposition thereto.

Efforts to identify candidate bipolar susceptibility genes based upon function alone have also met with limited success. This is partly because the understanding of bipolar disorder etiology is poor.

Schizophrenia

Schizophrenia is a disorder that affects approximately one percent of the world population. Three general symptoms of schizophrenia are often referred to as positive symptoms, negative symptoms, and disorganized symptoms. Positive symptoms include, for example delusions (or abnormal beliefs), hallucinations (or abnormal perceptions), and disorganized thinking. The hallucinations of schizophrenia can be auditory, visual, olfactory, or tactile. Disorganized thinking generally manifests in schizophrenic patients by disjointed speech and the inability to maintain logical thought processes. Negative symptoms represent the absence of normal behavior. For example, negative symptoms include emotional flatness or lack of expression and can be characterized by social withdrawal, reduced energy, reduced motivation, and reduced activity.

The symptoms of schizophrenia generally persist continuously for a duration of about six months in order for the patient to be diagnosed as schizophrenic. Based on the types of symptoms a patient reveals, schizophrenia can be categorized into subtypes including catatonic schizophrenia, paranoid schizophrenia, and disorganized schizophrenia.

Despite a wealth of information about the epidemiology, neuroanatomy and pharmacology of schizophrenia, it is uncertain which molecular pathways are involved in the development of this disorder and how impairments in these pathways affect brain development and/or neuronal function. Despite an estimated heritability of about 60-80%, very little is known about the number or identity of genes involved in the development of schizophrenia. Notwithstanding that there has been recent progress in linkage and association studies, especially from genome-wide scans, these studies have yet to progress from the identification of susceptibility loci or candidate genes to the full characterization of disease-causing genes (Berrettini, Annu. Rev. Med. 51:465-79, 2000).

Myelin-Related Disorders

Multiple sclerosis (MS) is a neurological disease affecting more than one million people worldwide. MS is an inflammatory state in which the myelin of the central nervous system (CNS) is destroyed, resulting in neurological impairment and frequently serious disabilities. The etiology of MS is currently unknown. Rather, it is generally assumed that the disease is mediated an autoimmune process which may be induced by an infection and/or a genetic predisposition. Following a mostly insidious, rarely sub-acute or acute beginning in the 20^{th} to 40^{th} year of life, a chronic-progressive or polycentric disorder develops. The different stages and forms of multiple sclerosis are subdivided into relapsing-remitting (RR) MS (at present approximately 80-85% of MS patients), primary progressive (PP) MS, with more than 50% of the patients with RR-MS finally developing a permanent deterioration with or without superimposed relapses (secondary progressive (SP) form of MS) over time. It is not clear whether the different disease progressions are based on the same or different pathophysiological processes.

Diagnosis of multiple sclerosis is difficult and generally requires a detailed analysis of a subject's medical history, a clinical examination, an MRI to detect plaques and/or scarring that may be caused by MS, an evoked potential test, and immunological analysis of cerebrospinal fluid (CSF) to detect particular immune system proteins and for the presence of a staining pattern of antibodies called oligoclonal bands. Generally, MS is diagnosed following exclusion of all other conditions that may have caused the symptoms displayed by a subject. Accordingly, there is no test for the specific diagnosis or prediction of MS.

Based on the discussion herein it is clear that there is a need to develop improved diagnostic methods for determining a predisposition to development of a neurological disorder in a subject, and for the early diagnosis of a neurological disorder. Preferably, the diagnostic method also permits the identification of the specific disorder that a subject suffers from or is likely to develop. Diagnostic assays that rapidly and reliably diagnose a neurological disorder prior to onset of the disease are particularly desirable as are indicators of whether or not a subject will respond to a particular treatment.

### SUMMARY OF INVENTION

This specification contains nucleotide and amino acid sequence information prepared using PatentIn Version 3.4, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

### SPECIFIC EMBODIMENTS

The scope of the invention will be apparent from the claims as filed with the application that follow the examples. The claims as filed with the application are hereby incorporated into the description. The scope of the invention will also be apparent from the following description of specific embodiments.

In work leading up to the present invention the inventors sought to identify polymorphisms that are significantly associated with development of a neurological disorder for use in the development of a new diagnostic and/or prognostic method. The inventors studied polymorphisms associated with Parkinson's disease, Alzheimer's disease, bipolar affective disorder, schizophrenia and multiple sclerosis as models of neurological disorders generally. Accordingly, any markers identified by the inventors are also markers of a neurodegenerative disease, a behavioral disorder and/or a myelin-associated disorder.

As exemplified herein, the present inventors identified polymorphisms in the glycogen synthase kinase 3β (GSK-3β) gene and the microtubule-associated protein tau (MAPT) gene that are associated with a neurological disorder. For example, the inventors found that the presence of a polymorphism in a GSK-3β gene associated with modified expression of an expression product of a GSK-3β gene and the presence of a polymorphism in a MAPT gene associated with modified expression of an expression product of a MAPT gene is indicative of a neurological disorder in a subject or an increased risk of developing a neurological disorder.

The present inventors have also demonstrated that the combination of markers from a GSK-3β gene and a MAPT gene significantly increases the accuracy of diagnosis/prediction of a neurological disorder relative to the use of one of these markers alone.

For example, the present inventors have demonstrated that the presence of one or more polymorphisms in a GSK-3β gene associated with in increased expression of an isoform of a GSK-3β gene and the presence of one or more polymorphisms in a MAPT gene associated with decreased expression of MAPT is indicative of a neurodegenerative disorder or an increased risk of developing a neurodegenerative disorder or a bipolar affective disorder or multiple sclerosis. The present inventors have also demonstrated that the presence of one or more polymorphisms in a GSK-3β gene associated with reduced expression of an isoform of a GSK-3β gene and the presence of one or more polymorphisms in a MAPT gene associated with increased expression of MAPT is indicative of schizophrenia or an increased risk of developing schizophrenia.

Accordingly, the present invention provides a method for diagnosing a neurological disorder in a subject or determining the predisposition of a subject to developing a neurological disorder or determining the risk of a subject developing a neurological disorder, the method comprising:
(i) detecting in a sample from the subject a marker within a glycogen synthase kinase-3β (GSK-3β) gene that is associated with a neurological disorder; and
(ii) detecting in a sample from the subject a marker within a microtubule-associated protein tau (MAPT) gene that is associated with a neurological disorder,
   wherein detection of the marker at (i) and (ii) is indicative of a neurological disorder or a predisposition to a neurological disorder or an increased risk of developing a neurological disorder.

Preferably, the present invention provides a method for diagnosing a neurological disorder in a subject or determining the predisposition of a subject to developing a neurological disorder or determining the risk of a subject developing a neurological disorder, the method comprising:
(i) detecting in a sample from the subject a marker in a GSK-3β gene associated with modified expression of a GSK-3β expression product; and
(ii) detecting in a sample from the subject a marker in a MAPT gene associated with modified expression of a MAPT gene product,
   wherein detection of the marker at (i) and (ii) is indicative of a neurological disorder or a predisposition to a neurological disorder or an increased risk of developing a neurological disorder.

For the purposes of nomenclature, the nucleotide sequence of a human GSK-3β gene is exemplified herein as SEQ ID NO: 1. For the purposes of nomenclature, a MAPT gene preferably encodes a nucleic acid comprising a nucleotide sequence set forth in SEQ ID NO: 11.

As used herein, the term “marker” shall be taken to mean a nucleic acid that comprises a nucleotide sequence associated with a neurological disorder. Preferably, the marker comprises a nucleotide sequence associated with modified expression of an expression product of a GSK-3β gene or a MAPT gene.

Alternatively, or in addition, the marker is linked to a polymorphism in a genome wherein said polymorphism is associated with a neurological disorder. Preferably, the marker is linked to a polymorphism associated with modified expression of an expression product of a GSK-3β gene or a MAPT gene.

In those embodiments related to detection of a marker that is in a region of the genome that is transcribed or that controls transcription, the term “marker” shall also be taken to mean an expression product of a gene or an allele of GSK-3β or MAPT that is associated with a neurological disorder. For example, the marker may comprise or be within a pre-mRNA molecule, a 5′capped mRNA, a polyadenylated mRNA and/or a mature or processed mRNA.

In those embodiments related to antigen-based or antibody-based assays, those skilled in the art will appreciate that the term “marker” also means a peptide, polypeptide or protein that comprises an amino acid sequence encoded by an allele of a GSK-3β or MAPT gene that is associated with a neurological disorder. Alternatively, or in addition, the proteinaceous marker is associated with a polymorphism in a GSK-3β gene or a MAPT gene wherein said polymorphism is associated with a neurological disorder.

As used herein, the term “associated with a disease or disorder” shall be taken to mean that the detection of a marker is significantly correlated with the development of a disease or disorder in a subject or that the absence of a marker is significantly correlated with the development of a disease. For example, a marker that is positively associated with a disease is a polymorphism, the detection of which is associated with the development of the disease.

In a preferred embodiment, a marker is significantly correlated with the development of a disease or disorder in a plurality of subjects. Even more preferably, the marker is significantly correlated with the development of a disease or disorder in a plurality of unrelated subjects.

As used herein, the term “associated with modified expression” shall be taken to mean that the presence of a marker in a GSK-3β gene or a MAPT gene is significantly correlated with increased or decreased expression of an expression product of the relevant gene (e.g., an mRNA or protein) or that the absence of is significantly correlated with increased or decreased expression of an expression product of the relevant gene (e.g., an mRNA or protein). For example, the presence of a marker in a GSK-3β gene is associated with increased expression of one or more isoforms of said gene.

By “detection is indicative of the neurological disorder” is meant that the detected marker is indicative of the neurological disorder by virtue of the association between the marker and the disease or disorder.

As used herein, the term “predisposition to a neurological disorder” shall be taken to mean that a subject is susceptible to a form of a neurological disorder or is more likely to develop the disease or disorder than a normal individual or a normal population of individuals. In this regard, a marker that is indicative of a predisposition to a disease or disorder may itself cause the disease or disorder or, alternatively, be correlated with the development of the disease or disorder.

While the present invention contemplates any marker in a GSK-3β nucleic acid or polypeptide or MAPT nucleic acid or polypeptide, it is preferred that the marker comprises or consists of a polymorphism within a GSK-3β gene or a polymorphism within a MAPT gene. By “polymorphism” is meant a difference in the nucleotide sequence of a specific site or region of the genome of a subject that occurs in a population of individuals. Accordingly, a “polymorphism that is associated with a neurological disorder” means that a particular form of the polymorphism is correlated with the development of the neurological disorder in a subject, i.e., in a population of subjects, those that suffer from the neurological disorder are more likely to comprise the specific polymorphism that those subjects that do not suffer from the neurological disorder.

Preferably, a marker in a GSK-3β gene that is associated with a neurological disorder comprises a polymorphism that is in its homozygous form. By “homozygous form” is meant that the same form of the marker associated with a neurological disorder occurs at the same locus on homologous chromosomes in a subject or sample derived therefrom. For example, the same nucleotide is found at the site of a single nucleotide polymorphism (SNP) located within a GSK-3β gene on both copies of chromosome 3 in a subject.

Preferably, a marker in a MAPT gene that is associated with a neurological disorder comprises a polymorphism that is in its heterozygous form or in its homozygous form. By “heterozygous form” is meant that a different form of the marker associated with a neurological disorder occurs at the same locus on homologous chromosomes in a subject or sample derived therefrom. For example, a different nucleotide is found at the site of a single nucleotide polymorphism (SNP) located within a MAPT gene on both copies of chromosome 17 in a subject.

In a preferred embodiment, a marker that is associated with a neurological disorder comprises a polymorphism or a single nucleotide polymorphism (SNP). Methods for determining a polymorphism SNP that is associated with a specific disorder are known in the art and/or described herein. Preferred SNPs in the GSK-3β gene for use in the method of the present invention include, for example, a thymidine or a cytosine at a position corresponding to nucleotide position 181,700 of SEQ ID NO: 1 (also designated rs6438552 in the NCBI SNP database at May 6, 2005) and/or a cytosine or a thymidine at a position corresponding to nucleotide position 231 of SEQ ID NO: 1 (also designated rs334558 in the NCBI SNP database at May 6, 2005); or an adenosine or a thymidine at a position corresponding to nucleotide position 1679 of SEQ ID NO: (also designated rs3755557 in the NCBI SNP database at May 6, 2005) or mixtures thereof.

Preferred polymorphisms in the MAPT gene for use in performing a method as described herein according to any embodiment include, for example, a polymorphism selected from the group consisting of:
(i) AATTT at a position equivalent to nucleotide position 763-767 of SEQ ID NO: 13;
(ii) TT at a position equivalent to nucleotide positions 793 and 794 of SEQ ID NO: 13;
(iii) A at a position equivalent to nucleotide position 817 of SEQ ID NO: 13;
(iv) G at a position equivalent to nucleotide position 1011 of SEQ ID NO: 13;
(v) T at a position equivalent to nucleotide position 1036 of SEQ ID NO: 13;
(vi) C at a position equivalent to nucleotide position 1159 of SEQ ID NO: 13;
(vii) A at a position equivalent to nucleotide position 1340 of SEQ ID NO: 13; and
(viii) any combination of the above polymorphisms.

For example, the method of the invention comprises detecting nucleic acid comprising a sequence set forth in SEQ ID NO: 13 in a sample from a subject.

In the case of a nucleic acid marker associated with a disease or disorder, the marker is preferably detected by hybridizing a nucleic acid probe comprising the sequence of the marker to a marker linked to nucleic acid in a sample from a subject under moderate to high stringency hybridization conditions and detecting the hybridization using a detection means, wherein hybridization of the probe to the sample nucleic acid indicates that the subject suffers from a neurological disorder or a has a predisposition to a neurological disorder or has an increased risk of developing a neurological disorder. Preferably, the detection means is a nucleic acid hybridization or amplification reaction, such as, for example, a polymerase chain reaction (PCR).

Not only is such a method useful for, for example, detecting a specific polymorphism in a sample from a subject, but also for detecting a marker in an expression product of a GSK-3β gene or a MAPT gene, for example, an alternate splice form of a GSK-3β transcript. For example, the present inventors have shown an association between the expression of an alternate splice form of a GSK-3β gene and a disease or disorder associate with aberrant GSK-3β expression and/or activity. For example, in one embodiment, the method of the invention as described herein according to any embodiment comprises detecting a modified level of an alternate splice form encoded by a GSK-3β gene, for example, comprising a nucleotide sequence set forth in SEQ ID NO: 4 or SEQ ID NO: 8. For example, the method of the invention as described herein according to any embodiment comprises detecting an enhanced level of an alternate splice form lacking exon 9 encoded by a GSK-3β gene (e.g., comprising a sequence set forth in SEQ ID NO: 4) or detecting an enhanced level of an alternate splice form lacking exons 9 and 11 encoded by a GSK-3β gene (e.g., comprising a sequence set forth in SEQ ID NO: 8)

In an alternative embodiment, the marker is within a GSK-3β polypeptide or a MAPT polypeptide (for example, the marker is encoded by an alternative splice form of a GSK-3β transcript) and is determined by detecting a polypeptide encoded by the transcript. Such a marker is detected, for example, by contacting a biological sample derived from a subject with an antibody or ligand capable of specifically binding to said marker for a time and under conditions sufficient for an antibody/ligand complex to form and then detecting the complex wherein detection of the complex indicates that the subject being tested suffers from a neurological disorder or a has a predisposition to a neurological disorder or has an increased risk of developing a neurological disorder. A suitable method for detecting the antibody-antigen complex will be apparent to the skilled person and/or described herein.

The polymorphisms identified by the present inventors are also associated with increased expression of a GSK-3β gene or an isoform encoded by the GSK-3β gene or an expression product of a MAPT gene. Accordingly, a subject at risk of developing or that suffers from a neurological disorder may equally be determined by determining an enhanced level of a GSK-3β expression product or a MAPT expression product in a sample from the subject. Suitable methods for determining the level of a GSK-3β expression product or a MAPT expression product will be apparent to the skilled person and/or described herein.

For example, in one embodiment, the method of the invention comprises detecting an enhanced level of a GSK-3β expression product, e.g., transcript (e.g., a transcript lacking exon 9 or lacking exons 9 and 11) and detecting reduced expression of a MAPT transcript. Such a method is useful for, for example, diagnosing or predicting a predisposition to a neurological disorder, such as, for example, a neurodegenerative disorder or a bipolar affective disorder or multiple sclerosis.

In another embodiment, the method of the invention comprises detecting an enhanced level of a MAPT expression product (e.g., transcript) and detecting a reduced level of a GSK-3β transcription (e.g., a transcript lack exon 9). Such a method is useful, for example, for diagnosing or predicting a predisposition to a neurological disorder, such as, for example, schizophrenia.

The present invention also contemplates, for example, detecting a marker in a GSK-3β gene that is associated with modified expression of an isoform of an expression product of a GSK-3β gene and detecting modified expression of a MAPT expression product. Alternatively, the present invention contemplates detecting a marker in a MAPT gene that is associated with modified expression of an expression product of a MAPT gene and detecting modified expression of a GSK-3β expression product.

The markers of the present invention may also be used in a multiplex reaction to determine the predisposition of a subject to a neurological disorder or to diagnose a neurological disorder. In this regard, such a multiplexed assay may detect two or more nucleic acid markers that are associated with a neurological disorder, for example, two or more markers described herein. Alternatively, or in addition, a multiplexed assay may detect two or more peptide, polypeptide or protein markers that are associated with a neurological disorder. Clearly, the combination of nucleic acid-based and antigen-based detection methods is contemplated by the invention.

The present invention also contemplates detecting one or more additional marker(s) associated with a neurological disorder. Suitable additional marker(s) will be apparent to the skilled artisan and/or described herein.

In one embodiment, the method of the invention additionally comprises determining an association between the marker and the disease or disorder. Suitable methods for determining an association between a marker and a disease or disorder are known in the art and/or described herein.

The present inventors have also demonstrated that the level of expression of a GSK-3β gene and the level of expression of a MAPT gene is indicative of a neurological disorder. For example, the present inventors have found that the balance between the level of expression of GSK-3β and MAPT is indicative of a neurological disease. For example, an enhanced level of GSK-3β expression and a reduced level of MAPT expression are indicative of a neurological disease, e.g., a neurodegenerative disease or a bipolar affective disorder. A reduced level of GSK-3β expression and an enhanced level of MAPT expression are indicative of a neurological disease, e.g., schizophrenia. These findings provide the basis for a method for diagnosing a neurological disorder in a subject or determining the predisposition of a subject to developing a neurological disorder, said method comprising detecting a modified level of a GSK-3β expression product and/or a marker associated therewith and detecting a modified level of a MAPT expression product and/or a marker associated therewith, wherein said detection indicates that the subject suffers from or is predisposed to developing a neurological disorder.

For example, the present invention provides a method for the diagnosis of a neurological disorder in a subject or determining the predisposition of a subject to developing a neurological disorder or determining the risk of a subject developing a neurological disorder, the method comprising:
(i) detecting in a sample from the subject a marker in a GSK-3β gene associated with modified expression of a GSK-3β expression product; and
(ii) detecting in a sample from the subject a marker in a MAPT gene associated with modified expression of a MAPT gene product,
   wherein detection of the marker at (i) and (ii) is indicative of a neurodegenerative disorder or a predisposition to a neurological disorder or a predisposition to a neurological disorder or an increased risk of developing a neurological disorder.

For example, the method comprises detecting a marker in a GSK-3β gene associated with increased expression of a GSK-3β expression product and a marker in a MAPT gene associated with reduced expression of a MAPT gene product. Alternatively, the method comprises detecting a marker in a GSK-3β gene associated with decreased expression of a GSK-3β expression product and a marker in a MAPT gene associated with increased expression of a MAPT gene product.

The present invention additionally provides a method for the diagnosis of a neurodegenerative disorder in a subject or determining the predisposition of a subject to developing a neurodegenerative disorder or determining the risk of a subject developing a neurodegenerative disorder, the method comprising:
(i) detecting in a sample from the subject a marker in a GSK-3β gene associated with increased expression of a GSK-3β expression product; and
(ii) detecting in a sample from the subject a marker in a MAPT gene associated with reduced expression of a MAPT gene product,
   wherein detection of the marker at (i) and (ii) is indicative of a neurodegenerative disorder or a predisposition to a neurodegenerative disorder or an increased risk of developing a neurodegenerative disorder.

Preferably, the marker in a GSK-3β gene is associated with increased expression of a transcript of a GSK-3β gene, the transcript lacking exons 9 and 11 (e.g., comprising a sequence set forth in SEQ ID NO: 8.

In a preferred embodiment, the method comprises detecting a thymidine at a position corresponding to nucleotide position 181,700 of SEQ ID NO: 1 (also designated rs6438552 in the NCBI SNP database at May 6, 2005) and/or a thymidine at a position corresponding to nucleotide position 231 of SEQ ID NO: 1 (also designated rs334558 in the NCBI SNP database at May 6, 2005). Optionally, the method additionally comprises detecting a thymidine at a position corresponding to nucleotide position 1679 of SEQ ID NO: 10 (also designated rs3755557 in the NCBI SNP database at May 6, 2005).

Preferably, the method additionally comprises detecting a polymorphism selected from the group consisting of:
(i) AATTT at a position equivalent to nucleotide position 763-767 of SEQ ID NO: 13;
(ii) TT at a position equivalent to nucleotide positions 793 and 794 of SEQ ID NO: 13;
(iii) A at a position equivalent to nucleotide position 817 of SEQ ID NO: 13;
(iv) G at a position equivalent to nucleotide position 1011 of SEQ ID NO: 13;
(v) T at a position equivalent to nucleotide position 1036 of SEQ ID NO: 13;
(vi) C at a position equivalent to nucleotide position 1159 of SEQ ID NO: 13;
(vii) A at a position equivalent to nucleotide position 1340 of SEQ ID NO: 13; and
(viii) any combination of the above polymorphisms.

More preferably, the method of the invention comprises detecting a marker in a sample from a subject, the marker indicating that the genomic DNA of the subject comprises a sequence set forth in SEQ ID NO: 13.

The method of the previous embodiment of the invention shall be taken to apply mutatis mutandis to the diagnosis of multiple sclerosis in a subject or determining the predisposition of a subject to developing multiple sclerosis or determining the risk of a subject developing multiple sclerosis.

The present invention additionally provides a method for the diagnosis of a bipolar affective disorder in a subject or determining the predisposition of a subject to developing a bipolar affective disorder or determining the risk of a subject developing a bipolar affective disorder, the method comprising:
(i) detecting in a sample from the subject a marker in a GSK-3β gene associated with increased expression of a GSK-3β expression product; and
(ii) detecting in a sample from the subject a marker in a MAPT gene associated with reduced expression of a MAPT gene product,
   wherein detection of the marker at (i) and (ii) is indicative of a bipolar affective or a predisposition to a bipolar affective or an increased risk of developing a bipolar affective.

Preferably, the marker in a GSK-3β gene is associated with increased expression of a transcript of a GSK-3β gene, the transcript lacking exon 9 (e.g., comprising a sequence set forth in SEQ ID NO: 4).

In a preferred embodiment, the method comprises detecting a cytosine at a position corresponding to nucleotide position 181,700 of SEQ ID NO: 1 (also designated rs6438552 in the NCBI SNP database at May 6, 2005) and/or a cytosine at a position corresponding to nucleotide position 231 of SEQ ID NO: 1 (also designated rs334558 in the NCBI SNP database at May 6, 2005) and/or detecting a thymidine at a position corresponding to nucleotide position 1679 of SEQ ID NO: 10 (also designated rs3755557 in the NCBI SNP database at May 6, 2005).

Preferably, the method additionally comprises detecting a polymorphism selected from the group consisting of:
(i) AATTT at a position equivalent to nucleotide position 763-767 of SEQ ID NO: 13;
(ii) TT at a position equivalent to nucleotide positions 793 and 794 of SEQ ID NO: 13;
(iii) A at a position equivalent to nucleotide position 817 of SEQ ID NO: 13;
(iv) G at a position equivalent to nucleotide position 1011 of SEQ ID NO: 13;
(v) T at a position equivalent to nucleotide position 1036 of SEQ ID NO: 13;
(vi) C at a position equivalent to nucleotide position 1159 of SEQ ID NO: 13;
(vii) A at a position equivalent to nucleotide position 1340 of SEQ ID NO: 13; and
(viii) any combination of the above polymorphisms.

More preferably, the method of the invention comprises detecting a marker in a sample from a subject, the marker indicating that the genomic DNA of the subject comprises a sequence set forth in SEQ ID NO: 13.

The present invention additionally provides a method for the diagnosis of schizophrenia in a subject or determining the predisposition of a subject to developing schizophrenia or determining the risk of a subject developing schizophrenia, the method comprising:
(i) detecting in a sample from the subject a marker in a GSK-3β gene associated with reduced expression of a GSK-3β expression product; and
(ii) detecting in a sample from the subject a marker in a MAPT gene associated with increased expression of a MAPT gene product,
   wherein detection of the marker at (i) and (ii) is indicative of schizophrenia or a predisposition to schizophrenia or an increased risk of developing schizophrenia.

Preferably, the marker in a GSK-3β gene is associated with reduced expression of a transcript of a GSK-3β gene, the transcript lacking exon 9 (e.g., comprising a sequence set forth in SEQ ID NO: 4).

In a preferred embodiment, the method comprises detecting a cytosine at a position corresponding to nucleotide position 181,700 of SEQ ID NO: 1 (also designated rs6438552 in the NCBI SNP database at May 6, 2005) and/or a cytosine at a position corresponding to nucleotide position 231 of SEQ ID NO: 1 (also designated rs334558 in the NCBI SNP database at May 6, 2005) and/or detecting an adenosine at a position corresponding to nucleotide position 1679 of SEQ ID NO: 10 (also designated rs3755557 in the NCBI SNP database at May 6, 2005).

Preferably, the method additionally comprises detecting a polymorphism in a MAPT gene, the marker indicating that the genomic DNA of the subject comprises a sequence set forth in SEQ ID NO: 14

Clearly, the methods of the present invention are also useful for determining a subject that is a carrier of a marker that is associated with a neurological disorder. Such an assay is useful, for example, for determining the likelihood, or susceptibility of a child of the subject/s being tested to develop a neurological disease disorder.

The diagnostic method of the present invention is also useful in a method of treatment. For example, a method of treatment comprising:
(i) performing a method described herein according to any embodiment for diagnosing a neurological disorder in a subject or determining the predisposition of a subject to developing a neurological disorder; and
(ii) administering or recommending a therapeutic for the treatment of the neurological disorder.

Accordingly, the present invention contemplates a pharmacogenomic method for predicting or determining a suitable treatment of a neurological disorder. For example, the method comprises:
(i) performing a method described herein according to any embodiment for diagnosing a particular type of neurological disorder in a subject or determining the predisposition of a subject to developing a neurological disorder; and
(ii) administering or recommending a therapeutic for the treatment of the particular type of neurological disorder based on the diagnosis and/or the determination of the predisposition.

For example, the method comprises diagnosing a neurodegenerative disease or determining a predisposition to a neurodegenerative disease using a method described herein according to any embodiment and administering or recommending a therapeutic for the treatment of the neurodegenerative disease. Alternatively, the method comprises diagnosing bipolar affective disorder or determining a predisposition to bipolar affective disorder using a method described herein according to any embodiment and administering or recommending a therapeutic for the treatment of bipolar affective disorder. Alternatively, the method comprises diagnosing schizophrenia or determining a predisposition to schizophrenia using a method described herein according to any embodiment and administering or recommending a therapeutic for the treatment of schizophrenia. Alternatively, the method comprises diagnosing multiple sclerosis or determining a predisposition to multiple sclerosis using a method described herein according to any embodiment and administering or recommending a therapeutic for the treatment of multiple sclerosis.

Alternatively, a pharmacogenomic method of the present invention comprises:
(i) detecting in a sample from the subject a marker within a glycogen synthase kinase-3β (GSK-3β) gene that is associated with a neurological disorder; and
(ii) detecting in a sample from the subject a marker within a microtubule-associated protein tau (MAPT) gene that is associated with a neurological disorder,
   wherein the marker detected at (i) and (ii) is indicative of a subject that will respond to treatment with a therapeutic compound.

For example, as will be apparent from the disclosure herein, particular combinations of markers in GSK-3β and MAPT are indicative of a specific neurological disorder, thereby providing for the means to predict a subject suffering from that specific neurological disorder and/or that will respond to treatment for that disorder.

In one embodiment, the administration or recommendation of a therapeutic for the treatment of the neurological disorder is based upon the diagnosis of the neurological disorder or the diagnosis of a predisposition to the neurological disorder.

As will be apparent to the skilled artisan, the present invention also encompasses methods for the prophylaxis or prophylactic therapy of a neurological disorder. For example, a preferred embodiment of the invention provides a method for the prophylaxis of a neurological disorder, said method comprising:
(i) performing a method described herein according to any embodiment for determining the predisposition of a subject to developing a neurological disorder or determining a subject having a high risk of developing a neurological disorder; and
(ii) administering or recommending a compound that delays or prevents onset of the disease or disorder.

Preferably, the administration or recommendation of the compound is based upon the determination of a subject at risk of developing (i.e., predisposed to developing) the disease or disorder.

The present invention also provides a method for determining a subject likely to respond to a treatment for a neurological disease, said method comprising:
(i) detecting in a sample from the subject a marker within a glycogen synthase kinase-3β (GSK-3β) gene that is associated with a neurological disorder; and
(ii) detecting in a sample from the subject a marker within a microtubule-associated protein tau (MAPT) gene that is associated with a neurological disorder,
   wherein at least one of said markers is indicative of a subject that will respond to treatment with a therapeutic compound, and wherein detection of said markers is indicative of a subject that will suffer from a neurological disorder and that will respond to treatment for said neurological disorder.

As used herein, the term “respond to treatment” shall be taken to mean that the symptoms of a neurological disease in a subject are reduced or ameliorated as a result of treatment with a therapeutic compound. For example, in the case of BAD, a subject that responds to treatment may experience fewer depressive episodes and/or fewer manic episodes.

In one example, both markers are indicative of a subject that will respond to treatment with a therapeutic compound.

In one example, the present invention provides a method for determining a subject suffering from a neurological disorder or having a predisposition to a neurological disorder or having an increased risk of developing a neurological disorder who is likely to respond to a treatment with a therapeutic and/or prophylactic compound for a neurological disease, said method comprising:
(i) detecting in a sample from the subject a marker within a glycogen synthase kinase-3β (GSK-3β) gene that is associated with a neurological disorder; and
(ii) detecting in a sample from the subject a marker within a microtubule-associated protein tau (MAPT) gene that is associated with a neurological disorder,
   wherein at least one of said markers is indicative of a subject that will respond to treatment with a therapeutic compound, and wherein detection of said markers is indicative of a subject that suffers from a neurological disorder or has a predisposition to a neurological disorder or has an increased risk of developing a neurological disorder and who will respond to treatment for said neurological disorder.

Suitable neurological disorders will be apparent to the skilled artisan based on the description herein and include for example a neurological disorder selected from the group consisting of a neurodegenerative disorder, a behavioral disorder and multiple sclerosis. For example, the neurological disorder is a bipolar affective disorder.

In one example, the therapeutic compound is lithium.

The present invention also provides a method for determining a subject likely to respond to treatment with a therapeutic and/or prophylactic compound for a neurological disease, said method comprising:
(i) detecting in a sample from the subject a marker within a glycogen synthase kinase-3β (GSK-3β) gene that is associated with a response of a subject to a therapeutic compound for treatment or prophylaxis of a neurological disorder; and
(ii) detecting in a sample from the subject a marker within a microtubule-associated protein tau (MAPT) gene that is associated with a response of a subject to a therapeutic compound for treatment or prophylaxis of a neurological disorder,
   wherein detection of said markers is indicative of a subject that will respond to treatment for said neurological disorder.

In one example, the present invention provides a method for determining a subject suffering from a neurological disorder or having a predisposition to a neurological disorder or having an increased risk of developing a neurological disorder who is likely to respond to a treatment with a therapeutic and/or prophylactic compound for a neurological disease, said method comprising:
(i) detecting in a sample from the subject a marker within a glycogen synthase kinase-3β (GSK-3β) gene that is associated with a response of a subject to a therapeutic compound for treatment or prophylaxis of a neurological disorder; and
(ii) detecting in a sample from the subject a marker within a microtubule-associated protein tau (MAPT) gene that is associated with a response of a subject to a therapeutic compound for treatment or prophylaxis of a neurological disorder,
   wherein detection of said markers is indicative of a subject who will respond to treatment for said neurological disorder.

The present invention also provides a method for determining a subject suffering from a bipolar affective disorder or having a predisposition to a bipolar affective disorder or having an increased risk of developing a bipolar affective disorder who will respond to a treatment with lithium, said method comprising detecting in a sample from a subject:
(i) detecting markers within a glycogen synthase kinase 3β (GSK-3β) gene, said markers comprising a cytosine at a position corresponding to nucleotide position 181,700 of SEQ ID NO: 1, a thymidine at a position corresponding to nucleotide position 231 of SEQ ID NO: 1 and an adenosine at a position corresponding to nucleotide position 1679 of SEQ ID NO: 10; and
(ii) detecting a marker within a microtubule-associated protein tau (MAPT) gene that is associated with reduced expression of a MAPT expression product,
   wherein detection of said markers is indicative of a subject who will respond to treatment with lithium.

The present invention also provides a method for determining a subject suffering from a bipolar affective disorder or having a predisposition to a bipolar affective disorder or having an increased risk of developing a bipolar affective disorder who will respond to a treatment with lithium, said method comprising detecting in a sample from a subject:
(i) detecting markers within a glycogen synthase kinase 3β (GSK-3β) gene, said markers comprising cytosine at a position corresponding to nucleotide position 181,700 of SEQ ID NO: 1, a thymidine at a position corresponding to nucleotide position 231 of SEQ ID NO: 1 and an adenosine at a position corresponding to nucleotide position 1679 of SEQ ID NO: 10; and
(ii) detecting a marker within a microtubule-associated protein tau (MAPT) gene that is associated with reduced expression of a MAPT expression product,
   wherein detection of said markers is indicative of a subject who will respond to treatment with lithium.

The present invention also provides a method for determining a subject suffering from a bipolar affective disorder or having a predisposition to a bipolar affective disorder or having an increased risk of developing a bipolar affective disorder who will respond to a treatment with lithium, said method comprising detecting in a sample from a subject:
(i) detecting markers within a glycogen synthase kinase 3β (GSK-3β) gene, said markers comprising a cytosine at a position corresponding to nucleotide position 181,700 of SEQ ID NO: 1, cytosine at a position corresponding to nucleotide position 231 of SEQ ID NO: 1 and a thymidine at a position corresponding to nucleotide position 1679 of SEQ ID NO: 10; and
(ii) detecting a marker within a microtubule-associated protein tau (MAPT) gene that is associated with reduced expression of a MAPT expression product,
   wherein detection of said markers is indicative of a subject who will respond to treatment with lithium.

The present invention also provides a method for identifying a subject suffering from a neurological disease or having a predisposition to a neurological disease or at risk of developing a neurological disease who will not experience a side effect resulting from treatment with a therapeutic compound, said method comprising:
(i) detecting in a sample from the subject a marker in a GSK-3β gene associated with modified expression of a GSK-3β expression product; and
(ii) detecting in a sample from the subject a marker in a MAPT gene associated with modified expression of a MAPT gene product,
   wherein detection of said marker at (i) and (ii) indicates that the subject will not experience a side effect resulting from treatment with a therapeutic compound.

In one example, the side-effect is hand tremor.

Preferably, the therapeutic compound is lithium.

In another embodiment, the present invention provides a method for determining a subject that is likely to respond to a specific therapy (or therapeutic compound) for treatment of a neurological disorder. Preferably, the method comprises performing a method described herein to determine a subject that will suffer from a neurological disorder, preferably a specific neurological disorder (e.g., a neurodegenerative disorder or a behavioral disorder or a myelin-associated disorder); and administering or recommending administration of a compound for the treatment of the neurological disorder. Such a method is useful for pharmacogenomic analysis to determine an effective treatment for a subject.

Any probes and/or primers useful in determining a disease or disorder associated with aberrant GSK-3β expression and/or activity are also contemplated by the present invention.

### BRIEF DESCRIPTION OF THE DRAWINGS

FIG. 1 is a diagrammatic representation of the genomic structure of the GSK-3β gene. The relative position of several polymorphisms in GSK-3β is indicated (vertical arrows). The NCBI SNP database designation for each SNP is also indicated.

FIG. 2A is a diagrammatic representation of the exon trap pSPL3 vector with the region spanning GSK-3β exon 6 and flanking intronic sequence. A chimeric exon is generated by the fusion of a cryptic splice donor site from pSPL3 (CSD) and two alternative cryptic splice acceptor sites. Exon trap transcripts are generated by splicing of the vector's exon boundaries (TAT) with splice donor and acceptor sites within the subcloned genomic fragment.

FIG. 2B is a copy of a photographic representation showing electrophoresis of exon trap products on a 2% agarose gel. Exon trapping was performed in two cell lines, HEK293 (upper panel) and SK-N-MC (lower panel), transfected with the parental pSPL3 vector (vec), or constructs carrying genomic fragments with the each allele of the three intronic polymorphisms of GSK-3β (shown in FIG. 1A). Schematic diagrams of the three exon trap products associated with the utilization of three possible splice acceptor sites A (native splice acceptor site), B (IVS 6+212), and C (IVS 6+181) are shown for the exon trap analysis of GSK3 SNP. The relevant nucleotide at the site of the SNP is indicated at the top of the gel in the upper panel.

FIG. 2C is a graphical representation of semi-quantitative analysis of exon trap products isolated from HEK-293 cells transfected with either the C allele (open bars) or T allele (black bars) of the GSK2 SNP (rs6438552) (i.e. the SNP located in intron 5 of GSK-3β, shown in FIG. 1A). Mean values ±SD obtained from four separate transfections. Pairwise Student's t test comparisons were performed between the T and C allele exon trap products. Statistical significance is indicated (*=p<0.05).

2D is a graphical representation of semi-quantitative analysis of exon trap products isolated from SK-N-MC cells transfected with either the C allele (open bars) or T allele (black bars) of the GSK2 SNP (rs6438552) (i.e. the SNP located in intron 5 of GSK-3β, shown in FIG. 1A). Mean values ±SD obtained from four separate transfections. Pairwise Student's t test comparisons were performed between the T and C allele exon trap products. Statistical significance is indicated (*=p<0.05).

FIG. 3A is a copy of a photographic representation showing electrophoresis of ³³P-labelled RT-PCR products from GSK-3β transcripts produced using RNA isolated from patient lymphocytes on a 6% PAGE gel. The major bands correspond to GSKΔexon9 and GSKΔexon9+11 transcripts. The identity of each of the bands is indicated on the right hand side of the figure.

FIG. 3B is a graphical representation showing semi-quantitative analysis of GSK-3β splice isoform transcripts from RNA isolated from patient lymphocytes. The relative level of each splice isoform is indicated for C/C genotype (open bars) or T/T genotype (black bars). Pairwise comparisons were made between two genotype groups (n=22) using Student's t test. Significance is indicated (*=p<0.05, **=p<0.005).

FIG. 3C is a copy of a photographic representation showing a Western blot of COS-7 cells transfected with GSK-3β cDNAs. Lane 1 GSK.Full length cDNA; lane 2=GSKΔexon9 cDNA; lane 3=GSKΔexon11 cDNA; lane 4=GSKΔexon9+11 cDNA; and lane 5=Lymphocyte control. The results obtained with the lymphocyte sample indicate that the two major protein bands which to GSKΔexon9 and GSKΔexon9+11.

FIG. 4A is a graphical representation showing quantification of Tau [pSER396] levels in COS-7 cells transfected with a GSK-3β cDNA. Results were normalized for GSK-3β levels by Western blot from three separate transfections. Significant differences were observed for the GSKΔexon9+11 isoform (***=p<0.0005) following ANOVA and post-hoc analysis.

FIG. 4B is a graphical representation showing the correlation between GSKΔexon9+11 transcript levels and Tau [pSER396] levels. Lymphocyte cells were analyzed for relative de novo GSKΔexon9+11 transcript levels and Tau [pSER396] levels in individuals with T/T (black circles) and C/C (open circles) genotypes for the GSK2 SNP (rs6438552). Significance for the coefficient of determination (r²) was calculated using linear regression analysis with the null hypothesis that there is no correlation within the population (r²=0).

FIG. 4C is a graphical representation showing relative levels of Tau [pSER396] phosphorylation when cells expressing GSKΔexon9 or GSKΔexon9+11 were incubated in the presence of increasing concentrations of lithium chloride. Values were normalized against the level of Tau [p396] phosphorylation detected in the absence of inhibitor. Dose response curves and mean values (±SEM) are plotted against a semi-logarithmic axis.

FIG. 4D is a graphical representation showing relative levels of Tau [pSER396] phosphorylation when cells expressing GSKΔexon9 or GSKΔexon9+11 were incubated in the presence of increasing concentrations of TDZD8. Values were normalized against the level of Tau [p396] phosphorylation detected in the absence of inhibitor. Dose response curves and mean values (±SEM) are plotted against a semi-logarithmic axis.

FIG. 5 is a graphical representation showing the effect of the SNPs designated rs3755557, rs334558 and rs6438552 in the NCBI SNP database on expression levels of different splice-forms of GSK-3β. GSKD9, GSK3β splice form lacking exon 9; GSKD9+11, GSK3β splice form lacking exons 9 and 11.

FIG. 6A is a copy of a photographic representation showing Western blot analysis of protein extract from HEK293 cells transfected with vectors expressing β-galactosidase (LacZ) or Tau cDNA. Levels of expression of β-catenin, GSK-3β or Tau were detected with antibodies described in the text of the specification and are indicated at the right-hand side of the figure. 4 repeat Tau—full-length Tau protein; 3 repeat Tau—Tau protein encoded by a cDNA lacking exon 10.

FIG. 6B is a graphical representation showing the amount of β-catenin in transfected HEK293 or SK-N-MC cells transfected with expression vectors expressing β-galactosidase (LacZ), GSK-3β or Tau. The level of total β-catenin is expressed as a ratio of β-catenin over GSK-3β levels to control for differences in gel loading. Error bars are derived from multiple transfections (n=6). Significance at level p<0.05 is indicated (*).

FIG. 7 is a graphical representation showing the mean overall side-effect score (SE score) determined as described in Example 11 of subjects having a “risk haplotype” (i.e., subjects having at least one H2 haplotype in MAPT and additionally having (i) a cytosine at a position corresponding to nucleotide position 181,700 of SEQ ID NO: 1, a thymidine at a position corresponding to nucleotide position 231 of SEQ ID NO: 1 and an adenosine at a position corresponding to nucleotide position 1679 of SEQ ID NO: 10; or (ii) a cytosine at a position corresponding to nucleotide position 181,700 of SEQ ID NO: 1, cytosine at a position corresponding to nucleotide position 231 of SEQ ID NO: 1 and a thymidine at a position corresponding to nucleotide position 1679 of SEQ ID NO: 10; or (iii) a thymidine at a position corresponding to nucleotide position 181,700 of SEQ ID NO: 1, a cytosine at a position corresponding to nucleotide position 231 of SEQ ID NO: 1 and an adenosine at a position corresponding to nucleotide position 1679 of SEQ ID NO: 10) (black bars) or subjects that do not have such a risk haplotype (white bars).

### DETAILED DESCRIPTION OF THE PREFERRED EMBODIMENTS

### Neurological Disorders

As used herein, the term “neurological disorder” shall be taken to include any disorder characterized by neuronal dysfunction and/or neuronal cell death. For example, a neurological disorder is a neurodegenerative disorder or a behavioral disorder or a myelin-associated disorder.

In a preferred embodiment, the method of the invention is useful for determining a subject that suffers from or is predisposed to or at risk of developing a neurodegenerative disorder.

As used herein, the term “neurodegenerative disorder” shall be taken to mean a disease that is characterized by neuronal cell death. The neuronal cell death observed in a neurological disorder is often preceded by neuronal dysfunction, sometimes by several years. Accordingly, the term “neurological disorder” includes a disease or disorder that is characterized by neuronal dysfunction and eventually neuronal cell death. Often neurological disorders are also characterized by increased gliosis (e.g., astrocytosis or microgliosis) in the region/s of neuronal death.

The cellular events observed in a neurological disorder often manifest as a behavioral change (e.g., deterioration of thinking and/or memory) and/or a movement change (e.g., tremor, ataxia, postural change and/or rigidity). Examples of neurological disorder include, for example, Alzheimer's disease, amyotrophic lateral sclerosis, ataxia (e.g., spinocerebellar ataxia or Friedreich's Ataxia), Creutzfeldt-Jakob Disease, a polyglutamine disease (e.g., Huntington's disease or spinal bulbar muscular atrophy), Hallervorden-Spatz disease, idiopathic torsion disease, Lewy body disease, multiple system atrophy, neuroanthocytosis syndrome, olivopontocerebellar atrophy, Parkinson's disease, Pelizaeus-Merzbacher disease, Pick's disease, progressive supranuclear palsy, syringomyelia, torticollis, spinal muscular atrophy or a trinucleotide repeat disease (e.g., Fragile X Syndrome).

In one embodiment, the neurological disorder is associated with aberrant deposition or tau and/or hyperphosphorylation of tau. For example, the neurological disorder is selected from the group consisting of frontotemporal dementia, corticobasal degeneration, progressive supranuclear palsy, a Parkinson's disease or an Alzheimer's disease. Preferably, the method of the invention is useful for diagnosing or determining a predisposition to a neurological disorder selected from the group consisting of a Parkinson's disease and an Alzheimer's disease.

By “Parkinson's disease” is meant a chronic progressive nerve disease characterized by muscle tremors, weakness, rigid movements, halting gait, drooping posture and expressionless facial appearance. The pathology of a Parkinson's disease is characterized by loss of neuromelanin-containing monoamine neurons, particularly dopamine (DA) neurons in the substantia nigra pars compacta. A pathologic hallmark is the presence of cytoplasmic eosinophilic inclusions (Lewy bodies) in monoamine neurons. The term “a Parkinson's disease” shall be understood to encompass early onset Parkinson's disease and late onset Parkinson's disease, juvenile onset Parkinson's disease, idiopathic Parkinson's disease and monogenic Parkinson's disease.

In another embodiment, a neurological disorder is a dementing neurological disorder. By “dementing neurological disorder” is meant a disease that is characterized by chronic loss of mental capacity, particularly progressive deterioration of thinking, memory, behavior, personality and motor function, and may also be associated with psychological symptoms such as depression and apathy. Preferably, a dementing neurological disorder is not caused by, for example, a stroke, an infection or a head trauma. Examples of a dementing neurological disorder include, for example, an Alzheimer's disease, vascular dementia, dementia with Lewy bodies, frontotemporal dementia and prion disease, amongst others.

Preferably, the dementing neurological disorder is an Alzheimer's disease. By “an Alzheimer's disease” is meant a neurological disorder characterized by progressive impairments in memory, behavior, language and/or visuo-spatial skills. Pathologically, an Alzheimer's disease is characterized by neuronal loss, gliosis, neurofibrillary tangles, senile plaques, Hirano bodies, granulovacuolar degeneration of neurons, amyloid angiopathy and/or acetylcholine deficiency. The term “an Alzheimer's disease” shall be taken to include early onset Alzheimer's disease (e.g., with an onset earlier than the sixth decade of life), a late onset Alzheimer's disease (e.g., with an onset later then, or in, the sixth decade of life) and a juvenile onset Alzheimer's disease.

In another embodiment, the neurological disorder is a behavioral disorder or a psychiatric disorder. By “behavioral disorder” or “psychiatric disorder” is meant a disorder that is associated with behavioral changes in an individual with or without neurodegeneration (or without obvious neurodegeneration). A suitable psychiatric disorder includes, for example, a somatoform disorder, an anxiety disorder, a dissociative disorder, a mood disorder, a personality disorder, a psycho-sexual disorder and a schizophrenia. For example, a psychiatric disorder is a disorder such as, for example, a bipolar affective disorder, a cyclothymic disorder, schizophrenia, a schizoaffective disorder, a schizophreniform disorder and a brief psychotic disorder.

In a preferred embodiment, the behavioral disorder or psychiatric disorder is a bipolar affective disorder. As used herein, the term “a bipolar affective disorder” shall be taken to include all forms of bipolar affective disorder, including bipolar I disorder (severe bipolar affective (mood) disorder), schizoaffective disorder, bipolar II disorder or unipolar disorder.

In another embodiment, the behavioral disorder or psychiatric disorder is schizophrenia.

In a further embodiment, the neurological disorder is a myelin-associated disorder. Preferred myelin-associated disorders are those disorders characterized by a reduction in the amount of or the production of scars or scleroses associated with myelin associated with or surrounding neuronal fibers. Preferably, the myelin-associated disorder is multiple sclerosis.

### GSK-3β Gene and Expression Products

In one embodiment, the marker that is associated with a neurological disorder comprises, consists of or is located within a GSK-3β genomic gene. Preferably, a GSK-3β genomic gene comprises a nucleotide sequence at least about 80% identical to the nucleotide sequence set forth in SEQ ID NO: 1 or the complement thereof. More preferably, the degree of identity is at least about 85% to about 90%, more preferably, about 90% to about 95%, even more preferably about 95% to about 99%.

A genomic gene of GSK-3β shall be understood to include the coding region of a GSK-3β protein (e.g., codons required to encode various isozymes of GSK-3β) in addition to intervening intronic sequences in addition to regulatory regions that control the expression of said gene, e.g., a promoter or fragment thereof.

In another embodiment, a marker that is associated with a neurological disorder comprises or is within an expression product of a GSK-3β gene. For example, the marker comprises, consists of or is located within a nucleic acid that comprises a nucleotide sequence at least about 80% identical to a GSK-3β cDNA or GSK-3β mRNA. For example, the nucleotide sequence of isoforms of a GSK-3β cDNA are set forth in SEQ ID NOs: 2, 4, 6, and 8. Preferably, the degree of identity is at least about 85% to about 90%, more preferably, about 90% to about 95%, even more preferably about 95% to about 99%.

In a still further embodiment, a marker that is associated with a neurological disorder comprises, consists of or is located within a GSK-3β polypeptide. Preferably, the marker comprises, consists of or is located within a polypeptide comprising an amino acid sequence at least about 80% identical to the amino acid sequence set forth in SEQ ID NOs: 3, 5, 7, or 9. Preferably, the degree of identity is at least about 85% to about 90%, more preferably, about 90% to about 95%, even more preferably about 95% to about 99%.

In determining whether or not two amino acid sequences fall within the defined percentage identity limits supra, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Madison, Wis., United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximize the number of identical/similar residues and to minimize the number and/or length of sequence gaps in the alignment.

Alternatively, a suite of commonly used and freely available sequence comparison algorithms is provided by the National Center for Biotechnology Information (NCBI) Basic Local Alignment Search Tool (BLAST) (Altschul et al. J. Mol. Biol. 215: 403-410, 1990), which is available from several sources, including the NCBI, Bethesda, Md. The BLAST software suite includes various sequence analysis programs including “blastn,” that is used to align a known nucleotide sequence with other polynucleotide sequences from a variety of databases and “blastp” used to align a known amino acid sequence with one or more sequences from one or more databases. Also available is a tool called “BLAST 2 Sequences” that is used for direct pairwise comparison of two nucleotide sequences.

As used herein the term “NCBI” shall be taken to mean the database of the National Center for Biotechnology Information at the National Library of Medicine at the National Institutes of Health of the Government of the United States of America, Bethesda, Md., 20894.

In determining whether or not two nucleotide sequences fall within a particular percentage identity limitation recited herein, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art. For example, nucleotide sequences may be aligned and their identity calculated using the BESTFIT program or other appropriate program of the Computer Genetics Group, Inc., University Research Park, Madison, Wis., United States of America (Devereaux et al, Nucl. Acids Res. 12, 387-395, 1984). As discussed supra BLAST is also useful for aligning nucleotide sequences and determining percentage identity.

### MAPT Gene and Expression Products

Preferably, a marker associated with a neurological disorder comprises, consists of or is located within a MAPT genomic gene. Preferably, a GSK-3β genomic gene encodes a nucleotide sequence at least about 80% identical to the nucleotide sequence set forth in SEQ ID NO: 11 or the complement thereof. More preferably, the degree of identity is at least about 85% to about 90%, more preferably, about 90% to about 95%, even more preferably about 95% to about 99%.

A genomic gene of MAPT shall be understood to include the coding region of a MAPT protein (e.g., codons required to encode various isozymes of GSK-3β) in addition to intervening intronic sequences in addition to regulatory regions that control the expression of said gene, e.g., a promoter or fragment thereof.

Preferably, a marker associated with a neurological disorder comprises, consists of or is located within a MAPT gene promoter. Preferably, a MAPT gene promoter comprises a nucleotide sequence at least about 80% identical to the nucleotide sequence set forth in SEQ ID NO: 13 or 14 or the complement thereof. More preferably, the degree of identity is at least about 85% to about 90%, more preferably, about 90% to about 95%, even more preferably about 95% to about 99%.

In another embodiment, a marker that is associated with a neurological disorder comprises or is within an expression product of a MAPT gene. For example, the marker comprises, consists of or is located within a nucleic acid that comprises a nucleotide sequence at least about 80% identical to a MAPT cDNA or MAPT mRNA. For example, the nucleotide sequence of a MAPT cDNA is set forth in SEQ ID NO: 11. Preferably, the degree of identity is at least about 85% to about 90%, more preferably, about 90% to about 95%, even more preferably about 95% to about 99%.

In a still further embodiment, a marker that is associated with a neurological disorder comprises, consists of or is located within a MAPT polypeptide. Preferably, the marker comprises, consists of or is located within a polypeptide comprising an amino acid sequence at least about 80% identical to the amino acid sequence set forth in SEQ ID NO: 12. Preferably, the degree of identity is at least about 85% to about 90%, more preferably, about 90% to about 95%, even more preferably about 95% to about 99%.

Markers Associated with a Disease or Disorder

In one embodiment, a marker associated with a neurological disorder is a nucleic acid marker. Preferably, the marker comprises or consists of a nucleotide sequence at least about 80% identical to at least about 20 nucleotides in length, more preferably at least about 30 nucleotides in length, of a sequence selected from the group consisting of:
(i) a sequence at least about 80% identical to a sequence selected from the group consisting of SEQ ID NO: 1, SEQ ID NO: 2, SEQ ID NO: 4, SEQ ID NO: 6, SEQ ID NO: 8, SEQ ID NO: 10, SEQ ID NO: 11, SEQ ID NO: 13 and SEQ ID NO: 14;
(ii) a sequence capable of encoding an amino acid sequence at least 80% identical to the sequence set forth in SEQ ID NO: 3, SEQ ID NO: 5, SEQ ID NO: 7, SEQ ID NO: 9 and SEQ ID NO: 12; and
(iii) a sequence complementary to a sequence set forth in (i) or (ii).

Such a nucleic acid marker may be or comprise, for example, a polymorphism, an insertion into a GSK-3β gene or a MAPT gene, a deletion from a GSK-3β gene or a MAPT gene, a transcript of a GSK-3β gene or a MAPT gene or a fragment thereof or an alternatively spliced transcript of a GSK-3β gene or a fragment thereof.

In one embodiment, a polymorphism associated with a disease or disorder is a simple nucleotide polymorphism. As used herein, the term “simple nucleotide polymorphism” shall be taken to mean a polymorphism that comprises or consists of a small change (e.g., an insertion, a deletion, a transition or a transversion) in the genome of a subject or an expression product thereof compared to the nucleotide sequence observed in a normal subject or the majority of a normal population of subjects. For example, a simple nucleotide polymorphism comprises or consists of, a single nucleotide insertion or deletion, insertion or deletion of two, three or four or more nucleotides, transition of one or more nucleotides, or transversion of one or more nucleotides.

Preferably, the marker in a GSK-3β gene associated with a neurological disorder or a predisposition to the neurological disorder is a single nucleotide polymorphism in its homozygous state.

In another preferred embodiment, a polymorphism in a GSK-3β gene associated with a neurological disorder is additionally associated with or causes alternative splicing of a GSK-3β mRNA. As used herein, the term “alternative splicing” shall be taken to mean the insertion or removal of exons into/from a GSK-3β mRNA. Accordingly, an alternatively spliced GSK-3β mRNA comprises additional exons, or lack exons (e.g., nucleotides) compared to the sequence of a GSK-3β cDNA set forth in SEQ ID NO: 2.

In one embodiment, the presence of a polymorphism that is associated with alternative splicing of a GSK-3β mRNA is correlated with modulated levels of alternatively spliced GSK-3β mRNA. Accordingly, the level of a specific splice form of GSK-3β is increased or decreased when the specific polymorphism is present and is useful for detecting a marker associated with a disease or disorder.

Preferably, an alternatively spliced GSK-3β transcript comprises a nucleotide sequence at least about 80% identical to a nucleotide sequence selected from the group consisting of SEQ ID NO: 4, SEQ ID NO: 6 and SEQ ID NO: 8. Preferably, the degree of sequence identity is at least about 90% or 95% or 98% or 99%.

In one embodiment, the polymorphism is associated with or causes the splicing of exon 9 in a GSK-3β mRNA (e.g., produces a cDNA that comprises a nucleotide sequence at least about 80% identical to the nucleotide sequence set forth in SEQ ID NO: 5.

In another embodiment, the polymorphism is associated with or causes the splicing of exon 9 and exon 11 in a GSK-3β mRNA (e.g., produces a cDNA that comprises a nucleotide sequence at least about 80% identical to the nucleotide sequence set forth in SEQ ID NO: 9.

In one embodiment, the polymorphism comprises, consists of or is located within intron 5 of the GSK-3β gene. In this context, the term “intron 5” shall be taken to mean the intron occurring between exons 5 and 6 of the cDNA sequence set forth in SEQ ID NO: 2. Preferably, the term “intron 5” shall be taken to mean a nucleic acid comprising a nucleotide sequence at least abut 80% identical or 90% identical or 98% identical to the nucleotides in the region spanning from nucleotide position 178,624 to nucleotide position 181,858 of SEQ ID NO: 1.

Preferably, a marker that is associated with a disease or disorder associated with aberrant GSK-3β expression and/or activity comprises a thymidine at a position corresponding to nucleotide position 181,700 of SEQ ID NO: 1. Preferably, the polymorphism is in a homozygous form.

Alternatively, a marker that is associated with a disease or disorder associated with aberrant GSK-3β expression and/or activity comprises a cytosine at a position corresponding to nucleotide position 181,700 of SEQ ID NO: 1. Preferably, the polymorphism is in a homozygous form.

The present inventors have additionally shown association of two polymorphisms in the promoter region of a GSK-3β gene and the development of a neurological disorder. Accordingly, in another embodiment of the invention, the polymorphism comprises, consists of or is located within the promoter region of GSK-3β. As used herein the term “promoter region of GSK-3β” shall be taken to mean a nucleic acid that comprises one or more elements that are associated with control of expression of one or more isoforms of GSK-3β.

Preferably, a polymorphism located within the promoter region of a GSK-3β gene comprises, consists of or is located within a nucleic acid that comprises a nucleotide sequence corresponding to the region spanning from approximately nucleotide position 1 to nucleotide position 1232 of SEQ ID NO: 1 or that is set forth in SEQ ID NO: 49.

In a preferred embodiment a polymorphism located within the promoter region of a GSK-3β gene is also associated with or causes increased expression of a GSK-3β expression product. Methods for determining expression of a GSK-3β mRNA or polypeptide are known in the art and/or described herein.

In a preferred embodiment, the polymorphism comprises or consists of a thymidine or a cytosine at a position corresponding to nucleotide position 232 of SEQ ID NO: 1.

In another embodiment, the polymorphism comprises or consists of a cytosine at a position corresponding to nucleotides position 3356 of SEQ ID NO: 49. In another embodiment, the polymorphism comprises or consists of a thymidine at a position corresponding to nucleotides position 3356 of SEQ ID NO: 49.

In another preferred embodiment, the polymorphism comprises an adenosine or a cytosine at a position corresponding to nucleotide position 1679 of SEQ ID NO: 49.

As for markers in a MAPT gene, it is preferable that the marker is located within a promoter region of a MAPT gene. Preferably, the marker comprises a polymorphism selected from the group consisting of:
(i) AATTT at a position equivalent to nucleotide position 763-767 of SEQ ID NO: 13;
(ii) TT at a position equivalent to nucleotide positions 793 and 794 of SEQ ID NO: 13;
(iii) A at a position equivalent to nucleotide position 817 of SEQ ID NO: 13;
(iv) G at a position equivalent to nucleotide position 1011 of SEQ ID NO: 13;
(v) T at a position equivalent to nucleotide position 1036 of SEQ ID NO: 13;
(vi) C at a position equivalent to nucleotide position 1159 of SEQ ID NO: 13;
(vii) A at a position equivalent to nucleotide position 1340 of SEQ ID NO: 13; and
(viii) any combination of (i) to (vii).

As will be apparent from the disclosure herein, the method of the invention comprises detecting at least one marker in a GSK-3β gene and at least one marker in a MAPT gene.

### Detection of Nucleic Acid Changes in a Gene or Expression Product

In one embodiment the method for determining the predisposition of a subject to a neurological disorder or diagnosing the neurological disorder disease comprises determining the presence of the marker within a GSK-3β gene or transcript thereof and/or a MAPT gene or transcript thereof that is associated with the disease or disorder in a test sample derived from a subject, wherein the presence of the markers indicates that the subject being tested is predisposed to or suffers from the neurological disorder.

As will be apparent to the skilled artisan, a marker that is associated with a disease or disorder is preferably detectable by standard procedures, for example by nucleic acid hybridization. Accordingly, a nucleic acid marker is preferably at least about 8 nucleotides in length (for example, for detection using a locked nucleic acid (LNA) probe). To provide more specific hybridization, a marker is preferably at least about 15 nucleotides in length or more preferably at least 20 to 30 nucleotides in length. Such markers are particularly amenable to detection by nucleic acid hybridization-based detection means assays, such as, for example any known format of PCR or ligase chain reaction.

Generally, a method for detecting a nucleic acid marker comprises hybridizing an oligonucleotide to the marker linked to nucleic acid in a sample from a subject under moderate to high stringency conditions and detecting hybridization of the oligonucleotide using a detection means, such as for example, an amplification reaction or a hybridization reaction.

For the purposes of defining the level of stringency to be used in these diagnostic assays, a low stringency is defined herein as being a hybridization and/or a wash carried out in 6×SSC buffer, 0.1% (w/v) SDS at 28° C., or equivalent conditions. A moderate stringency is defined herein as being a hybridization and/or washing carried out in 2×SSC buffer, 0.1% (w/v) SDS at a temperature in the range 45° C. to 65° C., or equivalent conditions. A high stringency is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample DNA, and/or the type of hybridization probe used.

In another embodiment, stringency is determined based upon the temperature at which a probe or primer dissociates from a target sequence (i.e., the probe or primers melting temperature or Tm). Such a temperature may be determined using, for example, an equation or by empirical means. Several methods for the determination of the Tm of a nucleic acid are known in the art. For example the Wallace Rule determines the G+C and the T+A concentrations in the oligonucleotide and uses this information to calculate a theoretical Tm (Wallace et al., Nucleic Acids Res. 6, 3543, 1979). Alternative methods, such as, for example, the nearest neighbour method are known in the art, and described, for example, in Howley, et al., J. Biol. Chem. 254, 4876, Santa Lucia, Proc. Natl. Acad. Sci. USA, 95: 1460-1465, 1995 or Bresslauer et al., Proc. Natl. Acad. Sci. USA, 83: 3746-3750, 1986. A temperature that is similar to (e.g., within 5° C. or within 10° C.) or equal to the proposed denaturing temperature of a probe or primer is considered to be high stringency. Medium stringency is to be considered to be within 10° C. to 20° C. or 10° C. to 15° C. of the calculated Tm of the probe or primer.

As will be apparent to the skilled artisan a probe or primer capable of specifically detecting a marker associated with a neurological disorder is any probe or primer that is capable of selectively hybridizing to the region of the genome that comprises said marker, or an expression product thereof. As used herein, the term “selectively hybridizes” means that the nucleic acid used as a probe hybridizes to a target nucleic acid (e.g., a nucleic acid comprising the marker) at a level significantly above background. The background hybridization may be due to other nucleic acids present, for example, in the sample being screening. Accordingly, background hybridization is a level of hybridization that occurs between the probe and a non-specific nucleic acid in the sample being assayed. Preferably, the a background interaction occurs less than 10 fold, preferably less than 100 fold as often as the specific (target) interaction observed with the target nucleic acid. The degree of interaction is measured, for example, by labeling the probe with a detectable marker, e.g. with ³²P or by performing an amplification reaction and determining the approximate level of amplification product produced, e.g., using gel electrophoresis.

In one embodiment, a preferred probe or primer comprises, consists of or is located within a nucleic acid comprising a nucleotide sequence at least about 80% identical to a sequence selected from the group consisting of:
(i) a sequence at least about 80% identical to a sequence selected from the group consisting of SEQ ID NO: 1, SEQ ID NO: 2, SEQ ID NO: 4, SEQ ID NO: 6, SEQ ID NO: 8, SEQ ID NO: 10, SEQ ID NO: 11, SEQ ID NO: 13 and SEQ ID NO: 14;
(ii) a sequence capable of encoding an amino acid sequence at least 80% identical to the sequence set forth in SEQ ID NO: 3, SEQ ID NO: 5, SEQ ID NO: 7, SEQ ID NO: 9 and SEQ ID NO: 12; and
(iii) a sequence complementary to a sequence set forth in (i) or (ii).

Probe/Primer Design and Production

As will be apparent to the skilled artisan, the specific probe or primer used in an assay of the present invention will depend upon the assay format used. Clearly, a probe or primer that is capable of specifically hybridizing to or detecting the marker of interest is preferred. Methods for designing probes and/or primers for, for example, PCR or hybridization are known in the art and described, for example, in Dieffenbach and Dveksler (Eds) (In: PCR Primer: A Laboratory Manual, Cold Spring Harbor Laboratories, NY, 1995). Furthermore, several software packages are publicly available that design optimal probes and/or primers for a variety of assays, e.g. Primer 3 available from the Center for Genome Research, Cambridge, Mass., USA. Probes and/or primers useful for detection of a marker associated with a disease or disorder associated with aberrant GSK-3β activity and/or expression are assessed to determine those that do not form hairpins, self-prime or form primer dimers (e.g. with another probe or primer used in a detection assay).

Furthermore, a probe or primer (or the sequence thereof) is assessed to determine the temperature at which it denatures from a target nucleic acid (i.e. the melting temperature of the probe or primer, or Tm). Methods of determining Tm are known in the art and described, for example, in Santa Lucia, Proc. Natl. Acad. Sci. USA, 95: 1460-1465, 1995 or Bresslauer et al., Proc. Natl. Acad. Sci. USA, 83: 3746-3750, 1986.

A primer or probe useful for detecting a polymorphism, e.g., a SNP in an allele specific PCR assay or a ligase chain reaction assay is designed such that the 3′ terminal nucleotide(s) hybridize(s) to the site of the polymorphism. The 3′ terminal nucleotide may be any of the nucleotides known to be present at the site of the polymorphism. When complementary nucleotides occur in the probe or primer and at the site of the polymorphism the 3′ end of the probe or primer hybridizes completely to the marker of interest and facilitates, for example, PCR amplification or ligation to another nucleic acid. Accordingly, a probe or primer that completely hybridizes to the target nucleic acid produces a positive result in an assay.

In another embodiment, a primer useful for a primer extension reaction is designed such that it specifically hybridizes to a region adjacent to a specific nucleotide of interest, e.g. a SNP. While the specific hybridization of a probe or primer may be estimated by determining the degree of homology of the probe or primer to any nucleic acid using software, such as, for example, BLAST, the specificity of a probe or primer can only be determined empirically using methods known in the art.

A locked nucleic acid (LNA) or protein-nucleic acid (PNA) probe or a molecular beacon useful, for example, for detection of a SNP or other polymorphism by hybridization is at least about 8 to 12 nucleotides in length. Preferably, the nucleic acid, or derivative thereof, that hybridizes to the site of the SNP or polymorphism is positioned at approximately the centre of the probe, thereby facilitating selective hybridization and accurate detection.

Methods for producing/synthesizing a probe or primer of the present invention are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (e.g. by digestion of a nucleic acid with a restriction endonuclease) or by chemical synthesis. For short sequences (up to about 100 nucleotides) chemical synthesis is preferable.

For longer sequences standard replication methods employed in molecular biology are useful, such as, for example, the use of M13 for single stranded DNA as described by J. Messing (1983) Methods Enzymol, 101, 20-78.

Other methods for oligonucleotide synthesis include, for example, phosphotriester and phosphodiester methods (Narang, et al. Meth. Enzymol 68: 90, 1979) and synthesis on a support (Beaucage, et al Tetrahedron Letters 22: 1859-1862, 1981) as well as phosphoramidate technique, Caruthers, M. H., et al., “Methods in Enzymology,” Vol. 154, pp. 287-314 (1988), and others described in “Synthesis and Applications of DNA and RNA,” S. A. Narang, editor, Academic Press, New York, 1987, and the references contained therein.

LNA synthesis is described, for example, in Nielsen et al, J. Chem. Soc. Perkin Trans., 1: 3423, 1997; Singh and Wengel, Chem. Commun. 1247, 1998. While, PNA synthesis is described, for example, in Egholm et al., Am. Chem. Soc., 114: 1895, 1992; Egholm et al., Nature, 365: 566, 1993; and Orum et al., Nucl. Acids Res., 21: 5332, 1993.

In one embodiment, the probe or primer comprises one or more detectable markers. For example, the probe or primer comprises a fluorescent label such as, for example, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

Alternatively, the probe or primer is labeled with, for example, a fluorescent semiconductor nanocrystal (as described, for example, in U.S. Pat. No. 6,306,610), a radiolabel or an enzyme (e.g. horseradish peroxidase (HRP), alkaline phosphatase (AP) or β-galactosidase).

Such detectable labels facilitate the detection of a probe or primer, for example, the hybridization of the probe or primer or an amplification product produced using the probe or primer. Methods for producing such a labeled probe or primer are known in the art. Furthermore, commercial sources for the production of a labeled probe or primer will be known to the skilled artisan, e.g., Sigma-Genosys, Sydney, Australia.

Clearly, the present invention encompasses a probe or primer comprising at least 20 nucleotides that is capable of selectively hybridizing to the nucleotide sequence set forth in SEQ ID NO: 1, SEQ ID NO: 10, SEQ ID NO: 11 or SEQ ID NO: 13 and detecting a marker that is associated with a neurological disorder.

The present invention additionally contemplates the use a probe or primer produced according to the methods described herein in the manufacture of a diagnostic reagent for diagnosing or determining a predisposition to a neurological disorder.

Detection Methods

Methods for detecting nucleic acids are known in the art and include for example, hybridization based assays, amplification based assays and restriction endonuclease based assays. For example, a change in the sequence of a region of the genome or an expression product thereof, such as, for example, an insertion, a deletion, a transversion, a transition, alternative splicing or a change in the preference of or occurrence of a splice form of a gene is detected using a method, such as, polymerase chain reaction (PCR) strand displacement amplification, ligase chain reaction, cycling probe technology or a DNA microarray chip amongst others.

Methods of PCR are known in the art and described, for example, in Dieffenbach (Ed) and Dveksler (Ed) (In: PCR Primer: A Laboratory Manual, Cold Spring Harbor Laboratories, NY, 1995). Generally, for PCR two non-complementary nucleic acid primer molecules comprising at least about 20 nucleotides, and more preferably at least 30 nucleotides are hybridized to different strands of a nucleic acid template molecule, and specific nucleic acid molecule copies of the template are amplified enzymatically. PCR products may be detected using electrophoresis and detection with a detectable marker that binds nucleic acids. Alternatively, one or more of the oligonucleotides are labeled with a detectable marker (e.g. a fluorophore) and the amplification product detected using, for example, a lightcycler (Perkin Elmer, Wellesley, Mass., USA). Alternatively, PCR products are detected, for example, using mass spectrometry. Clearly, the present invention also encompasses quantitative forms of PCR, such as, for example, a Taqman assay.

Strand displacement amplification (SDA) utilizes oligonucleotides, a DNA polymerase and a restriction endonuclease to amplify a target sequence. The oligonucleotides are hybridized to a target nucleic acid and the polymerase used to produce a copy of this region. The duplexes of copied nucleic acid and target nucleic acid are then nicked with an endonuclease that specifically recognizes a sequence of nucleotides at the beginning of the copied nucleic acid. The DNA polymerase recognizes the nicked DNA and produces another copy of the target region at the same time displacing the previously generated nucleic acid. The advantage of SDA is that it occurs in an isothermal format, thereby facilitating high-throughput automated analysis.

Ligase chain reaction (described in, for example, EU 320,308 and U.S. Pat. No. 4,883,750) uses two or more oligonucleotides that hybridize to adjacent target nucleic acids. A ligase enzyme is then used to link the oligonucleotides. In the presence of one or more nucleotide(s) that is(are) not complementary to the nucleotide at an end of one of the primers that is adjacent to the other primer, the ligase is unable to link the primers, thereby failing to produce a detectable amplification product. Using thermocycling the ligated oligonucleotides then become a target for further oligonucleotides. The ligated fragments are then detected, for example, using electrophoresis, or MALDI-TOF. Alternatively, or in addition, one or more of the probes is labeled with a detectable marker, thereby facilitating rapid detection.

Cycling Probe Technology uses chimeric synthetic probe that comprises DNA-RNA-DNA that is capable of hybridizing to a target sequence. Upon hybridization to a target sequence the RNA-DNA duplex formed is a target for RNase H that cleaves the probe. The cleaved probe is then detected using, for example, electrophoresis or MALDI-TOF.

In a preferred embodiment, a detection method detects the presence or absence of a specific allele at the site of a SNP or other polymorphism. Methods for detecting polymorphisms, e.g., SNPs are known in the art, and reviewed, for example, in Landegren et al, Genome Research 8: 769-776, 1998.

For example, a polymorphism (e.g., SNP) that introduces or alters a sequence that is a recognition sequence for a restriction endonuclease is detected by digesting DNA with the endonuclease and detecting the fragment of interest using, for example, Southern blotting (described in Ausubel et al (In: Current Protocols in Molecular Biology. Wiley Interscience, ISBN 047 150338, 1987) and Sambrook et al (In: Molecular Cloning: Molecular Cloning: A Laboratory Manual, Cold Spring Harbor Laboratories, New York, Third Edition 2001)). Alternatively, a nucleic acid amplification method described supra, is used to amplify the region surrounding the polymorphism. The amplification product is then incubated with the endonuclease and any resulting fragments detected, for example, by electrophoresis, MALDI-TOF or PCR.

The direct analysis of the sequence of polymorphisms of the present invention can be accomplished using either the dideoxy-chain termination method or the Maxam-Gilbert method (see Sambrook et al., Molecular Cloning, A Laboratory Manual (2nd Ed., CSHP, New York 1989); and Zyskind et al., Recombinant DNA Laboratory Manual, (Acad. Press, 1988)). As will be apparent to the skilled artisan, the presence of a polymorphism in a homozygous or heterozygous form may be detected using standard sequencing methods.

Alternatively, a polymorphism (e.g., SNP) is detected using single stranded conformational polymorphism (SSCP) analysis. SSCP analysis relies upon the formation of secondary structures in nucleic acids and the sequence dependent nature of these secondary structures. In one form of this analysis an amplification method, such as, for example, a method described herein, is used to amplify a nucleic acid that comprises a polymorphism. The amplified nucleic acids are then denatured, cooled and analyzed using, for example, non-denaturing polyacrylamide gel electrophoresis, mass spectrometry, or liquid chromatography (e.g. HPLC or dHPLC). Regions that comprise different sequences form different secondary structures, and as a consequence migrate at different rates through, for example, a gel and/or a charged field. Accordingly, both homozygous forms of a polymorphism and a heterozygous form of the polymorphism may be detected using such analysis. Clearly, a detectable marker may be incorporated into a probe/primer useful in SSCP analysis to facilitate rapid marker detection. SSCP analysis is also particularly suited to determining whether a polymorphism is in a homozygous or a heterozygous form, as it detects and differentiates between both forms of the polymorphism.

Alternatively, any nucleotide changes are detected using, for example, mass spectrometry or capillary electrophoresis. For example, amplified products of a region of DNA comprising a polymorphism (e.g., a SNP) from a test sample are mixed with amplified products from a normal/healthy individual. The products are denatured and allowed to re-anneal. Clearly, those samples that comprise a different nucleotide at the position of the polymorphism will not completely anneal to a nucleic acid molecule from a normal/healthy individual thereby changing the charge and/or conformation of the nucleic acid, when compared to a completely annealed nucleic acid. Such incorrect base pairing is detectable using, for example, mass spectrometry. As with SSCP, methods for detecting a polymorphism using, for example, mass spectrometry or capillary electrophoresis are particularly suited for determining whether a polymorphism is in a homozygous form or a heterozygous form.

Mass spectrometry is also useful for detecting the molecular weight of a short amplified product, wherein a nucleotide change causes a change in molecular weight of the nucleic acid molecule (such a method is also described, for example, in U.S. Pat. No. 6,574,700).

Allele specific PCR (as described, for example, In Liu et al, Genome Research, 7: 389-398, 1997) is also useful for determining the presence of one or other allele of a polymorphism, e.g., a SNP. An oligonucleotide is designed, in which the most 3′ base of the oligonucleotide hybridizes with the SNP. During a PCR reaction, if the 3′ end of the oligonucleotide does not hybridize to a target sequence, little or no PCR product is produced, indicating that a base other than that present in the oligonucleotide is present at the site of SNP in the sample. PCR products are then detected using, for example, gel or capillary electrophoresis or mass spectrometry.

Primer extension methods (described, for example, in Dieffenbach (Ed) and Dveksler (Ed) (In: PCR Primer: A Laboratory Manual, Cold Spring Harbor Laboratories, NY, 1995)) are also useful for the detection of a SNP. An oligonucleotide is used that hybridizes to the region of a nucleic acid adjacent to the SNP. This oligonucleotide is then used in a primer extension protocol with a polymerase and a free nucleotide diphosphate or dideoxynucleotide triphosphate that corresponds to either or any of the possible bases that occur at the SNP. Preferably the nucleotide-diphosphate is labeled with a detectable marker (e.g. a fluorophore). Following primer extension, unbound labeled nucleotide diphosphates are removed, e.g. using size exclusion chromatography or electrophoresis, or hydrolyzed, using for example, alkaline phosphatase, and the incorporation of the labeled nucleotide into the oligonucleotide is detected, indicating the base that is present at the site of the SNP. Alternatively, or in addition, as exemplified herein primer extension products are detected using mass spectrometry (e.g. MALDI-TOF).

By using a plurality of different free nucleotide diphosphates, wherein each of the free nucleotide diphosphates is labeled with a different detectable marker, the heterozygosity or homozygosity of a polymorphism is determined.

Clearly, the present invention extends to high-throughput forms primer extension analysis, such as, for example, minisequencing (Sy Vämen et al., Genomics 9: 341-342, 1995). In such a method, a probe or primer (or multiple probes or primers) are immobilized on a solid support (e.g. a glass slide). A biological sample comprising nucleic acid is then brought into direct contact with the probe/s or primer/s, and a primer extension protocol performed with each of the free nucleotide bases labeled with a different detectable marker. The nucleotide present at the site of a polymorphism or a number of polymorphisms is then determined by determining the detectable marker bound to each probe and/or primer.

Fluorescently labeled locked nucleic acid (LNA) molecules or fluorescently labeled protein-nucleic acid (PNA) molecules are useful for the detection of a polymorphism (as described in Simeonov and Nikiforov, Nucleic Acids Research, 30(17): 1-5, 2002). LNA and PNA molecules bind, with high affinity, to nucleic acid, in particular, DNA. Fluorophores (in particular, rhodomine or hexachlorofluorescein) conjugated to the LNA or PNA probe fluoresce at a significantly greater level upon hybridization of the probe to target nucleic acid. However, the level of increase of fluorescence is not enhanced to the same level when even a single nucleotide mismatch occurs. Accordingly, the degree of fluorescence detected in a sample is indicative of the presence of a mismatch between the LNA or PNA probe and the target nucleic acid, such as, in the presence of a SNP. Preferably, fluorescently labeled LNA or PNA technology is used to detect a single base change in a nucleic acid that has been previously amplified using, for example, an amplification method known in the art and/or described herein.

As will be apparent to the skilled artisan, LNA or PNA detection technology is amenable to a high-throughput detection of one or more markers by immobilizing an LNA or PNA probe to a solid support, as described in Orum et al., Clin. Chem. 45: 1898-1905, 1999.

Molecular Beacons™ are also useful for detecting polymorphism directly in a sample or in an amplified product (see, for example, Mhlang and Malmberg, Methods 25: 463-471, 2001). Molecular Beacons™ are single stranded nucleic acid molecules with a stem-and-loop structure. The loop structure is complementary to the region surrounding the SNP of interest. The stem structure is formed by annealing two “arms” complementary to each other that are on either side of the probe (loop). A fluorescent moiety is bound to one arm and the other arm comprises a quenching moiety that suppresses any detectable fluorescence when the molecular beacon is not bound to a target sequence. Upon binding of the loop region to its target nucleic acid the arms are separated and fluorescence is detectable. However, even a single base mismatch significantly alters the level of fluorescence detected in a sample. Accordingly, the presence or absence of a particular base at the site of a polymorphism is determined by the level of fluorescence detected.

A polymorphism can also be identified by hybridization to nucleic acid arrays, an example of which is described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Clearly, the present invention encompasses other methods of detecting a polymorphism within a GSK-3β gene or within a MAPT gene and associated with neurological disorder, such as, for example, a SNP microarray (for example, as commercially available from Affymetrix, and/or described, for example, in U.S. Pat. No. 6,468,743 or Hacia et al, Nature Genetics, 14: 441, 1996), a Taqman assay (as described, for example, in Livak et al, Nature Genetics, 9: 341-342, 1995), solid phase minisequencing (as described in Syvämen et al, Genomics, 13: 1008-1017, 1992), minisequencing with FRET (as described in Chen and Kwok, Nucleic Acids Res. 25: 347-353, 1997) or pyrominisequencing (as reviewed in Landegren et al., Genome Res., 8(8): 769-776, 1998).

### Detection of Altered Splicing of a GSK-3β Gene—Positive Assays

As exemplified herein, a SNP in a GSK-3β gene that is associated with a neurological disorder is also associated and/or causes a change in splicing of a GSK-3β mRNA. Accordingly, such an alternate splice form provides a marker associated with a disease or disorder. Alternatively, a marker associated with a disease or disorder is detected by detecting the alternate splice form of a GSK-3β transcript. For example, an increased level of a transcript lacking exons 9 and 11 is indicative of a neurodegenerative disease or a predisposition thereto and/or multiple sclerosis or a predisposition thereto. An increased level of a transcript lacking exon 9 is indicative of a bipolar affective disorder or a predisposition thereto. A reduced level of a transcript lacking exon 9 is indicative of schizophrenia or a predisposition thereto.

For example, an alternatively spliced form of GSK-3βay comprise additional nucleotides compared to, for example, a transcript comprising a nucleotide sequence set forth in SEQ ID NO: 2. Such alternate splice forms may be determined using a positive read-out assay. In the present context, the term “positive read-out assay” shall be taken to mean that a positive result in an assay indicates that a biological sample comprises an alternatively spliced GSK-3β transcript.

A positive read-out assay that detects such a marker associated with a disease or disorder or a predisposition to a disease or disorder comprises detecting the presence of an additional exon in the transcript.

In one embodiment, the marker is detected by hybridizing a nucleic acid probe or primer comprising, consisting of or located within the region of alternate splicing of a GSK-3β transcript to a nucleic acid that is linked to the marker in a biological sample derived from a subject and detecting the hybridization by a detection means, wherein hybridization of the probe or primer indicates that the subject being tested is predisposed to or suffers from a disease or disorder disease. Preferably, the detection means is an amplification reaction, or a nucleic acid hybridization reaction, such as, for example, as described herein.

In an alternative embodiment, the marker is detected by amplifying (e.g., using PCR, RT-PCR, NASBA, TMA or ligase chain reaction amongst other methods) an alternatively spliced region of a GSK-3β transcript. In accordance with this embodiment, one or more probe(s) or primer(s) (or two or more probes or primers) that flank or abut the region of a GSK-3β transcript that is alternatively spliced are hybridized to a nucleic acid linked to the marker in a biological sample. The marker is then detected using an amplification or primer extension protocol. Clearly, such a method is useful for detecting insertion of additional nucleic acid or removal of nucleic acid (i.e., splicing-out of nucleic acid e.g., an exon). Detection of an alternatively spliced region of a GSK-3β transcript indicates that a subject is at risk of developing a neurological disorder or suffers from such a neurological disorder.

As will be apparent to the skilled artisan, an alternatively spliced region of a GSK-3β transcript may be detected using a probe or primer that hybridizes to a region that flanks or is adjacent to a region of alternative splicing of a GSK-3β transcript and a probe or primer that hybridizes to a region of the GSK-3β transcript that is alternatively spliced. Furthermore, a probe or primer may hybridize to an alternative splice site (i.e., a region of the probe or primer hybridizes to the region adjacent to the alternative splice site and another region of the probe or primer hybridizes to the region of the GSK-3β transcript that is alternatively spliced).

As alternatively spliced forms of GSK-3β are detected using mRNA or cDNA derived therefrom, assays that detect changes in mRNA are particular preferred (e.g. RT-PCR, NASBA, TMA or ligase chain reaction).

Methods of RT-PCR are known in the art and described, for example, in Dieffenbach (ed) and Dveksler (ed) (In: PCR Primer: A Laboratory Manual, Cold Spring Harbor Laboratories, NY, 1995).

Methods of TMA or self-sustained sequence replication (3SR) use two or more oligonucleotides that flank a target sequence, a RNA polymerase, RNase H and a reverse transcriptase. One oligonucleotide (that also comprises a RNA polymerase binding site) hybridizes to an RNA molecule that comprises the target sequence and the reverse transcriptase produces cDNA copy of this region. RNase H is used to digest the RNA in the RNA-DNA complex, and the second oligonucleotide used to produce a copy of the cDNA. The RNA polymerase is then used to produce a RNA copy of the cDNA, and the process repeated.

NASBA systems relies on the simultaneous activity of three enzymes (a reverse transcriptase, RNase H and RNA polymerase) to selectively amplify target mRNA sequences. The mRNA template is transcribed to cDNA by reverse transcription using an oligonucleotide that hybridizes to the target sequence and comprises a RNA polymerase binding site at its 5′ end. The template RNA is digested with RNase H and double stranded DNA is synthesized. The RNA polymerase then produces multiple RNA copies of the cDNA and the process is repeated.

Clearly, the hybridization to and/or amplification of a marker associated with a disease or disorder associated with aberrant GSK-3β activity and/or expression using any of these methods is detectable using, for example, electrophoresis and/or mass spectrometry. In this regard, one or more of the probes/primers and/or one or more of the nucleotides used in an amplification reactions may be labeled with a detectable marker to facilitate rapid detection of a marker, for example, a fluorescent label (e.g. Cy5 or Cy3) or a radioisotope (e.g. ³²P).

Alternatively, amplification of a nucleic acid may be continuously monitored using a melting curve analysis method, such as that described in, for example, U.S. Pat. No. 6,174,670. Such methods are suited to determining the level of an alternative splice form in a biological sample.

Clearly, the present invention also encompasses the use of any nucleic acid detection method to detect alternative splicing in a GSK-3β mRNA or cDNA derived therefrom.

In a preferred embodiment, an alternatively spliced GSK-3β transcript comprises fewer exons that a wild-type GSK-3β (i.e. a GSK-3β transcript comprising the sequence set forth in SEQ ID NO: 2), e.g., a nucleic acid comprising or consisting of the nucleotide sequence set forth in SEQ ID NO: 4, SEQ ID NO: 6 or SEQ ID NO: 8. Preferably, an alternatively spliced GSK-3β lack exon 9 or both exon 9 and exon 11.

A positive read-out assay that detects an alternate splice-form of a GSK-3β transcript that lacks an exon comprises hybridizing a probe or primer to a region of a GSK-3β transcript adjacent to the splice site and determining whether or not the alternatively-spliced exon is present.

For example a PCR primer is hybridized to a region 5′ to the splice site, and another PCR primer is hybridized to a region 3′ of the splice site. Following amplification of the intervening sequence using a DNA polymerase, the size of the PCR product indicates whether or not the exon is present. Clearly, any amplification assay, e.g. RT-PCR, NASBA, TMA or ligase chain reaction is amenable to this form of analysis.

Alternatively, a probe or primer hybridizes to the region of the GSK-3β transcript comprising the splice site of interest. Accordingly, such a probe or primer will only hybridize to a sample that has been alternatively spliced. A Molecular Probe and/or a LNA probe and/or a PNA probe is particularly amenable to this form of analysis.

Detection of Altered Splicing of a GSK-3β Gene—Negative Assays

As will be apparent the skilled artisan, an alternate splice form of a GSK-3β transcript is also detectable using a negative read-out assay. As used herein, the term “negative read-out assay” shall be taken to mean that a negative result in an assay indicates that a biological sample comprises an alternatively spliced GSK-3β transcript.

For example, a negative read-out assay for the detection of an alternative splice-form of a GSK-3β transcript comprises hybridizing a probe or primer to a region of a GSK-3β transcript that is “spliced-out” or absent in the alternative splice form of interest. In one embodiment, such a probe or primer hybridizes to exon 9 or exon 11 of a GSK-3β transcript. As will be apparent to the skilled artisan, such a probe or primer will only hybridize to a form of a GSK-3β transcript that includes or comprises the region that is spliced. Accordingly, an assay that fails to detect hybridization of the probe or primer to nucleic acid in a biological sample (or detects a reduced amount of hybridization compared to a suitable control) indicates the presence of an alternative splice form of a GSK-3β transcript.

The detection of the hybridization of a probe or primer to nucleic acid in a biological sample is detected using any method known in the art, such as, for example, a method described supra. For example, a PCR primer is designed such that it hybridizes to exon 9 of GSK-3β. A second PCR primer is designed that hybridizes to exon 10 of GSK-3β. Use of such primers in a PCR reaction facilitates detection of a GSK-3β transcript that has spliced out exon 9. As will be apparent to the skilled artisan, the first oligonucleotide will not hybridize to nucleic acid in a biological sample in which there is a GSK-3β transcript that lacks exon 9. Accordingly, a reduced level of PCR product will be produced. Such a method may also be developed for the detection of alternate splicing of exon 11 and/or both exon 9 and exon 11 (e.g. using an oligonucleotides that hybridizes to exon 9 and an oligonucleotides that hybridizes to exon 11).

As will be apparent to the skilled artisan a negative read-out assay for the detection of an alternative splice-form of a GSK-3β transcript may also include a positive control Such a control is useful as failure to detect the alternative splice-form of a GSK-3β transcript may indicate that the detection reaction has failed. Such a positive control may involve, for example, using a probe or primer that hybridizes to a region of GSK-3β that is not alternatively spliced to determine the presence of GSK-3β encoding nucleic acid in a biological sample. Alternatively, a positive control may comprise a probe or primer that hybridizes to a transcript of a gene other than GSK-3β (that is known to be expressed in the biological sample) to determine the presence of nucleic acid in a biological sample. Examples of such genes include, actin, glyceraldehyde 3-phosphate dehydrogenase (GAPDH), β2 microglobulin, hydroxy-methylbilane synthase, hypoxanthine phosphoribosyl-transferase 1 (HPRT), ribosomal protein L13c, succinate dehydrogenase complex subunit A and TATA box binding protein (TBP) amongst others.

### Detection of Amino Acid Changes in a GSK-3β Polypeptide

In one embodiment, a marker associated with a disease or disorder associated with aberrant GSK-3β activity and/or expression is within a GSK-3β polypeptide. Such a marker is, for example, a single amino acid change, or the addition of or deletion of regions of a GSK-3β polypeptide (e.g. as a result of alternate splicing of a GSK-3β transcript.

Preferably, a protein marker encoded by nucleic acid that is associated with a disease or disorder associated with aberrant GSK-3β expression or activity is suitable for antigen-based detection. As will be apparent to the skilled artisan, even a single amino acid change in a polypeptide is detectable in an antigen-based assay, using, for example a monoclonal antibody, a single chain antibody, or an F′ab antibody fragment. However, for an antibody or ligand to detect such a change, a marker is preferably at least about 6 amino acids in length, more preferably at least about 8 to 10 amino acids in length, even more preferably at least about 14 amino acids in length. A protein marker may also be an entire protein, e.g. wherein the protein that is associated with a disease or disorder is, for example, a conformation different to the protein in a normal or healthy individual.

Generally, a polypeptide marker is detected in an assay that utilizes a ligand and/or antibody capable of binding to the marker.

As used herein the term “ligand” shall be taken in its broadest context to include any chemical compound, polynucleotide, peptide, protein, lipid, carbohydrate, small molecule, natural product, polymer, etc. that is capable of selectively binding, whether covalently or not, to one or more specific sites on a GSK-3β polypeptide. The ligand may bind to its target via any means including hydrophobic interactions, hydrogen bonding, electrostatic interactions, van der Waals interactions, pi stacking, covalent bonding, or magnetic interactions amongst others. It is preferred that a ligand is capable of specifically binding to a particular form of a GSK-3β polypeptide (e.g., a polypeptide encoded by an alternatively spliced GSK-3β mRNA).

As used herein the term “antibody” refers to intact monoclonal or polyclonal antibodies, immunoglobulin (IgA, IgD, IgG, IgM, IgE) fractions, humanized antibodies, or recombinant single chain antibodies, as well as fragments thereof, such as, for example Fab, F(ab)2, and Fv fragments.

Antibodies referred to herein are obtained from a commercial source, or alternatively, produced by conventional means. For example, a polyclonal anti-GSK-3β antibody is available from Genex Bioscience Inc., Hayward, Calif., USA.

High titer antibodies are preferred, as these are more useful commercially in kits for analytical, diagnostic and/or therapeutic applications. By “high titer” is meant a titer of at least about 1:103 or 1:104 or 1:105. Methods of determining the titer of an antibody will be apparent to the skilled artisan. For example, the titer of an antibody in purified antiserum may be determined using an ELISA assay to determine the amount of IgG in a sample. Typically an anti-IgG antibody or Protein G is used in such an assay. The amount detected in a sample is compared to a control sample of a known amount of purified and/or recombinant IgG. Alternatively, a kit for determining antibody may be used, e.g. the Easy TITER kit from Pierce (Rockford, Ill., USA).

Antibodies may be prepared by any of a variety of techniques known to those of ordinary skill in the art, and described, for example in, Harlow and Lane (In: Antibodies: A Laboratory Manual, Cold Spring Harbor Laboratory, 1988). In one such technique, an immunogen comprising the antigenic polypeptide is initially injected into any one of a wide variety of animals (e.g., mice, rats, rabbits, sheep, humans, dogs, pigs, chickens and goats). The immunogen is derived from a natural source, produced by recombinant expression means, or artificially generated, such as by chemical synthesis (e.g., BOC chemistry or FMOC chemistry). In this step, the polypeptides or fragments thereof of this invention may serve as the immunogen.

A peptide, polypeptide or protein is joined to a carrier protein, such as bovine serum albumin or keyhole limpet hemocyanin. The immunogen and optionally a carrier for the protein is injected into the animal host, preferably according to a predetermined schedule incorporating one or more booster immunizations, and blood collected from said the animals periodically. Optionally, the immunogen may be injected in the presence of an adjuvant, such as, for example Freund's complete or incomplete adjuvant, lysolecithin and dinitrophenol to enhance the immune response to the immunogen. Monoclonal or polyclonal antibodies specific for the polypeptide may then be purified from the blood isolated from an animal by, for example, affinity chromatography using the polypeptide coupled to a suitable solid support.

Monoclonal antibodies specific for the antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described supra. The spleen cells are immortalized by, for example, fusion with a myeloma cell fusion partner, preferably one that is syngenic with the immunized animal. A variety of fusion techniques may be employed, for example, the spleen cells and myeloma cells may be combined with a nonionic detergent or electrofused and then grown in a selective medium that supports the growth of hybrid cells, but not myeloma cells. A preferred selection technique uses HAT (hypoxanthine, aminopterin, and thymidine) selection. After a sufficient time, usually about 1 to 2 weeks, colonies of hybrids are observed. Single colonies are selected and growth media in which the cells have been grown is tested for the presence of binding activity against the polypeptide (immunogen). Hybridomas having high reactivity and specificity are preferred.

Monoclonal antibodies are isolated from the supernatants of growing hybridoma colonies using methods such as, for example, affinity purification as described supra. In addition, various techniques may be employed to enhance the yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction. The marker associated with neurodegeneration of this invention may be used in the purification process in, for example, an affinity chromatography step.

It is preferable that an immunogen used in the production of an antibody is one which is sufficiently antigenic to stimulate the production of antibodies that will bind to the immunogen and is preferably, a high titer antibody. In one embodiment, an immunogen may be an entire protein.

In another embodiment, an immunogen consists of a peptide representing a fragment of a polypeptide, for example a region of a GSK-3β polypeptide that is alternatively spliced. Preferably an antibody raised to such an immunogen also recognizes the full-length protein from which the immunogen was derived, such as, for example, in its native state or having native conformation.

Alternatively, or in addition, an antibody raised against a peptide immunogen will recognize the full-length protein from which the immunogen was derived when the protein is denatured. By “denatured” is meant that conformational epitopes of the protein are disrupted under conditions that retain linear B cell epitopes of the protein. As will be known to a skilled artisan linear epitopes and conformational epitopes may overlap.

Alternatively, a monoclonal antibody capable of binding to a form of a GSK-3β polypeptide of interest or a fragment thereof is produced using a method such as, for example, a human B-cell hybridoma technique (Kozbar et al., Immunol. Today 4:72, 1983), a EBV-hybridoma technique to produce human monoclonal antibodies (Cole et al. Monoclonal Antibodies in Cancer Therapy, 1985 Allen R. Bliss, Inc., pages 77-96), or screening of combinatorial antibody libraries (Huse et al., Science 246:1275, 1989).

Such an antibody is then particularly useful in detecting the presence of a marker of a disease or disorder associated with aberrant GSK-3β activity and/or expression.

Positive Read-Out Assay

In one embodiment, the method of the invention detects the presence of a marker in a polypeptide that is associated with a disease or disorder associated with aberrant GSK-3β expression and/or activity. Such a marker may comprise, for example, an amino acid change encoded by a polymorphism.

In another embodiment, the marker associated with a disease or disorder associated with aberrant GSK-3β activity and/or expression comprises consists of or is within a GSK-3β polypeptide that is encoded by an alternatively spliced GSK-3β transcript. For example, a GSK-3β polypeptide comprising the amino acid sequence selected from the group consisting of SEQ ID NO: 5, SEQ ID NO: 7 and SEQ ID NO: 9. Clearly, a positive read-out assay is useful for the detection of a GSK-3β polypeptide that includes additional amino acids compared to the native GSK-3β (e.g., a polypeptide comprising the amino acid sequence set forth in SEQ ID NO: 3). Such a GSK-3β polypeptide may be encoded, for example by a transcript that comprises an additional exon compared to native GSK-3β.

A positive read-out assay is also amenable to the detection of a GSK-3β that comprises fewer amino acids than a native GSK-3β polypeptide (e.g., a polypeptide encoded by a GSK-3β transcript that has spliced out an exon). For example, a ligand or antibody that is capable of specifically binding to the region of the GSK-3β polypeptide that include the amino acids encoded by nucleic acids on either side of the splice site only binds those forms of the polypeptide that lack the alternatively spliced exon.

Alternatively, a conformational specific ligand/antibody is useful for detecting a change in a GSK-3β polypeptide that causes a change in the conformation of the polypeptide. Such a ligand/antibody preferably only detects the GSK-3β polypeptide encoded by the alternatively spliced transcript.

The amount, level or presence of a polypeptide is determined using any of a variety of techniques known to the skilled artisan such as, for example, a technique selected from the group consisting of, immunohistochemistry, immunofluorescence, an immunoblot, a Western blot, a dot blot, an enzyme linked immunosorbent assay (ELISA), radioimmunoassay (RIA), enzyme immunoassay, fluorescence resonance energy transfer (FRET), matrix-assisted laser desorption/ionization time of flight (MALDI-TOF), electrospray ionization (ESI), mass spectrometry (including tandem mass spectrometry, e.g. LC MS/MS), biosensor technology, evanescent fiber-optics technology or protein chip technology.

In one embodiment the assay used to determine the amount or level of a protein is a semi-quantitative assay.

In another embodiment the assay used to determine the amount or level of a protein in a quantitative assay.

Immunoassays

Preferably, the amount of antibody or ligand bound to a marker of a disease or disorder within a GSK-3β polypeptide is determined using an immunoassay. Preferably, using an assay selected from the group consisting of, immunohistochemistry, immunofluorescence, enzyme linked immunosorbent assay (ELISA), fluorescence linked immunosorbent assay (FLISA) Western blotting, RIA, a biosensor assay, a protein chip assay, a mass spectrometry assay, a fluorescence resonance energy transfer assay and an immunostaining assay (e.g. immunofluorescence).

Standard solid-phase ELISA or FLISA formats are particularly useful in determining the concentration of a protein from a variety of samples.

In one form such an assay involves immobilizing a biological sample onto a solid matrix, such as, for example a polystyrene or polycarbonate microwell or dipstick, a membrane, or a glass support (e.g. a glass slide).

An antibody that specifically binds to a marker of a neurological disorder within a GSK-3β polypeptide is brought into direct contact with the immobilized biological sample, and forms a direct bond with any of its target protein present in said sample. This antibody is generally labeled with a detectable reporter molecule, such as for example, a fluorescent label (e.g. FITC or Texas Red) or a fluorescent semiconductor nanocrystal (as described in U.S. Pat. No. 6,306,610) in the case of a FLISA or an enzyme (e.g. horseradish peroxidase (HRP), alkaline phosphatase (AP) or β-galactosidase) in the case of an ELISA, or alternatively a second labeled antibody can be used that binds to the first antibody. Following washing to remove any unbound antibody the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal) in the case of an enzymatic label.

Such ELISA or FLISA based systems are particularly suitable for quantification of the amount of a protein in a sample, by calibrating the detection system against known amounts of a protein standard to which the antibody binds, such as for example, an isolated and/or recombinant GSK-3β polypeptide or immunogenic fragment thereof or epitope thereof.

In another form, an ELISA consists of immobilizing an antibody or ligand that specifically binds a marker of a disease or disorder within a GSK-3β polypeptide on a solid matrix, such as, for example, a membrane, a polystyrene or polycarbonate microwell, a polystyrene or polycarbonate dipstick or a glass support. A sample is then brought into physical relation with said antibody, and said marker within a GSK-3β polypeptide is bound or ‘captured’. The bound protein is then detected using a labeled antibody. For example, if the marker is captured from a human sample, a labeled anti-human GSK-3β antibody that binds to an epitope that is distinct from the first (capture) antibody is used to detect the captured protein. Alternatively, a third labeled antibody can be used that binds the second (detecting) antibody.

It will be apparent to the skilled person that the assay formats described herein are amenable to high throughput formats, such as, for example automation of screening processes or a microarray format as described in Mendoza et al., Biotechniques 27(4): 778-788, 1999. Furthermore, variations of the above-described assay will be apparent to those skilled in the art, such as, for example, a competitive ELISA.

Alternatively, the presence of a marker of a disease or disorder within a GSK-3β polypeptide is detected using a radioimmunoassay (RIA). The basic principle of the assay is the use of a radiolabeled antibody or antigen to detect antibody-antigen interactions. An antibody or ligand that specifically binds to the marker within a GSK-3β polypeptide is bound to a solid support and a sample brought into direct contact with said antibody. To detect the level of bound antigen, an isolated and/or recombinant form of the antigen is radiolabeled and brought into contact with the same antibody. Following washing, the level of bound radioactivity is detected. As any antigen in the biological sample inhibits binding of the radiolabeled antigen the level of radioactivity detected is inversely proportional to the level of antigen in the sample. Such an assay may be quantitated by using a standard curve using increasing known concentrations of the isolated antigen.

As will be apparent to the skilled artisan, such an assay may be modified to use any reporter molecule, such as, for example, an enzyme or a fluorescent molecule, in place of a radioactive label.

In another embodiment, Western blotting is used to determine the level of a marker of a neurological disorder within a GSK-3β polypeptide in a sample. In such an assay protein from a sample is separated using sodium doedecyl sulphate polyacrylamide gel electrophoresis (SDS-PAGE) using techniques known in the art and described in, for example, Scopes (In: Protein Purification: Principles and Practice, Third Edition, Springer Verlag, 1994). Separated proteins are then transferred to a solid support, such as, for example, a membrane (e.g., a PVDF membrane), using methods known in the art, for example, electrotransfer. This membrane is then blocked and probed with a labeled antibody or ligand that specifically binds to a marker of a disease or disorder within a GSK-3β polypeptide. Alternatively, a labeled secondary, or even tertiary, antibody or ligand is used to detect the binding of a specific primary antibody. The level of label is then determined using an assay appropriate for the label used. An appropriate assay will be apparent to the skilled artisan.

For example, the level or presence a marker of a disease or disorder within a GSK-3β polypeptide is determined using methods known in the art, such as, for example, densitometry. In one embodiment, the intensity of a protein band or spot is normalized against the total amount of protein loaded on a SDS-PAGE gel using methods known in the art. Alternatively, the level of the marker detected is normalized against the level of a control/reference protein. Such control proteins are known in the art, and include, for example, actin, glyceraldehyde 3-phosphate dehydrogenase (GAPDH), β2 microglobulin, hydroxy-methylbilane synthase, hypoxanthine phosphoribosyl-transferase 1 (HPRT), ribosomal protein L13c, succinate dehydrogenase complex subunit A and TATA box binding protein (TBP).

Biosensor devices generally employ an electrode surface in combination with current or impedance measuring elements to be integrated into a device in combination with the assay substrate (such as that described in U.S. Pat. No. 5,567,301). An antibody/ligand that specifically binds to a marker of a disease or disorder associated with aberrant GSK-3β activity and/or expression is preferably incorporated onto the surface of a biosensor device and a biological sample contacted to said device. A change in the detected current or impedance by the biosensor device indicates protein binding to said antibody. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several proteins or peptides in a small amount of body fluids.

Evanescent biosensors are also preferred as they do not require the pretreatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the target polypeptide to the antibody or ligand.

Micro- or nano-cantilever biosensors are also preferred as they do not require the use of a detectable label. A cantilever biosensor utilizes a ligand and/or antibody capable of specifically detecting the analyte of interest that is bound to the surface of a deflectable arm of a micro- or nano-cantilever. Upon binding of the analyte of interest (e.g. a marker within a GSK-3β polypeptide) the deflectable arm of the cantilever is deflected in a vertical direction (i.e. upwards or downwards). The change in the deflection of the deflectable arm is then detected by any of a variety of methods, such as, for example, atomic force microscopy, a change in oscillation of the deflectable arm or a change in pizoresistivity. Exemplary micro-cantilever sensors are described in USSN 20030010097.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example U.S. Patent Application No. 20020136821, 20020192654, 20020102617 and U.S. Pat. No. 6,391,625. To bind a protein to a solid support it is often necessary to treat the solid support so as to create chemically reactive groups on the surface, such as, for example, with an aldehyde-containing silane reagent. Alternatively, an antibody or ligand may be captured on a microfabricated polyacrylamide gel pad and accelerated into the gel using microelectrophoresis as described in, Arenkov et al. Anal. Biochem. 278:123-131, 2000.

A protein chip may comprise only one protein, ligand or antibody, and be used to screen one or more patient samples for the presence of one polypeptide of interest. Such a chip may also be used to simultaneously screen an array of patient samples for a polypeptide of interest.

Preferably, a protein sample to be analyzed using a protein chip is attached to a reporter molecule, such as, for example, a fluorescent molecule, a radioactive molecule, an enzyme, or an antibody that is detectable using methods known in the art. Accordingly, by contacting a protein chip with a labeled sample and subsequent washing to remove any unbound proteins the presence of a bound protein is detected using methods known in the art, such as, for example, using a DNA microarray reader.

Alternatively, biomolecular interaction analysis-mass spectrometry (BIA-MS) is used to rapidly detect and characterize a protein present in complex biological samples at the low- to sub-fmole level (Nelson et al. Electrophoresis 21: 1155-1163, 2000). One technique useful in the analysis of a protein chip is surface enhanced laser desorption/ionization-time of flight-mass spectrometry (SELDI-TOF-MS) technology to characterize a protein bound to the protein chip. Alternatively, the protein chip is analyzed using ESI as described in U.S. Patent Application 20020139751.

As will be apparent from the preceding discussion, it is particularly preferred to employ a detection system that is antibody or ligand based as such assays are amenable to the detection of a marker of a disease or disorder within a GSK-3β polypeptide. Immunoassay formats are even more particularly preferred.

Negative Read-Out Assays

In one embodiment, a marker of a disease or disorder associated with aberrant GSK-3β activity and/or expression within a GSK-3β polypeptide is detected by contacting a biological sample derived from a subject with an antibody or ligand capable of specifically binding to said marker for a time and under conditions sufficient for an antibody/ligand complex to form and then detecting the complex wherein lack of detection of the complex indicates that the subject being tested is predisposed to or suffers from the disease or disorder.

In one embodiment, the marker associated with a disease or disorder comprises, consists of or is within a GSK-3β polypeptide that is encoded by an alternatively spliced GSK-3β transcript. For example, such a GSK-3β polypeptide comprising the amino acid sequence set forth in SEQ ID NO: 5, SEQ ID NO: 7 or SEQ ID NO: 9. A negative read-out assay is useful for the detection of a GSK-3β polypeptide that has fewer amino acids compared to the native GSK-3β (e.g., a polypeptide comprising the amino acid sequence set forth in SEQ ID NO: 3). Such a GSK-3β polypeptide may be encoded, for example by a transcript that comprises has fewer exons (e.g. has an exon spliced out) compared to a native GSK-3β.

A negative read-out assay is also amenable to the detection of a GSK-3β polypeptide that comprises additional amino acids compared to a native GSK-3β polypeptide (e.g., a polypeptide encoded by a GSK-3β transcript that has spliced out an exon). For example, a ligand or antibody that is capable of specifically binding to the region of the GSK-3β polypeptide that flank the splice site only binds those forms of the polypeptide that are encoded by a GSK-3β transcript that includes an alternatively spliced exon.

In accordance with this embodiment of the invention such a negative read-out assay will not produce a detectable result or will detect a reduced level of detection of a marker associated with a disease or disorder in a GSK-3β polypeptide. For example, an assay that uses an antibody capable of specifically binding an epitope within exon 11 of a GSK-3β polypeptide will not detect a form of GSK-3β that is encoded by a transcript that lack exon 11. Accordingly, a negative result indicates that the subject suffers from a disease or disorder and/or is at risk of developing a disease or disorder.

As will be apparent to the skilled artisan a negative read-out assay for the detection of a polypeptide encoded by an alternative splice-form of a GSK-3β transcript may also include a positive control Such a control is useful as failure to detect the polypeptide may indicate that the detection reaction has failed. Such a positive control may involve, for example, using an antibody or ligand that binds to a region of GSK-3β that is not alternatively spliced, to determine the presence of GSK-3β encoding nucleic acid in a biological sample. Alternatively, a positive control may comprise using an antibody or ligand that binds to a polypeptide other than GSK-3β (that is known to be expressed in the biological sample) to determine the presence of polypeptide in a biological sample. Suitable proteins are described supra.

Suitable methods for detecting a polypeptide are known in the art and/or described supra.

### Detection of a Modified Level of a GSK-3β Transcript or a MAPT Transcript

The present inventors have also shown that polymorphisms in the GSK-3β gene are associated with a modified level of expression of a transcript of the GSK-3β gene in a subject suffering from a neurological disorder. The inventors have also shown that polymorphisms in the MAPT gene are associated with a modified level of expression of a transcript of the MAPT gene in a subject suffering from a neurological disorder

Accordingly, in one embodiment, a marker that is associated with a disease or disorder is detected by determining an enhanced or reduced level of a GSK-3β transcript and/or a MAPT transcript in a sample from a subject, wherein said enhanced or reduced level of the GSK-3β transcript and/or MAPT transcript is indicative of a neurological disorder.

In one embodiment, the GSK-3β transcript comprises the nucleotide sequence set forth in SEQ ID NO: 2. Alternatively, the GSK-3β transcript is an alternatively spliced GSK-3β transcript, for example, a nucleic acid comprising a nucleotide sequence at least 80% identical one or more nucleotide sequences selected from the group selected from SEQ ID NO: 4, SEQ ID NO: 6 and SEQ ID NO: 8.

In another embodiment, the MAPT transcript comprises a nucleotide sequence set forth in SEQ ID NO: 11.

Methods for detecting a transcript of a GSK-3β gene are described supra and are to be taken to apply mutatis mutandis to the detection of a modified level of a GSK-3β transcript and/or a modified level of a MAPT transcript. For example, the level of a GSK-3β transcript and/or a MAPT transcript is determined by performing a process comprising hybridizing a nucleic acid probe that selectively hybridizes to the GSK-3β transcript or MAPT transcript to nucleic acid in a sample derived from the subject under moderate to high stringency hybridization conditions and detecting the hybridization using a detection means, wherein the level of hybridization of the probe to the sample nucleic acid is indicative of the level of the GSK-3β transcript or MAPT transcript in the sample.

In one embodiment, an enhanced or reduced level of a transcript is detected by performing a process comprising:
(i) determining the level of the transcript in a sample derived from the subject;
(ii) comparing the level of the transcript at (i) to the level of the transcript in a suitable control sample,
   wherein an enhanced or reduced level of the GSK-3β transcript at (i) compared to (ii) is indicative of a neurological disorder and/or a predisposition thereto. A suitable control sample will be apparent to the skilled artisan and/or described herein.

Preferably, the level of a transcript comprising a nucleotide sequence set forth in SEQ ID NO: 8 is enhanced in a subject suffering from a neurodegenerative disorder or a myelin-associated disorder and/or the level of a transcript comprising a nucleotide sequence set forth in SEQ ID NO: 11 is reduced in a subject suffering from a neurodegenerative disorder or a myelin-associated disorder.

Preferably, the level of a transcript comprising a nucleotide sequence set forth in SEQ ID NO: 4 is enhanced in a subject suffering from a bipolar affective disorder and/or the level of a transcript comprising a nucleotide sequence set forth in SEQ ID NO: 11 is reduced in a subject suffering from a bipolar affective disorder.

Preferably, the level of a transcript comprising a nucleotide sequence set forth in SEQ ID NO: 4 is reduced in a subject suffering from schizophrenia and/or the level of a transcript comprising a nucleotide sequence set forth in SEQ ID NO: 11 is enhanced in a subject suffering from schizophrenia.

Detection of a Modified Level of a GSK-3β Polypeptide and/or a MAPT Polypeptide

In one embodiment, a marker associated with a neurological disorder is detected by determining an enhanced or reduced level of a GSK-3β polypeptide and/or a MAPT polypeptide in a sample from a subject, wherein said enhanced or reduced level of the GSK-3β polypeptide and/or MAPT polypeptide is indicative of a disease or disorder and/or a predisposition to a disease or disorder.

Preferably, the polypeptide is encoded by an alternatively spliced GSK-3β transcript, such as, for example, a GSK-3β transcript comprising the nucleotide sequence set forth in SEQ ID NO: 4, SEQ ID NO: 6 or SEQ ID NO: 8. Accordingly, in one embodiment, the method comprises detecting the level of a polypeptide comprising an amino acid sequence set forth in SEQ ID NO: 5, SEQ ID NO: 7 or SEQ ID NO: 9 or a polypeptide at least about 80% identical thereto.

Alternatively, the method comprises detecting the level of a polypeptide comprising an amino acid sequence set forth in SEQ ID NO: 12 or a polypeptide at least about 80% identical thereto in a sample from a subject.

Methods for determining the level of expression of a polypeptide are described supra and are to be taken to apply mutatis mutandis to the present aspect of the invention. For example, the level of the GSK-3β polypeptide and/or a MAPT polypeptide is detected by performing a process comprising contacting a biological sample derived from the subject with an antibody or ligand capable of selectively binding to the GSK-3β polypeptide or MAPT polypeptide for a time and under conditions sufficient for an antibody/ligand complex to form and then detecting the complex wherein the level of the complex is indicative of the level of the GSK-3β polypeptide or MAPT polypeptide in the subject.

Preferably, a method for detecting or determining an enhanced or reduced level of the GSK-3β polypeptide or MAPT polypeptide in a sample comprises performing a process comprising:
(i) detecting the level of the GSK-3β polypeptide or MAPT polypeptide in the sample;
(ii) comparing the level detected at (i) to the level of the GSK-3β polypeptide or MAPT polypeptide in a suitable control sample,

Preferably, the level of a polypeptide comprising a sequence set forth in SEQ ID NO: 9 is enhanced in a subject suffering from a neurodegenerative disorder or a myelin-associated disorder and/or the level of a polypeptide comprising a sequence set forth in SEQ ID NO: 12 is reduced in a subject suffering from a neurodegenerative disorder or a myelin-associated disorder.

Preferably, the level of a polypeptide comprising a sequence set forth in SEQ ID NO: 5 is enhanced in a subject suffering from a bipolar affective disorder and/or the level of a polypeptide comprising a sequence set forth in SEQ ID NO: 12 is reduced in a subject suffering from a bipolar affective disorder.

Preferably, the level of a polypeptide comprising a sequence set forth in SEQ ID NO: 5 is reduced in a subject suffering from schizophrenia and/or the level of a polypeptide comprising a sequence set forth in SEQ ID NO: 12 is enhanced in a subject suffering from schizophrenia.

Determining a Subject with a Reduced Risk of Developing a Disease or Disorder

As will be apparent to the skilled person, the present invention additionally provides a method for determining a subject that has a reduced risk of developing a neurological disorder. Such a method comprises detecting a marker that is associated with a reduced risk of developing the neurological disorder in a subject.

For example, a subject having a reduced risk of developing a neurodegenerative disease and/or a myelin-associated disorder comprises a marker within a GSK-3β gene associated with reduced expression of a GSK-3β transcript lacking exons 9 and 11 (e.g., comprising a sequence set forth in SEQ ID NO: 8) and a marker within a MAPT gene associated with increased expression of a MAPT expression product.

A subject having a reduced risk of developing a bipolar affective disorder comprises a marker within a GSK-3β gene associated with reduced expression of a GSK-3β transcript lacking exon 9 (e.g., comprising a sequence set forth in SEQ ID NO: 4) and a marker within a MAPT gene associated with increased expression of a MAPT expression product.

A subject having a reduced risk of developing schizophrenia comprises a marker within a GSK-3β gene associated with enhanced expression of a GSK-3β transcript lacking exon 9 (e.g., comprising a sequence set forth in SEQ ID NO: 4) and a marker within a MAPT gene associated with reduced expression of a MAPT expression product.

Suitable markers are described herein and are taken to apply mutatis mutandis to the present embodiment of the invention.

### Monitoring the Efficacy of Treatment

The methods described herein are also to be taken to apply mutatis mutandis to a method for monitoring the efficacy of treatment of a neurological disorder.

In one embodiment, the present invention provides a method for monitoring the efficacy of treatment of a subject undergoing treatment for a disease or disorder comprising:
(i) determining the level of expression of a GSK-3β expression product and a MAPT expression product in a sample from the subject; and
(ii) determining the level of expression of the GSK-3β expression product in a suitable control sample,
   wherein a similar level of expression of the GSK-3β expression product at (i) compared to (ii) indicates that the treatment is effective for the treatment of the disease or disorder.

### Biological Samples

As embodiments of the present invention are based upon detection of a marker in genomic DNA any cell or sample that comprises genomic DNA is useful for determining a disease or disorder and/or a predisposition to a disease or disorder. Preferably, the cell or sample is derived from a human. Preferably, comprises a nucleated cell.

Preferred biological samples include, for example, whole blood, serum, plasma, peripheral blood mononuclear cells (PBMC), a buffy coat fraction, saliva, urine, a buccal cell, urine, fecal material, sweat or a skin cell.

In a particularly preferred embodiment, a biological sample comprises a white blood cell, more preferably, a lymphocyte cell.

Alternatively, the biological sample is a cell isolated using a method selected from the group consisting of amniocentesis, chorionic villus sampling, fetal blood sampling (e.g. cordocentesis or percutaneous umbilical blood sampling) and other fetal tissue sampling (e.g. fetal skin biopsy). Such biological samples are useful for determining the predisposition of a developing embryo to a disease or disorder.

As will be apparent to the skilled artisan, the size of a biological sample will depend upon the detection means used. For example, an assay, such as, for example, PCR or single nucleotide primer extension may be performed on a sample comprising a single cell, although greater numbers of cells are preferred. Alternative forms of nucleic acid detection may require significantly more cells than a single cell. Furthermore, protein-based assays require sufficient cells to provide sufficient protein for an antigen based assay.

Preferably, the biological sample has been derived previously from the subject. Accordingly, in one embodiment, the method of the invention additionally comprises providing the biological sample.

In one embodiment, the method is performed using an extract from a biological sample, such as, for example, genomic DNA, mRNA, cDNA or protein.

As will be apparent from the preceding description, such an assay may require the use of a suitable control, e.g. a normal individual or a typical population, e.g., for quantification.

As used herein, the term “normal individual” shall be taken to mean that the subject is selected on the basis that they do not comprise or express a marker that comprises, consists of or is within a GSK-3β gene or expression product thereof and that is associated with a neurological disorder and/or do not comprise or express a marker that comprises, consists of or is within a MAPT gene or expression product thereof and that is associated with a neurological disorder. Nor does the subject suffer from the disease or disorder.

For example, the normal subject has not been diagnosed with any form of neurological disorder, using, for example, clinical analysis. For example, a subject may be tested for a neurological disorder using a neuropsychological test (e.g. a Wechsler Adult Intelligence Scale test, MDRS or GDS), an EEG, a CAT scan or a MRI scan.

Alternatively, or in addition, a suitable control sample is a control data set comprising measurements of the marker being assayed for a typical population of subjects known not to suffer from a neurological disorder Preferably the subject is not at risk of developing such a disease or disorder, and, in particular, the subject does not have a family history of the disease or disorder.

In the present context, the term “typical population” with respect to subjects known not to suffer from a disease or disorder and/or comprise or express a marker of a disease or disorder shall be taken to refer to a population or sample of subjects tested using, for example, known methods for determining the disease or disorder and determined not to suffer from the disease or disorder and/or tested to determine the presence or absence of a marker of the disease or disorder, wherein said subjects are representative of the spectrum of healthy subjects or subjects known not to suffer from the disease or disorder.

Given that many diseases or disorders are quantitative traits, a subject may suffer from the disease or disorder and not comprise or express a marker of the disease or disorder described herein. Alternatively, a subject may not suffer from the disease or disorder, yet comprise or express a marker of as described herein. However, a suitable control sample for the instant invention is a sample derived from a subject that does not suffer from the disease or disorder and does not comprise or express a marker of the disease or disorder (e.g., as described herein).

In one embodiment, a reference sample is not included in an assay. Instead, a suitable reference sample is derived from an established data set previously generated from a typical population. Data derived from processing, analyzing and/or assaying a test sample is then compared to data obtained for the sample population.

Data obtained from a sufficiently large number of reference samples so as to be representative of a population allows the generation of a data set for determining the average level of a particular parameter. Accordingly, the amount of a protein that is diagnostic of a disease or disorder or a predisposition to a disease or disorder can be determined for any population of individuals, and for any sample derived from said individual, for subsequent comparison to levels of the expression product determined for a sample being assayed. Where such normalized data sets are relied upon, internal controls are preferably included in each assay conducted to control for variation.

### Therapeutics of Neurological Disorders

As will be apparent from the disclosure herein, the present invention provides a method of treatment of a neurological disease comprising diagnosing a neurological disease in a subject or determining a susceptibility to a neurological disease in a subject using a method described herein according to any embodiment and administering or recommending a therapeutic treatment based on that diagnosis or determination.

Moreover, the present invention provides a method for determining a subject that will respond to a treatment with a compound used in the treatment of a neurological disease, said method comprising:
(i) detecting in a sample from the subject a marker within a glycogen synthase kinase-3β (GSK-3β) gene that is associated with a neurological disorder; and
(ii) detecting in a sample from the subject a marker within a microtubule-associated protein tau (MAPT) gene that is associated with a neurological disorder,
   wherein the marker detected at (i) and (ii) is indicative of a subject that will respond to treatment with a therapeutic compound.

For example, as will be apparent from the disclosure herein, particular combinations of markers in GSK-3β and MAPT are indicative of a specific neurological disorder, thereby providing for the means to predict a subject suffering from that specific neurological disorder and/or that will respond to treatment for that disorder.

Suitable therapeutic compounds will be apparent to the skilled artisan. For example, a compound suitable for the treatment of a bipolar affective disorder includes, for example, lithium or valproate. A suitable treatment for a neurodegenerative disease includes, for example, L-dopa, lithium or TDZD8. Therapeutics for the treatment of multiple sclerosis include, for example, interferon β-1A or interferon β-1B. Therapeutics for the treatment of schizophrenia include, for example, Aripiprazole, Clozapine, ziprasidone, respiradone, quetiapine or olanzapine.

The present invention is further described in the following non-limiting examples.

### EXAMPLE 1

### SNPs in GSK-3β that Affect Splicing of Transcripts Thereof

### 1.1 Samples

The Caucasian PD cohort used in this analysis comprises 302 cases (128 male and 174 female cases; average age, 66±0.5 years) and 302 control subjects (128 male and 174 female subjects; average age, 66±0.5 years). The cases were referral based and prospectively recruited from hospitals, private neurology clinics, and community support groups throughout the state of Queensland, Australia. PD was diagnosed according to standard criteria if the subject had a combination of three of the following features: resting tremor, rigidity, bradykinesia, and postural instability. The diagnosis was also made when at least two of these features were present with asymmetry in tremor, rigidity, or bradykinesia (Calne et al., Ann Neurol 32:S125-S127, 1992). The control group comprised nondemented healthy individuals who were spouses, siblings, caregivers, or unrelated individuals from various community groups. None of the control subjects was admitted to the hospital for other related illnesses at the time of collection, and a neurologist with a special interest in movement disorders examined both patients and control subjects.

The Hong Kong Chinese PD cohort used in these studies was recruited from outpatient departments from two major hospitals in Hong Kong (Prince of Wales and United Christian) and comprises 184 cases (96 male and 88 female cases; average age, 70±0.8 years) and 184 control subjects (96 male and 88 female subjects; average age, 70±0.7 years). All PD patients were assessed by a neurologist or geriatrician to confirm the diagnosis of PD. Inclusion criteria for PD were at least two of the following: resting tremor, bradykinesia, and rigidity in the absence of other causes of parkinsonism. A good response to L-dopa was used to confirm the diagnosis (Maranganore et al. Mov Disord., 6.205-211, 1991). The exclusion criteria were all other causes of parkinsonism. Control subjects were chosen carefully from outpatient clinics within the two Hong Kong Hospitals. The spectrum (and relative frequencies) of illness within the control group ranged from diabetes (15%), hypertension (27%), stroke (9%), ischemic heart disease (21%), musculoskeletal disorder (11%), gastrointestinal diseases (10%), neoplasm (2%), chronic obstructive airway disease (15%), and other medical diagnosis (28%). A proportion of subjects within the control group had more than one illness. However, none of the illnesses was a neurodegenerative disorder. In the selection process, 10-year age group, sex, and locality of residence were taken into consideration to make the control group as comparable with the PD group as possible. They were also examined by a neurologist or a geriatrician, and those with signs of PD or parkinsonism were excluded.

1.2 Single Nucleotide Polymorphism Detection within GSK3B and MAPT

All coding exons and flanking intronic sequences of GSK3β were amplified by polymerase chain reaction (PCR) from genomic DNA, and nucleotide variants were detected by direct sequencing of PCR products from 10 PD patients. The four polymorphisms detected in the present screen were subsequently identified in SNP database using the SNPper tool available from the Children's Hospital Informatics Program (Boston, Mass., USA) and amplified using the following primers:
(i) rs33455819 using GSKPromF TTTATAGACGCCCTCCCTTCGCTT (SEQ ID NO: 15) and GSKPromR TCCTTCCTTCCTTTGTCACTTGGC (SEQ ID NO: 16);
(ii) rs13312998 using TATAAAAGCTCTAAACACTCTCAA (SEQ ID NO: 17) and GSKEx3R AATGCTTTCCTGATATAACTAATG (SEQ ID NO: 18);
(iii) rs6438552 using GSKEx6F GCTTTTGGTGCCTTCTTAGGTGAC (SEQ ID NO: 19) and GSKEx6R CGAAACATTGGGTTCTCCTCG (SEQ ID NO: 20); and
(iv) rs7633279 using GSKEx9F GCCATGTCTGTGGATGATGAATC (SEQ ID NO: 21) and GSKEx9R CAATGAATCACCCAAGAGGCTG (SEQ ID NO: 22).

Each SNP was detected by direct sequencing of PCR products or restriction length polymorphism assay using the enzyme AluI (New England Biolabs, Beverley, Mass.) for rs334558 and Hpy188I (New England Biolabs) for rs6438552.

The Tau promoter haplotype was determined by PCR amplification using primers TauPromF ACTGAGTTAGCTTGCTTTAAG (SEQ ID NO: 23) and TauPromR TAGTTGGAGTCTTTGTGTCGTTGCGA (SEQ ID NO: 24) followed by MspI (New England Biolabs) restriction digest that detects the −373 G/C SNP within the haplotype.

### 1.3 Exon Trapping Assay

Each allele from the three GSK3β intronic polymorphisms identified was subcloned in the exon trap vector pSPL3 ((GIBCO BRL, Gaithersburg, Md.). For the exon 3 SNP, a 506 bp genomic fragment with 239 bp 5′ and 183 bp 3′ intronic sequences was used. For the exon 6 SNP, a 600 bp genomic fragment with 250 bp 5′ and 243 bp 3′ intronic sequences was used. For the exon 9 SNP, a 549 bp genomic fragment with 406 bp 5′ and 206 bp 3′ intronic sequences was used. Each recombinant vector was transfected into the human neuroblastoma cell line, SK-N-MC (ATCC HTB 10), and human embryonic kidney 293 cells (ATCC CRL 1573) using Lipofectamine 2000 (Invitrogen, La Jolla, Calif.). Cells were left for 48 hours before total RNA was extracted and the exon trap products were detected by reverse transcriptase PCR, essentially as described previously (Stanford et al., Brain, 123:880-893, 2000).

### 1.4 Results

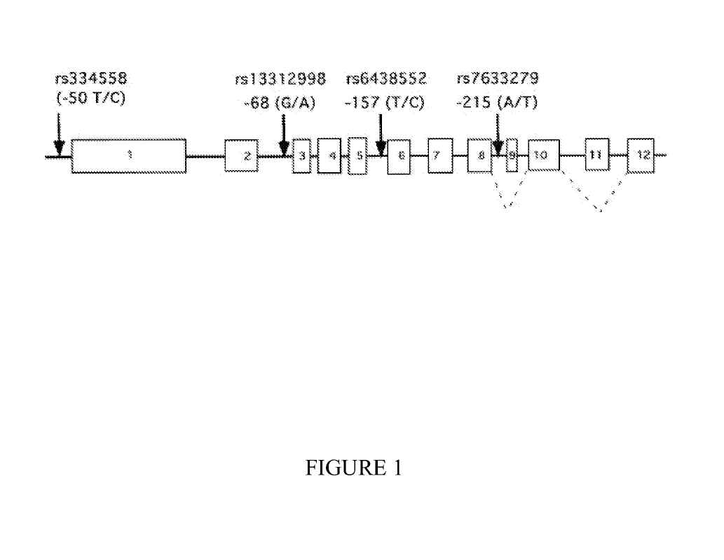

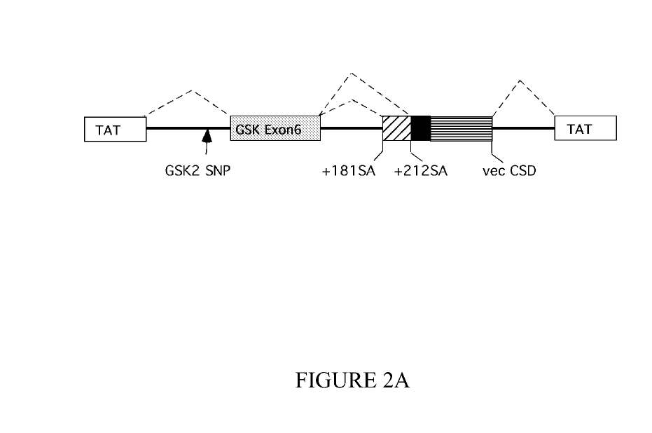

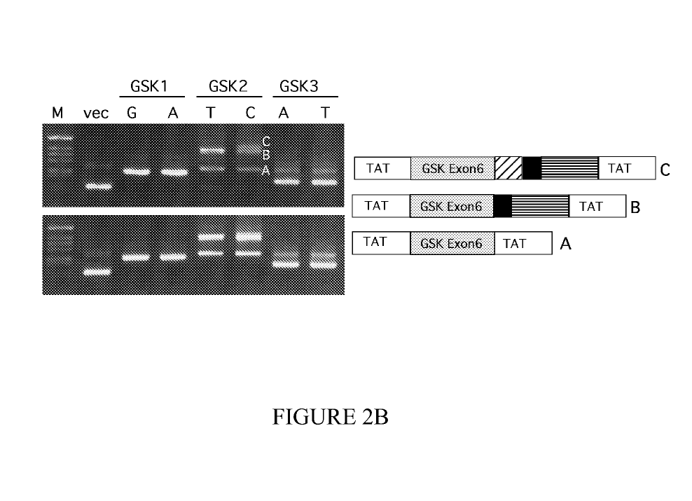

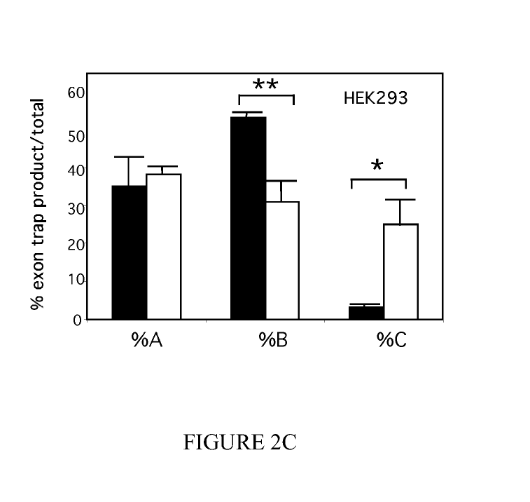

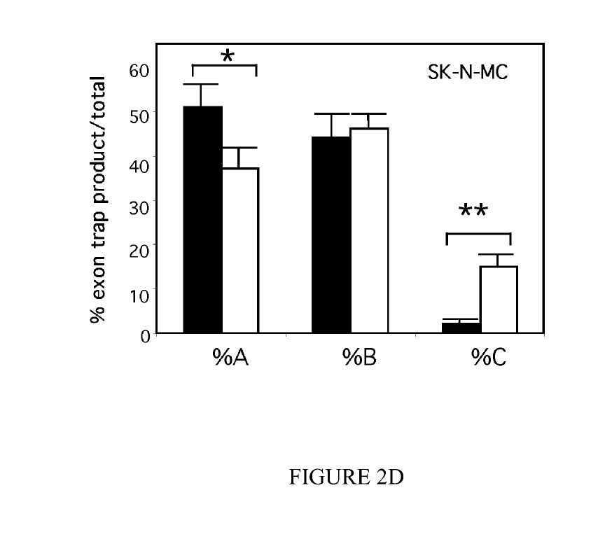

Three potential regulatory SNPs located within intronic sequences 2, 5, and 8 (IVS 2, acceptor −68 G/A=rs13312998; IVS 5, acceptor −157 T/C=rs6438552; and IVS 8, acceptor −215 A/T=rs7633279) and a promoter polymorphism (rs334558) were detected in a panel of 10 PD patients (FIG. 1A). The three intronic GSK3β SNPs were examined by exon trapping to determine whether they have an effect on splicing of their cognate exons in two human cell lines (FIG. 2A). In both HEK293 and SK-N-MC cell lines, rs6438552 resulted in altered ratios of exon trap products, indicating that the variant has an effect on splicing (see FIG. 2B). The exon trap products associated with rs6438552 were sequenced. These products correspond to differential use of three splice acceptor sites: the de novo exon 6 splice acceptor site and two cryptic splice acceptor sites in intron 6 (IVS 6+181 and IVS 6+212) (see FIG. 2B). In both cell lines, there was an eightfold decrease in utilization of the IVS 6+181 cryptic splice acceptor site (p<0.013 and 0.003 for HEK293 and SK-N-MC, respectively) for the T allele compared with the C allele (see FIG. 2C). The exon trap assays demonstrate that rs6438552 can modulate the utilization of splice acceptor sites in downstream introns.

We examined whether the promoter rs334558 and intronic rs6438552 SNPs were associated with idiopathic PD in two cohorts derived from two ethnic groups (Table 1). There was a consistent effect of the T/T genotypes of both polymorphisms as risk factors in the PD groups after stratification by MAPT haplotypes. In individuals with either the MAPT H1/H2 or H2/H2.haplotypes, the T/T genotypes of rs334558 and rs6438552 were increased in PD patients compared with control subjects (odds ratio=1.66 ([p<0.047] and 1.55 ([p<0.091], respectively). Conversely, in individuals who are homozygous for the AMPT H1 haplotype (H1/H1), there was a trend toward decreased frequency in the T/T genotypes of rs334558 and rs6438552 (odds ratio=0.69 ([p<0.082] and 0.59 ([p<0.065], respectively). A similar result was observed in the Chinese PD cohort (n=184 PD cases; n=184 control subjects; all were homozygous for the H1/H1 haplotype) with a trend toward decreased frequency in the T/T genotypes of rs334558 and rs6438552 (odds ratio=0.91 ([p<0.760] and 0.59 ([p<0.065], respectively). Similar trends were also seen in the smaller Caucasian brain samples cohort (see Table 1). These results indicate a gene-gene interaction between GSK3β and MAPT, with an opposing effect of GSK3β genotypes within MAPT haplotype subgroups. Conditional logistic regression was used to investigate this within the PD (Caucasian) cohort. Using a backward elimination strategy, it was found that the interaction model was the best model for both GSK3β genotypes. A significant interaction was found between AMPT and GSK3β rs334558 genotypes (p<0.009). After taking into account MAPT and GSK3β rs334558 genotypes and their interaction, the odds ratios for H1/H1 MAPT and T/T rs334558 genotypes were 1.70 (p<0.020) and 1.71 (p<0.044), respectively. When both the H1/H1 MAPT and T/T GSK3B genotypes were present, the negative effect of their interaction reduced the combined odds ratio to 1.19 (p<0.009). The relation between H1/H1 MAPT and GSK3B rs6438552 genotypes was similar. (p<0.074-0.097; see Table 1).

Because both rs334558 and rs6438552 have biological effects on transcription and splicing, respectively, haplotypes derived from both functional SNPs were studied to determine whether or not they would be more informative in the cohorts studied. The haplotype comprising the T allele of rs334558 and T allele of rs6438552 was studied, because this was the functional haplotype predicted to cause the greatest GSKΔexon9 and 11 isoform activity. The summary odds ratios for GSK3β T/T haplotype were 0.68 (p<0.001) in individuals with H1/H1 haplotype and 1.64 (p<0.007) in individuals with H1/H2 or H2/H2 haplotypes (Table 2).

TABLE 1
Association between Two GSK3B Polymorphisms in Parkinson Disease Cohorts

| | | | | | | | IVS 5 |
|---|---|---|---|---|---|---|---|
| | | | | | | | Acceptor - |
| | | | | | | | 157 T/C |
| | | | | | | | (rs6438552) |
| | | | | | | | T/T vs |
| | GSK3B Promoter -50 T/C | | | | | | T/C + |
| | (rs334558) | | | | | | C/C, Odds |
| Polymorphism Disease Cohort | T/T | | T/C | | C/C | | Ratio (p) |
| Parkinson (white) | | | | | | | |
| PD (n = 302) | 130 | (43%) | 134 | (44%) | 38 | (13%) | 0.99 |
| Control (n = 302)^{a} | 131 | (43%) | 134 | (44%) | 37 | (13%) | (0.935) |
| PD H1/H1 (n = 183) | 68 | (37%) | 84 | (46%) | 31 | (17%) | 0.69 |
| Control H1/H1 (n = 173) | 80 | (46%) | 70 | (41%) | 23 | (13%) | (0.082) |
| PD H1/H2/H2 (n = 119) | 62 | (52%) | 50 | (42%) | 7 | (6%) | 1.66 |
| Control H1/H2/H2 (n = 129) | 51 | (39%) | 64 | (50%) | 14 | (11%) | (0.047) |
| Parkinson (Chinese) | | | | | | | |
| PD All H1/H1 (n = 184) | 23 | (13%) | 81 | (44%) | 80 | (43%) | 0.91 |
| Control All H1/H1 (n = 184)^{a} | 25 | (13%) | 95 | (52%) | 64 | (35%) | (0.760) |
| Brain samples | | | | | | | |
| PD (n = 12) | 7 | (58%) | 4 | (34%) | 1 | (8%) | 2.33 |
| Control (n = 8) | 3 | (38%) | 4 | (50%) | 1 | (12%) | (0.361) |
| PD H1/H1 (n = 9) | 4 | (44%) | 4 | (44%) | 1 | (12%) | 0.80 |
| Control H1/H1 (n = 4) | 2 | (50%) | 1 | (25%) | 1 | (25%) | (0.850) |
| PD H1/H2/H2 (n = 3) | 3 | (100%) | 0 | (0%) | 0 | (0%) | Not tested^{b} |
| Control H1/H2/H2 (n = 4) | 1 | (25%) | 3 | (75%) | 0 | (0%) | (0.140) |
| | IVS 5 Acceptor -157 T/C (rs6438552) | | | | | | |
| | | | | | | | T/T vs |
| | | | | | | | T/C + |
| | | | | | | | C/C, Odds |
| Polymorphism Disease Cohort | T/T | | T/C | | C/C | | Ratio (p) |
| Parkinson (white) | | | | | | | |
| PD (n = 302) | 119 | (39%) | 133 | (44%) | 50 | (17%) | 1.07 |
| Control (n = 302)^{a} | 114 | (37%) | 141 | (47%) | 47 | (16%) | (0.676) |
| PD H1/H1 (n = 183) | 64 | (35%) | 80 | (44%) | 39 | (21%) | 0.83 |
| Control H1/H1 (n = 173) | 68 | (39%) | 79 | (46%) | 26 | (15%) | (0.400) |
| PD H1/H2/H2 (n = 119) | 55 | (46%) | 53 | (46%) | 11 | (9%) | 1.55 |
| Control H1/H2/H2 (n = 129) | 46 | (36%) | 62 | (48%) | 21 | (16%) | (0.091) |
| Parkinson (Chinese) | | | | | | | |
| PD All H1/H1 (n = 184) | 23 | (13%) | 81 | (44%) | 80 | (43%) | 0.59 |
| Control All H1/H1 (n = 184)^{a} | 36 | (20%) | 92 | (50%) | 56 | (30%) | (0.065) |
| Brain samples | | | | | | | |
| PD (n = 12) | 7 | (58%) | 5 | (42%) | 0 | (0%) | 2.33 |
| Control (n = 8) | 3 | (50%) | 2 | (33%) | 1 | (17%) | (0.361) |
| PD H1/H1 (n = 9) | 4 | (44%) | 5 | (56%) | 0 | (0%) | 0.80 |
| Control H1/H1 (n = 4) | 2 | (50%) | 1 | (25%) | 1 | (25%) | (0.850) |
| PD H1/H2/H2 (n = 3) | 3 | (100%) | 0 | (0%) | 0 | (0%) | Not tested^{b} |
| Control H1/H2/H2 (n = 4) | 1 | (25%) | 1 | (25%) | 2 | (50%) | (0.140) |

| Gene-gene interaction | | | | |
|---|---|---|---|---|
| for the parkinson (white) | | | | |
| cohort | Odds Ratio | p | Odds Ratio | p |
| H1/H1 MAPT haplotype | 1.70 | 0.020 | 1.47 | 0.074 |
| GSK3B polymorphism | 1.71 | 0.044 | 1.53 | 0.097 |
| Interaction term | 0.41 | 0.009 | 0.55 | 0.073 |

^{a}All control groups in Hardy-Weinberg equilibrium
^{b}Zero cells thus unable to calculate odds ratio. p value calculated using Fisher's exact test.

### EXAMPLE 2

### Linkage Disequilibrium of Markers in GSK-3β

### Linkage Disequilibrium Analysis

The Celera database (available from Celera Genomics Rockville, Rockville, Md., USA) and the UCSC Genome Bioinformatics Site (described in Kent et al., Genome Res. 12:996-1006, 2002) were used to identify genes adjacent to GSK-3β. Each flanking gene was examined for known SNPs using the CHUP Bioinformatics Tool (available from the Children's Hospital Informatics Program, Children's Hospital Boston, Boston, Mass., USA). For each gene, two verified SNPs were amplified by PCR and sequenced.

### Results

To determine the extent of linkage disequilibrium (LD) around GSK3β in Caucasian and Chinese populations, 24 individuals from each ethnic group were genotyped for the 5 GSK3β SNPs (rs3755557, rs334558, rs6438552, rs13312998, and 7633279), as well as validated SNPs from 4 flanking genes. Pairwise Lewontin's linkage disequilibrium (LD) coefficient (D′) were calculated (Devlin and Risch, Genomics; 29:311-322, 1995) from Expectation-Maximization algorithm-derived haplotype frequencies as implemented by the Haploview program (Barrett et al., Bioinformatics; 21:263-265, 2005) rs334558 and rs6438552 were in LD with each other (D′=0.9 in the Caucasian sample; D′=1.0 in the Chinese sample) to form a discernible haplotype in both ethnic groups. The flanking genes did not appear to be in LD with GSK3β, with most D′ values less than 0.2 (see FIG. 1C). Thus, rs334558 and rs6438552, located within GSK3β, fulfil a key criterion required for regulatory SNPs as predicted in the common disease-common variant hypothesis, namely, that the pathogenic polymorphism is ancient and LD around it is low (Weiss et al., Trends Genet; 18: 19-24, 2002).

### EXAMPLE 3

### Analysis of Splicing of GSK-3β in Patient Samples

### Analysis of GSK-3β Transcripts

Total RNA was extracted from lymphocyte cells derived from subjects homozygous for either the T or C allele at the SNP in intron 5 of GSK-3β. RNA was extracted using the SV Total RNA Isolation System (Promega). 2 μg of RNA was reverse transcribed using the Superscript II RT enzyme (GibcoBRL) and a random hexamer primer (GibcoBRL), followed by PCR amplification using the primers GSKRT-2F (5′-TGTTGGAGTTCCCAGGACCTTG-3′) (SEQ ID NO: 25) and GSKRT-R (5′-AGTAACTGGTGGTTTTTCCTGTGC-3′) (SEQ ID NO: 26).

The relative ratio of PCR products with or without exon 9 and exon 11 sequences was determined semi-quantitatively by PCR amplification essentially as described by Stanford et al., Brain, 123, 880-893, 2000) using 0.2 μg of cDNA template and ³³P end-labeled GSKRT-F primer.

### Western Blot Analysis of GSK-3β

Approximately 25 μg of protein lysates from lymphocytes were heated to 95° C. for 10 minutes prior to electrophoresis on a 7.5% SDS-PAGE gel and transfer to a nitrocellulose membrane (Trans-blot transfer medium, Biorad). A mouse monoclonal antibody raised against the amino terminus of GSK-3β (BD Bioscience, CA, USA) was used to detect GSK-3β protein.

### Results

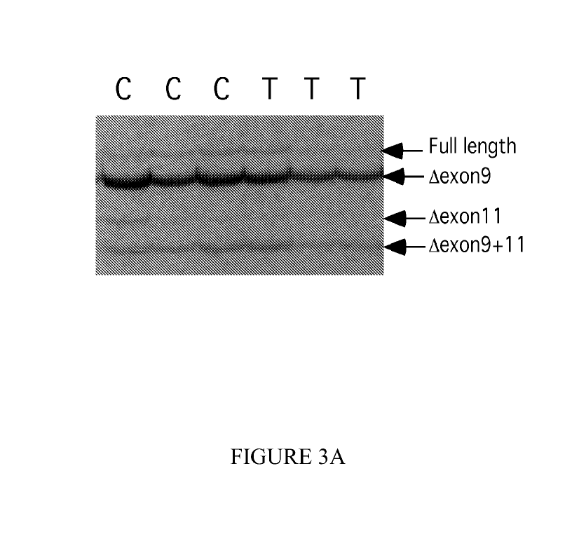

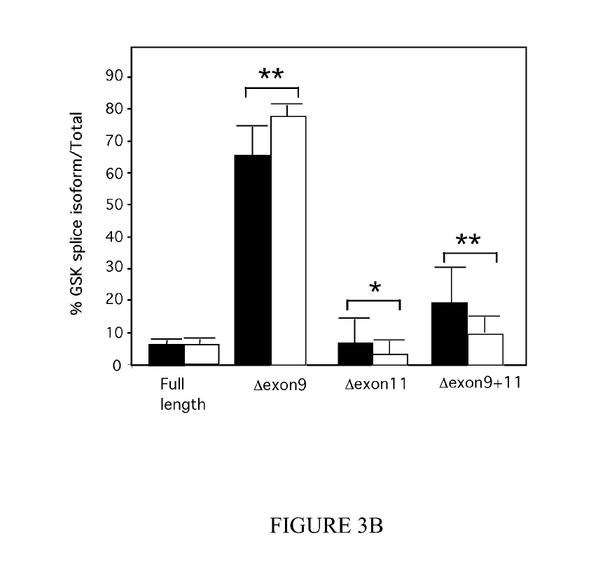

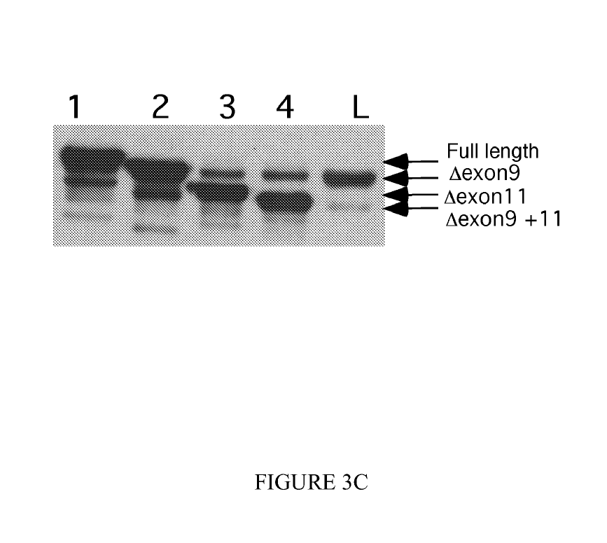

The effect of rs6438552 SNP was examined in a panel of lymphocyte cell lines (n=21) derived from individuals homozygous for either the T or C allele. The relative levels of all four GSK3β splice isoforms was also examined by reverse transcriptase PCR of total RNA using primers that spanned exons 6 to 12 of GSK3β. This amplification permitted the detection of the full-length GSK3β transcript (GSK.full) and transcripts lacking the alternatively spliced exons 9 (GSKΔexon9), 11 (GSKΔexon11), or both (GSKΔexon9 and 11) (FIG. 3A). When the levels of the GSK-3β transcripts were stratified according to genotype, there was a significant 1.8-fold increase in GSKΔexon11 (p=0.006, Student's t test) and 1.9-fold increase in GSKΔexon9+11 (p=0.003) when comparing individuals with T/T versus C/C genotypes (FIG. 3B).

When GSK-3β protein levels were examined by Western blotting, only two bands were detected in lymphocytes corresponding to the major splice isoforms GSKΔexon9 and GSKΔexon9 and 11 (see FIG. 3C).

### EXAMPLE 4

### Effect of Alternate Splicing on Tau Phosphorylation

### Construction of Expression Constructs

The four GSK-3β splice isoforms cDNAs amplified by PCR using lymphocyte cDNA (produced as described in Example 3) using the primer GSKRT-1F (5′-CGAGGGACACTAAATACAGTTCAA-3′) (SEQ ID NO: 27) and GSKRT-R (5′-AGTAACTGGTGGTTTTTCCTGTGC-3′) (SEQ ID NO: 28), and each product was subcloned into the mammalian expression vector pcDNA3.1 (Invitrogen).

COS-7 cells were then transfected with the gene constructs.

### Detection of Tau Species

Lymphocytes (as described in Example 3) or transfected cells were lysed in 1× Lysis buffer (50 mM Tris.HCl (pH 7.4), 150 mM NaCl, 1 mM PMSF, 1× complete cocktail protease inhibitor (Boehringer Mannheim) and 0.05% Triton X-100. Approximately 2-25 μg of total protein was used to assay for total Tau or Tau phosphorylated at serine residue 396 using the Human Tau or Human Tau [pS396] ELISA kit respectively (Biosource International, CA, USA).

### Results

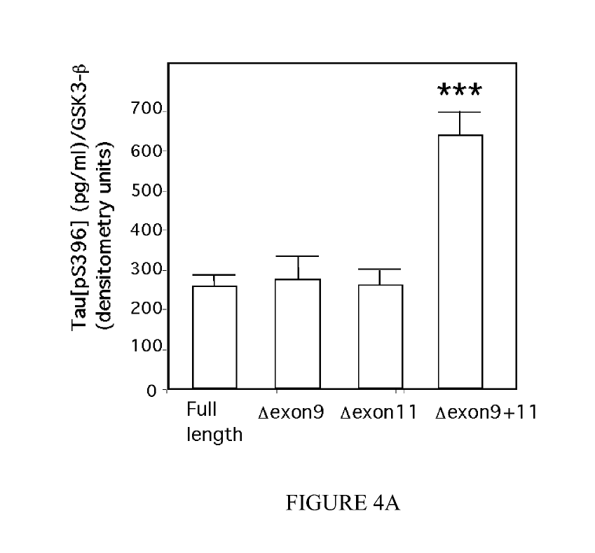

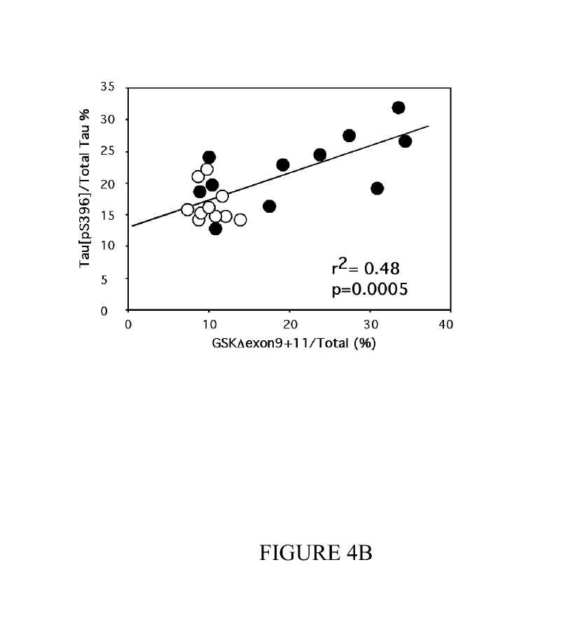

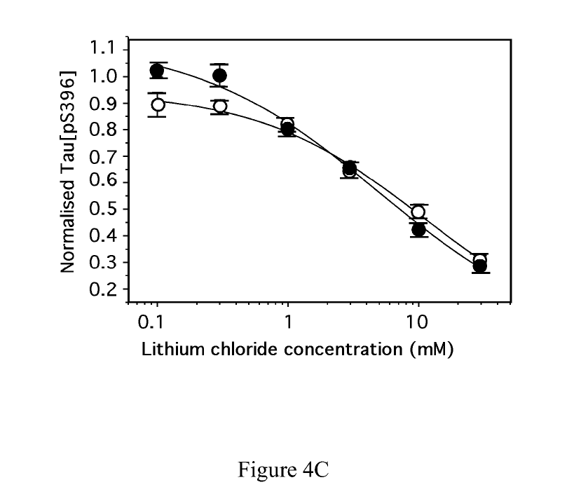

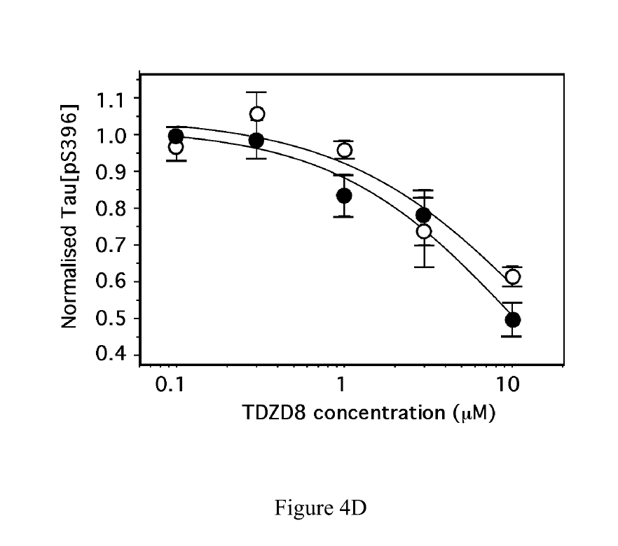

The ability of each GSK-3β splice isoform to phosphorylate Tau at a serine residue 396 (Tau [pSer396]) was examined. COS-7 cells were transfected with each cDNA and endogenous Tau phosphorylation was measured by ELISA (FIG. 4A). GSKΔexon9+11 showed the highest Tau phosphorylation, resulting in a 2.5-fold increase in levels of Tau [pSer396] compared with the other cDNAs (p<0.0001, ANOVA and post-hoc analysis). This increase in the ability of GSKΔexon9+11 to phosphorylate Tau explains the correlation (r²=0.48, p=0.0005) between the relative levels of endogenous GSKΔexon9+11 and Tau [pSer396] in lymphocyte cell lines (FIG. 4B). A 2.7-fold increase in Tau [pSer396] (p=0.021, Student's t test) protein was also observed in lymphocytes from individuals with the T/T genotype compared to the C/C genotype (data not shown).

Thus, the data suggests that the inheritance of the T allele of the GSK2 SNP (rs6438552) results in altered splicing to give rise to increased levels of GSK3Δexon9+11, which in turn leads to increased phosphorylation of Tau. The converse decrease in Tau phosphorylation is seen for the C allele.

### EXAMPLE 5

### Transcriptional Activity of the GSK-3β promoter is influenced by the rs334558 SNP

### Production of an Expression Construct Containing Luciferase Under Control of the GSK-3β Promoter

A 1510 bp DNA fragment comprising the promoter and transcription start site of GSK3β was amplified by PCR using primers comprising the sequences AAAAGATCCAAAGCTACCACGGC (SEQ ID NO: 29) and TCCTTCCTTCCTTTGTCACTTGGC (SEQ ID NO: 30). The amplification product was subcloned into the pGL3-Basic Luciferase vector (Promega). Each promoter haplotype was assayed for transcriptional efficiency. Each recombinant vector was transfected into the SK-N-MC (ATCC HTB 10) and 293 cells as described supra. Cells were lysed with 1× Lysis buffer (Promega) after 48 hours and assayed for luciferase activity using the Bright-Glo Luciferase assay system (Promega).

### 6.2 Results

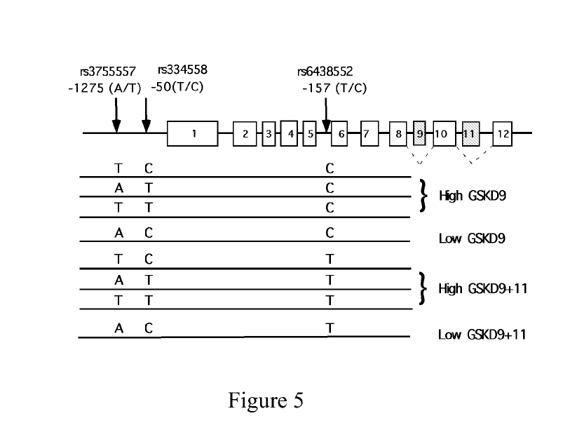

The promoter sequence of GSK3B was analyzed to determine possible binding sites of transcription factors using the MatInspector v2.2 software and the TRANSFAC 4.0 database (Quandt et al., Nucleic Acids Res. 23, 4878-48840, 1995). Using a high stringency of selection (maximal ‘Core similarity’ setting of 1 and ‘Matrix similarity’ of 0.85), a series of binding sites were detected for common transcription factors including AP-1 and SP-1 (FIG. 5A). The rs334558 SNP (located at a site corresponding to nucleotide position 232 of SEQ ID NO: 1) is located within a binding site for the AP4 transcription factor (FIG. 5A). The C allele was predicted by to abrogate binding of the transcription factor to the site.

The ability of each promoter haplotype to drive the expression of a luciferase reporter gene was examined. The T allele was associated with a significant 1.4 fold increase in transcriptional efficiency relative to the C allele in 293 (p=0.002, Student t test) and SK-N-MC (p=0.015, Student t test) respectively (FIGS. 5B and 5C, respectively).

### EXAMPLE 7

### Detection of Endogenous Tau Species

Lymphocytes or transfected cells were lysed in 1× Lysis buffer (50 mM tris[hydroxymethyl]aminomethane HCl ([pH 7.4], 150 mM NaCl, 1 mM phenylmethyl sulfonyl fluoride, 1× complete cocktail protease inhibitor ([Boehringer Mannheim, Mannheim, Germany], and 0.05% Triton X-100 [Sigma Labs, St. Louis, Mo.]). Soluble protein was extracted from brain tissue using the Trizol reagent (Invitrogen). Approximately 2 to 25 μg total protein was used to assay for total Tau or phosphoserine 396 Tau using the Human Tau or Human Tau [pS396] enzyme-linked immunosorbent assay kit, respectively (Biosource International, Camarillo, Calif.).

The ability of each GSK-3β isoform to phosphorylate endogenous Tau at a serine residue 396 (Tau [pSer396]) was examined by transient transfection and expression in COS-7 cells (FIG. 4A). GSKΔexon9 and 11 showed the highest Tau phosphorylation, resulting in a 2.5-fold increase in levels of Tau phosphorylation compared with the other isoforms (ρ0.001). The greater activity of GSKΔexon9 and 11 explains the 1.3-fold increase (ρ_{—}0.022) in Tau phosphorylation when comparing individuals with T/T versus C/C (rs6638552) genotypes (data not shown) and the positive correlation (Γ²=0.48; p<0.001) between relative levels of endogenous GSKΔexon9 and 11 and Tau phosphorylation in lymphocyte cell lines (see FIG. 4B). A 4.5-fold increase in Tau phosphorylation (p<0.039) was observed in a panel of PD (n=12) compared with control brains (n=8). A greater difference in Tau phosphorylation was found in the brain samples when the data were stratified by rs6438552 genotype rather than by disease state, which showed a 7.4-fold increase (p<0.074) in individuals with T/T versus C/C genotypes (data not shown). When brain samples with homozygous rs6438552 genotypes were used, as with the lymphocyte samples, the positive correlation was significant (Γ=0.31; p<0.048) (see FIG. 4C). Thus, these data suggest that the inheritance of the T allele of rs6438552 results in altered splicing in vivo to cause increased levels of GSKΔexon9 and 11, which, in turn, leads to increased phosphorylation of Tau.

### EXAMPLE 8

### GSK-3β and MAPT Haplotypes are Predictive of a Variety of Neurological Disorders

### Methods and Materials

### Subjects

(i) Parkinson Disease—The Caucasian PD cohort is described in Example 1.

(ii) Alzheimer's Disease—Clinical data and DNA samples from a UK case/control cohort were collected from 718 individuals (68% females, 32% males) with Alzheimer's disease and 718 control subjects (68% females, 32% males). Age of onset ranged from 55 to 95 years (mean=76 years, SD=7). Controls were matched for gender and ethnicity. The sample was recruited from both community and hospital settings and an assessment battery was used to form a diagnosis of probable AD according to NINCDS-ADRDA criteria. Controls were selected in two ways. Spouse controls were selected on the basis of the participation of their AD partner. Controls were also collected from general practices in the same areas as the AD patients. The Sydney Older Person Study (SOPS) cohort is a population-based cohort of community living elderly people (>75 years old) within a specific geographical region of Sydney. Probable AD and senile dementia status was determined using both DSM-III-R and NINCDS-ADRDA criteria.

(iii) Bipolar Disorder—Australian cohort individuals were almost entirely of British or Irish descent. Cases were recruited as part of an ongoing bipolar genetics study via the Mood Disorders Unit, Prince of Wales Hospital/School of Psychiatry, University of New South Wales. Thirty-six percent of cases were male, and 98% were older than 40 years of age. One RDC-defined bipolar I disorder case was selected from each of 65 bipolar pedigrees, previously recruited for linkage analyses. In addition, six cases were selected from a specialized bipolar disorder clinic sample, each of whom had no known family history of the disorder. All patients were assessed using the Diagnostic Interview for Genetic Studies (DIGS). The spouse of each case individual was selected as a control, each control individual being age and ethnically matched to the corresponding case. (Spouses were also all interviewed using the DIGS). A family history of psychiatric illness in first or second degree relative was present in 87% of cases. A lifetime psychotic episode was evident in 47% of cases.

(iv) The cohort of schizophrenic patients (n=68) and control individuals (n=77) are collected as part of an ongoing study by the Mental Health Research Institute of Victoria. The patients were diagnosed for chronic schizophrenia according to DSM-IV criteria by two senior psychiatrists and a psychologist following extensive case history review using the Diagnostic Instrument for Brain Studies (Hill et al., Am. J. Psychiatry 153: 533-537, 1996; Roberts et al., Aust. N. Z. J. Psychiatry 32: 73-76, 1998). Control individuals had no contact with any psychiatric service prior to death, had not received anti-psychotic medication, had not died by suicide or had any neurological disorders. The mean age of the control group was 46 years and comprise 74% males.

(v) Multiple Sclerosis (MS) Patient/Parent Trios—324 randomly selected MS patients and their unaffected parents (trios) were analyzed. Approximately 90% of patients and controls were of Northern European origin, while the remainder were largely of Southern European origin. Diagnosis was overseen by a neurologist; all patients had MS as defined by the Poser criteria. All patients provided written, informed consent, and were recruited by the Institute for Immunology and Allergy Research, Westmead Hospital (Australia).

### Genotyping of GSK3B and MAPT Single Nucleotide Polymorphisms.

The three polymorphisms were amplified using the following primers: rs3755557 using the primers GSKProm1F GCCGCCATCCTGATTGTAATCCAGTGG (SEQ ID NO: 31) and GSKProm1R GCTTACTTTGTTCTGTCCCAAGTCC (SEQ ID NO: 32); rs334558-F using GSKProm2F TTTATAGACGCCCTCCCTTCGCTT (SEQ ID NO: 33) and GSKProm2R TTCCTTCCTTCCTTTGTCACTTGGC (SEQ ID NO: 34); and rs6438552 using GSKEx6F GCTTTTGGTGCCTTCTTAGGTGAC (SEQ ID NO: 35) and GSKEx6R CGAAACATTGGGTTCTCCTCG (SEQ ID NO: 36). Each SNP was detected by restriction length fragment polymorphism using the following restriction enzymes: the enzyme Mse I (New England Biolabs, Beverly, Mass., USA) which cleaves the A allele of rs3755557; the enzyme Hpy188I (New England Biolabs) which cleaves the T allele of rs6438552 and the enzyme Alu I (New England Biolabs) which cleaves the C allele of rs334558. Primary Tau haplotype based on the promoter region was determined by examining the −373 G/T single nucleotide polymorphism (SNP) using primers ACTGAGTTAGCTTGCTTTAAGCCG (SEQ ID NO: 37) and TAGTTGGAGTCTTTGTGTCGTTGC (SEQ ID NO: 38) followed by restriction enzyme digest with MspI.

### Statistics

Chi square contingency tables were used to test for significance of association between the GSK3B SNPs and individual case/control cohorts. Haplotype frequencies were estimated from population genotype data using the Expectation-maximisation algorithm as implemented by the Arlequin program.

### Results

### Overall Summary of Interaction Between GSK3B and MAPT

From functional analysis of the promoter polymorphisms and the previously characterised intronic polymorphism (rs6438552), the following eight GSK3β haplotypes are defined into four functional groups that are predicted to relatively high or low levels of the two major splice isoforms, GSK3BΔ9 (lacking exon 9) and GSKΔ9+11 (lacking exon 9 and 11) as shown in FIG. 5. Altered levels of GSK3BΔ9 is associated with increased risk of psychiatric diseases (Psychosis and schizophrenia) and altered levels of GSKΔ9+11 is associated with increase risk of neurodegenerative diseases (Parkinson's Disease, Alzheimer's Disease and Multiple sclerosis).

In all the diseases examined thus far, the mode of interaction between GSK3β and MAPT is the same. There is an increase risk of disease in individuals who have GSK3β and MAPT haplotypes which result in discordant levels of the two proteins ie low levels of MAPT and high levels of GSK3B and vice versa.

Results for the Parkinson's Disease analysis are shown in Table 2. Results for each of the remaining disorders disorder are shown in Tables 3-7.

TABLE 3
Interaction between GSK3B and MAPT Polymorphisms in Alzheimer's
Disease Cohort (Sydney Older Person Study)

| | GSKB Haplotype Frequencies (rs334558-rs6438552) | | | | | Odds ratio T-T vs Others |
|---|---|---|---|---|---|---|
| Disease | T-T | | T-C | C-T | C-C | (p value) |
| All AD | 157 | (62%) | 13 (5%) | 3 (1%) | 81 (32%) | 1.27 |
| All Control | 188 | (56%) | 17 (5%) | 7 (2%) | 124 (37%) | (0.153) |
| AD H1 | 96 | (59%) | 7 (5%) | 2 (1%) | 57 (35%) | 1.05 |
| Control H1 | 128 | (58%) | 9 (4%) | 2 (1%) | 81 (37%) | (0.833) |
| AD H2 | 62 | (68%) | 5 (5%) | 0 (0%) | 25 (27%) | 1.93 |
| Control H2 | 60 | (52%) | 9 (8%) | 4 (3%) | 43 (37%) | (0.022) |

TABLE 4
Interaction between GSK3B and MAPT Polymorphisms
in Alzheimer's Disease Cohort 2 (UK COHORT)

| | GSKB Haplotype Frequencies (rs334558-rs6438552) | | | | Odds ratio T-T vs Others |
|---|---|---|---|---|---|
| Disease | T-T | T-C | C-T | C-C | (p value) |
| All AD | 871 (61%) | 73 (5%) | 24 (2%) | 468 (32%) | 1.02 |
| All Control | 861 (60%) | 100 (7%) | 27 (2%) | 448 (31%) | (0.703) |
| AD H1 | 500 (59%) | 46 (5%) | 15 (2%) | 289 (34%) | 0.86 |
| Control H1 | 515 (62%) | 56 (7%) | 12 (1%) | 243 (30%) | (0.139) |
| AD H2 | 371 (63%) | 27 (5%) | 9 (2%) | 179 (30%) | 1.32 |
| Control H2 | 346 (57%) | 44 (7%) | 15 (2%) | 205 (34%) | (0.020)* |

TABLE 5
Interaction between GSK3B and MAPT Haplotypes in psychosis subtype of Bipolar Disorder

| | | | | | Odds |
|---|---|---|---|---|---|
| Functional | Low | High | Low | High | Ratio * |
| Haplotypes | GSKΔexon9 | GSKΔexon9 | GSKΔexon9 + 11 | GSKΔexon9 + 11 | (p value) |
| Control | 65 (22%) | 56 (19%) | 12 (4%) | 163 (55%) | |
| (n = 296) | | | | | |
| BP | 64 (22%) | 56 (19%) | 13 (4%) | 163 (55%) | 1.02 |
| (n = 296) | | | | | (0.922) |
| BP-psychosis | 14 (18%) | 17 (21%) | 0 (0%) | 49 (61%) | 0.85 |
| (n = 80) # | | | | | (0.610) |
| Control (H1/H1) | 42 (24%) | 35 (20%) | 6 (3%) | 93 (53%) | |
| (n = 176) | | | | | |
| BP (H1/H1) | 38 (22%) | 30 (18%) | 8 (5%) | 92 (55%) | 0.78 |
| (n = 168) | | | | | (0.881) |
| BP-psychosis | 9 (20%) | 9 (20%) | 0 (0%) | 28 (60%) | 1.28 |
| (H1/H1) # | | | | | (0.561) |
| (n = 46) | | | | | |
| Control | 23 (19%) | 20 (17%) | 6 (5%) | 71 (59%) | |
| (H1/H1 + H2/H2) | | | | | |
| (n = 120) | | | | | |
| BP (H1/H2 + H2/H2) | 27 (21%) | 25 (20%) | 4 (3%) | 72 (56%) | 1.06 |
| (n = 128) | | | | | (0.851) |
| BP-psychosis | 3 (9%) | 10 (29%) | 2 (6%) | 19 (56%) | 2.47 |
| (H1/H2 + H2/H2) | | | | | (0.040) |
| (n = 34) # | | | | | |

# Odds ratio for psychosis subtype calculated between BP patients with and without pyschosis.
* Odds ratio calculated from pooled frequencies of High GSKΔexon9 and Low GSKΔexon9 + 11 haplotypes vs others.

TABLE 6
Interaction between GSK3β and MAPT Polymorphisms in Schizophrenia Cohort

| | | | | | Odds ratio |
|---|---|---|---|---|---|
| | GSKB Functional Haplotype Frequencies (rs3755557- rs334558 - rs6438552) | | | | High GSKΔexon9 |
| | High | Low | High | Low | vs Others |
| Disease | GSKΔexon9 | GSKΔexon9 | GSKΔexon9+11 | GSKΔexon9+11 | (p value) |
| All Scz | 16 (12%) | 34 (25%) | 84 (61%) | 1 (2%) | 0.56 |
| All Control | 30 (19%) | 30 (20%) | 89 (58%) | 5 (3%) | (0.077) |
| Scz H1 | 7 (10%) | 22 (27%) | 46 (59%) | 3 (3%) | 0.36 |
| Control H1 | 19 (21%) | 15 (17%) | 52 (58%) | 4 (4%) | (0.030)* |
| Scz H2 | 8 (14%) | 13 (21%) | 38 (63%) | 1 (2%) | 0.74 |
| Control H2 | 11 (17%) | 15 (23%) | 37 (58%) | 1 (2%) | (0.552) |

Note that in Schizophrenia, it is the haplotypes that result in low levels of GSK3Δexon9 is associated with disease risk. Accordingly, in schizophrenia the increase in disease risk is observed in individuals with H1 MAPT haplotype, which would result in even lower bioavailability of GSK3B.

TABLE 7
Multiple sclerosis patient-parental trio data

Tau

| | | H2H2 | Others |
|---|---|---|---|
| GSK3B | TT | 11 | 114 |
| (rs6438552) | Others | 5 | 194 |

### EXAMPLE 9

Without being bound by any theory or mode of action the genetic interaction studies described supra, suggest that the functional haplotypes that determine the level of Tau and GSK-3β proteins relative to one another is important in determining the disease risk associated with both genes. A corollary of this observation is that excess Tau should be able to sequester GSK-3β and shunt it towards cellular pools that will have functional consequences on other substrates of GSK-3β.

β-catenin is a member of the armadillo repeat family of proteins and exists as two functional reservoirs in the cell—the majority exists as a membrane-bound fraction that is associated with cadherin molecules and function in cell adhesion, and a smaller cytoplasmic pool that is able to translocate to the nucleus as transcription factors (Nelson and Nusse, Science 303: 1483-1487, 2004). The level of β-catenin is tightly controlled by GSK-3β phosphorylation and leads to the ubiquitination and degradation of the molecule. The present study aims to discover whether the transient over-expression of Tau in two human cell lines affects the overall level of β-catenin.

### Materials and Methods

Expression vectors containing cDNA clones corresponding to full-length Tau protein with (4 repeat Tau) or without exon 10 (3 repeat Tau) sequences were constructed essentially as described previously (Stanford et al., Brain 126: 814-826, 2003). Each recombinant vector was transfected into the human neuroblastoma cell line, SK-N-MC (ATCC HTB 10) and human embryonic kidney 293 cells (ATCC CRL 1573). Cells were plated onto 6 well plates at concentration of 2×10⁵ cells/well and allowed to recover for 24 hours. Each well was transfected with a control vector expressing the LacZ or Tau cDNAs using Lipofectamine 2000 (Invitrogen). After 48 hours, growth media were removed and cells were lysed in 1× Lysis buffer (50 mM Tris.HCl (pH 7.4), 150 mM NaCl, 1 mM, 1× complete cocktail protease inhibitor (Roche) and 0.05% Triton X-100, snap frozen at −80 degrees Celsius and microfuged at 13,000 rpm for 20 minutes.

Approximately 25 μg of protein lysates were heated to 95° C. for 10 minutes prior to electrophoresis on a 7.5% SDS-PAGE gel and transferred to a nitrocellulose membrane (Trans-blot transfer medium, Biorad). 1/2000 dilution of a mouse monoclonal antibody raised against the amino terminus of GSK-3β (BD Bioscience) was used to detect GSK-3β protein. A 1/2000 dilution of a mouse monoclonal antibody raised against recombinant β-catenin (BD Bioscience) was used to detect overall β-catenin levels in transfected samples. A 1/5000 dilution of a mouse monoclonal antibody raised against bovine microtubule associated protein (Neomarkers) was used to confirm the expression of the transfected tau proteins. Relative intensities of chemiluminescent bands on the blots were quantified directly using the Molecular Imager ChemiDoc XRS System (BioRad).

### Results

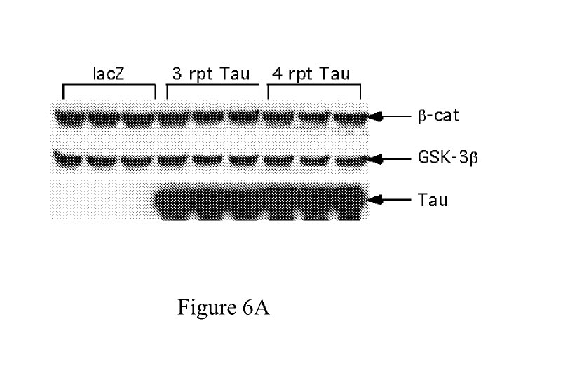

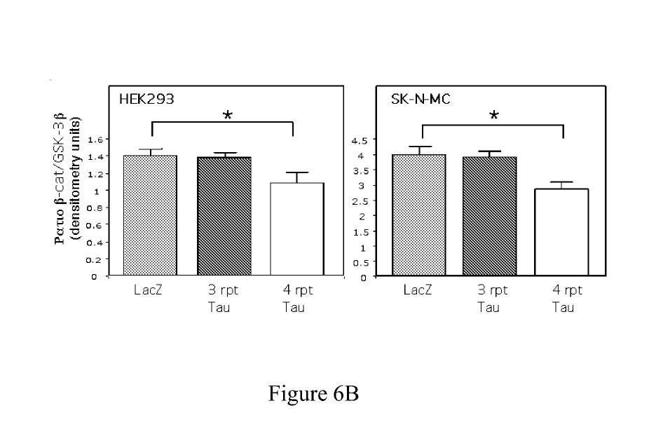

In both HEK293 and SK-N-MC cells, the transient over-expression of Tau led to the significant decrease in total β-catenin levels (FIG. 6). Of interest, the effect was only seen with the 4 repeat Tau isoform which resulted in a 1.3 (p=0.043, Student's t test) to 1.4 fold (p=0.008, Student's t test) decrease in β-catenin levels, suggesting that GSK-3β was able to phosphorylate and degrade more β-catenin in the presence of 4 repeat Tau. These results indicate that Tau may be able to sequester GSK-3β and modify its bioavailability for other substrates.

### EXAMPLE 10

### Determining a Subject Likely to Respond to Treatment

Samples from 38 patients suffering from a bipolar affective disorder are analyzed to determine the genotype present at the site of the rs3755557, rs334558 and rs6438552 SNPs and MAPT haplotype. Genotype is determined essentially as described in Example 9.

The medical records from these 38 bipolar patients are also examined to determine the effectiveness of treatment with lithium (ranked as highly responsive to lithium; variably responsive to lithium or poorly responsive to lithium).

An association between a particular genotype and likelihood to respond to treatment with lithium is then determined using standard methods.

### EXAMPLE 11

### GSK3B and MAPT Haplotypes Predict Side Effects of Lithium Treatment

Participants (n=84) suffering from a neurological disease who had been administered lithium as a therapeutic were assessed for the prevalence and pattern of lithium-induced side-effects using a self-administered questionnaire. This questionnaire was a modified version of the data collection form from a clinical trial, asking participants to rate 24 side-effect items as ‘Not present’, ‘Mild’, ‘Moderate’, ‘Severe’ and ‘Very Severe’. The 24 items listed include: nausea, vomiting, diarrhea, constipation, weight gain, weight loss, dizziness or light-headedness, blurred vision, hand tremor, muscle weakness, slurred speech, lack of coordination or unsteady gait, poor concentration mental slowing or confusion, forgetfulness or difficulty with memory/recall, drowsiness or sleepiness, worsening mood symptoms, skin rash, hair loss, thirst, dry mouth, metallic taste in mouth, loss of ability to taste food, need to pass urine much more frequently or more urgently, urinary incontinence. An overall side-effect score (SE score) was calculated for each patient based on the above 24 items and their respective grading. A score of zero was given for an absent side effect and increased to a score of 4 for a very severe side effect to give a maximum total score of 96 for each patient.

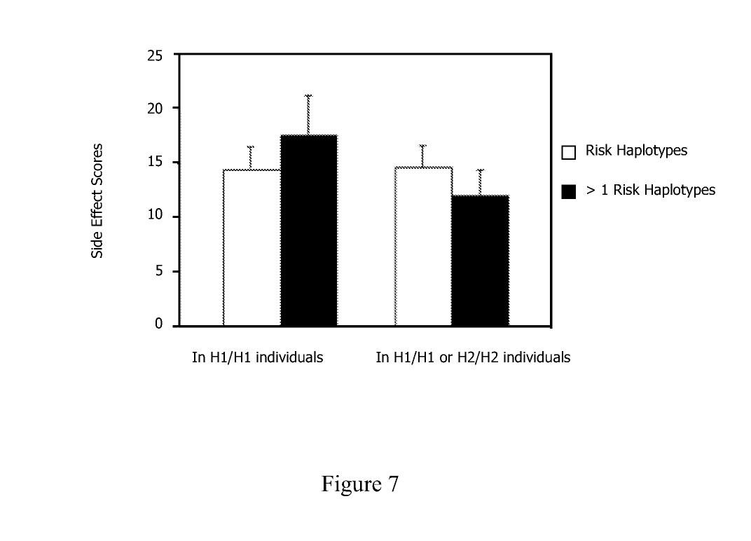

Each individual was genotyped and classified as having 0, 1 or 2 copies of the disease ‘risk’ GSK3B haplotypes (A-T-C, T-C-C and A-C-T) based on the three functional polymorphisms rs375557, rs334558 and rs6438552. Relationship between the number of copies of risk GSK3B haplotypes and mean SE score is shown in FIG. 7. In the subset of patients having a GSK-3B ‘risk’ haplotype and at least one H2 MAPT haplotype, a lower SE score was observed (12.3±2.2) compared to patients with no ‘risk’ haplotype (14.6±2.0).

The relationship between the ‘risk’ haplotypes in GSK3B and common side effect of lithium therapy, hand tremor, was then examined, the results of which are shown in Table 8. The GSK3B ‘risk’ haplotypes were significantly associated with a reduced risk of hand tremor (odds ratio 0.24, p=0.010) in individuals with at least one MAPT H2 haplotype. This information is pertinent, because lithium, which is a therapeutic agent for a number of disorders including bipolar affective disorders and Alzheimer's disease, has significant side effects. Accordingly, assays that detect the presence of a GSK-3B ‘risk’ haplotype optionally combined with detection of a MAPT H2 haplotype are useful for determining subjects likely to respond to treatment and/or that are unlikely to develop a side-effect as a result of treatment.

Without being bound by any theory or mode of action, the side effect data presented herein may result from discordant levels of GSK3B and MAPT. Lithium treatment effectively reduces the amount of GSK-3β. Individuals with the disease ‘risk’ haplotypes (A-T-C, T-C-C and A-C-T) have a higher level of GSK3BΔexon9 and thus have a reserve against the inhibitory effect of lithium. Moreover, in the subset of patients with the H2 haplotype, the lithium-induced reduction of GSK-3β would adjust the stoichiometry of Tau and GSK-3β to concordant levels. This may explain the paradoxical observation that the ‘risk’ haplotypes are protective against the lithium-induced side effects, and that the effect was stronger in patients with at least one H2 haplotype (FIG. 7, Table 8).

These results indicates that subjects with MAPT haplotypes (H1 and H1 sub haplotypes) that lead to high expression of MAPT and GSK3B haplotypes that lead to low expression of GSK3BΔexon9 will be least afflicted with lithium-induced side effects.

TABLE 8
Association of GSK3B Haplotypes and Side Effects of Lithium Treatment

| | | | | | Odds |
|---|---|---|---|---|---|
| | Low | High | Low | High | Ratio * |
| Functional Haplotypes | GSKΔexon9 | GSKΔexon9 | GSKΔexon9 + 11 | GSKΔexon9 + 11 | (p value) |
| Hand Tremor - Present | 20 (18%) | 17 (16%) | 0 (0%) | 73 (66%) | 0.57 |
| (n = 110) | | | | | (0.168) |
| Hand Tremor - Absent | 10 (17%) | 12 (21%) | 2 (4%) | 34 (58%) | |
| (n = 58) | | | | | |
| Hand Tremor - Present | 11 (20%) | 10 (19%) | 0 (0%) | 33 (61%) | 1.32 |
| (H1/H1) (n = 54) | | | | | (0.643) |
| Hand Tremor - Absent | 3 (20%) | 4 (16%) | 1 (6%) | 26 (58%) | |
| (H1/H1) (n = 34) | | | | | |
| Hand Tremor - Present | 9 (16%) | 7 (13%) | 0 (0%) | 40 (71%) | 0.24 |
| (H1/H2 + H2/H2)(n = 56) | | | | | (0.010) |
| Hand Tremor - Absent | 7 (29%) | 8 (34%) | 1 (4%) | 8 (33%) | |
| (H1/H2 + H2/H2)(n = 24) | | | | | |

* Odds ratio calculated from pooled frequencies of High GSKAexon9 and Low GSKΔexon9 + 11 haplotypes vs others

We claim:

1. A method for diagnosing a neurological disorder in a subject or determining the predisposition of a subject to developing a neurological disorder or determining the risk of a subject developing a neurological disorder, the method comprising:
(i) detecting in a sample from the subject a marker within a glycogen synthase kinase-3β (GSK-3β) gene or expression product thereof that is associated with a neurological disorder; and
(ii) detecting in a sample from the subject a marker within a microtubule-associated protein tau (MAPT) gene or expression product thereof that is associated with a neurological disorder,
wherein detection of the marker at (i) and (ii) is indicative of a neurological disorder or a predisposition to a neurological disorder or an increased risk of developing a neurological disorder.

2. The method according to claim 1 comprising:
(i) detecting in a sample from the subject a marker in a GSK-3β gene associated with modified expression of a GSK-3β expression product; and
(ii) detecting in a sample from the subject a marker in a MAPT gene associated with modified expression of a MAPT gene product,
wherein detection of the marker at (i) and (ii) is indicative of a subject that suffers from a neurological disorder or has a predisposition to a neurological disorder or has an increased risk of developing a neurological disorder and who will respond to treatment for said neurological disorder.

3. The method according to claim 4, wherein the marker within the GSK-3β gene is a single nucleotide polymorphism (SNP).

4. The method according to claim 3 wherein the SNP is selected from the group consisting of a thymidine at a position corresponding to nucleotide position 181,700 of SEQ ID NO: 1, a cytosine at a position corresponding to nucleotide position 181,700, a cytosine at a position corresponding to nucleotide position 231 of SEQ ID NO: 1, a thymidine at a position corresponding to nucleotide position 231 of SEQ ID NO: 1, an adenosine at a position corresponding to nucleotide position 1679 of SEQ ID NO: 10, a thymidine at a position corresponding to nucleotide position 1679 of SEQ ID NO: 10 and mixtures thereof.

5. The method according to claim 1 comprising:
(i) detecting in a GSK-3β gene a cytosine at a position corresponding to nucleotide position 181,700 of SEQ ID NO: 1, a thymidine at a position corresponding to nucleotide position 231 of SEQ ID NO: 1 and an adenosine at a position corresponding to nucleotide position 1679 of SEQ ID NO: 10; or
(ii) detecting in a GSK-3β gene a cytosine at a position corresponding to nucleotide position 181,700 of SEQ ID NO: 1, cytosine at a position corresponding to nucleotide position 231 of SEQ ID NO: 1 and a thymidine at a position corresponding to nucleotide position 1679 of SEQ ID NO: 10; or
(iii) detecting in a GSK-3β gene a thymidine at a position corresponding to nucleotide position 181,700 of SEQ ID NO: 1, a cytosine at a position corresponding to nucleotide position 231 of SEQ ID NO: 1 and an adenosine at a position corresponding to nucleotide position 1679 of SEQ ID NO: 10.

6. The method according to claim 3, wherein a marker within a MAPT gene is selected from the group consisting of:
(i) AATTT at a position equivalent to nucleotide position 763-767 of SEQ ID NO: 13;
(ii) TT at a position equivalent to nucleotide positions 793 and 794 of SEQ ID NO: 13;
(iii) an adenosine at a position equivalent to nucleotide position 817 of SEQ ID NO: 13;
(iv) a guanine at a position equivalent to nucleotide position 1011 of SEQ ID NO: 13;
(v) a thymidine at a position equivalent to nucleotide position 1036 of SEQ ID NO: 13;
(vi) a cytosine at a position equivalent to nucleotide position 1159 of SEQ ID NO: 13;
(vii) and adenosine at a position equivalent to nucleotide position 1340 of SEQ ID NO: 13; and
(viii) any combination of (i) to (viii).

7. The method according to claim 1 wherein the marker in a GSK-3β gene or expression product is detected by detecting a modified level of expression of an isoform of the GSK-3β gene or wherein the marker in a MAPT gene or expression product is detected by detecting a modified level of expression of a MAPT expression product.

8. The method according to claim 1 comprising detecting an enhanced level of a GSK-3β expression product comprising a sequence set forth in SEQ ID NO: 4 or SEQ ID NO: 5 or SEQ ID NO: 8 or SEQ ID NO: 9 and detecting reduced expression of a MAPT expression product.

9. The method according to claim 1, wherein a marker in a GSK-3β gene or expression product and/or a MAPT gene or expression product is detected by hybridizing a nucleic acid probe comprising the sequence of the marker to a marker linked to nucleic acid in a sample from a subject under moderate to high stringency hybridization conditions and detecting the hybridization using a detection means.

10. The method according to claim 1, wherein a marker is within a GSK-3β polypeptide or a MAPT polypeptide and the marker is detected by contacting a sample from the subject with an antibody or ligand capable of specifically binding to said marker for a time and under conditions sufficient for an antibody/ligand complex to form and then detecting the complex.

11. The method according to claim 1 for diagnosing a neurodegenerative disorder or multiple sclerosis or a bipolar affective disorder in a subject or determining the predisposition of a subject to developing a neurodegenerative disorder or multiple sclerosis or a bipolar affective disorder or determining the risk of a subject developing a neurodegenerative disorder or multiple sclerosis or a bipolar affective disorder, the method comprising:
(i) detecting in a sample from the subject a marker in a GSK-3β gene associated with increased expression of a GSK-3β expression product; and
(ii) detecting in a sample from the subject a marker in a MAPT gene associated with reduced expression of a MAPT gene product,
wherein detection of the marker at (i) and (ii) is indicative of a neurodegenerative disorder or multiple sclerosis or a bipolar affective disorder or a predisposition to a neurodegenerative disorder or multiple sclerosis or a bipolar affective disorder or an increased risk of developing a neurodegenerative disorder or multiple sclerosis or a bipolar affective disorder.

12. The method according to claim 11, for diagnosing a neurodegenerative disorder or multiple sclerosis or determining the predisposition of a subject to a neurodegenerative disorder or multiple sclerosis or determining the risk of a subject developing a neurodegenerative disorder or multiple sclerosis, wherein the marker in a GSK-3β gene is associated with increased expression of GSK-3β expression product comprising a sequence set forth in SEQ ID NO: 8 or 9.

13. The method according to claim 11 for diagnosing a bipolar affective disorder or determining the predisposition of a subject to a bipolar affective disorder or determining the risk of a subject developing a bipolar affective disorder, wherein the marker in a GSK-3β gene is associated with increased expression of GSK-3β expression product comprising a sequence set forth in SEQ ID NO: 4 or 5.

14. The method according to claim 1 for diagnosing schizophrenia in a subject or determining the predisposition of a subject to developing schizophrenia or determining the risk of a subject developing schizophrenia, the method comprising:
(i) detecting in a sample from the subject a marker in a GSK-3β gene associated with reduced expression of a GSK-3β expression product; and
(ii) detecting in a sample from the subject a marker in a MAPT gene associated with increased expression of a MAPT gene product,
wherein detection of the marker at (i) and (ii) is indicative of schizophrenia or a predisposition to schizophrenia or an increased risk of developing schizophrenia.

15. The method according to claim 14, wherein the marker in a GSK-3β gene is associated with reduced expression of a GSK-3β expression product comprising a sequence set forth in SEQ ID NO: 4 or 5.

16. A method for determining the likelihood that a subject will respond to treatment with a therapeutic compound, wherein said subject suffers from a neurological disorder or has a predisposition to a neurological disorder or has an increased risk of developing a neurological disorder, said method comprising:
(i) detecting in a sample from the subject a marker within a glycogen synthase kinase-3β (GSK-3β) gene that is associated with a response of a subject to treatment with a therapeutic compound; and
(ii) detecting in a sample from the subject a marker within a microtubule-associated protein tau (MAPT) gene that is associated with a response of a subject to treatment with a therapeutic compound,
and wherein detection of said markers indicates that the subject will respond to treatment with a therapeutic compound.

17. A method for determining a subject suffering from a neurological disorder or having a predisposition to a neurological disorder or having an increased risk of developing a neurological disorder who is likely to respond to a treatment with a therapeutic compound for a neurological disease, said method comprising:
(i) detecting in a sample from the subject a marker within a glycogen synthase kinase-3β (GSK-3β) gene that is associated with a neurological disorder; and
(ii) detecting in a sample from the subject a marker within a microtubule-associated protein tau (MAPT) gene that is associated with a neurological disorder,
wherein at least one of said markers is indicative of a subject that will respond to treatment with a therapeutic compound, and wherein detection of said markers is indicative of a subject that suffers from a neurological disorder or has a predisposition to a neurological disorder or has an increased risk of developing a neurological disorder and who will respond to treatment for said neurological disorder.

18. The method according to claim 16, wherein both markers are indicative of a subject who will respond to treatment with a therapeutic compound.

19. The method according to claim 16, wherein the neurological disorder is selected from the group consisting of a neurodegenerative disorder, a behavioral disorder and multiple sclerosis.

20. The method according to claim 16, wherein the neurological disorder is a bipolar affective disorder.

21. The method according to claim 16, wherein the therapeutic compound is lithium.

22. A method for identifying a subject suffering from a neurological disease or having a predisposition to a neurological disease or at risk of developing a neurological disease who will not experience a side effect resulting from treatment with a therapeutic compound, said method comprising:
(i) detecting in a sample from the subject a marker in a GSK-3β gene associated with modified expression of a GSK-3β expression product; and
(ii) detecting in a sample from the subject a marker in a MAPT gene associated with modified expression of a MAPT gene product,
wherein detection of said marker at (i) and (ii) indicates that the subject will not experience a side effect resulting from treatment with a therapeutic compound.

23. The method according to claim 22, wherein the side-effect is hand tremor.

24. The method according to claim 22, wherein the therapeutic compound is lithium.

25. A method of treatment or prophylaxis of a subject suffering from a neurological disorder or having a predisposition to a neurological disorder, said method comprising:
(i) performing a method described herein according to claim 1 to diagnose a neurological disorder in a subject or determine a predisposition of a subject to developing a neurological disorder; and
(ii) administering or recommending a therapeutic agent or prophylactic agent for the neurological disorder.
